(19) United States
(12) Patent Application Publication
(10) Pub. No.: US 2015/0183908 A1
(43) Pub. Date: Jul. 2, 2015

(54) COMPOSITIONS COMPRISING CROSSLINKED CATION-BINDING POLYMERS AND USES THEREOF

(71) Applicant: Relypsa, Inc., Redwood City, CA (US)

(72) Inventors: Linda De Young, Sunnyvale, CA (US); Stephen F. Carroll, Sunnyvale, CA (US)

(21) Appl. No.: 14/415,434

(22) PCT Filed: Jul. 19, 2013
(86) PCT No.: PCT/US13/51258

§ 371 Date: Jan. 16, 2015

Related U.S. Application Data
(60) Provisional application No. 61/673,722, filed on Jul. 19, 2012.

(51) Int. Cl. C08F 220/06; A61K 45/06; A61K 9/14; A61K 31/78

(52) U.S. Cl. CPC C08F 220/06; A61K 31/78; A61K 45/06; A61K 9/14

(57) ABSTRACT

The present disclosure relates generally to crosslinked cation-binding polymers comprising monomers containing carboxylic acid groups and pKa decreasing groups, wherein the polymer contains i) calcium cations that are counterions to about 5% to about 75% of the carboxylic acid groups in the polymer; or ii) calcium cations and magnesium cations that are counterions to about 5% to about 75% of the carboxylic acid groups in the polymer, wherein the magnesium cations are counterions to no more than about 35% of the carboxylate groups in the polymer. The present disclosure also relates to methods of preparation of the poly mers, and compositions, formulations, and dosage forms containing the polymers, and methods of using the polymers, compositions, formulations, and/or dosage forms to treat various diseases or disorders.

### CROSS-REFERENCE TO RELATED APPLICATIONS

This application claims the benefit of U.S. Provisional Application No. 61/673,722, filed on Jul. 19, 2012, which is incorporated by reference herein in its entirety.

### FIELD

The present disclosure relates generally to compositions comprising crosslinked cation-binding polymer comprising monomers containing carboxylic acid groups and pKa decreasing groups, wherein the polymer contains i) calcium cations that are counterions to about 5% to about 75% of the carboxylic acid groups in the polymer; or ii) calcium cations and magnesium cations that are counterions to about 5% to about 75% of the carboxylic acid groups in the polymer, wherein the magnesium cations are counterions to no more than about 35% of the carboxylate groups in the polymer. The present disclosure also relates to methods of preparation of said compositions and methods of using such compositions in dosage forms and to treat various diseases or disorders.

### BACKGROUND

Numerous diseases and disorders are associated with ion imbalances (e.g., hyperkalemia, hypernatremia, hypercalcemia, and hypermagnesia) and/or increased retention of fluid (e.g., heart failure and end stage renal disease (ESRD)). For example, patients afflicted with an increased level of potassium (e.g., hyperkalemia) may exhibit a variety of symptoms ranging from malaise, palpitations, muscle weakness and, in severe cases, cardiac arrhythmias. Patients afflicted with increased levels of sodium (e.g., hypernatremia) may exhibit a variety of symptoms including, lethargy, weakness, irritability, edema and in severe cases, seizures and coma. Patients afflicted with retention of fluid often suffer from edema (e.g., pulmonary edema, peripheral edema, edema of the legs, etc.) and the buildup of waste products in the blood (e.g., urea, creatinine, other nitrogenous waste products, and electrolytes or minerals such as sodium, phosphate and potassium).

Treatments for diseases or disorders associated with ion imbalances and/or an increased retention of fluid attempt to restore the ion balance and decrease the retention of fluid. For example, treatment of diseases or disorders associated with ion imbalances may employ the use of ion exchange resins to restore ion balance. Treatment of diseases or disorders associated with an increased retention of fluid may involve the use of diuretics (e.g., administration of diuretic agents and/or dialysis, such as hemodialysis or peritoneal dialysis and remediation of waste products that accumulate in the body). Additionally or alternatively, treatment for ion imbalances and/or increased retention of fluid may include restrictions on dietary consumption of electrolytes and water. However, the effectiveness and/or patient compliance with present treatments is less than desired.

### SUMMARY

The present disclosure is directed to compositions comprising crosslinked cation-binding polymers comprising monomers containing carboxylic acid groups and pKa decreasing groups.

The present disclosure is directed to crosslinked cation-binding polymers comprising monomers containing carboxyate groups and pKa decreasing groups, wherein the polymers further comprise calcium cations, wherein the calcium cations are counterions to about 5% to about 75% of the carboxylate groups in the polymer (alternatively, the polymers comprise calcium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations are optionally counterions to no more than about 5% (alternatively, no more than about 4%, about 3%, about 2%, or about 1%, about 0.5%, about 0.05%, or about 0.01%) of the carboxylate groups on the polymer. In some embodiments, the composition comprises a crosslinked cation-binding polymer comprising monomers wherein the pKa-decreasing group (e.g., the electron-withdrawing substituent) is located adjacent to the carboxylic acid group and preferably located in the alpha or beta position of the carboxylic acid group. In some embodiments, the composition comprises a crosslinked cation-binding polymer comprising monomers wherein the electron-withdrawing substituent is a hydroxyl group, an ethereal group, an ester group or a halide atom and most preferably fluorine (F). In some embodiments, the composition comprises a crosslinked cation-binding polymer derived from fluoroacrylic acid (or methyl-2-flouroacrylic acid) monomers or a mixture of such monomers with acrylic acid monomers or acrylic acid derivative monomers.

The present disclosure also relates generally to crosslinked cation-binding polymers comprising monomers containing carboxyate groups and pKa decreasing groups, wherein the polymers further comprise calcium cations and magnesium cations (e.g., a mixture of calcium cations and magnesium cations), wherein the calcium and magnesium cations are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations are optionally counterions to no more than about 5% (alternatively, no more than about 4%, about 3%, about 2%, or about 1%, about 0.5%, about 0.05%, or about 0.01%) of the carboxylate groups on the polymer. In some embodiments, the composition comprises a crosslinked cation-binding polymer comprising monomers wherein the pKa-decreasing group (e.g., the electron-withdrawing substituent) is located adjacent to the carboxylic acid group and preferably located in the alpha or beta position of the carboxylic acid group. In some embodiments, the composition comprises a crosslinked cation-binding polymer comprising monomers wherein the electron-withdrawing substituent is a hydroxyl group, an ethereal group, an ester group or a halide atom and most preferably F. In some embodiments, the composition comprises a crosslinked cation-binding polymer derived from fluoroacrylic acid (or methyl-2-flouroacrylic acid) monomers or a mixture of such monomers with acrylic acid monomers or acrylic acid derivative monomers.

The present disclosure also relates to methods of preparation of crosslinked cation-binding polymers comprising monomers that comprise carboxylate groups and pKa decreasing groups, for example, crosslinked polyacrylic acid, wherein the polymers further comprise calcium cations, wherein the calcium cations are counterions to about 5% to about 75% of the carboxylate groups in the polymer (alternatively, the polymers comprise calcium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein the polymers may optionally comprise sodium cations that are counterions to no more than about 5% (alternatively, no more than about 4%, about 3%, about 2%, or about 1%, about 0.5%, about 0.1%, or about 0.05%) of the carboxylate groups in the polymer. Any suitable carboxylic acid-containing monomer, or monomer which can be chemically converted to a carboxylic acid-containing monomer with a pKₐ-decreasing group such as an electron-withdrawing substituent (e.g., a halide such as F) known in the art may be used to prepare the compositions as disclosed herein, such as fluoroacrylic acid and methyl-2-fluoroacrylate or derivatives thereof. Acrylic acid or methacrylate monomers may be mixed with such monomers for co-polymerization.

The present disclosure also relates to methods of preparation of crosslinked cation-binding polymers comprising monomers that comprise carboxylate groups and pKa decreasing groups, for example, crosslinked polyacrylic acid, wherein the polymers further comprise calcium cations cations and magnesium cations (e.g., a mixture of calcium and magnesium cations), wherein the calcium and magnesium cations are counterions to about 5% to about 75% of the carboxylate groups in the polymer (alternatively, the polymers comprise calcium and magnesium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than 35% of the carboxylate groups in the polymer, and wherein the polymers may optionally comprise sodium cations that are counterions to no more than about 5% (alternatively, no more than about 4%, about 3%, about 2%, or about 0.1%, about 0.5%, about 0.1%, or about 0.05%) of the carboxylate groups in the polymer. Any suitable carboxylic acid-containing monomer or monomer which can be chemically converted to a carboxylic acid-containing monomer with a pKₐ-decreasing group such as an electron-withdrawing substituent (e.g., a halide such as F) known in the art may be used to prepare the compositions as disclosed herein, such as fluoroacrylic acid and methyl-2-fluoroacrylate or derivatives thereof. Acrylic acid and methacrylate monomers may be mixed with such monomers for co-polymerization.

In some embodiments, the crosslinked cation-binding polymer is a crosslinked polymer comprising monomers containing carboxylic acid groups and pKa-decreasing groups such as electron-withdrawing substituents (e.g., a halide atom such as F). For example, the polymer (e.g., polyfluoroacrylic acid or polymethyl-fluoroacrylate polymer) may be crosslinked with about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker, and for example, may comprise an in vitro saline absorption capacity of at least about 20 times its weight (e.g., at least about 20 grams of saline per gram of polymer, or “g/g”), at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the crosslinked polymer (e.g., polyfluoroacrylic acid or polymethyl-fluoroacrylate polymer) is in the form of individual particles (e.g., beads) or particles that are agglomerated (for example, flocculated) to form a larger particle, wherein the diameter of individual particles or agglomerated particles is about 1 micron to about 10,000 microns, such as, for example, about 212 microns to about 500 microns, about 75 microns to about 150 microns (e.g., about 100 microns) or about 75 microns or less (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns). In one embodiment, the polymer is in the form of small particles that flocculate to form agglomerated particles with a diameter of about 1 micron to about 10 microns.

The present disclosure also relates to dosage forms (e.g., oral dosage forms) comprising one or more of the compositions disclosed herein.

The present disclosure also relates to methods of using such compositions, to treat various diseases or disorders. In some embodiments, the disease is heart failure. In some embodiments, the disease is heart failure with chronic kidney disease. In some embodiments, the disease is end stage renal disease. In some embodiments, the disease is end stage renal disease with heart failure. In some embodiments, the disease is chronic kidney disease. In some embodiments, the disease is hypertension. In some embodiments, the disease is salt-sensitive hypertension. In some embodiments, the disease is refractory hypertension. In some embodiments, the disease involves an ion imbalance such as hyperkalemia, hypernatremia, hypercalcemia, etc. In some embodiments, the disease or disorder involves a fluid maldistribution or fluid overload state such as edema or ascites.

In some embodiments, the disease or disorder is the result of, or is associated with, administration of another agent (e.g., drug). For example, compositions according to the present disclosure are useful in treating an increase in a subject's potassium level when co-administered with an agent (e.g., drug) known to cause increases in potassium levels, such as an alpha-adrenergic agonist, a RAAS inhibitor, an ACE inhibitor, an angiotensin II receptor blocker, a beta blocker, an aldosterone antagonist, etc. For example, compositions according to the present disclosure are useful in treating an increase in a subject's sodium level when co-administered with an agent (e.g., drug) known to cause increases in sodium levels, such as an anabolic steroid, a birth control pill, an antibiotic, clonidine, a corticosteroid, a laxative, lithium, a nonsteroidal anti-inflammatory drug (NSAID), etc.

These and other embodiments will be described more fully by the detailed description and examples that follow.

### DETAILED DESCRIPTION

The present disclosure relates generally to crosslinked cation-binding polymers comprising monomers that comprise carboxylate groups, e.g., crosslinked polyacrylic acid, and pKa decreasing groups such as an electron-withdrawing substituents, and compositions, formulations, and/or dosage forms that contain the polymers, wherein the polymers further comprise calcium cations, wherein the calcium cations are counterions to about 5% to about 75% of the carboxylate groups in the polymer (e.g., the polymer comprises an amount of calcium cations sufficient to provide calcium counterions to about 5% to about 75% of the carboxylate groups in the polymer). Alternatively, the polymer comprises calcium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer. In some embodiments, the polymer comprises calcium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In some embodiments, the polymer is crosslinked with about 0.025% mol % to about 3% of crosslinker. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium cations as counterions to about 5% to about 75% of the carboxylate groups on the polymer, sodium cations as counterions up to about 5% of the carboxylate groups on the polymer, and hydrogen cations (e.g., protons) as counterions to about 20% to about 90% of the carboxylate groups on the polymer. In some embodiments, the polymer comprises calcium cations as counterions to about 5% to about 75% of the carboxylate groups on the polymer and hydrogen cations (e.g., protons) are counterions to the remainder or substantially the remainder of the carboxylate groups on the polymer (e.g., counterions that are not calcium or sodium are hydrogen). It is understood by those of skill in the art that these hydrogen cations are essentially hydrogen and may include small amounts (e.g., less than about 10,000 ppm of non-hydrogen elements), such as iron, copper, aluminum, arsenic, mercury, manganese, phosphorous, lead, selenium, titanium, and/or zinc.

The present disclosure also relates generally to crosslinked cation-binding polymers comprising monomers that comprise carboxylate groups, e.g., crosslinked polyacrylic acid, and pKa decreasing groups, and compositions, formulations, and/or dosage forms that contain the polymers, wherein the polymers further comprise calcium cations and magnesium cations (e.g., a mixture of calcium and magnesium cations), wherein the calcium and magnesium cations are counterions to about 5% to about 75% of the carboxylate groups in the polymer (e.g., the polymer comprises an amount of calcium and magnesium cations sufficient to provide calcium and magnesium counterions to about 5% to about 75% of the carboxylate groups in the polymer), and wherein the magnesium cations are counterions to no more than about 35% of the carboxylate groups in the polymer. Alternatively, the polymer comprises calcium and magnesium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer. In some embodiments, the polymer comprises calcium and magnesium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In some embodiments, the polymer is crosslinked with about 0.025% mol % to about 3% of crosslinker. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium cations and magnesium cations as counterions to about 5% to about 75% of the carboxylate groups on the polymer, sodium cations as counterions up to about 5% of the carboxylate groups on the polymer, and hydrogen cations (e.g., protons) as counterions to about 20% to about 90% of the carboxylate groups on the polymer, wherein magnesium cations are counterions to no more than about 35% of the carboxylate groups on the polymer. In some embodiments, the polymer comprises calcium cations and magnesium cations as counterions to about 5% to about 75% of the carboxylate groups on the polymer, and hydrogen cations (e.g., protons) are counterions to the remainder or substantially the remainder of the carboxylate groups on the polymer (e.g., counterions that are not calcium or sodium are hydrogen), wherein magnesium cations are counterions to no more than about 35% of the carboxylate groups on the polymer. It is understood by those of skill in the art that these hydrogen cations are essentially hydrogen and may include small amounts (e.g., less than about 10,000 ppm of non-hydrogen elements), such as iron, copper, aluminum, arsenic, mercury, manganese, phosphorous, lead, selenium, titanium, and/or zinc.

In some embodiments, crosslinked cation-binding polymers are provided that comprise monomers that comprise carboxylate groups and pKa decreasing groups, e.g., crosslinked polyacrylic acid, and compositions, formulations, and/or dosage forms that contain the polymers, wherein the polymers further comprise calcium cations, wherein the calcium cations are counterions to about 5% to about 75% of the carboxylate groups in the polymer (e.g., the polymer comprises an amount of calcium cations sufficient to provide calcium counterions to about 5% to about 75% of the carboxylate groups in the polymer). Alternatively, the polymer comprises calcium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer. In some embodiments, the polymer comprises calcium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In some embodiments, the polymer is crosslinked with about 0.025 mol % to about 3.0 mol % of the crosslinker. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium cations as counterions to about 5% to about 75% of the carboxylate groups on the polymer, sodium cations as counterions up to about 5% of the carboxylate groups on the polymer, and hydrogen cations (e.g., protons) as counterions to about 20% to about 90% of the carboxylate groups on the polymer. In some embodiments, the polymer comprises calcium cations as counterions to about 5% to about 75% of the carboxylate groups on the polymer and hydrogen cations (e.g., protons) are counterions to the remainder or substantially the remainder of the carboxylate groups on the polymer (e.g., counterions that are not calcium or sodium are hydrogen). It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, crosslinked cation-binding polymers are provided that comprise monomers that comprise carboxylate groups and pKa decreasing groups, e.g., crosslinked polyacrylic acid, and compositions, formulations, and/or dosage forms that contain the polymers, wherein the polymers further comprise calcium cations and magnesium cations (e.g., a mixture of calcium and magnesium cations), wherein the calcium and magnesium cations are counterions to about 5% to about 75% of the carboxylate groups in the polymer (e.g., the polymer comprises an amount of calcium and magnesium cations sufficient to provide calcium and magnesium counterions to about 5% to about 75% of the carboxylate groups in the polymer), and wherein the magnesium cations are counterions to no more than about 35% of the carboxylate groups in the polymer. Alternatively, the polymer comprises calcium and magnesium cations that are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer. In some embodiments, the polymer comprises calcium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In some embodiments, the polymer is crosslinked with about 0.025 mol % to about 3.0 mol % of the crosslinker. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium and magnesium cations as counterions to about 5% to about 75% of the carboxylate groups on the polymer, sodium cations as counterions up to about 5% of the carboxylate groups on the polymer, and hydrogen cations (e.g., protons) as counterions to about 20% to about 90% of the carboxylate groups on the polymer, wherein the magnesium cations are counterions to no more than about 35% of the carboxylate groups on the polymer. In some embodiments, the polymer comprises calcium and magnesium cations as counterions to about 5% to about 75% of the carboxylate groups on the polymer, and hydrogen cations (e.g., protons) are counterions to the remainder or substantially the remainder of the carboxylate groups on the polymer (e.g., counterions that are not calcium or sodium are hydrogen), wherein the magnesium cations are counterions to no more than about 35% of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium cations as counterions to about 5% to about 75% of the carboxylate groups on the polymer, wherein counterions that are not calcium are hydrogen. In some embodiments, the polymer comprises calcium cations as counterions to about 5% to about 75% of the carboxylate groups on the polymer and hydrogen cations (e.g., protons) are counterions to the remainder or substantially the remainder of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium cations and magnesium cations as counterions to about 5% to about 75% of the carboxylate groups on the polymer, wherein counterions that are not calcium or magnesium are hydrogen, and wherein the magnesium cations are counterions to no more than about 35% of the carboxylate groups on the polymer. In some embodiments, the polymer comprises calcium cations and magnesium cations as counterions to about 5% to about 75% of the carboxylate groups on the polymer, and hydrogen cations (e.g., protons) are counterions to the remainder or substantially the remainder of the carboxylate groups on the polymer, wherein the magnesium cations are counterions to no more than about 35% of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium cations as counterions to about 5% to about 75% of the carboxylate groups on the polymer, and sodium cations as counterions to no more than about 5% of the carboxylate groups on the polymer. In such embodiments, the polymer further comprises hydrogen cations (e.g., protons) as counterions to all or substantially all of the carboxylate groups to which calcium and sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 50% to about 60% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, the polymer comprises calcium and magnesium cations as counterions to about 5% to about 75% of the carboxylate groups on the polymer, and sodium cations as counterions to no more than about 5% of the carboxylate groups on the polymer, and further comprises hydrogen cations (e.g., protons) as counterions to all or substantially all of the carboxylate groups to which calcium, magnesium, or sodium are not counterions (e.g., “free carboxylates”), for example, about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates, wherein magnesium cations are counterions to no more than about 35% of the carboxylate groups on the polymer. In some embodiments, the polymer comprises calcium and magnesium cations as counterions to about 50% to about 60% of the carboxylate groups on the polymer, sodium cations as counterions to no more than about 5% of the carboxylate groups on the polymer, and hydrogen cations (e.g., protons) as counterions to all or substantially all of the free carboxylates, wherein magnesium cations are counterions to no more than about 35% of the carboxylate groups on the polymer.

In some embodiments, compositions and/or dosage forms comprising a base and a cross-linked cation-binding polymer, including a cross-linked acrylic acid polymer, absorb about 10-fold, 20-fold, 30-fold, or 40-fold or more of their mass in a buffer solution (e.g., a solution of sodium salts at 0.154 molar total sodium concentration, for example, a saline solution or a physiological saline solution. For example, saline holding capacity for a disclosed cross-linked cation-binding polymer may be determined in a buffered saline solution, e.g., a buffered saline solution that maintains pH at about 7.

For the purposes of this disclosure, saline absorption capacity (e.g., saline holding capacity) is measured for the polymer as the sodium salt (for example the sodium salt of polyacrylate, or the acid form of the polymer (e.g. polyacrylic acid) converted to the sodium salt (e.g. by incubating in one or more exchanges of pH 7 sodium phosphate buffer to convert the polymer to the sodium salt)), in a saline solution, physiologic isotonic buffer, or a sodium phosphate buffer pH 7 with a sodium concentration of 154 mM.

Polymers as described herein and compositions that contain the polymers have unexpected cation binding or removal, and/or fluid binding or removal properties when administered to an individual (e.g., a mammal, such as a human) while minimizing any acidosis or alkylosis effects from the administration, and therefore are useful for the treatment of a variety of diseases or disorders, including those involving ion and/or fluid imbalances (e.g., overloads). Surprisingly, ranges of polymer in the compositions have been discovered and are disclosed herein that are optimized for maintaining the cation binding and/or removal properties of the polymer (e.g., for potassium and/or sodium) and the fluid binding and/or removal properties of the polymer in humans, while neutralizing hydrogen cations released from administration of the polymer. In some embodiments, a neutral or substantially neutral acid/base status) (e.g., acid/base balance) is maintained in the body of a subject, for example, a human subject. In some embodiments, an acid/base status (e.g., acid/base balance) associated with the subject does not change, for example, as measured by serum total bicarbonate, serum total CO₂, arterial blood pH, urine pH, urine phosphorous, urine ammonium, and/or anion gap. An acid/base status that does not change includes one that does not change outside the normal range or outside the normal range for the subject.

The present disclosure also relates to methods of preparation of such compositions. The present disclosure also relates to methods of using such compositions, for example, in dosage forms, for the treatment of various diseases or disorders as disclosed herein, including, for example, heart failure (e.g., with or without chronic kidney disease), end stage renal disease (e.g., with or without heart failure), chronic kidney disease, hypertension (including, e.g., salt sensitive and refractory), hyperkalemia (e.g., any origin), hypernatremia (e.g., any origin), and/or fluid overload states (e.g., edema or ascities).

In some embodiments, the polymer is a polycarboxylic acid polymer comprising monomers with a pKa-decreasing group such as an electron-withdrawing substituent (e.g., a hydroxyl group, an ethereal group, an ester group or a halide atom such as F), such as polyfluoroacrylic acid polymer. In some embodiments, the polymer is derived from polymerization of carboxylic acid-containing monomers with pKa-decreasing groups such as electron-withdrawing substituents (e.g., hydroxyl groups, ethereal groups, ester groups or halide atoms such as F). Non-limiting examples of suitable carboxylic acid-containing monomers include, for example: monomers of acrylic acid and its salts, methacrylic acid and its salts, crotonic acid and its salts, tiglinic acid and its salts, 2-methyl-2-butenoic acid (Z) and its salts, 3-butenoic acid (vinylacetic acid) and its salts, 1-cyclopentene carboxylic acid and its salts, 2-cyclopentene carboxylic acid and its salts; and unsaturated dicarboxylic acids and their salts, such as maleic acid, fumaric acid, itaconic acid, glutaconic acid, and their salts, wherein the monomers further comprise a pKa-decreasing group such as an electron-withdrawing substituent (e.g., a hydroxyl group, en ethereal group, an ester group, or a halide atom such as F). Copolymers of the above monomers may be included in the polymers. Exemplary monomers include fluoroacrylic acid and methyl-2-fluoroacrylate. Such monomers may be mixed with acrylic acid monomers for co-polymerization. Other cross-linked cation-binding polyelectrolyte polymers may be based on sulfonic acids and their salts, or phosphonic acids and their salts-and amines and their salts, for example, acrylic acid with sulfonic acids or salts thereof, phosphonic acids or salts thereof, or amines and their salts thereof. Regardless of the choice of monomer, the polymers useful in the present disclosure contain a plurality of carboxylic acid (—C(O)OH) groups.

Polymers of the present disclosure are crosslinked. Any crosslinker known in the art may be used. Crosslinking agents contemplated for use in the present disclosure, include, for example, diethelyeneglycol diacrylate (diacryl glycerol), triallylamine, tetraallyloxyethane, allylmethacrylate, 1,1,1-trimethylolpropane triacrylate (TMPTA), divinylglycol, divinylbenzene (DVB), ethylene bisacrylamide, N,N′-bis(vinylsulfonylacetyl) ethylene diamine, 1,3-bis (vinylsulfonyl) 2-propanol, vinylsulfone, N,N′-methylenebisacrylamide, epichlorohydrin (ECH), 1,7-octadiene (ODE), 1,5-hexadiene (HDE), or a combination thereof. An exemplary combination of crosslinkers is divinylbenzene (DVB) and 1,7, octadience (ODE). The amount of crosslinking agent used may vary depending on the absorbent characteristics desired. In general, increasing amounts of crosslinking agent will yield polymers with increasing degrees of crosslinking. Such polymers with higher degrees of crosslinking may be preferred over less crosslinked polymers when fluid absorption is unnecessary. For polymers of the present disclosure, an amount of crosslinking may be chosen that yields a polymer with an in vitro saline absorption capacity (e.g., saline holding capacity) of greater than about 20 times its own weight. For the purposes of this disclosure, saline absorption capacity (e.g., saline holding capacity) is measured in a saline solution buffered to pH7 including, for example, as described in Examples 8 to 10. For example, the amount of crosslinker used to crosslink polymers according to the present disclosure may range from about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol %. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers.

In certain exemplary embodiments, the crosslinked cation-binding polymer, as described, for example, for inclusion in compositions, formulations, and/or dosage forms and/or for use in methods for treatment of various diseases or disorders as described herein, and/or for use in methods for cation binding and/or removal, and/or fluid binding and/or removal, as described herein, is a crosslinked polymer. comprising monomers containing carboxylic acid groups and pKa-decreasing groups such as electron-withdrawing substituents (e.g., a halide such as F) (e.g., derived from fluoroacrylic acid or methyl fluoroacrylic acid monomers or salts or anhydrides thereof). For example, the polymer (e.g., polyfluoroacrylic acid or polymethyl-fluoroacrylate polymer) may be crosslinked with about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker, and for example, may comprise an in vitro saline absorption capacity (e.g., saline holding capacity) of at least about 20 times its weight (e.g., at least about 20 grams of saline per gram of polymer, or “g/g”), at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. For the purposes of this disclosure, saline absorption capacity (e.g., saline holding capacity) is measured in a saline solution buffered to pH7, including, for example, as described in Examples 8 to 10. In some embodiments, the crosslinked polymer (e.g., polyfluoroacrylic acid or polymethyl-fluoroacrylate polymer) comprises individual particles (e.g., beads) or particles that are agglomerated (for example, flocculated) to form a larger particle, wherein the individual or agglomerated particle diameter is about 1 to about 10,000 microns such as, for example, about 212 microns to about 500 microns, about 75 microns to about 150 microns (e.g., about 100 microns) or about 75 microns or less (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns). In one embodiment, the polymer is in the form of small particles that flocculate to form agglomerated particles with a diameter of about 1 micron to about 10 microns.

In certain exemplary embodiments, the crosslinked cation-binding polymer, as described, for example, for inclusion in compositions, formulations, and/or dosage forms and/or for use in methods for treatment of various diseases or disorders as described herein, and/or for use in methods for cation binding and/or removal, and/or fluid binding and/or removal, as described herein, is a crosslinked polyacrylate polymer (e.g., derived from acrylic acid monomers or a salt thereof) that comprises calcium cations and magnesium cations (e.g., a mixture of calcium and magnesium cations), wherein the calcium and magnesium cations are counterions to about 5% to about 75% of the carboxylate groups in the polymer, wherein the magnesium cations are counterions to no more than about 35% of the carboxylate groups in the polymer, and wherein sodium cations (if present) are counterions to no more than about 5% of the carboxylate groups in the polymer. For example, the polymer may be a polyacrylate polymer crosslinked with about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker or alternatively from about 0.025 mol.% to about 3.0 mol.% including, for example, from about 0.025 mol.% to about 0.3 mol.%, and for example, may comprise an in vitro saline absorption capacity (e.g., saline holding capacity) of at least about 20 times its weight (e.g., at least about 20 grams of saline per gram of polymer, or “g/g”), at least about at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. Additionally, for example, the polymer (e.g., polyfluoroacrylic acid) may be crosslinked with about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers. In some embodiments, the crosslinked polyacrylate polymer comprises individual particles or particles that are agglomerated (for example, flocculated) to form a larger particle, wherein the individual or agglomerated particle diameter is about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 20 microns to about 125 microns, about 20 microns to about 150 microns, about 20 microns to about 200 microns, about 35 microns to about 150 microns, about 35 microns to about 125 microns, about 50 microns to about 125 microns, about 50 microns to about 200 microns, about 50 microns to about 1000 microns, about 500 microns to about 1000 microns, about 1000 to about 5000 microns, or about 5000 microns to about 10,000 microns). For example, a polymer, including a polyacrylate polymer, may have a particle size of about 212 microns to about 500 microns, a particle size from about 75 microns to about 150 microns, or a particle size of about 75 microns or less. In some embodiments, the crosslinked polyacrylate polymer comprises individual particles or particles that are agglomerated (for example, flocculated) to form a larger particle, wherein the individual or agglomerated particle diameter is about 100 microns. In some embodiments, no less than about 70% of the polymer has a particle size of about 212 microns to about 500 microns, a particle size from about 75 microns to about 150 microns, or a particle size of about 75 microns or less. In one embodiment, the polyacrylate polymer is in the form of small particles that flocculate to form agglomerated particles with a diameter of about 1 micron to about 10 microns or about 0.1 microns to about 20 microns. Such polyacrylate polymers may include polycarbophil.

In some embodiments, a composition comprises a crosslinked cation-binding polymer (e.g., a polyfluoroacrylic acid), comprising monomers (e.g., fluoroacrylic acid) that comprise carboxylate groups and pKa decreasing groups and calcium cations or calcium and magnesium cations, wherein the calcium cations or calcium and magnesium cations are counterions to about 5% to about 75% of the carboxylate groups in the polymer, wherein the polymer contains no more than about 20 ppm of heavy metals, no more than about 1,000 ppm of residual monomer, no more than about 20 wt. % of soluble polymer, and loses less than about 20% of its weight upon drying; the polymer contains no more than about 1,000 ppm of sodium, no more than about 20 ppm of heavy metals, no more than about 500 ppm of residual monomer, no more than about 10 wt. % of soluble polymer, and loses less than about 20% of its weight upon drying; the polymer contains no more than about 500 ppm of sodium, no more than about 20 ppm of heavy metals, no more than about 100 ppm of residual monomer, no more than about 10 wt. % of soluble polymer, and loses less than about 10% of its weight upon drying; the polymer contains no more than about 500 ppm of sodium, no more than about 20 ppm of heavy metals, no more than about 50 ppm of residual monomer, no more than about 10 wt. % of soluble polymer, and loses less than about 5% of its weight upon drying; the polymer contains about 430 ppm of sodium, less than about 20 ppm of heavy metals, less than about 2 ppm of residual monomer, about 3 wt. % of soluble polymer, and loses about 20% of its weight upon drying; the polymer contains about 160 ppm of sodium, less than about 20 ppm of heavy metals, about 4 ppm of residual monomer, about 4 wt. % of soluble polymer, and loses about 3% of its weight upon drying; the polymer contains about 335 ppm of sodium, less than about 20 ppm of heavy metals, about 36 ppm of residual monomer, about 4 wt. % of soluble polymer, and loses about 10% of its weight upon drying; the polymer contains about 300 ppm of sodium, less than about 20 ppm of heavy metals, about 14 ppm of residual monomer, about 7 wt. % of soluble polymer, and loses about 2% of its weight upon drying; or the polymer contains about 153 ppm of sodium, less than about 20 ppm of heavy metals, less than about 40 ppm of residual monomer, about 3 wt. % of soluble polymer, and loses about 5% of its weight upon drying.

In some embodiments, a crosslinked polymer is administered with an added base (e.g., up to about 0.90 equivalents of added base per equivalents of carboxylate groups in the polymer).

In some embodiments, the base is one or more of: an alkali metal hydroxide, an alkali metal acetate, an alkali metal carbonate, an alkali metal bicarbonate, an alkali metal oxide, an alkaline earth metal hydroxide, an alkaline earth metal acetate, an alkaline earth metal carbonate, an alkaline earth metal bicarbonate, an alkaline earth metal oxide, and an organic base. In some embodiments, the base is choline, lysine, arginine, histidine, a pharmaceutically acceptable salt thereof, or a combination thereof. In some embodiments, the base is an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, or a combination thereof. In some embodiments, the base is a bicarbonate, a carbonate, an oxide, or a hydrochloride. In related embodiments, the base is one or more of: calcium bicarbonate, calcium carbonate, calcium oxide, and calcium hydroxide. In some embodiments, the base is a lithium salt, a sodium salt, a potassium salt, a magnesium salt, a calcium salt, an aluminum salt, a rubidium salt, a barium salt, a chromium salt, a manganese salt, an iron salt, a cobalt salt, a nickel salt, a copper salt, a zinc salt, an ammonium salt, a lanthanum salt, a choline salt, or a serine salt of any of the foregoing anions or anion combinations.

In some embodiments, the base may be selected to avoid increasing a level of a particular cation associated with the subject. For example, a composition according to the present disclosure intended to treat hyperkalemia in a subject would preferably contain a base that does not include potassium cations. Similarly, a composition according to the present disclosure intended to treat hypernatremia in a subject would preferably contain a base that does not include sodium cations.

As used herein, the term “cation” or “cations” refers to atomic, polyatomic, or molecular ions having a net positive charge, and may include one such cation or a combination of more than one cation. Non-limiting examples of cations include: hydrogen cations (H⁺), sodium cations (Na⁺), potassium cations (K⁺), magnesium cations (Mg²⁺), calcium cations (Ca²⁺), iron cations (e.g., Fe²⁺, Fe³⁺), and combinations thereof. As used herein, the term “non-hydrogen cation” or “non-hydrogen cations” refers to cation(s) (e.g., as defined above) other than hydrogen (Hⁱ; proton). Mixtures of more than one cation are within the scope of the terms cation or cations, as used herein. Counterions to carboxylate groups on the polymers described herein are cations. Crosslinked cation-binding polymers as disclosed herein can be described by the percentage of carboxylate groups for which one or more cation serves as a counterion. For example, a polymer according to the present disclosure may be referred to as “50% calcium” to indicate that calcium cations are counterions to about 50% of the carboxylate groups in the polymer. Or, when expressed as a molar ratio, a “50% calcium” polymer according to the present disclosure includes about 25 moles of calcium cations (e.g., divalent Ca²⁺ cations) per 100 moles of carboxylate groups in the polymer (e.g., a mole fraction with respect to calcium of 0.25). In another example, a “35% magnesium” polymer according to the present disclosure indicates that magnesium cations (e.g., divalent Mg²⁺ cations) are counterions to about 35% of the carboxylate groups in the polymer (e.g., a mole fraction with respect to magnesium of 0.175). In some embodiments, hydrogen cations (e.g., protons) may be counterions to all or substantially all of the carboxylate groups for which calcium and/or magnesium are not counterions.

In some embodiments, crosslinked cation-binding polymers according to the present disclosure comprise: i) calcium cations that are counterions to about 5% to about 75% of the carboxylate groups in the polymer, and further comprise one or more additional cations; or ii.) calcium and magnesium cations that are counterions to about 5% to about 75% of the carboxylate groups in the polymer, wherein the magnesium cations are counterions to no more than about 35% of the carboxylate groups in the polymer, and further comprise one or more additional cations. In some embodiments, the one or more additional cations are monovalent cations such as sodium, potassium, ammonium, arginine, lysine, choline, histidine, serine, and the like. In some embodiments, the one or more additional cations are divalent cations such as iron(II), zinc, a lanthanide, and the like. In some embodiments, the one or more cations are trivalent cations such as aluminum, iron(III), and the like. Nomenclature of a polymer which comprises one or more additional cations thus depends on the identity of the one or more additional cations, the amount of each of the one or more additional cations, and the valency of each of the one or more additional cations. For example, a polymer according to the present disclosure denoted as “50% calcium/9% iron (III)” would indicate that calcium cations are counterions to about 50% of the carboxylate groups in the polymer, trivalent iron cations (Fe³⁺ cations) are counterions to about 9% of the carboxylate groups in the polymer, and hydrogen cations (e.g., protons) are counterions to about 41% of the carboxylate groups in the polymer. To avoid any doubt, a “50% calcium/9% trivalent iron” polymer according to the present disclosure comprises about 25 moles of calcium cations and about 3 moles of trivalent iron cations per 100 moles of carboxylate groups in the polymer (e.g., 0.50 equivalents of calcium cations and 0.09 equivalents of iron).

Determination of the percentage of cations that serve as counterions to carboxylate groups in a polymer as disclosed herein (e.g., parts per million, weight percent, etc.) can be accomplished by any suitable ICP means known in the art (e.g., ICP spectroscopy). When the polymer comprises calcium as a counterion to the carboxylate groups of the polymer, the polymer may be referred to as Ca-CLP. When the polymer comprises calcium and magnesium as counterions to the carboxylate groups of the polymer, the polymer may be referred to as Ca/Mg-CLP. When the polymer comprises sodium as a counterion to the carboxylate groups of the polymer, the polymer may be referred to as Na-CLP. For example and without limitation, the polymer may be analyzed with an inductively coupled plasma (“ICP”) spectrometer (e.g., by mass spectroscopy (ICP-MS), atomic emission spectroscopy (ICP-AES), or optical emission spectroscopy (ICP-OES)) using methods known to those skilled in the art. The percentage of cations serving as counterions to carboxylate groups in the polymer (e.g., calcium and/or magnesium counterions to the carboxylate groups in the polymer) may be confirmed by ICP spectroscopy. Such methods include methods of sample preparation wherein the polymer is substantially or completely digested.

Cation content of polymers disclosed herein may be determined by ICP, including ICP-AES, ICP-MS, or ICP-OES (see, e.g., Example 6). In some exemplary embodiments, content of calcium, magnesium, sodium, potassium, and/or iron may be determined. The ICP analysis may be reported in μg cation/g polymer, which may then be converted to weight percent (wt. %). Weight percent may be converted to % of cations that are counterions to the carboxylate groups in the polymer. The % of cations that are counterions to the carboxylate groups in the polymer determined in different measurements may vary by ±5% or less. For example, the determination of 5% to 75% calcium cations or calcium and magnesium cations as counterions to carboxylate groups in the polymer may vary in different measurements by ICP [e.g., 5%±5% to 75%±5% (4.75 to 5.25 or 71.25 to 78.75)].

For example, an ICP analysis of a crosslinked cation-binding polyacrylate polymer as disclosed herein, comprising hydrogen cations (e.g., protons), calcium cations and acrylic acid monomers having carboxylate groups, reporting about 16 wt. % calcium cations corresponds to a polyacrylate polymer wherein about 70% of the carboxylate groups are bound to calcium counterions, as calculated by the following formula:

[x]% Ca-CLP=(72.06)(wt. % Ca)/(20.05−(0.19)(wt. % Ca))

In an embodiment, an ICP analysis that reports calcium content at 16.4 wt. % calcium represents a polyacrylate polymer with about 70% of the carboxylate groups bound to calcium (e.g., calcium cations are counterions to about 70% of the carboxylate groups in the polymer), as calculated by the following formula:

[x]% Ca-CLP=(72.06)(16.4)/(20.05−(0.19)(16.4))=70% Ca-CLP

In another embodiment, an ICP analysis that reports calcium content at 17.4 wt. % represents a 75% Ca-CLP) polymer (e.g., calcium cations are counterions to about 75% of the carboxylate groups in the polymer).

For example, an ICP analysis that reports magnesium content at 5.6 wt. % magnesium represents about 35% magnesium as counterions to carboxylate groups in a polyacrylate polymer (e.g., about 35% Mg-CLP), as calculated by the following formula for a polyacrylate polymer:

[x]% Mg-CLP=(72.06)(wt. % Mg)/(12.15−(0.11)(wt. % Mg))

[x]% Mg-CLP=(72.06)(5.6)/(12.15−(0.11)(5.6))=35% Mg-CLP

For example, an ICP analysis that reports sodium content at 0.030 wt. % sodium represents about 0.09% sodium cations as counterions to carboxylate groups in the polymer, as calculated by the following formula for a polyacrylate polymer:

[x]% Na-CLP=(72.06)(wt. % Na)/(23.0−(0.23)(wt. % Na))

For 0.03 wt % sodium, the calculation is as follows:

[x]% Na-CLP=(72.06)(0.030)/(23.0−(0.23)(0.030))=0.09% sodium counterions to the carboxylate groups in the polymer.

In an embodiment, an ICP analysis that reports sodium content at 0.031 wt. % sodium represents a polacrylate polymer having sodium counterions to about 0.10% of the carboxylate groups in the polymer.

In another embodiment, an ICP analysis that reports sodium content at 0.04 wt. % represents a polyacrylate polymer having sodium counterions to about 0.13% sodium of the carboxylate groups in the polymer.

The above equations are useful for determination of percentage cation when a single type of non-hydrogen cation (e.g., calcium) is present as a counterion to carboxylate groups in the polymer. As understood by persons skilled in the art, the above equations are modified when combinations of types cations are present (e.g., calcium, magnesium, and/or sodium).

Compositions, formulations, and/or dosage forms comprising a polymer as disclosed herein may optionally additionally comprise and/or be co-administered with an additional base (alternatively termed an alkali). As used herein, the term base may refer to a suitable compound or mixture of compounds that is capable of increasing the pH of the blood or other bodily fluids. Exemplary bases include, but are not limited to, calcium carbonate, magnesium carbonate, magnesium hydroxide, magnesium hydroxide, sodium bicarbonate, potassium bicarbonate, aluminum hydroxide, calcium citrate. Exemplary bases also include calcium acetate, calcium oxide, potassium citrate, and potassium acetate. Generally, inorganic and organic bases can be used, provided they are physiologically and/or clinically acceptable. To be acceptable, the dose and route of administration of the specific base are important considerations. For example, oral administration of even small amounts of sodium hydroxide would cause local tissue damage and would not be acceptable on this basis while administration of intermittent, small amounts of sodium hydroxide intravenously is performed routinely. Similarly, though lithium carbonate or rubidium acetate would be an acceptable base, only small amounts could be used due to the effects of the lithium or the rubidium, regardless of the route of administration.

In some embodiments, compositions, formulations, and/or dosage forms comprising a polymer as disclosed herein additionally comprise a base that may be present in an amount sufficient to provide up to about 0.90 equivalents of base per equivalent of carboxylate groups in the polymer. For example, the composition, formulation, and/or dosage form may contain a disclosed polymer that contains calcium and/or magnesium cations that are counterions to about 5% to about 75% of the carboxylate groups (e.g., calcium and/or magnesium counterions that are added during manufacture of the polymer) and base that is added during preparation or formulation of the composition, formulation, and/or dosage form in an amount sufficient to provide up to about 0.90 equivalents per equivalent of carboxylate groups in the polymer.

In some embodiments, the additional base is present, for example, in a composition, formulation, and/or dosage form comprising a disclosed polymer and/or is co-administered with a disclosed polymer, in an amount sufficient to provide about 0.20 equivalents to about 0.90 equivalents of base per equivalent (e.g., mole) of carboxylic acid groups in the polymer. A monobasic base provides one equivalent of base per mole of monobasic base. A dibasic base provides two equivalents of base per mole of dibasic base. A tribasic base provides three equivalents of base per mole of tribasic base. For example, a composition comprising a polymer derived from polymerization and crosslinking of 1.0 mole of acrylic acid monomers may contain about 0.20 moles to about 0.90 moles of a monobasic base, such as a bicarbonate. If a dibasic base is used, such as a carbonate, a composition comprising 1.0 mole of carboxylic acid groups may contain about 0.10 to about 0.45 moles of the dibasic base. If a tribasic base is used, such as a phosphate, a composition comprising a polymer derived from polymerization and crosslinking of 1.0 mole of acrylic acid monomers may contain about 0.065 moles to about 0.3 moles of the tribasic base.

In some embodiments, compositions of the present disclosure comprise a monobasic base (e.g., one equivalent per mole) present in an amount sufficient to provide from about 0.20 moles to about 0.90 moles of base per mole of carboxylic acid groups in the polymer, for example about 0.2 moles of base, about 0.25 moles of base, about 0.3 moles of base, about 0.35 moles of base, about 0.4 moles of base, about 0.45 moles of base, about 0.50 moles of base, about 0.55 moles of base, about 0.60 moles of base, about 0.65 moles of base, about 0.70 moles of base, about 0.75 moles of base, about 0.80 moles of base, about 0.85 moles of base, or about 0.90 moles of base per mole of carboxylic acid groups in the polymer. In some embodiments, compositions of the present disclosure comprise a monobasic base present in an amount sufficient to provide from about 0.2 moles of base to about 0.90 moles of base, for example about 0.2 moles of base, about 0.25 moles of base, about 0.3 moles of base, about 0.35 moles of base, about 0.4 moles of base, about 0.45 moles of base, about 0.50 moles of base, about 0.55 moles of base, about 0.60 moles of base, about 0.65 moles of base, about 0.70 moles of base, about 0.75 moles of base, about 0.80 moles of base, about 0.85 moles of base, or about 0.90 moles of base per mole of carboxylate groups in the polymer. In some embodiments, compositions of the present disclosure comprise a monobasic base present in an amount sufficient to provide about 0.90 moles of base per mole of carboxylate groups in the polymer.

In some embodiments, compositions of the present disclosure comprise a dibasic base (e.g., two equivalents per mole) present in an amount sufficient to provide from about 0.10 moles to about 0.45 moles of base per mole of carboxylic acid groups in the polymer, for example about 0.1 moles of base, about 0.125 moles of base, about 0.15 moles of base, about 0.175 moles of base, about 0.2 moles of base, about 0.225 moles of base, about 0.25 moles of base, about 0.275 moles of base, about 0.3 moles of base, about 0.325 moles of base, about 0.35 moles of base, about 0.375 moles of base, about 0.4 moles of base, about 0.425 moles of base, or about 0.45 moles of base per mole of carboxylic acid groups in the polymer. In some embodiments, compositions of the present disclosure comprise a dibasic base present in an amount sufficient to provide from about 0.1 moles of base to about 0.3 moles of base, for example about 0.1 moles of base, about 0.125 moles of base, about 0.15 moles of base, about 0.175 moles of base, about 0.2 moles of base, about 0.225 moles of base, 0.25 moles of base, about 0.275 moles of base, or about 0.3 moles of base per mole of carboxylate groups in the polymer. In some embodiments, compositions of the present disclosure comprise a dibasic base present in an amount sufficient to provide from about 0.15 moles of base to about 0.25 moles of base, for example about 0.15 moles of base, about 0.175 moles of base, about 0.2 moles of base, about 0.225 moles of base, or about 0.25 moles of base per mole of carboxylate groups in the polymer. In some embodiments, compositions of the present disclosure comprise a dibasic base present in an amount sufficient to provide from about 0.2 moles of base to about 0.25 moles of base, for example about 0.2 moles of base, about 0.225 moles of base, or about 0.25 moles of base per mole of carboxylate groups in the polymer. In some embodiments, compositions of the present disclosure comprise a dibasic base present in an amount sufficient to provide about 0.225 moles of base per mole of carboxylate groups in the polymer.

In some embodiments, compositions of the present disclosure comprise a tribasic base (e.g., three equivalents per mole) present in an amount sufficient to provide about 0.065 moles to about 0.3 moles of base per mole of carboxylic acid groups in the polymer, for example about 0.065 moles of base, about 0.067 moles of base, about 0.07 moles of base, about 0.075 moles of base, about 0.08 moles of base, about 0.085 moles of base, about 0.09 moles of base, about 0.095 moles of base, about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, about 0.15 moles of base, about 0.155 moles of base, about 0.16 moles of base, about 0.165 moles of base, about 0.17 moles of base, about 0.175 moles of base, about 0.18 moles of base, about 0.185 moles of base, about 0.19 moles of base, about 0.195 moles of base, about 0.2 moles of base, about 0.205 moles of base, about 0.21 moles of base, about 0.215 moles of base, about 0.22 moles of base, about 0.225 moles of base, about 0.23 moles of base, about 0.235 moles of base, about 0.24 moles of base, about 0.245 moles of base, about 0.25 moles of base, about 0.255 moles of base, about 0.26 moles of base, about 0.265 moles of base, about 0.27 moles of base, about 0.275 moles of base, about 0.28 moles of base, about 0.285 moles of base, about 0.29 moles of base, about 0.295 moles of base, or about 0.3 moles of base per mole of carboxylic acid groups in the polymer. In some embodiments, compositions of the present disclosure comprise a tribasic base present in an amount sufficient to provide from about 0.065 moles of base to about 0.25 moles of base, for example about 0.065 moles of base, about 0.067 moles of base, about 0.07 moles of base, about 0.075 moles of base, about 0.08 moles of base, about 0.085 moles of base, about 0.09 moles of base, about 0.095 moles of base, about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, about 0.15 moles of base, about 0.155 moles of base, about 0.16 moles of base, about 0.165 moles of base, about 0.17 moles of base, about 0.175 moles of base, about 0.18 moles of base, about 0.185 moles of base, about 0.19 moles of base, about 0.195 moles of base, about 0.2 moles of base, about 0.205 moles of base, about 0.21 moles of base, about 0.215 moles of base, about 0.22 moles of base, about 0.225 moles of base, about 0.23 moles of base, about 0.235 moles of base, about 0.24 moles of base, about 0.245 moles of base, or about 0.25 moles of base per mole of carboxylate groups in the polymer. In some embodiments, compositions of the present disclosure comprise a tribasic base present in an amount sufficient to provide from about 0.1 moles of base to about 0.15 moles of base, for example about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, or about 0.15 moles of base per mole of carboxylate groups in the polymer. In some embodiments, compositions of the present disclosure comprise a tribasic base present in an amount sufficient to provide about 0.067 moles of base per mole of carboxylate groups in the polymer. In some embodiments, compositions of the present disclosure comprise a tribasic base present in an amount sufficient to provide about 0.3 moles of base per mole of carboxylate groups in the polymer.

In some embodiments, compositions, formulations, and/or dosage forms of the present disclosure comprise one or more than one base (e.g., one or more monobasic bases, one or more dibasic bases, one or more tribasic bases, etc.). In such embodiments, the compositions comprise an amount of each base such that the total number of equivalents of base present is up to about 0.90 equivalents per mole of carboxylic acid groups in the polymer, for example, about 0.20 equivalents to about 0.90 equivalents per mole of carboxylic acid groups in the polymer. For example, a composition comprising 1.0 moles of carboxylic acid groups in the polymer may further comprise a total amount of base according to the following Equation 1:

about 0.2)(N_{COOH})≦(N_{monobasic})+(2)(N_{dibasic})+(3)(N_{tribasic})+(4)(N_{tetrabasic})+ . . . ≦(about 0.95)(N_{COOH}),

wherein:

N_{COOH} is the number of moles of carboxylate groups in the polymer;

N_{monobasic} is the number of moles of all monobasic bases present in the composition;

N_{dibasic} is the number of moles of all dibasic bases present in the composition;

N_{tribasic} is the number of moles of all tribasic bases present in the composition; and

N_{tetrabasic} is the number of moles of all tetrabasic bases present in the composition.

Thus, as one example embodiment, a composition according to the present invention that comprises 1.0 mole of carboxylic acid groups and 0.2 moles of sodium bicarbonate may also comprise from about 0.35 moles of a dibasic base such as magnesium carbonate. In such an embodiment, the total equivalents of base would be equal to 0.2+(2)(about 0.35), or about 0.9 equivalents of base.

In some embodiments, the base is present in an amount sufficient to provide from up to about 0.90 equivalents of base, for example about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylic acid groups in the polymer. In some embodiments, the base is present in an amount sufficient to provide from about 0.20 to about 0.25, about 0.20 to about 0.30, about 0.20 to about 0.35, about 0.20 to about 0.40, about 0.20 to about 0.45, about 0.20 to about 0.50, about 0.20 to about 0.55, about 0.20 to about 0.60, about 0.20 to about 0.65, about 0.20 to about 0.70, about 0.20 to about 0.75, about 0.20 to about 0.80, about 0.20 to about 0.85, about 0.20 to about 0.90, about 0.25 to about 0.30, about 0.25 to about 0.35, about 0.25 to about 0.40, about 0.25 to about 0.45, about 0.25 to about 0.50, about 0.25 to about 0.55, about 0.25 to about 0.60, about 0.25 to about 0.65, about 0.25 to about 0.70, about 0.25 to about 0.75, about 0.25 to about 0.80, about 0.25 to about 0.85, about 0.25 to about 0.90, about 0.30 to about 0.35, about 0.30 to about 0.40, about 0.30 to about 0.45, about 0.30 to about 0.50, about 0.30 to about 0.55, about 0.30 to about 0.60, about 0.30 to about 0.65, about 0.30 to about 0.70, about 0.30 to about 0.75, about 0.30 to about 0.80, about 0.30 to about 0.85, about 0.30 to about 0.90, about 0.35 to about 0.40, about 0.35 to about 0.45, about 0.35 to about 0.50, about 0.35 to about 0.55, about 0.35 to about 0.60, about 0.35 to about 0.65, about 0.35 to about 0.70, about 0.35 to about 0.75, about 0.35 to about 0.80, about 0.35 to about 0.85, about 0.35 to about 0.90, about 0.40 to about 0.45, about 0.40 to about 0.50, about 0.40 to about 0.55, about 0.40 to about 0.60, about 0.40 to about 0.65, about 0.40 to about 0.70, about 0.40 to about 0.75, about 0.40 to about 0.80, about 0.40 to about 0.85, about 0.40 to about 0.90, about 0.45 to about 0.50, about 0.45 to about 0.55, about 0.45 to about 0.60, about 0.45 to about 0.65, about 0.45 to about 0.70, about 0.45 to about 0.75, about 0.45 to about 0.80, about 0.45 to about 0.85, about 0.45 to about 0.90, about 0.50 to about 0.55, about 0.50 to about 0.60, about 0.50 to about 0.65, about 0.50 to about 0.70, about 0.50 to about 0.75, about 0.50 to about 0.80, about 0.50 to about 0.85, about 0.50 to about 0.90, about 0.55 to about 0.60, about 0.55 to about 0.65, about 0.55 to about 0.70, about 0.55 to about 0.75, about 0.55 to about 0.80, about 0.55 to about 0.85, about 0.55 to about 0.90, about 0.60 to about 0.65, about 0.60 to about 0.70, about 0.60 to about 0.75, about 0.60 to about 0.80, about 0.60 to about 0.85, about 0.60 to about 0.90, about 0.65 to about 0.70, about 0.65 to about 0.75, about 0.65 to about 0.80, about 0.65 to about 0.85, about 0.65 to about 0.90, about 0.70 to about 0.75, about 0.70 to about 0.80, about 0.70 to about 0.85, about 0.70 to about 0.90, about 0.75 to about 0.80, about 0.75 to about 0.85, about 0.75 to about 0.90, about 0.80 to about 0.85, about 0.80 to about 0.90, about or 0.85 to about 0.90 of base per equivalent of carboxylate groups in the polymer.

In some embodiments, the base is one or more of: an alkali metal hydroxide, an alkali metal acetate, an alkali metal carbonate, an alkali metal bicarbonate, an alkali metal oxide, an alkaline earth metal hydroxide, an alkaline earth metal acetate, an alkaline earth metal carbonate, an alkaline earth metal bicarbonate, an alkaline earth metal oxide, and an organic base. In some embodiments, the base is choline, lysine, arginine, histidine, a pharmaceutically acceptable salt thereof, or a combination thereof. In some embodiments, the base is an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, or a combination thereof. In some embodiments, the base is a bicarbonate, a carbonate, an oxide, or a hydrochloride. In related embodiments, the base is one or more of: calcium bicarbonate, calcium carbonate, calcium oxide, and calcium hydroxide. In some embodiments, the base is a lithium salt, a sodium salt, a potassium salt, a magnesium salt, a calcium salt, an aluminum salt, a rubidium salt, a barium salt, a chromium salt, a manganese salt, an iron salt, a cobalt salt, a nickel salt, a copper salt, a zinc salt, an ammonium salt, a lanthanum salt, a choline salt, or a serine salt of any of the foregoing anions or anion combinations.

In some embodiments, the base may be selected to avoid increasing a level of a particular cation associated with the subject. For example, a method of treatment for hyperkalemia in a subject would preferably include administering a base that does not include potassium cations, in conjunction with a polymer or composition, formulation or dosage form containing a polymer as disclosed herein. Similarly, a composition according to the present disclosure intended to treat hypernatremia in a subject would preferably contain a base that does not include sodium cations

In some embodiments, the base may be selected to specifically increase the amount of a particular cation important in the disease or condition of the subject. For example, a method of treatment for hyponatremia, for example, in a subject suffering simultaneously from hyperkalemia and hyponatremia, would preferably contain a base that either includes sodium cations or alters the polymer binding in such a manner that fewer sodium cations are removed by the polymer.

In some embodiments, the disclosed polymers and compositions, formulations, and/or dosage forms containing the polymers described herein have superior manufacturing and processing properties in comparison to analogous polymers, compositions, formulations, and/or dosage forms wherein the carboxylate groups of the polymer are bound to hydrogen cations (e.g., protons; H⁺) instead of calcium cations at the levels described herein. Typically, polymers with predominantly unneutralized carboxylate groups (e.g., more than about 95% of the carboxylates are bound to hydrogen cations) are very adhesive, which may result in manufacturing difficulties and poor oral delivery or mucoadhesive properties. For example, certain methods for preparing the disclosed polymers, compositions, formulations, and/or dosage forms require transferring the polymer from one vessel to another, drying the polymer, grinding or milling to form a powder, filtering the polymer, etc. The adhesive properties associated with polymer having predominantly unneutralized carboxylate groups may render one these exemplary processes difficult, time-consuming, cost-inefficient, or sub-optimal for scale up.

Generally speaking, processing of polycarboxylate polymers, handling of carboxylate polymer, and oral delivery properties of polycarboxylate polymers improve with increasing levels of bound non-hydrogen cations, e.g., calcium ions or calcium and magnesium ions. For example, polymers disclosed herein have calcium counterions to about 5% to about 75% of the carboxylate groups in the polymer. Alternatively, for example, polymers disclosed herein have about calcium and magnesium counterions to about 5% to about 75% of the carboxylate groups in the polymer, wherein the magnesium cations are counterions to no more than about 35% of the carboxylate groups in the polymer. Such polymers are characterized by dramatically improved manufacturability due to greatly reduced adhesive properties. In addition, the adhesive properties associated with polymers having predominantly unneutralized carboxylate groups lead to poor oral delivery properties as they generally hydrate rapidly when exposed to saliva, becoming bioadhesive. The hydrated form of the polymer causes the material to adhere to oral tissue, including teeth, which can lead to irritation. In contrast, polymers disclosed herein, which have calcium counterions to about 5% to about 75% of the carboxylate groups in the polymer, or which have calcium and magnesium counterions to about 5% to about 75% of the carboxylate groups in the polymer, wherein the magnesium cations are counterions to no more than about 35% of the carboxylate groups in the polymer, possess improved oral delivery properties.

In some embodiments, a polymer and/or composition of the present disclosure has an in vitro saline absorption capacity (e.g., saline holding capacity) of greater than 20 times its own weight (e.g., greater than about 20 grams of saline per gram of composition, or “g/g”). In related embodiments, the polymer and/or composition has an in vitro saline absorption capacity (e.g., saline holding capacity) of about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, or about 100 times its own weight, or more.

In one embodiment, a composition comprises a crosslinked cation-binding polymer comprising monomers that comprise carboxylic acid groups and pKa-decreasing groups such as electron-withdrawing substituents (e.g., a halide atom such as F); and calcium cations or calcium and magnesium cations to about 5% to about 75% of the carboxylate groups in the polymer, wherein the polymer is crosslinked with about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker, or about 4.0 mol % to about 20.0 mol % including, about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol %, wherein, the monomers are fluoroacrylic acid or methylfluoracrylic acid then salts or anhydrides.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 10% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 15% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 20% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 25% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 15% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 20% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 25% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 20% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 25% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 25% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 10% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 15% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 20% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 25% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 15% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 20% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 25% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 20% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 25% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 25% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 10% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 15% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 20% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 25% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 15% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 20% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 25% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 20% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 25% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 25% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 10% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 15% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 20% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 25% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 15% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 20% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 25% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 20% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 25% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 25% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 10% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 15% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 20% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 25% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 15% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 20% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 25% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 20% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 25% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 25% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 75% of the carboxylate groups of said polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 75% of the carboxylate groups of said polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 75% of the carboxylate groups of said polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 75% of the carboxylate groups of said polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 5% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 10% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 15% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 20% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 25% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 30% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 35% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 40% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 45% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 50% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 55% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 60% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 65% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 70% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations that are counterions to about 75% of the carboxylate groups of said polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 10% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 10% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 10% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 10% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 10% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 15% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 20% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 25% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 30% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 35% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 40% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 45% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 50% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 55% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 60% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 65% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 70% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% to about 75% of the carboxylate groups of said polymer, wherein the magnesium cations are counterions to no more than 35% of the carboxylate groups in the polymer, wherein the polymer is crosslinked with about 0.025 mol % to about 3.0 mol %, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the magnesium cations are counterions to no more than 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the magnesium cations are counterions to no more than 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the magnesium cations are counterions to no more than 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the magnesium cations are counterions to no more than 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 5% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the magnesium cations are counterions to no more than 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the calcium cations are counterions to about 5% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 10% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 15% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 20% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 25% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 30% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 35% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 40% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 45% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 50% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 55% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 60% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 65% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 70% of the carboxylate groups in the polymer, wherein the magnesium cations are counterions to about 5% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 5% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 10% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 15% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 20% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 25% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 30% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 35% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 40% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 45% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 50% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 55% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 60% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 65% of the carboxylate groups in the polymer, wherein the magnesium cations are counterions to about 10% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 5% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 10% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 15% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 20% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 25% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 30% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 35% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 40% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 45% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 50% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 55% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 60% of the carboxylate groups in the polymer, wherein the magnesium cations are counterions to about 15% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 5% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 10% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 15% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 20% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 25% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 30% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 35% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 40% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 45% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 50% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 55% of the carboxylate groups in the polymer, wherein the magnesium cations are counterions to about 20% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 5% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 10% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 15% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 20% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 25% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 30% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 35% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 40% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 45% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 50% of the carboxylate groups in the polymer, wherein the magnesium cations are counterions to about 25% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 5% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 10% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 15% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 20% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 25% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 30% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 35% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 40% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 45% of the carboxylate groups in the polymer, wherein the magnesium cations are counterions to about 30% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 5% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 10% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 15% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 20% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 25% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 30% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 35% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 40% of the carboxylate groups in the polymer, wherein the magnesium cations are counterions to about 35% of the carboxylate groups in the polymer, and wherein the polymer comprises no more than about 5% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the calcium cations are counterions to about 5% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 10% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 15% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 20% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 25% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 30% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 35% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 40% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 45% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 50% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 55% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 60% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 65% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 70% of the carboxylate groups in the polymer, wherein the magnesium cations are counterions to about 5% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 5% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 10% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 15% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 20% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 25% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 30% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 35% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 40% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 45% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 50% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 55% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 60% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 65% of the carboxylate groups in the polymer, wherein the magnesium cations are counterions to about 10% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 5% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 10% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 15% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 20% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 25% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 30% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 35% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 40% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 45% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 50% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 55% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 60% of the carboxylate groups in the polymer, wherein the magnesium cations are counterions to about 15% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 5% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 10% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 15% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 20% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 25% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 30% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 35% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 40% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 45% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 50% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 55% of the carboxylate groups in the polymer, wherein the magnesium cations are counterions to about 20% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 5% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 10% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 15% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 20% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 25% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 30% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 35% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 40% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 45% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 50% of the carboxylate groups in the polymer, wherein the magnesium cations are counterions to about 25% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 5% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 10% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 15% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 20% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 25% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 30% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 35% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 40% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 45% of the carboxylate groups in the polymer, wherein the magnesium cations are counterions to about 30% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 5% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 10% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 15% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 20% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 25% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 30% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 35% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 40% of the carboxylate groups in the polymer, wherein the magnesium cations are counterions to about 35% of the carboxylate groups in the polymer, and wherein the polymer comprises no more than about 4% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the calcium cations are counterions to about 5% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 10% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 15% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 20% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 25% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 30% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 35% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 40% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 45% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 50% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 55% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 60% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 65% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 70% of the carboxylate groups in the polymer, wherein the magnesium cations are counterions to about 5% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 5% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 10% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 15% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 20% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 25% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 30% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 35% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 40% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 45% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 50% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 55% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 60% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 65% of the carboxylate groups in the polymer, wherein the magnesium cations are counterions to about 10% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 5% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 10% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 15% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 20% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 25% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 30% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 35% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 40% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 45% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 50% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 55% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 60% of the carboxylate groups in the polymer, wherein the magnesium cations are counterions to about 15% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 5% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 10% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 15% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 20% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 25% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 30% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 35% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 40% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 45% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 50% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 55% of the carboxylate groups in the polymer, wherein the magnesium cations are counterions to about 20% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 5% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 10% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 15% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 20% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 25% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 30% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 35% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 40% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 45% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 50% of the carboxylate groups in the polymer, wherein the magnesium cations are counterions to about 25% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 5% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 10% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 15% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 20% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 25% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 30% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 35% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 40% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 45% of the carboxylate groups in the polymer, wherein the magnesium cations are counterions to about 30% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 5% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 10% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 15% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 20% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 25% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 30% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 35% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 40% of the carboxylate groups in the polymer, wherein the magnesium cations are counterions to about 35% of the carboxylate groups in the polymer, and wherein the polymer comprises no more than about 3% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the calcium cations are counterions to about 5% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 10% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 15% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 20% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 25% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 30% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 35% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 40% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 45% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 50% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 55% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 60% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 65% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 70% of the carboxylate groups in the polymer, wherein the magnesium cations are counterions to about 5% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 5% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 10% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 15% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 20% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 25% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 30% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 35% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 40% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 45% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 50% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 55% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 60% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 65% of the carboxylate groups in the polymer, wherein the magnesium cations are counterions to about 10% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 5% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 10% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 15% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 20% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 25% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 30% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 35% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 40% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 45% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 50% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 55% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 60% of the carboxylate groups in the polymer, wherein the magnesium cations are counterions to about 15% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 5% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 10% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 15% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 20% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 25% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 30% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 35% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 40% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 45% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 50% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 55% of the carboxylate groups in the polymer, wherein the magnesium cations are counterions to about 20% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 5% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 10% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 15% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 20% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 25% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 30% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 35% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 40% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 45% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 50% of the carboxylate groups in the polymer, wherein the magnesium cations are counterions to about 25% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 5% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 10% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 15% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 20% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 25% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 30% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 35% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 40% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 45% of the carboxylate groups in the polymer, wherein the magnesium cations are counterions to about 30% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 5% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 10% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 15% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 20% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 25% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 30% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 35% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 40% of the carboxylate groups in the polymer, wherein the magnesium cations are counterions to about 35% of the carboxylate groups in the polymer, and wherein the polymer comprises no more than about 2% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 10% of the carboxylate groups of said polymer, wherein the calcium cations are counterions to about 5% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 10% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 15% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 20% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 25% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 30% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 35% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 40% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 45% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 50% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 55% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 60% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 65% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 70% of the carboxylate groups in the polymer, wherein the magnesium cations are counterions to about 5% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 15% of the carboxylate groups of said polymer, wherein the calcium cations are counterions to about 5% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 10% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 15% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 20% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 25% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 30% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 35% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 40% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 45% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 50% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 55% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 60% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 65% of the carboxylate groups in the polymer, wherein the magnesium cations are counterions to about 10% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 20% of the carboxylate groups of said polymer, wherein the calcium cations are counterions to about 5% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 10% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 15% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 20% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 25% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 30% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 35% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 40% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 45% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 50% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 55% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 60% of the carboxylate groups in the polymer, wherein the magnesium cations are counterions to about 15% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 25% of the carboxylate groups of said polymer, wherein the calcium cations are counterions to about 5% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 10% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 15% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 20% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 25% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 30% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 35% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 40% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 45% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 50% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 55% of the carboxylate groups in the polymer, wherein the magnesium cations are counterions to about 20% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 30% of the carboxylate groups of said polymer, wherein the calcium cations are counterions to about 5% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 10% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 15% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 20% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 25% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 30% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 35% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 40% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 45% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 50% of the carboxylate groups in the polymer, wherein the magnesium cations are counterions to about 25% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 35% of the carboxylate groups of said polymer, wherein the calcium cations are counterions to about 5% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 10% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 15% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 20% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 25% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 30% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 35% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 40% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 45% of the carboxylate groups in the polymer, wherein the magnesium cations are counterions to about 30% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 40% of the carboxylate groups of said polymer, wherein the calcium cations are counterions to about 5% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 45% of the carboxylate groups of said polymer, wherein the calcium cations are counterions to about 10% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 50% of the carboxylate groups of said polymer, wherein the calcium cations are counterions to about 15% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 55% of the carboxylate groups of said polymer, wherein the calcium cations are counterions to about 20% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 60% of the carboxylate groups of said polymer, wherein the calcium cations are counterions to about 25% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 65% of the carboxylate groups of said polymer, wherein the calcium cations are counterions to about 30% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 70% of the carboxylate groups of said polymer, wherein the calcium cations are counterions to about 35% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

In one embodiment, a crosslinked cation-binding polymer comprising monomers containing carboxylate groups and pKa decreasing groups (e.g., fluoroacrylic acid) is a crosslinked polyfluoroacrylic acid, wherein said polymer contains calcium cations and magnesium cations that are counterions to about 75% of the carboxylate groups of said polymer, wherein the calcium cations are counterions to about 40% of the carboxylate groups in the polymer, wherein the magnesium cations are counterions to about 35% of the carboxylate groups in the polymer, and wherein the polymer comprises no more than about 1% sodium cations as counterions to the carboxylate groups of said polymer.

The present disclosure also relates to methods of using the polymers, and compositions, formulations, and/or dosage forms containing the polymers disclosed herein, with or without added base, to treat various diseases and disorders, ion imbalances, and fluid imbalances.

In some embodiments, the disease or disorder is one or more of: heart failure (for example, heart failure with or without chronic kidney disease, diastolic heart failure (heart failure with preserved ejection fraction), heart failure with reduced ejection fraction, cardiomyopathy, or congestive heart failure), a renal insufficiency disease, end stage renal disease, liver cirrhosis, chronic renal insufficiency, chronic kidney disease, fluid overload, fluid maldistribution, edema, pulmonary edema, peripheral edema, angioneurotic edema, lymphedema, nephrotic edema, idiopathic edema, ascites, (for example, general ascites or cirrhotic ascites), chronic diarrhea, excessive interdialytic weight gain, high blood pressure, hyperkalemia, hypernatremia, abnormally high total body sodium, hypercalcemia, tumor lysis syndrome, head trauma, an adrenal disease, Addison's disease, salt-wasting congenital adrenal hyperplasia, hyporeninemic hypoaldosteronism, hypertension, salt-sensitive hypertension, refractory hypertension, hyperparathyroidism, renal tubular disease, rhabdomyolysis, electrical burns, thermal burns, crush injuries, renal failure, acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, aspiration inhalation, neurogenic pulmonary edema, allergic pulmonary edema, high altitude sickness, Adult Respiratory Distress Syndrome, traumatic edema, cardiogenic edema, allergic edema, urticarial edema, acute hemorrhagic edema, papilledema, heatstroke edema, facial edema, eyelid edema, angioedema, cerebral edema, scleral edema, nephritis, nephrosis, nephrotic syndrome, glomerulonephritis, renal vein thrombosis, and/or premenstrual syndrome.

In some embodiments, the disease or disorder is the result of, or is associated with, administration of another drug. For example, compositions and/or dosage forms as disclosed herein are useful in treating an increase in a subject's potassium level when co-administered with a drug known to cause increases in potassium levels. In some embodiments, such a drug is an alpha-adrenergic agonist, a RAAS inhibitor, an ACE inhibitor, an angiotensin II receptor blocker, a beta blocker, an aldosterone antagonist, etc.

### Preparation of Crosslinked Cation-Binding Polymers

Crosslinked cation-binding polymers, including, for example, cation-binding polymer comprising monomers that comprise carboxylic acid groups and pKa decreasing groups, such as polyacrylate polymers, etc., may be prepared by methods known in the art, including by suspension methods (e.g. oil-in-water and water-in-oil methods), aqueous one-phase methods (e.g., Buchholz, F. L. and Graham, A. T., “Modern Superabsorbent Polymer Technology,” John Wiley & Sons (1998)) and by precipitation polymerization (see, e.g., European Patent Application No. EPO459373A2). Polymers with differential properties may be prepared that are useful as therapeutics for different diseases and disorders, including those involving an ion imbalance and/or a fluid imbalance. For example, methods are provided for washing the cross-linked polymer with an acid to replace bound counterions other than hydrogen with hydrogen. The polymeric material, including for example polymeric beads, may be further processed by milling or grinding the polymeric material into particles. A polymer as described herein may contain many carboxylic acid groups, for example, polyacrylic acid, which may be reacted with alkali metals to produce a polycarboxylate, for example, polyacrylate. Many of these polycarboxylates act as superabsorbent polymers and have a saline holding capacity of over twenty times their mass in vitro (e.g., about 40 times its mass) as measured in 0.9% saline solution (e.g., 0.15 M sodium chloride solution) buffered to pH 7 (see, e.g., Examples 8 to 10). Exemplary methods are provided below.

As one who is skilled in the art will understand, the selection of materials for manufacture of a polymer as provided herein, including, monomer, crosslinker, initiator, surfactant, polymerization stabilizer, chelator, catalyst and other excipients will depend on the desired polymer properties and the manufacturing method used to produce this polymer. For example, to make a polymer using a water-in-oil suspension polymerization process or a aqueous polymerization process, monomer, crosslinker, and initiator that are preferentially soluble in water and a surfactant with an HLB appropriate value would be used, for example, acrylic acid, TMPTA, sodium persulfate and Aerosil, respectively. For an oil-in-water suspension polymerization a monomer, crosslinker and initiator that are preferentially soluble in oil and a surfactant with an HLB appropriate value would be used, for example, methyl-2-fluoroacrylate, divinyl benzene, 1,7-ocatadiene, lauroyl peroxide and polyvinylalcohol-co-polyvinylacetate.

1. Materials for Manufacture of Crosslinked Cation-Binding Polymers

Exemplary materials, including monomers, surfactants, crosslinking agents, initiators, bases, acids, water and chelating agents, and catalysts used to manufacture the polymers disclosed herein are provided below.

a. Monomers

Monomers contemplated for use in the present disclosure include those monomers that comprise carboxylic acid groups and pKa decreasing groups such as electron-withdrawing substituents. Such pKa decreasing groups may be located adjacent to the carboxylic acid or carboxylate group, preferably in the alpha or beta position of the acid group. The preferred position for the electron-withdrawing group is attached to the carbon atom alpha to the acid group. Generally, electron-withdrawing substituents are a hydroxyl group, an ether group, an ester group, an acid group, or a halide atom. More preferably, the electron-withdrawing substituent is a halide atom. Most preferably, the electron-withdrawing group is fluoride and is attached to the carbon atom alpha to the acid group, for example, 2-fluoroacrylic acid or its salts, methyl-2-fluoroacrylate, difluoromaleic acid or its salts, or an anhydride thereof. The crosslinked cation-binding polymers as disclosed herein may comprise one or more types of monomer (e.g., acrylic acid, fluoroacrylic acid, or acrylic acid and fluoroacrylic acid).

Exemplary monomers contemplated for use in the present disclosure, include, for example, acrylic acid and its salts, methacrylate, crotonic acid and its salts, tiglinic acid and its salts, -methyl-2-butenoic acid (Z) and its salts, 3-butenoic acid (vinylacetic acid) and its salts, 1-cyclopentene carboxylic acid, and 2-cyclopentene carboxylic acid and their salts; and unsaturated dicarboxylic acids and their salts, such as maleic acid, fumaric acid, itaconic acid, glutaconic acid, and their salts. In other non-limiting embodiments, additional monomers may be contemplated for use.

Further additional monomers are those from which the desired carboxylic acid functionality may be derived by known chemical reactions, for example by hydrolysis. In these embodiments, the monomer, for example, acrylonitrile, acrylamide, methacrylamide, lower alcohol esters of unsaturated, polymerizable carboxylic acids (such as those mentioned in the paragraphs above), or their mixtures, and the like may be polymerized with a suitable crosslinker to an intermediate crosslinked polymer, which is then subjected to chemical reaction (so-called “polymer analogous reaction”) to convert the functional groups of the polymer into carboxylic functionality. For example, ethyl acrylate may be polymerized with a non-hydrolysis-susceptible crosslinker (e.g. tetraallyloxyethane) to form a crosslinked intermediate polymer, which is then subjected to hydrolysis conditions to convert the ester functionality to carboxylic acid functionality by means known in the art. In another example a crosslinked methyl-2-fluoroacrylate polymer can hydrolyzed with base to form the 2-fluoroacrylate polymer. In another example, acrylonitrile is graft polymerized to starch with a crosslinker as necessary to form a crosslinked starch-graft intermediate polymer, which is then treated with aqueous base to hydrolyze the nitrile functionality to carboxylic acid functionality (see, e.g., U.S. Pat. Nos. 3,935,099, 3,991,100, 3,997,484, and 4,134,863).

A variety of fluoridated carboxylate monomers may be useful in the preparation of cation-binding polymers of the present disclosure. Examples of fluoridated carboxylate monomers include, but are not limited to compounds such as (alternative names in parentheses) monocarboxylic acids and their salts; 2-fluoroacrylic acid (2-fluoropropenoic acid), 3-fluoroacrylic acid (3-fluoropropenoic acid), 3-fluoromethacrylic acid (2-methyl-3-fluoropropenoic acid), 3-fluoroethacrylic acid (2-ethyl-3-fluoropropenoic acid), fluorocrotonic acid (trans-2-fluoro-3-methylpropenoic acid, trans-3-fluoro-2-butenoic acid), tiglic acid (trans-2,3-dimethyl-3-fluoropropenoic acid, 2-methyl-3-fluoro-2-butenoic acid), angelic acid (cis-2,3-dimethyl-3-fluoropropenoic acid), 2-fluoro-3,3-dimethylacrylic acid (2-fluoro-3,3-dimethylpropenoic acid), 2-fluoro-3-butenoic acid (2-fluorovinylacetic acid), 2-fluoro-1-cyclopentene carboxylic acid, 2-fluoro-3-cyclopentene carboxylic acid, 2-fluoro-3-propylacrylic acid, trans-2-methyl-3-ethyl-3-fluoroacrylic acid, cis-2-methyl-3-ethyl-3-fluoroacrylic acid, 2-fluoro-3-isopropylacrylic acid, trans-3-methyl-3-ethyl-3-fluoroacrylic acid, cis-2-methyl-3-ethyl-3-fluoroacrylic acid, 2-ethyl-3-fluoro-trans-crotonic acid, 2-ethyl-2-fluoro-cis-2-butenoic acid, 2-isopropyl-3-fluoroacrylic acid, 2-fluoro-3-butylacrylic acid, 2-butyl-3-fluoroacrylic acid, 2-methyl-3-fluoro-2-hexenoic acid, 2-fluoro-3-methyl-3-propylacrylic acid, 3-fluoro-2,3-diethylacrylic acid, 2-fluoro-4-methyl-2-hexenoic acid, 3-fluoro-4-methyl-2-hexenoic acid, 2-fluoro-3,3-diethylacrylic acid, 2-fluoro-3-tert-butylacrylic acid, 2-fluoro-3-methyl-3-isopropylacrylic acid, 2-methyl-3-fluoro-3-isopropylacrylic acid.

Other carboxylate monomers useful in the preparation of cation-binding polymers of the present disclosure include unsaturated dicarboxylic acids and their salts such as; fluoromaleic acid (2-fluoro-butenedioic acid), difluoromaleic acid (cis-difluorobutenedioic acid, cis-2,3-difluorobutenedioic acid), difluorofumaric acid (trans-difluorobutenedioic acid, trans-2,3-difluorobutenedioic acid), 3-fluoroitaconic acid (2-carboxymethyl-3-fluoropropenoic acid, 2-fluoroglutaconic acid; 2-fluoro-2-pentenedioic acid, 2-fluoro-3-carboxymethylpropenoic acid), 3-fluoroglutaconic acid; (3-fluoro-2-pentenedioic acid, 3-fluoro-3-carboxymethylpropenoic acid), fluorocitraconic acid (2-fluoro-3-methylmaleic acid).

Other carboxylate monomers useful in the preparation of cation-binding polymers of the present disclosure include unsaturated dicarboxylic acid anhydrides such as: fluoromaleic anhydride (2-fluoro-butenedioic anhydride), difluoromaleic anhydride (cis-difluorobutenedioic anhydride, cis-2,3-difluorobutenedioic anhydride), fluoroitaconic anhydride (2-carboxymethyl-3-fluoropropenoic anhydride), fluorocitraconic anhydride (2-fluoro-3-methylmaleic anhydride).

Other carboxylate monomers useful in the preparation of cation-binding polymers of the present disclosure include unsaturated monocarboxylic acid esters and amides such as:methyl-2-fluoroacrylate(methyl-2-fluoropropenoate), methyl-3-fluoroacrylate (methyl-3-fluoropropenoate), methyl-3-fluoromethacrylate (methyl-2-methyl-3-fluoropropenoate), methyl-3-fluoroethacrylate (methyl-2-ethyl-3-fluoropropenoate). methyl-2-fluoro-3-methylpropenoate (methyl-2-fluoro-2-butenoate), methyl-2-fluoro-3-ethylpropenoate (methyl-2-fluoro-2-pentenoate), and the analogous ethyl-, propyl-, butyl-esters of the above, 2-fluoroacrylamide(2-fluoropropenamide), 3-fluoroacrylamide(3-fluoropropenamide), 3-fluoromethacrylamide (2-fluoro-3-fluoropropenamide), 3-fluoroethacrylamide (2-ethyl-3-fluoropropenamide), N-methyl-2-fluoroacrylamide (N-methyl-2-fluoropropenamide), N-methyl-2-fluoromethacrylamide (N-methyl-2-fluoro-3-methylpropenamide), N-methyl-3-fluoroethacrylamide (N-methyl-3-fluoro-2-ethylpropenamide), N,N-dimethyl-2-fluoroacrylamide (N,N-dimethyl-2-fluoropropenamide), N,N-dimethyl-3-fluoroacrylamide (N,N-dimethyl-3-fluoropropenamide), N,N-dimethyl-3-fluoromethacrylamide (N,N-dimethyl-2-methyl-3-fluoropropenamide), N,N-dimethyl-3-fluoroethacrylamide (N,N-dimethyl-2-ethyl-3-fluoropropenamide) and analogous N- or N,N-diethyl-, dipropyl-, dibutyl-, or mixed alkyl amides of the above.

Other carboxylate monomers useful in the preparation of cation-binding polymers of the present disclosure include unsaturated dicarboxylic acid esters and amides such as: dimethylfluoromaleate (dimethyl-2-fluorobutendioate), analogous dialkyl esters of the above such as diethyl-, dipropyl-, dibutyl esters, dimethylfluoroitaconate (dimethyl-3-fluoroitaconate; dimethyl-2-carboxymethyl-3-fluoropropenoate), dimethyl-2-fluoroglutaconate (dimethyl-2-fluoro-2-pentenedioate; dimethyl-2-fluoro-3-carboxymethylpropenoate), dimethyl-3-fluoroglutaconate (dimethyl-3-fluoro-2-pentenedioate; dimethyl-3-fluoro-3-carboxymethylpropenoate), dimethyl-fluorocitraconate (dimethyl-2-fluoro-3-methylmaleate) and analogous dialkyl esters such as ethyl-, propyl-, butyl-, etc. of the above.

Preferred carboxylate monomers useful in the preparation of cation-binding polymers of the present disclosure include fluorinated alpha, beta-unsaturated carboxylic acids and derivatives such as the 2-fluoro unsaturated acids. Examples include unsaturated monocarboxylic acids and salts such as: 2-fluoroacrylic acid (2-fluoropropenoic acid), fluorocrotonic acid (trans-2-fluoro-3-methylpropenoic acid, trans-3-fluoro-2-butenoic acid), 2-fluoro-3,3-dimethylacrylic acid (2-fluoro-3,3-dimethylpropenoic acid), 2-fluoro-3-butenoic acid (2-fluorovinylacetic acid), 2-fluoro-1-cyclopentene carboxylic acid, 2-fluoro-3-cyclopentene carboxylic acid, 2-fluoro-3-propylacrylic acid, 2-fluoro-3-isopropylacrylic acid, 2-ethyl-2-fluoro-cis-2-butenoic acid, 2-fluoro-3-butylacrylic acid, 2-fluoro-3-methyl-3-propylacrylic acid, 2-fluoro-4-methyl-2-hexenoic acid, 2-fluoro-3,3-diethylacrylic acid, 2-fluoro-3-tert-butylacrylic acid, 2-fluoro-3-methyl-3-isopropylacrylic acid; unsaturated dicarboxylic acids and their salts such as fluoromaleic acid (2-fluoro-butenedioic acid), difluoromaleic acid (cis-difluorobutenedioic acid, cis-2,3-difluorobutenedioic acid), difluorofumaric acid, trans-difluorobutenedioic acid, trans-2,3-difluorobutenedioic acid), 2-fluoroglutaconic acid (2-fluoro-2-pentenedioic acid; 2-fluoro-3-carboxymethylpropenoic acid), fluorocitraconic acid (2-fluoro-3-methylmaleic acid); unsaturated dicarboxylic acid anhydrides such as fluoromaleic anhydride (2-fluoro-butenedioic anhydride), difluoromaleic anhydride (cis-difluorobutenedioic anhydride, cis-2,3-difluorobutenedioic anhydride), fluorocitraconic anhydride (2-fluoro-3-methylmaleic anhydride); unsaturated monocarboxylic acid esters and amides such as methyl-2-fluoroacrylate (methyl-2-fluoropropenoate), methyl-2-fluorocrotonate (methyl-2-fluoro-3-methylpropenoate, methyl-2-fluoro-2-butenoate), analogous ethyl-, propyl-, butyl-, etc. esters of the above, 2-fluoroacrylamide(2-fluoropropenamide), N-methyl-2-fluoroacrylamide (N-methyl-2-fluoropropenamide), N-methyl-2-fluoro-3-methylpropenamide, N,N-dimethyl-2-fluoroacrylamide (N,N-dimethyl-2-fluoropropenamide) and analogous N- or N,N-diethyl, or mixed methyl/ethyl amides of the above; unsaturated dicarboxylic acid esters and amides, dimethylfluoromaleate (dimethyl-2-fluorobutendioate) and analogous dialkyl esters of above, diethyl-, dipropyl-, dibutyl- etc., dimethyl-2-fl uoroglutaconate (dimethyl-2-fluoro-2-pentenedioate), dimethyl-2-fluoro-3-carboxymethylpropenoate, dimethyl-fluorocitraconate (dimethyl-2-fluoro-3-methylmaleate) and analogous dialkyl esters, e.g. ethyl-, propyl-, butyl-, etc. of the above.

Additional preferred carboxylate monomers useful in the preparation of cation-binding polymers of the present disclosure include 2-fluoro unsaturated acids with no more than one methyl group substituent at the double bond. Such preferred monomers include the unsaturated monocarboxylic acids and salts 2-fluoroacrylic acid (2-fluoropropenoic acid), fluorocrotonic acid (trans-2-fluoro-3-methylpropenoic acid, trans-2-fluoro-2-butenoic acid), 2-fluoro-3-butenoic acid (2-fluorovinylacetic acid); the unsaturated dicarboxylic acids and their salts fluoromaleic acid (2-fluoro-butenedioic acid), difluoromaleic acid (cis-difluorobutenedioic acid, cis-2,3-difluorobutenedioic acid), difluorofumaric acid (trans-difluorobutenedioic acid, trans-2,3-difluorobutenedioic acid), 2-fluoroglutaconic acid (2-fluoro-2-pentenedioic acid; 2-fluoro-3-carboxymethylpropenoic acid), fluorocitraconic acid (2-fluoro-3-methylmaleic acid); the unsaturated dicarboxylic acid anhydrides fluoromaleic anhydride (2-fluoro-butenedioic anhydride), difluoromaleic anhydride (cis-difluorobutenedioic anhydride, cis-2,3-difluorobutenedioic anhydride), fluorocitraconic anhydride (2-fluoro-3-methylmaleic anhydride); the unsaturated monocarboxylic acid esters and amides methyl-2-fluoroacrylate (methyl-2-fluoropropenoate), ethyl-2-fluoroacrylate (ethyl-2-fluoropropenoate), methyl-2-fluoro-3-methylacrylate (2-fluorocrotonate, methyl-2-fluoro-3-methylpropenoate, methyl-2-fluoro-2-butenoate), ethyl-2-fluoro-3-methylacrylate (ethyl-2-fluoro-3-methylpropenoate), 2-fluoroacrylamide(2-fluoropropenamide), N-methyl-2-fluoroacrylamide (N-methyl-2-fluoropropenamide), N-methyl-2-fluoro-3-methylpropenamide, N,N-dimethyl-2-fluoroacrylamide (N,N-dimethyl-2-fluoropropenamide); the unsaturated dicarboxylic acid esters and amides, dimethylfluoromaleate (dimethyl-2-fluorobutendioate), dimethyl-2-fluoroglutaconate (dimethyl-2-fluoro-2-pentenedioate, dimethyl-2-fluoro-3-carboxymethylpropenoate), dimethyl-fluorocitraconate (dimethyl-2-fluoro-3-methylmaleate).

Further additional monomers include those represented by Formula 1 where R₁ and R₂ are each independently hydrogen, alkyl, cycloalkyl, or aryl; R₃ is an optionally protected carboxylic group, R₄ is a hydrogen or electron withdrawing group such a hydroxyl group, an ether group, an ester group, an acid group, or a halide atom.

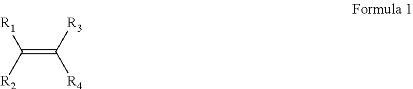

b. Surfactants

Exemplary surfactants contemplated for use in the present disclosure, include, for example, hydrophobic agents that are solids at room temperature, including, for example, hydrophobic silicas (such as Aerosil® or Perform-O-Sil™) and glycolipids (such as polyethylene glycol distearate, polyethylene glycol dioleate, sorbitan monostearate, sorbitan monooleate or octyl glucoside).

Additional surfactants may be selected from the group consisting of anionic, cationic, nonionic, amphoteric, zwitterionic surfactants, or a combination thereof. Anionic surfactants are typically based on sulfate, sulfonate or carboxylate anions. These surfactants include, sodium dodecyl sulfate (SDS), ammonium lauryl sulfate, other alkyl sulfate salts, sodium laureth sulfate (or sodium lauryl ether sulfate (SLES)), N-lauroylsarcosine sodium salt, lauryldimethylamine-oxide (LDAO), ethyltrimethylammoniumbromide (CTAB), bis(2-ethylhexyl)sulfosuccinate sodium salt, alkyl benzene sulfonate, soaps, fatty acid salts, or a combination thereof. Cationic surfactants, for example, contain quaternary ammonium cations. These surfactants are cetyl trimethylammonium bromide (CTAB or hexadecyl trimethyl ammonium bromide), cetylpyridinium chloride (CPC), polyethoxylated tallow amine (POEA), benzalkonium chloride (BAC), benzethonium chloride (BZT), or a combination thereof. Zwitterionic or amphoteric surfactants include dodecyl betaine, dodecyl dimethyl amine oxide, cocamidopropyl betaine, coco ampho glycinate, or a combination thereof. Nonionic surfactants include alkyl poly(ethylene oxide), copolymers of poly(ethylene oxide) and poly(propylene oxide) (commercially called Poloxamers or Poloxamines), alkyl polyglucosides (including octyl glucoside, decyl maltoside, fatty alcohols, cetyl alcohol, oleyl alcohol, cocamide MEA, cocamide DEA), or a combination thereof. Other pharmaceutically acceptable surfactants are well known in the art and are described in McCutcheon's Emulsifiers and Detergents, N. American Edition (2007). Additional surfactants useful, for example in oil-in-water suspensions, which may also act as polymerization reaction stabilizers may be selected from the group consisting of organic polymers and inorganic particulate stabilizers. Examples include polyvinyl alcoholco-vinylacetate and its range of hydrolyzed products, polyvinylacetate, polyvinylpyrolidinone, salts of polyacrylic acid, cellulose ethers, natural gums, or a combination thereof.

c. Crosslinking agents

Exemplary crosslinking agents contemplated for use in the present disclosure, include, for example, crosslinking agents with two or more vinyl groups, each group of which is independently polymerizable, may be used (e.g. divinylarylene, a divinylalkylene, a divinylether, and divinyl amide), allowing for a wide variety in molecular weight, aqueous solubility and/or lipid (e.g., oil) solubility. Crosslinking agents contemplated for use in the present disclosure, include, for example, difunctional arylene, difunctional alkylene, ether- or amide-containing agents, or a combination thereof, and, without limitation, diethyleneglycol diacrylate, diacryl glycerol, triallylamine, tetraallyloxyethane, allylmethacrylate, 1,1,1-trimethylolpropane triacrylate (TMPTA), TMPTA derivatives, divinyl benzene, 1,7-ocatadiene, and divinyl glycol.

Exemplary crosslinkers are one or more compounds having (in one molecule) 2-4 groups selected from the group consisting of CH₂═CHCO—, CH═C(CH₃)CO— and CH₂═CH—CH₂—, for example and without limitation: diacrylates and dimethacrylates of ethylene glycol, glycerol, diethylene glycol, triethylene glycol, tetraethyleneglycol, propylene glycol, dipropyleneglycol, tripropyleneglycol, tetrapropyleneglycol, polyoxyethylene glycols and polyoxypropylene glycols, 1,4-butanediol, 1,5-pentanediol, 1,6-hexanediol, neopentyl glycol, trimethylol propane, and pentaerythritol; triacrylates and trimethacrylates of trimethylolpropane and pentaerythritol; highly ethoxylated trimethylol propane triacrylate; tetracrylate and tetramethacrylate of pentaerythritol; allyl methacrylate, triallylamine, triallylcitrate and tetraallyloxyethane.

In some embodiments, a heat activated crosslinker may be used in the preparation of crosslinked polymers according to the present disclosure. Non-limiting examples of heat-activated crosslinkers include hydroxyl-containing crosslinking agents, amine-containing crosslinking agents, or epoxy-containing crosslinking agents containing at least one functionality suitable to react with a carboxyl group on the polymer and containing at least two functional groups capable of forming covalent bonds with the polymer. Some non-limiting examples of heat-activated crosslinkers suitable for such use is the class of compounds commonly referred to as polyols or polyhydroxy compounds. Some non-limiting examples of polyols include: glycerin, ethylene glycol, diethylene glycol, triethylene glycol, propylene glycol, 1,4-butanediol, 1,5-pentanediol, 1,6-hexanediol, neopentyl glycol, polyglycerin, trimethylolpropane, polyethylene glycol, and polypropylene glycol-polyethylene glycol copolymers. Masked polyols, such as ethyleneglycol diacetate may also be used. Some non-limiting examples of heat-activated crosslinkers containing amine functionality are ethylenediamine, diethylenetriamine, triethylenetetramine, monoethanolamine, and aminoethylethanolamine. Some non-limiting examples of heat-activated crosslinkers containing epoxy functionality are glycidyl acrylate, glycidylmethacrylate, ethyleneglycol and diglycidylether.

In some embodiments, dimodal crosslinkers may be used in the preparation of crosslinked polymers according to the present disclosure. Dimodal crosslinkers contain one or more carboxylic acid-reactive groups and one or more ethylenically unsaturated groups in the same compound. Non-limiting examples of dimodal crosslinkers suitable for use to crosslink polymers according to the present disclosure include: 2-hydroxyethyl(meth)acrylate, polyethylene glycol monomethacrylate, glycidyl methacrylate, allyl glycidyl ether, hydroxypropyl methacrylate, hydroxyethyl methacrylate, and hexapropylene glycol monomethacrylate.

In some embodiments, polyvinyl compounds may be used in the preparation of crosslinked polymers according to the present disclosure. Non-limiting examples of polyvinyl crosslinkers include divinyl compounds or polyvinyl compounds such as: divinyl glycol, divinyl benzene, 1,7-ocatadiene, divinyl toluene, divinyl xylene, divinyl ether, divinyl ketone, trivinyl benzene; unsaturated polyesters that can be obtained by reacting an unsaturated acid such as maleic acid with polyols such as: ethylene glycol, glycerol, diethylene glycol, triethylene glycol, tetraethyleneglycol, propylene glycol, dipropyleneglycol, tripropyleneglycol, tetrapropyleneglycol, polyoxyethylene glycols and polyoxypropylene glycols, 1,4-butanediol, 1,5-pentanediol, 1,6-hexanediol, neopentyl glycol, trimethylol propane, and pentaerythritol; diesters or polyesters of unsaturated mono-or polycarboxylic acids with polyols derived from reaction of C₂-C₁₀ polyhydric alcohols with 2-8 C₂-C₄ alkylene oxide units per hydroxyl group, such as tri methylol propane hexaethoxyl triacrylate; di-methacrylic acid or tri-methacrylic acid esters that can be obtained by reacting polyepoxide with methacrylic acid; bis(meth)acrylamides such as N,N-methylene-bisacrylamide; carbamyl esters that can be obtained by reacting polyisocyanates, such as tolylene diisocyanate, hexamethylene diisocyanate, 4,4′-diphenyl methane diisocyanate; and NCO-containing prepolymers obtained by reacting such diisocyanates with active hydrogen atom-containing compounds with hydroxyl group-containing monomers, such as di-methacrylic acid carbamyl esters obtainable by reacting the above-mentioned diisocyanates with hydroxyethyl(meth)acrylate; di(meth)allyl ethers or poly(meth)allyl ethers of polyols such as alkylene glycols, glycerol, polyalkylene glycols, polyoxyalkylene polyols and carbohydrates such as polyethylene glycol diallyl ether, allylated starch, and allylated cellulose; di-allyl or poly-allyl esters of polycarboxylic acids, such as diallyl phthalate and diallyl adipate; and esters of unsaturated monocarboxylic acids or polycarboxylic acids with mono(meth)allyl ester of polyols, such as allyl methacrylate or (meth)acrylic acid ester of polyethylene glycol monoallyl ether.

In some embodiments, the crosslinker may be one or more compound consistent with the following formula:

R¹-(—(R²O)ₙ—C(O)R³)ₓ,

wherein:
R¹ is a straight-chain or branched-chain C₁-C₁₀ polyalkoxy radical, optionally substituted with one or more oxygen atoms in the backbone, having x valences;
each R² is independently a C₂-C₄ alkylene group;
each R³ is independently a straight-chain or branched-chain C₂-C₁₀ alkenyl moiety;
n is a positive integer from 1-20; and
x is a positive integer from 2-8.

Those skilled in the art will recognize that the amounts of crosslinkers used in the polymerization reactions described herein may be expressed either in terms of weight percent (wt %) or mol percent (mol %). Based on the molecular weights and amounts used, the two measurements can be inter-converted by using appropriate formulas. For example, to convert wt % to mol % for a reaction containing up to any combination of three monomers and crosslinkers, the following formula can be used:

$$A mol \% = \frac{100 ( \frac{A wt \%}{{F}_{A}} )}{\frac{A wt \%}{{F}_{A}} + \frac{B wt \%}{{F}_{B}} + \frac{C wt \%}{{F}_{C}}}$$

where Awt %, Bwt % and Cwt % are the weight percents of components A, B, and C, and F_{A}, F_{B} and F_{C} are the molecular weights of components A, B and C. Similarly, the following formula can be used to convert mol % to wt %:

$$A wt \% = \frac{100 {F}_{A} A mol \%}{{F}_{A} A mol \% + {F}_{B} B mol \% + {F}_{C} C mol \%}$$

where Amol %, Bmol % and Cmol % are the mole percents of components A, B and C. By way of example, for a polymerization reaction containing the monomer methyl-2-fluoroacrylate (MW=104.1) and the crosslinker divinyl benzene (DVB, MW=130.2), 1,7-octadiene (ODE, MW=110.2), or a 1:1 combination of DVB and ODE and a final crosslinker concentration of 5 wt %, the corresponding mol % numbers are 4.04 mol % (for DVB alone), 4.74 mol % (for ODE alone) and 4.39 mol % (for the 1:1 mixture). Similarly, at a final crosslinker concentration of 10 wt %, the corresponding mol % numbers are 8.16 mol % (for DVB alone), 9.50 mol % (for ODE alone) and 8.83 mol % (for the 1:1 mixture); 15 wt % crosslinker corresponds to 12.36 mol % (for DVB alone), 14.29 mol % (for ODE alone) and 13.34 mol % (for the 1:1 mixture); 20 wt % crosslinker corresponds to 16.66 mol % (for DVB alone), 19.10 mol % (for ODE alone) and 17.90 mol % (for the 1:1 mixture).

d. Initiators

Initiation of the polymerization reaction is done by means known in the art. Chemical initiators may be added to the monomers, or the reactions may be initiated by exposure of the monomers to UV-radiation, optionally in the presence of a known UV activator. Generally, the initiators are added to the monomer-containing phase. In some embodiments such as dispersed phase polymerizations, one or more initiators, such as free radical producers, may be added to the dispersed monomer phase just before the monomer phase is mixed with the continuous phase. As will be appreciated by one of skill in the art, the initiator amount and type used in the polymerization reaction depends on oil versus water solubility and whether longer chain lengths are desired. For example, a lower amount of initiator may be used in the polymerization reaction when longer chain lengths are desired. Exemplary initiators contemplated for use in the present disclosure, are described below.

In some embodiments, one of the initiators may be a thermally sensitive compound such as a persulfate, 2,2′-azobis(2-amidino-propane)-dihydrochloride, 2,2′-azobis (2-amidino-propane)-dihydrochloride and/or 2,2′-azobis (4-cyanopentanoic acid). With thermally sensitive initiators polymerization does not begin until an elevated temperature is reached. For persulfates, this temperature is approximately 50 to 55° C. Since the reaction is highly exothermic, vigorous removal of the heat of reaction is required to prevent boiling of the aqueous phase. It is preferred that the reaction mixture be maintained at approximately 65° C. As will be appreciated by one of skill in the art, thermal initiators have the advantage of allowing control of the start of the reaction when the reaction mixture is adequately sparged of oxygen.

In some embodiments, one of the initiators may be a redox pair such as persulfate/bisulfate, persulfate/thiosulfate, persulfate/ascorbate, hydrogen peroxide/ascorbate, sulfur dioxide/tert-butylhydroperoxide, persulfate/erythorbate, tert-butylhydroperoxide/erythorbate and/or tert-butylperbenzoate/erythorbate. These initiators are able to initiate the reaction at room temperature, thereby minimizing the chance of heating the reaction mixture to the boiling point of the aqueous phase as heat is removed through the jacket around the reactor.

Water insoluble, or low water solubility initiators may be preferable, for example lauroyl peroxide, may be prefereable for oil-in-water suspensions.

e. Bases

Exemplary bases contemplated for use in methods of making the crosslinked polymers of the present disclosure include, for example, hydroxides, bicarbonates, or carbonates. Frequently, sodium bases are chosen in the method of making the crosslinked polymers. However, potassium bases, ammonium bases, and bases of other cations, including calcium bases, are contemplated for use in the present disclosure.

f. Acids

Exemplary acids contemplated for use in methods of making the crosslinked polymers of the present disclosure include, for example, hydrochloric acid, acetic acid and phosphoric acid.

g. Water and chelating Agents

The water used in a reaction in the manufacture of the crosslinked polymers of the present disclosure may include, for example, purified water or water from other sources such as city water or well water. If the water used is not purified water, chelating agents may be needed to control metals, e.g., heavy metal ions, such as iron, calcium, and/or magnesium from destroying the initiator. Chelating agents contemplated for use with the present disclosure include, for example, diethylenetriaminepentaacetic acid pentasodium (Versenex™ 80). The amount of chelating agent added to the reaction mixture may be determined by one of skill in the art from a determination of the amount of undesirable metal in the water.

h. Catalysts

A reaction for the manufacture of the polymers disclosed herein may include one or more metals to catalyze the polymerization reaction (e.g., iron).

An exemplary cross-linked cation-binding polymer may be formed by copolymerizing an ethylenically unsaturated carboxylic acid with a multifunctional cross-linking monomer. The acid monomer or polymer may be substantially or partially neutralized with an alkali metal salt such as an oxide, a hydroxide, a carbonate, or a bicarbonate and polymerized by the addition of an initiator. One such exemplary polymer gel is a copolymer of acrylic acid/sodium acrylate and any of a variety of cross-linkers.

2. Manufacture of Crosslinked Cation-Binding Polymers

Cross-linked cation-binding polymers, including cross-linked polyacrylate and/or polyacrylic acid polymers, may be prepared by commonly known methods in the art. In an exemplary method, cation-binding polymer comprising monomers that comprise carboxylic acid groups and pKa decreasing groups may be prepared as a suspension of drops of aqueous solution in a hydrocarbon, for example, a liquid hydrocarbon (e.g., by inverse suspension polymerization).

Cross-linked polyacrylate polymers may be prepared by polymerization of partially neutralized acrylic acid in an aqueous environment where an appropriate cross-linker is present in small quantities. Given that there is an inverse relationship between the amount of fluid the polymer will absorb and the degree of cross-linking of the polymer, it may be desirable to have a low level of cross-linking to obtain a saline holding capacity of at least 20 g/g (e.g. 20 g/g, 30 g/g, 40 g/g, 50 g/g, 60 g/g, 70 g/g, 80 g/g, 90 g/g, or 100 g/g polymer), for use in methods as described herein. However, there is also an inverse relationship between the degree of cross-linking and the percentage of polymer chains that do not cross-link. Non-crosslinked polymer is soluble and may not contribute to the absorbency of the polymer since it dissolves in the fluid. For example, polyacrylates can be designed to have a saline holding capacity of about 35 g/g in pH 7 buffered physiological saline as a compromise between high absorbency and minimal soluble polymer.

Since the amount of reactants used in a polymerization reaction varies depending upon the size of the reactor and other factors, the precise amount of each reactant used in the preparation of crosslinked cation-binding polymer comprising monomers that comprise carboxylic acid groups and pKa decreasing groups, such as polyacrylate, may be determined by one of skill in the art. For example, in a five-hundred gallon reactor, about 190 to 200 pounds (roughly 85 to 90 kg) of acrylic acid may be used while in a three liter reactor 150 to 180 g of acrylic acid may be used. Accordingly, the amount of each reactant used for the preparation of an exemplary cross-linked polyacrylate may be expressed as a weight ratio to acrylic acid. Thus, acrylic acid weight may be taken as 1.0000 and other compounds are represented in relation to this value. Exemplary amounts of reactants used for the preparation of such a cross-linked polyacrylate by an inverse suspension polymerization are presented in Table 1.

TABLE 1
Exemplary Amounts Of Reactants In An Inverse Suspension
Polymerization

| Substance | Low value | High Value |
|---|---|---|
| Acrylic acid | 1.0000 | 1.0000 |
| Water | 0.5000 | 3.0000 |
| Hydrophobic solvent | 1.2000 | 12.0000 |
| Base (expressed as 50% | 0.6600 (60% neutral) | 1.1100 |
| NaOH) | | (100% neutralized) |
| Crosslinker | 0.0030 | 0.0080 |
| Initiator | 0.0005 | 0.0200 |
| Chelating agent | 0.0000 | 0.0050 |
| Surfactant | 0.0050 | 0.0400 |

An exemplary inverse suspension reaction to form a crosslinked polymer may involve preparation of two mixtures (e.g., a hydrophobic mixture and an aqueous mixture) in two different vessels followed by combination of the mixtures to form a reaction mixture. One vessel may be designated as a hydrophobic compound vessel and the other may be designated as an aqueous solution vessel. The hydrophobic compounds may be mixed in a larger vessel that will become a reaction vessel, while an aqueous solution may be prepared in a smaller vessel that may be discharged into the reaction vessel. In an exemplary embodiment, the hydrophobic mixture may contain solvent, surfactant, and crosslinking agent, and the aqueous mixture may contain water, base, monomer (e.g., acrylic acid), initiator, and optional chelating agent.

A hydrophobic solvent may be introduced into the reaction vessel. As will be appreciated by one of skill in the art, a hydrophobic solvent (also referred to herein as the “oil phase”) may be chosen based upon one or more considerations, including, for example, the density and viscosity of the oil phase, the solubility of water in the oil phase, the partitioning of the neutralized and unneutralized ethylenically unsaturated monomers between the oil phase and the aqueous phase, the partitioning of the crosslinker and the initiator between the oil phase and the aqueous phase and/or the boiling point of the oil phase.

Hydrophobic solvents contemplated for use in the present disclosure include, for example, Isopar™ L (isoparaffin fluid), toluene, benzene, dodecane, cyclohexane, n-heptane and/or cumene. Preferably, Isopar™ L is chosen as a hydrophobic solvent due to its low viscosity, high boiling point and low solubility for neutralized monomers such as sodium acrylate and/or potassium acrylate. One of skill in the art will appreciate that a large enough volume of hydrophobic solvent is used to ensure that the aqueous phase is suspended as droplets in the oil rather than the reverse and that the aqueous phase droplets are sufficiently separated to prevent coalescence into large masses of aqueous phase.

One or more surfactants and one or more cross-linkers may be added to the oil (hydrophobic) phase. The oil phase may then be agitated and sparged with an inert gas, such as nitrogen or argon to remove oxygen from the oil phase. It will be appreciated that the amount of surfactant used in the reaction depends on the size of the desired polymer particles and the agitator stir rate. This addition of surfactant is designed to coat the water droplets formed in the initial reaction mixture before the reaction starts. Higher amounts of surfactant and higher agitation rates produce smaller droplets with more total surface area. It will be understood by those of skill in the art that an appropriate choice of cross-linker and initiator may be used to prepare spherical to ellipsoid shaped beads. One of skill in the art will be capable of determining an appropriate cross-linker for the preparation of a specified cross-linked cation-binding polymer. For example, cross-linker choice depends on whether it needs to be hydrophobic or hydrophilic polymer or whether it needs to resist acidic or basic external conditions. An amount of cross-linker depends on how much soluble polymer is permissible and how much saline holding capacity is desired.

An aqueous phase mixture may be prepared in another vessel (e.g., a vessel that is separate from that used to prepare the hydrophobic phase) that contains water. For example, preparation of neutralized or partially neutralized polymer, base and monomer are added to the water. For preparation of non-neutralized (acid form) polymer, monomer is added to the water without base. It will be appreciated by one of skill in the art that the amount of base used in the vessel is determined by the degree of neutralization of the monomer desired. For neutralized or partially neutralized polymer, a degree of neutralization between about 60% and 100% is preferred. Without wishing to be bound by a theory or mechanism, it is believed that one-hundred percent neutralization minimizes the chance of suspension failure, but the highly charged monomer may not react as rapidly and may not pull hydrophobic crosslinkers into the forming polymer. Considerations in choosing the degree of neutralization may be determined by one of skill in the art and include, for example, the effect of monomer charge (e.g., as determined by ionization of the cation from the neutralized molecules) on reaction rate, partitioning of the monomer and neutralized monomer between oil phase and aqueous phase and/or tendency of the aqueous droplets to coalesce during the reaction. The solubilities of sodium acrylate and sodium methacrylate in water are limited and are lower at lower temperatures (e.g., sodium acrylate is soluble at about 45% at 70° C. but less than 40% at 20° C.). This solubility may establish the lower limit of the amount of water needed in the neutralization step. The upper limit of the amount of water may be based on reactor size, amount of oil phase needed to reliably suspend the aqueous phase as droplets and/or the desired amount of polymer produced per batch.

Once base is added to the water, the aqueous phase solution may be cooled to remove the heat released from dilution of the base, and one or more classes of monomers may be added, to react with the base, for example, monomers which will be neutralized by the base. As will be appreciated by one of skill in the art, the monomers will be neutralized to the degree dictated by the amount of base in the reaction. The aqueous phase solution may be kept cool (e.g., below 35 to 40° C.) and preferably around 20° C. to prevent formation of prepolymer strands, dimers and/or possible premature polymerization.

Monomers are dissolved in water at concentrations of 10-70 wt % or 20-40 wt % and polymerization may subsequently be initiated by free radicals in the aqueous phase. Monomers may be polymerized either in the acid form or as a partially neutralized salt. For an inverse suspension process, monomers in the acid form may be less desirable due to high solubility in the oil phase. The amount of water used to dissolve the monomer is minimally set so that all of the monomer (e.g., sodium acrylate) is dissolved in the water rather than crystallizing and maximally set so that there is the smallest volume of reaction mixture possible (to minimize the amount of distillation and allow the maximum yield per batch).

In some embodiments, the reaction is not started immediately after the mixing of the aqueous phase into the oil phase in the final reactor because the aqueous phase still has an excessive amount of oxygen dissolved in the water. It will be appreciated by one of skill in the art that an excessive amount of oxygen may cause poor reactivity and inadequate mixing may prevent the establishment of uniform droplet sizes. Instead, the final reaction mixture is first sparged with an inert gas for ten to sixty minutes after all reagents (except the redox pair if that initiator system is used) have been placed in the reactor. The reaction may be initiated when a low oxygen content (e.g., below 15 ppm) is measured in the inert gas exiting the reactor.

It will be appreciated by those of skill in the art that with acrylate and methacrylate monomers, polymerization begins in the droplets and progresses to a point where coalescence of the particles becomes more likely (the “sticky phase”). It may be necessary that a second addition of surfactant (e.g., appropriately degassed to remove oxygen) be added during this phase or that the agitation rate be increased. For persulfate thermal initiation, this sticky phase may occur at about 50 to 55° C. For redox initiation systems, the need for additional surfactant may be lessened by the initial surface polymerization, but if additional surfactant is needed, it should be added as soon as an exotherm is noted.

The reaction may be continued for four to six hours after the peak exotherm is seen to allow for maximal consumption of the monomer into the polymer. Following the reaction, the polymeric material may be isolated by either transferring the entire reaction mixture to a centrifuge or filter to remove the fluids or by initially distilling the water and some of the oil phase (e.g., frequently as an azeotrope) until no further removal of water is possible and the distillation temperature rises significantly above 100° C., followed by isolating the polymeric material by either centrifugation or filtering. The isolated crosslinked cation-binding polymeric material is then dried to a desired residual moisture content (e.g., less than 5%).

An exemplary cross-linked cation-binding polymer may be formed by copolymerizing an ethylenically unsaturated carboxylic acid with a multifunctional cross-linking monomer. The acid monomer or polymer may be substantially or partially neutralized with an alkali metal salt such as an oxide, a hydroxide, a carbonate, or a bicarbonate and polymerized by the addition of an initiator. One such exemplary polymer gel is a copolymer of acrylic acid/sodium acrylate and any of a variety of cross-linkers.

The reactants for the synthesis of an exemplary cross-linked cation-binding polymer, cross-linked polyacrylate, is provided in Table 2 below. This cross-linked cation-binding polymer may be produced as a one-hundred kilogram batch in a five-hundred gallon vessel.

TABLE 2
List of Components Used in the Manufacture of an Exemplary Crosslinked
Polyacrylate Polymer

| | | Amount/batch |
|---|---|---|
| Component | Function | (kg) |
| Acrylic Acid | Monomer | 88 |
| Water | Solvent | 90 |
| 50% Sodium Hydroxide | Neutralization of acrylic | 79 |
| | acid monomer | |
| Naphtha [petroleum], | Continuous phase for | As needed |
| hydrotreated heavy, | | |
| (Isopar ™ L) | Suspension | |
| Fumed silica (Aerosil R972) | Suspending | 0.9 |
| | agent (Surfactant) | |
| Diethylenetriaminepentaacetic | Control of metal ions in | 0.9 |
| Acid | | |
| Pentasodium (Versenex ™ | reagents, solvents, or | |
| 80) | | |
| Sodium Persulfate | Polymerization initiator | 0.06 |
| Trimethylolpropane | Cross-linking agent | 0.3 |
| Triacrylate, (TMPTA) | | |

In addition to inverse (water-in-oil) suspension methods, cation-binding polymers may be prepared by other methods known in the art (e.g., Buchholz, F. L. and Graham, A. T., “Modern Superabsorbent Polymer Technology,” John Wiley & Sons (1998)), for example by oil-in-water suspensions, aqueous one-phase methods, by precipitation polymerization (see, e.g., European Patent Application No. EPO459373A2), and by crosslinking of soluble polymer using monomers, crosslinkers, surfactants, initiators, neutralizing agents, solvents, suspending agents, and chelators as described herein. For example, cation-binding polymers containing carboxyl groups formed from monomers as described herein may be polymerized to form soluble polymer which may then be crosslinked. In some embodiments, it may be possible to incorporate the crosslinker either into the intermediate polymer, or into the chemically-reacted carboxylic acid functional polymer. For example, crosslinker may be incorporated by copolymerization of the contemplated monomers with a crosslinker as described herein, and then the crosslinked polymer may be converted by, for example hydrolysis, to the desired crosslinked carboxylic acid-functional product. Alternatively, the contemplated additional monomers may be polymerized to a cross-linked polymer then converted to the carboxylic acid-functional polymer; or be polymerized to a non-crosslinked polymer, then converted to the carboxylic acid-functional polymer and subsequently reacted with a suitable crosslinker (for example, one of the heat-activated crosslinkers in the list) to provide the desired, crosslinked, carboxylic acid-functional polymer. Because it is difficult to thoroughly mix a small amount of crosslinker into a high molecular weight polymer, it is desirable to add a heat-activated crosslinker to the monomer-containing reaction mixture, under conditions in which the crosslinker is inactive toward reaction. The polymerization is accomplished in the normal way to yield an uncrosslinked polymer that also contains the molecularly dispersed, heat-activated crosslinker. When it is desired to form the crosslinks, the polymer system is heated to a temperature that is suitable to cause the reaction between polymer functional groups and the crosslinker molecules, thereby crosslinking the polymer.

For example, a 2-fluoroacrylate can be prepared in an oil-in-water suspension as follows. The monomer methyl-2-fluoracryate is the oil phase. Into the oil phase are dissolved the cross-linkers 1,7-octadiene and divinylbenzene, and the initiator lauroyl peroxide. A separate water phase is prepared, dissolving the surfactant/polymerization stabilizer polyvinylalcohol-co-polyvinyl acetate and sodium chloride. The two phases are then mixed, may be purged with nitrogen or other gas to remove oxygen, and stirred at a rate to produce the desired oil-in-water droplet size and heated to about 70° C. and incubated for 5 hours. The solid product can be collected (e.g. by filtration) and optionally washed with water. The polymer beads may be dried (e.g. by vacuum drying or freeze-drying). The polymethyl-2-fluoroacrylate beads may then be hydrolyzed with base to the sodium salt of the 2-fluoroacrylate polymer by suspending the beads in 10 wt % sodium hydroxide and heating and stirring at 95° C. oil for 20 hours. The solid product can then be washed with water and collected by filtration. The polymer beads may then be dried (e.g. by vacuum drying or freeze-drying).

As a further example, co-polymers of 2-fluoroacrylate and methacrylate can be manufactured using the same procedure with a monomer mole ratio of 0.01 to 0.99 of 2-fluoroacrylate to methacrylate by using a mixture of the monomers methyl-2-fluoroacrylate and methacrylate as the oil phase.

3. Preparation of Crosslinked Cation-Binding Polymers with Hydrogen Counterions from Neutralized or Partially Neutralized Crosslinked Cation-Binding Polymers

Partially neutralized or fully neutralized crosslinked cation-binding polymers may be acidified by washing the polymer with acid. Suitable acids contemplated for use with the present disclosure, include, for example, hydrochloric acid, acetic acid and phosphoric acid.

Those skilled in the art will recognize that the replacement of the counterions, including cations such as sodium atoms, by hydrogen atoms can be performed with many different acids and different concentrations of acid. However, care must be taken in choice of acid and concentration to avoid damage to the polymer or the cross-linkers. For instance, nitric and sulfuric acids would be avoided.

Acid-washed crosslinked cation-binding polymers may be additionally rinsed with water and then dried in, for example, a vacuum oven or inert atmosphere until, for example, less than 20% moisture remains (e.g. less than 5%), to produce a substantially free acid form of cross-linked polyacrylic acid. Any particle form of partially or fully neutralized cross-linked cation-binding polymer may be used as the starting point, for example, particles, powders, or bead-form particles, or milled bead-form particles.

Further additional monomers are those from which the desired carboxylic acid functionality may be derived by known chemical reactions, for example by hydrolysis, including acid and base hydrolysis. In these embodiments, the monomer, for example, acrylonitrile, acrylamide, methacrylamide, lower alcohol esters of unsaturated, polymerizable carboxylic acids (such as those mentioned in the paragraph above) or their mixtures, and the like may be polymerized with a suitable crosslinker to an intermediate crosslinked polymer, which is then subjected to chemical reaction (so-called “polymer analogous reaction”) to convert the functional groups of the polymer into carboxylic functionality. For example, ethyl acrylate may be polymerized with a non-hydrolysis-susceptible crosslinker (e.g. tetraallyloxyethane) to form a crosslinked intermediate polymer, which is then subjected to hydrolysis conditions to convert the ester functionality to carboxylic acid functionality by means known in the art. In another example, acrylonitrile is graft polymerized to starch with a crosslinker as necessary to form a crosslinked starch-graft intermediate polymer, which is then treated with aqueous base to hydrolyze the nitrile functionality to carboxylic acid functionality (see, e.g., U.S. Pat. Nos. 3,935,099, 3,991,100, 3,997,484, and 4,134,863).

4. Preparation of Crosslinked Cation-Binding Polymers with Hydrogen Counterions

Acid form cross-linked cation-binding polymers may be prepared by any method known by those skilled in the art (e.g., Buchholz, F. L. and Graham, A. T., “Modern Superabsorbent Polymer Technology,” John Wiley & Sons (1998)), for example by suspension polymerization (e.g. oil-in-water or water-in-oil suspensions), aqueous one-phase polymerization, by precipitation polymerization (see, e.g., European Patent Application No. EPO459373A2), and by crosslinking of soluble polymer.

Crosslinked cation-binding polymers may be prepared from monomers with unneutralized carboxylic acid groups. For example, a crosslinked polyacrylic acid can be prepared from acrylic acid. A monomer solution is prepared in a reactor by dissolving an unsaturated carboxylic acid monomer (e.g., acrylic acid) in water. Optionally, a chelating agent (e.g., Versenex™ 80) may be added to control metal ions and/or a metal added to catalyze the polymerization reaction (e.g., iron). A suitable crosslinking agent (e.g., trimethylolpropane triacrylate) is added to the reactor. The solution may be agitated and oxygen may be removed using nitrogen, argon or by other means known in the art. The temperature of the solution may be adjusted as desired. One or more polymerization initiators may be added to the reactor and the oxygen tension may be reduced or the temperature may be increased to initiate polymerization. The reaction is allowed to proceed through the exothermic heating that occurs during reaction. Reaction heat can be removed and/or controlled as desired by methods known to those skilled in the art. The reaction vessel may then be heated and oxygen tension in the reaction vessel may be kept low to continue the polymerization to low levels of residual monomer. Once the reaction is completed, the polymerization reaction product can be removed from the reactor and the wet polymer may be reduced in size (e.g. by cutting or by methods known to those skilled in the art) into pieces of appropriate size for drying. The polymer pieces can then be dried in a vacuum oven or other equipment known to those skilled in the art. Conditions during drying may be adjusted (e.g. humidity level, rate of drying) so that polymerization and reduction of residual monomer continues during the drying process. After drying, the particles can be separated by size and/or milled and/or sieved to produce the desired particle size. Other examples of the polymerization of aqueous acrylic acid solutions with crosslinkers are disclosed in Buchholz, F. L. and Graham, A. T., “Modern Superabsorbent Polymer Technology,” John Wiley & Sons (1998), U.S. Pat. No. 4,654,039; U.S. Pat. No. 4,295,987; U.S. Pat. No. 5,145,906; and U.S. Pat. No. 4,861,849.

As a further example, a crosslinked polyacrylic acid can be prepared from t-butyl-fluoroacrylate. An oil phase composed of t-butyl-fluoroacrylate, divinyl benzene, 1,7-octadiene and lauroyl peroxide is prepared. An aqueous phase of sodium chloride, polyvinylalcohol (e.g. polyvinylalcohol-co-polyvinylacetate), phosphate buffer and sodium nitrate is prepared. The oil phase is added to the aqueous phase, purged with nitrogen, stirred at a rate to produce the desired oil-in-water droplet size, and heated to about 70° C. After 12 hours the temperature is increased to 85° C. for 2 hours then cooled. The solid product can be collected (e.g. by filtration) and washed with isopropyl alcohol, ethanol and water and dried at room temperature under reduced pressure. The poly 2-fluoroacrylate, t-butyl ester beads may then be hydrolyzed in a 1:1 water:concentrated hydrochloric acid (3 moles acid/mol monomer in the polymer) solution. After addition of the acid the mixture purged with nitrogen, stirred and heating and stirring at 75° C. for 12 hours. The beads can then be washed with isopropyl alcohol, ethanol and water and collected by filtration. The polymer beads may then be dried (e.g. at room temperature under reduced pressure).

Exemplary crosslinked cation-binding polymers, including for example those prepared according to Examples 1-4, generally have a pH 7 buffered saline holding capacity of about 20 g/g or greater, including, for example, greater than about 40 g/g as described in Examples 8 to 10; and contain less than about 5,000 ppm of sodium, less than about 20 ppm of heavy metals, less than about 1000 ppm (e.g., less than about 500 ppm) of residual monomer, less than about 2,000 ppm of residual chloride, and less than about 20 wt % of soluble polymer. Preferably, acidified polymers useful as crosslinked cation-binding polymers prepared according to this disclosure have a saline holding capacity of preferably greater than about 40 g/g, contain less than about 500 ppm of sodium, less than about 20 ppm of heavy metals, less than about 500 ppm of residual monomer, less than about 1,500 ppm of residual chloride, and less than about 10 wt. % of soluble polymer.

The polymer particles may be reduced in size by milling or grinding or other means known to those skilled in the art. Particles of certain size ranges or a particle size distribution may be obtained by means known to those of skill in the art, for example, by sieving through sieves or screens. Sieves may be stacked vertically starting with the smallest pore size at the bottom (largest mesh size) to largest pore size at the top (smallest mesh size). The material is placed on top of the screen and the screens are shaken to allow particles to pass through screens until they are caught on a screen smaller than diameter. The material on each screen will then be smaller than the screen above, but larger than the screen below. For example, particles that pass through an 18 Mesh screen and are caught on a 20 Mesh screen are between 850 and 1000 microns in diameter. Screen mesh and the corresponding maximum particle size allowed to pass through the mesh include, 18 mesh, 1000 microns; 20 mesh, 850 microns; 25 mesh, 710 microns; 30 mesh, 600 microns; 35 mesh, 500 microns, 40 mesh, 425 microns; 45 mesh, 35 microns; 50 mesh, 300 microns; 60 mesh, 250 microns; 70 mesh, 212 microns; 80 mesh, 180 microns; 100 mesh, 150 microns; 120 mesh, 125 microns; 140 mesh, 106 microns; 170 mesh, 90 microns; 200 mesh, 75 microns; 230 mesh, 63 microns; and 270 mesh, 53 microns. Thus particles of varying sizes may be obtained through the use of one or more screens.

In some embodiments, a linear polyol is added to the cation exchange polymer containing an electron withdrawing halide (e.g. 2-fluoroacrylic acid) in a concentration sufficient to reduce the release of fluoride ion from the polymer upon storage as compared to an otherwise identical composition containing no stabilizing polyol at the same temperature and storage time. Performing this step can reduce inorganic fluoride in the composition.

In some embodiments a linear polyol (e.g. sorbitol) is added to the composition containing a crosslinked cation exchange polymer in an amount effective to stabilize the polymer salt, and generally from about 10 wt. % to about 40 wt. % linear polyol based on the total weight of the composition. The linear polyol is preferably a linear sugar (i.e, a linear sugar alcohol). The linear sugar alcohol is preferably selected from the group consisting of D-(+)arabitol, erythritol, glycerol, maltitol, D-mannitol, ribitol, D-sorbitol, xylitol, threitol, galactitol, isomalt, iditol, lactitol and combinations thereof, more preferably selected from the group consisting of D-(+)arabitol, erythritol, glycerol, maltitol, D-mannitol, ribitol, D-sorbitol, xylitol, and combinations thereof, and most preferably selected from the group consisting of xylitol, sorbitol, and a combination thereof. Preferably, the pharmaceutical composition contains from about 15 wt. % to about 35 wt. % stabilizing polyol based on the total weight of the composition. For example, the halide containing polymer (e.g 2-fluoroacrylate) is slurried with an aqueous solution of polyol (e.g., sorbitol), with the slurry containing an excess amount of polyol based on polymer weight. The slurry is maintained under conditions known to those of skill in the art, such as for at least 3 hours and ambient temperature and pressure. The solids are then filtered off and the polymer composition dried to desired moisture content.

5. Preparation of Crosslinked Cation-Binding Polymers with Calcium and/or Magnesium Counterions

Crosslinked, cation-binding polymers with calcium and/or magnesium ions may be prepared by ion-exchange from a partially neutralized crosslinked, cation-binding polymer, by the addition of a calcium and/or magnesium base to an acid form of a crosslinked, cation-binding polymer, by using a calcium or magnesium base to neutralize acrylic acid prior to polymerization, or by other methods known by those skilled in the art. Any particle form of partially or fully neutralized cross-linked cation-binding polymer may be used as the starting point, for example, particles, powders, or bead-form particles, or milled bead-form particles.

Partially neutralized crosslinked cation-binding polymers (e.g. sodium acrylate) may be hydrated and equilibrated with several washes of a salt solution of calcium and/or magnesium (e.g. CaCl₂, MgCl₂) of a concentration high enough to exchange the original counterions on the polymer with the calcium and/or magnesium cations and remove the original counterions from the solution. For polymer more highly neutralized than the desired calcium or magnesium polymer, an appropriate amount of acid may be added with the calcium and/or magnesium salt to bring the neutralization level down to the desired level. Those skilled in the art will recognize that the replacement of the counterions (ion-exchange), including cations such as sodium atoms, by calcium and/or magnesium cations can be performed with many different calcium and/or magnesium salts and salt concentrations. The calcium and/or magnesium crosslinked cation-binding polymers may be additionally rinsed with water or other liquid and then dried in, for example, a vacuum oven or inert atmosphere until, for example, less than 20% (e.g. less than 5%) moisture remains. As a further example, sodium fluoroacrylate beads (for example sodium 2-fluoroacrylate crosslinked with divinylbenzene and I, 7-octadiene) be converted to the calcium salt by exposing the polymer to an excess of aqueous calcium chloride solution to yield the cross-linked (calcium 2-fluoroacrylate)-divinylbenzene-1,7-octadiene copolymer. After the calcium ion exchange, the final product can be washed with ethanol and/or water and dried (e.g. in a vacuum oven or by freeze-drying), until less than 20% (e.g., less than 5%) moisture remains. Similarly a partial magnesium, partial calcium salt of 2-fluoroacrylate-divinylbenzene-1,7-octadiene copolymer can be prepared by reacting the sodium fluoroacrylate beads with a solution of magnesium and calcium salts (e.g. magnesium chloride and calcium chloride).

Crosslinked cation-binding polymers with calcium and/or magnesium counterions may be produced from the acid form of the polymer through the addition of a calcium and/or magnesium base (e.g, CaCO₃, MgO). The base may be added to the polymer as a solid, suspension or solution and the polymer may be hydrated prior to addition of the base. The polymer and base may be stirred and/or heated to facilitate neutralization of the polymer with the base. The calcium and/or magnesium crosslinked cation-binding polymers may be additionally rinsed with ethanol, and/or water, or other liquid, and then dried (e.g. freeze drying, in, for example, a vacuum oven or tray dryer) in an or inert or ambient atmosphere until, for example, less than 20% (e.g. less than 5%) moisture remains.

Crosslinked cation-binding polymers with calcium and magnesium counterions may be prepared from monomers with unneutralized carboxylic acid groups (acid form) and a calcium or magnesium base. A solution of the calcium or magnesium base is prepared by adding the appropriate base slowly to water (e.g. CaCO₃, MgO). Cooling may be used to control the solution temperature. The monomer solution is then prepared in a reactor by adding the monomer (e.g., 2-fluoroacrylic acid, acrylic acid), water and the base solution and then stirring. Alternatively, the solid base can be added to the reactor with the monomer and water. Care should be taken to ensure that the base does not precipitate. Optionally, a chelating agent (e.g., Versenex™ 80) may be added to control metal ions and/or a metal added to catalyze the polymerization reaction (e.g., iron). A suitable crosslinking agent (e.g., trimethylolpropane triacrylate) is added to the reactor and the mixture stirred. Oxygen may be removed using a nitrogen or argon sparge, or by other means known in the art. The temperature of the solution may be adjusted. One or more polymerization initiators (e.g. sodium persulfate) are added to the reactor and the reaction mixture is bubbled with an inert gas (e.g., nitrogen) and agitated until adequate removal of oxygen is achieved. The reaction is then initiated either by reaching an oxygen concentration where a redox couple (e.g., tertiary butylhydroperoxide/thiosulfate, or hydrogen peroxide/erythorbic acid) produces enough radicals that are not quenched by oxygen, or by adding heat to cause a temperature dependent initiator (e.g., sodium persulfate) to produce radicals. The reaction is allowed to proceed through the exothermic heating that occurs during reaction. Reaction heat can be removed and/or controlled as desired by methods known to those skilled in the art. The reaction vessel may then be heated and oxygen tension in the reaction vessel may be kept low to continue the polymerization to low levels of residual monomer. Once the reaction is completed, the polymerization reaction product can be removed from the reactor and the wet polymer may be reduced in size (e.g. by cutting or by methods known to those skilled in the art) into pieces of appropriate size for drying. The polymer pieces can then be dried in a vacuum oven or other equipment known to those skilled in the art. Conditions during drying may be adjusted (e.g. humidity level, rate of drying) so that polymerization and reduction of residual monomer continues during the drying process. Drying conditions may also be adjusted to preserve the polymer integrity for thermally labile polymers. After drying, the particles can be separated by size and/or milled and/or sieved to produce the desired particle size.

### Compositions, Formulations, and Dosage Forms

Compositions, formulations, and/or dosage forms, e.g., pharmaceutical compositions, formulations, and/or dosage forms, are disclosed comprising a cross-linked cation-binding polymer comprising monomers containing carboxylic acid groups and pKa decreasing groups (e.g., a cross-linked polyacrylic acid polymer) and wherein the polymer further comprises calcium cations, wherein the calcium cations are counterions to about 5% to about 75% of the carboxylic acid groups in the polymer (alternately, the polymer comprises calcium counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer). In some embodiments, the polymer comprises calcium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In related embodiments, the calcium cations are counterions to about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% of the carboxylate groups in the polymer. In some embodiments, the polymer is crosslinked with about 0.025 mol % to about 3.0 mol % of crosslinker. These polymers, compositions, formulations, and/or dosage forms may be delivered to a subject, including using a wide variety of routes or modes of administration. In some embodiments, the compositions, formulations, and/or dosage forms as disclosed herein optionally comprise an added base. Preferred routes for administration are oral or intestinal.

Compositions, formulations, and/or dosage forms, e.g., pharmaceutical compositions, formulations, and/or dosage forms, are also disclosed comprising a cross-linked cation-binding polymer comprising monomers containing carboxylic acid groups and pKa decreasing groups (e.g., a cross-linked polyacrylic acid polymer) and wherein the polymer further comprises calcium and magnesium cations, wherein the calcium and magnesium cations are counterions to about 5% to about 75% of the carboxylic acid groups in the polymer (alternately, the polymer comprises calcium and magnesium counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer. In some embodiments, the polymer comprises calcium cations that are counterions to about 5%, about 10%, about 15%, about 20%, about 25%, about 30%, about 35%, about 40%, about 45%, about 50%, about 55%, about 60%, about 65%, about 70%, or about 75% of the carboxylate groups in the polymer. In related embodiments, the calcium and magnesium cations are counterions to about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% of the carboxylate groups in the polymer. In some embodiments, the polymer is crosslinked with about 0.025 mol % to about 3.0 mol % of crosslinker. These polymers, compositions, formulations, and/or dosage forms may be delivered to a subject, including using a wide variety of routes or modes of administration. In some embodiments, the compositions, formulations, and/or dosage forms as disclosed herein optionally comprise an added base. Preferred routes for administration are oral or intestinal.

In some embodiments, a composition, formulation, or dosage form as disclosed herein comprises a polymer that comprises calcium cations as counterions to about 5% to about 75% of the carboxylate groups on the polymer, and sodium cations as counterions to no more than about 5% of the carboxylate groups on the polymer. In such embodiments, the polymer further comprises hydrogen cations (e.g., protons) as counterions to all or substantially all of the carboxylate groups to which calcium and sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 50% to about 60% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, a composition, formulation, or dosage form as disclosed herein comprises a polymer that comprises calcium and magnesium cations as counterions to about 5% to about 75% of the carboxylate groups on the polymer and sodium cations as counterions to no more than about 5% of the carboxylate groups on the polymer, wherein the magnesium cations are counterions to no more than about 35% of the carboxylate groups in the polymer, and further comprises hydrogen cations (e.g., protons) as counterions to all or substantially all of the carboxylate groups to which magnesium or sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium and magnesium cations as counterions to about 50% to about 60% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, a composition, formulation, or dosage form as described herein comprises a crosslinked cation-binding polymer comprising repeat units containing carboxylic acid groups and pKa decreasing groups, and wherein the polymer further comprises calcium cations that are counterions to about 5% to about 75% of the carboxylate groups in the polymer (alternatively, to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer) and further comprises an added base, wherein the base is present in an amount sufficient to provide up to about 0.90 equivalents of base per equivalent of carboxylic acid groups in the polymer. In a related example the composition, formulation, or dosage form contains about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylic acid groups in the polymer.

In some embodiments, a composition, formulation, or dosage form as described herein comprises a crosslinked cation-binding polymer comprising repeat units containing carboxylic acid groups and pKa decreasing groups, and wherein the polymer further comprises calcium and magnesium cations that are counterions to about 5% to about 75% of the carboxylate groups in the polymer (alternatively, to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and further comprises an added base, wherein the base is present in an amount sufficient to provide up to about 0.90 equivalents of base per equivalent of carboxylic acid groups in the polymer. In a related example the composition, formulation, or dosage form contains about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylic acid groups in the polymer.

In some embodiments, a composition, formulation, and/or dosage form as described herein comprises a crosslinked cation-binding polymer comprising carboxylic acid-containing monomers and calcium cations, wherein the calcium cations are present in an amount sufficient to act as counterions to about 5% to about 75% of the carboxylate groups in the polymer, and an amount of added base sufficient to provide up to about 0.90 equivalents of base per equivalent of carboxylate groups in the polymer (alternatively, 0.20 to about 0.25, about 0.20 to about 0.30, about 0.20 to about 0.35, about 0.20 to about 0.40, about 0.20 to about 0.45, about 0.20 to about 0.50, about 0.20 to about 0.55, about 0.20 to about 0.60, about 0.20 to about 0.65, about 0.20 to about 0.70, about 0.20 to about 0.75, about 0.20 to about 0.80, about 0.20 to about 0.85, about 0.20 to about 0.90, about 0.25 to about 0.30, about 0.25 to about 0.35, about 0.25 to about 0.40, about 0.25 to about 0.45, about 0.25 to about 0.50, about 0.25 to about 0.55, about 0.25 to about 0.60, about 0.25 to about 0.65, about 0.25 to about 0.70, about 0.25 to about 0.75, about 0.25 to about 0.80, about 0.25 to about 0.85, about 0.25 to about 0.90, about 0.30 to about 0.35, about 0.30 to about 0.40, about 0.30 to about 0.45, about 0.30 to about 0.50, about 0.30 to about 0.55, about 0.30 to about 0.60, about 0.30 to about 0.65, about 0.30 to about 0.70, about 0.30 to about 0.75, about 0.30 to about 0.80, about 0.30 to about 0.85, about 0.30 to about 0.90, about 0.35 to about 0.40, about 0.35 to about 0.45, about 0.35 to about 0.50, about 0.35 to about 0.55, about 0.35 to about 0.60, about 0.35 to about 0.65, about 0.35 to about 0.70, about 0.35 to about 0.75, about 0.35 to about 0.80, about 0.35 to about 0.85, about 0.35 to about 0.90, about 0.40 to about 0.45, about 0.40 to about 0.50, about 0.40 to about 0.55, about 0.40 to about 0.60, about 0.40 to about 0.65, about 0.40 to about 0.70, about 0.40 to about 0.75, about 0.40 to about 0.80, about 0.40 to about 0.85, about 0.40 to about 0.90, about 0.45 to about 0.50, about 0.45 to about 0.55, about 0.45 to about 0.60, about 0.45 to about 0.65, about 0.45 to about 0.70, about 0.45 to about 0.75, about 0.45 to about 0.80, about 0.45 to about 0.85, about 0.45 to about 0.90, about 0.50 to about 0.55, about 0.50 to about 0.60, about 0.50 to about 0.65, about 0.50 to about 0.70, about 0.50 to about 0.75, about 0.50 to about 0.80, about 0.50 to about 0.85, about 0.50 to about 0.90, about 0.55 to about 0.60, about 0.55 to about 0.65, about 0.55 to about 0.70, about 0.55 to about 0.75, about 0.55 to about 0.80, about 0.55 to about 0.85, about 0.55 to about 0.90, about 0.60 to about 0.65, about 0.60 to about 0.70, about 0.60 to about 0.75, about 0.60 to about 0.80, about 0.60 to about 0.85, about 0.60 to about 0.90, about 0.65 to about 0.70, about 0.65 to about 0.75, about 0.65 to about 0.80, about 0.65 to about 0.85, about 0.65 to about 0.90, about 0.70 to about 0.75, about 0.70 to about 0.80, about 0.70 to about 0.85, about 0.70 to about 0.90, about 0.75 to about 0.80, about 0.75 to about 0.85, about 0.75 to about 0.90, about 0.80 to about 0.85, about 0.80 to about 0.90, about or 0.85 to about 0.90 equivalents of base per equivalent of carboxylate groups in the polymer).

In some embodiments, a composition, formulation, and/or dosage form as described herein comprises a crosslinked cation-binding polymer comprising carboxylic acid-containing monomers and calcium and magnesium cations, wherein the calcium and magnesium cations are present in an amount sufficient to act as counterions to about 5% to about 75% of the carboxylate groups in the polymer, wherein the magnesium cations are counterions to no more than about 35% of the carboxylate groups in the polymer, and an amount of added base sufficient to provide up to about 0.90 equivalents of base per equivalent of carboxylate groups in the polymer (alternatively, about 0.20 to about 0.25, about 0.20 to about 0.30, about 0.20 to about 0.35, about 0.20 to about 0.40, about 0.20 to about 0.45, about 0.20 to about 0.50, about 0.20 to about 0.55, about 0.20 to about 0.60, about 0.20 to about 0.65, about 0.20 to about 0.70, about 0.20 to about 0.75, about 0.20 to about 0.80, about 0.20 to about 0.85, about 0.20 to about 0.90, about 0.25 to about 0.30, about 0.25 to about 0.35, about 0.25 to about 0.40, about 0.25 to about 0.45, about 0.25 to about 0.50, about 0.25 to about 0.55, about 0.25 to about 0.60, about 0.25 to about 0.65, about 0.25 to about 0.70, about 0.25 to about 0.75, about 0.25 to about 0.80, about 0.25 to about 0.85, about 0.25 to about 0.90, about 0.30 to about 0.35, about 0.30 to about 0.40, about 0.30 to about 0.45, about 0.30 to about 0.50, about 0.30 to about 0.55, about 0.30 to about 0.60, about 0.30 to about 0.65, about 0.30 to about 0.70, about 0.30 to about 0.75, about 0.30 to about 0.80, about 0.30 to about 0.85, about 0.30 to about 0.90, about 0.35 to about 0.40, about 0.35 to about 0.45, about 0.35 to about 0.50, about 0.35 to about 0.55, about 0.35 to about 0.60, about 0.35 to about 0.65, about 0.35 to about 0.70, about 0.35 to about 0.75, about 0.35 to about 0.80, about 0.35 to about 0.85, about 0.35 to about 0.90, about 0.40 to about 0.45, about 0.40 to about 0.50, about 0.40 to about 0.55, about 0.40 to about 0.60, about 0.40 to about 0.65, about 0.40 to about 0.70, about 0.40 to about 0.75, about 0.40 to about 0.80, about 0.40 to about 0.85, about 0.40 to about 0.90, about 0.45 to about 0.50, about 0.45 to about 0.55, about 0.45 to about 0.60, about 0.45 to about 0.65, about 0.45 to about 0.70, about 0.45 to about 0.75, about 0.45 to about 0.80, about 0.45 to about 0.85, about 0.45 to about 0.90, about 0.50 to about 0.55, about 0.50 to about 0.60, about 0.50 to about 0.65, about 0.50 to about 0.70, about 0.50 to about 0.75, about 0.50 to about 0.80, about 0.50 to about 0.85, about 0.50 to about 0.90, about 0.55 to about 0.60, about 0.55 to about 0.65, about 0.55 to about 0.70, about 0.55 to about 0.75, about 0.55 to about 0.80, about 0.55 to about 0.85, about 0.55 to about 0.90, about 0.60 to about 0.65, about 0.60 to about 0.70, about 0.60 to about 0.75, about 0.60 to about 0.80, about 0.60 to about 0.85, about 0.60 to about 0.90, about 0.65 to about 0.70, about 0.65 to about 0.75, about 0.65 to about 0.80, about 0.65 to about 0.85, about 0.65 to about 0.90, about 0.70 to about 0.75, about 0.70 to about 0.80, about 0.70 to about 0.85, about 0.70 to about 0.90, about 0.75 to about 0.80, about 0.75 to about 0.85, about 0.75 to about 0.90, about 0.80 to about 0.85, about 0.80 to about 0.90, about or 0.85 to about 0.90 equivalents of base per equivalent of carboxylate groups in the polymer).

The polymers in any of the compositions, formulations, and/or dosage forms disclosed herein may comprise sodium cations which (if present) are counterions to no more than about 5%, 4%, 3%, 2%, 1%, or 0.5% of the carboxylate groups in the polymer.

In some embodiments, the polymers disclosed herein for inclusion in a composition, formulation, or dosage form, e.g., for administration to an individual, e.g., for use in methods of treatment disclosed herein, are individual particles or particles agglomerated to form a larger particle (for example, flocculated particles), and have a diameter of about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 20 microns to about 125 microns, about 20 microns to about 150 microns, about 20 microns to about 200 microns, about 35 microns to about 150 microns, about 35 microns to about 125 microns, about 50 microns to about 125 microns, about 50 microns to about 200 microns, about 50 microns to about 1000 microns, about 500 microns to about 1000 microns, about 1000 to about 5000 microns, or about 5000 microns to about 10,000 microns). In some embodiments, the particles or agglomerated particles have a diameter of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 7000, about 7500, about 8000, about 8500, about 9000, about 9500, or about 10,000 microns.

In some embodiments, the crosslinked cation-binding polymer disclosed herein for inclusion in a composition, formulation, or dosage form, e.g., for administration to an individual, e.g., for use in methods of treatment disclosed herein is a crosslinked acrylic acid polymer. For example, the polymer may be a acrylic acid polymer crosslinked with about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker or alternatively from about 0.025 mol.% to about 3.0 mol.% including, for example, from about 0.025 mol.% to about 0.3 mol.%, and for example, may comprise an in vitro saline absorption capacity (e.g., saline holding capacity) of at least about 20 times its weight (e.g., at least about 20 grams of saline per gram of polymer, or “g/g”), at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked acrylic acid polymer is in the form of individual particles or particles that are agglomerated (for example, flocculated) to form a larger particle, wherein the diameter of individual particles or agglomerated particles is about 1 micron to about 10,000 microns (alternatively, about 1 micron to about 10 microns, about 1 micron to about 50 microns, about 10 microns to about 50 microns, about 10 microns to about 200 microns, about 50 microns to about 100 microns, about 20 microns to about 125 microns, about 20 microns to about 150 microns, about 20 microns to about 200 microns, about 35 microns to about 150 microns, about 35 microns to about 125 microns, about 50 microns to about 125 microns, about 50 microns to about 200 microns, about 50 microns to about 1000 microns, about 500 microns to about 1000 microns, about 1000 to about 5000 microns, or about 5000 microns to about 10,000 microns). For example, a polymer, including a acrylic acid polymer, may have a particle size of about 212 microns to about 500 microns, a particle size from about 75 microns to about 150 microns, or a particle size of about 75 microns or less. In some embodiments, the crosslinked acrylic acid polymer comprises individual particles or particles that are agglomerated (for example, flocculated) to form a larger particle, wherein the individual or agglomerated particle diameter is about 100 microns. In some embodiments, no less than about 70% of the polymer has a particle size of about 212 microns to about 500 microns, a particle size from about 75 microns to about 150 microns, or a particle size of about 75 microns or less. In one embodiment, the acrylic acid polymer is in the form of small particles that flocculate to form agglomerated particles with a diameter of about 1 micron to about 10 microns or about 0.1 microns to about 20 microns. In one embodiment, the acrylic acid polymer is in the form of small particles that flocculate to form agglomerated particles with a diameter of about 1 micron to about 10 microns.

In some embodiments, the present disclosure is also directed to pharmaceutical compositions comprising a crosslinked cation-binding polymer comprising monomers containing carboxylic acids and a pKa-reducing group such as an electron-withdrawing substituent including a halide atom such as F (e.g., fluoroacrylic acid or methyl-fluoroacrylic acid monomers) and an optional polyol. When the composition comprises a polyol, it may be present in an amount sufficient to reduce the release of the pKa-reducing group such as a fluoride ion from the cation-binding polymer during storage. In some embodiments, the pharmaceutical compositions of this disclosure additionally comprise water. When the composition comprises water, it also may be present in an amount sufficient to reduce or assist in the reduction of the release of the pKa-reducing group such as a fluoride ion from the cation-binding polymer during storage. A crosslinked cation-binding polymer comprising a fluoro group and a carboxylic acid group may be the product of the polymerization of optionally two or optionally three, different monomer units. For example, one monomer may comprise a fluoro group and a carboxylic acid group and the other monomer is a difunctional arylene monomer or a difunctional alkylene, ether- or amide-containing monomer, or a combination thereof. Compositions comprising such polymers may be useful to bind potassium in the gastrointestinal tract. In some embodiments, the linear polyol is a linear sugar alcohol. Increased efficacy and/or tolerability in different dosing regimens may be seen as compared to compositions without the linear polyol, and optionally including water.

A linear polyol may be optionally added to the compositions containing a crosslinked cation-binding polymers in an amount effective to stabilize the polymer, and generally from about 10 wt. % to about 40 wt. % linear polyol based on the total weight of the composition. The linear polyol may be a linear sugar (e.g., a linear sugar alcohol). Useful linear sugar alcohols may include D-(+)arabitol, erythritol, glycerol, maltitol, D-mannitol, ribitol, D-sorbitol, xylitol, threitol, galactitol, isomalt, iditol, lactitol and combinations thereof, wherein D-(+)arabitol, erythritol, glycerol, maltitol, D-mannitol, ribitol, D-sorbitol, xylitol, and combinations thereof may be preferred, and xylitol, sorbitol, and a combination thereof may be more preferred. Compositions comprising the polymers may contain from about 15 wt. % to about 35 wt. % stabilizing polyol based on the total weight of the composition. In some embodiments, the linear polyol concentration is sufficient to reduce the release of fluoride ion from the cation-binding polymer upon storage as compared to an otherwise identical composition containing no stabilizing polyol at the same temperature and storage time.

The moisture content of the composition may be balanced with the stabilizing linear polyol to provide a stabilized polymer within the composition. For example, as the moisture content of the composition increases, the concentration of polyol may be decreased. However, the moisture content should not rise so high as to prevent the composition from being free flowing during manufacturing or packaging operations. For example, the moisture content may range from about 1 to about 30 weight percent based on the total weight of the composition, or alternatively from about 10 to about 25 wt. % based on the total weight of the composition of polymer, linear polyol and water. In one specific case, the pharmaceutical composition comprises about 10-40 wt. % linear polyol, about 1-30 wt. % water and the remainder crosslinked cation-binding polymer, with the weight percents based on the total weight of linear polyol, water and polymer. In some embodiments, compositions comprise about 15 wt. % to about 35 wt. % linear polyol, about 10 wt. % to about 25 wt % water and the remainder crosslinked cation-binding polymer, with the weight percents based on the total weight of linear polyol, water and polymer. In other embodiments, the compositions comprise from about 10 wt. % to about 40 wt. % linear polyol and the remainder crosslinked cation-binding polymer, with the weight percents based on the total weight of linear polyol and polymer.

The moisture content may be measured in a manner known to those of skill in the art. For example, moisture content in the composition may be determined by several methods, such as thermogravimetric method via a moisture analyzer during in-process manufacturing or measuring loss on drying in accordance with US Pharmacopeia (USP)<731>. The operating condition for the thermogravimetric method via moisture analyzer may be 0.3 g of polymer composition heated at about 160° C. for about 45 min. Alternatively, the operating condition for the USP<731> method may be 1.5-2 g of polymer composition heated to about 130° C. for about 16 hours under 25-35 mbar vacuum.

From a stabilizing viewpoint, the concentration of inorganic fluoride (e.g., from fluoride ion) in the composition may be less than about 1000 ppm, less than about 500 ppm or less than about 300 ppm under typical storage conditions. For example, the concentration of inorganic fluoride in the composition may be less than about 1000 ppm after storage at accelerated storage conditions (about 40° C. for about 6 weeks), less than about 500 ppm after room temperature storage (about 25° C. for about 6 weeks), or less than about 300 ppm after refrigerated storage (about 5° C. for about 6 weeks). Additionally, the concentration of inorganic fluoride in the composition may be generally 50% less or 75% less than the concentration of inorganic fluoride in the otherwise identical composition containing no stabilizing polyol at the same temperature and storage time.

In some embodiments in which a composition, formulation, or dosage form comprises an optionally added base, the optionally added base component is one or more of: an alkali metal hydroxide, an alkali metal acetate, an alkali metal carbonate, an alkali metal bicarbonate, an alkali metal oxide, an alkali earth metal hydroxide, an alkali earth metal acetate, an alkali earth metal carbonate, an alkali earth metal bicarbonate, an alkali earth metal oxide, an organic base, choline, lysine, arginine, histidine, an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, calcium bicarbonate, calcium carbonate, calcium oxide, calcium hydroxide, magnesium oxide, magnesium carbonate, magnesium hydrochloride, sodium bicarbonate, and potassium citrate, or a combination thereof. In one embodiment, the added base is calcium carbonate.

In some embodiments, a composition, formulation, or dosage form disclosed herein comprises a polyacrylate polymer that comprises calcium counterions to about 5% to about 75% of the carboxylate groups in the polymer, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% of the carboxylate groups in the polymer, and wherein sodium cations (if present) are counterions to no more than about 5% of the carboxylate groups in the polyacrylate polymer. In some embodiments, the composition, formulation, or dosage form additionally comprises added base, for example, calcium carbonate base, present in an amount to provide up to about 0.90 equivalents of base per carboxylate group on the polyacrylate polymer, for example, about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylate groups in the polyacrylate polymer.

In some embodiments, a composition, formulation, or dosage form disclosed herein comprises a polyacrylate polymer that comprises calcium and magnesium counterions to about 5% to about 75% of the carboxylate groups in the polymer, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75% of the carboxylate groups in the polymer, wherein magnesium cations are counterions to no more than about 35% of the carboxylate groups in the polymer, and wherein sodium cations (if present) are counterions to no more than about 5% of the carboxylate groups in the polyacrylate polymer. In some embodiments, the composition, formulation, or dosage form additionally comprises added base, for example, calcium carbonate base, present in an amount to provide up to about 0.90 equivalents of base per carboxylate group on the polyacrylate polymer, for example, about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylate groups in the polyacrylate polymer.

In some embodiments, the above compositions, formulations, and/or dosage forms additionally comprise one or more excipients, carriers, or diluents. Compositions for use in accordance with the present disclosure may be formulated in conventional manner using one or more physiologically acceptable carriers comprising excipients, diluents, and auxiliaries which facilitate processing of the polymer into preparations which may be used pharmaceutically. Proper formulation is dependent upon the route of administration chosen. Such compositions may contain a therapeutically effective amount of polymer and may include a pharmaceutically acceptable carrier, excipient, and/or diluent. Pharmaceutically acceptable carriers, additives, and formulation ingredients include those approved by a regulatory agency of the Federal or a state government or listed in the U.S. Pharmacopeia or other generally recognized pharmacopeia for use in animals, and more particularly, in humans. Carriers can include an active ingredient in which the disclosed compositions are administered

In some embodiments, dosage forms according to the present disclosure comprise a crosslinked cation-binding polymer comprising monomers that comprise carboxylic acid groups and pKa decreasing groups; and calcium cations or calcium and magnesium cations, wherein the calcium cations or calcium and magnesium cations are counterions to about 5% to about 75% of the carboxylate groups in the polymer. In some embodiments, the dosage form comprises an amount of the base sufficient to provide from about 0.20 to about 0.90 equivalents of base per equivalent of carboxylic acid groups on the polymer. In some embodiments, the dosage form includes an amount of base sufficient to ameliorate or prevent acidosis in a subject to whom the polymer is administered. Monomers, crosslinkers, and bases useful in the preparation of the crosslinked cation-binding polymers as described above are also suitable for the dosage forms of the present disclosure.

In some embodiments, the dosage form is a tablet, a chewable tablet, a capsule, a suspension, an oral suspension, a powder, a gel block, a gel pack, a confection, a chocolate bar, a pudding, a flavored bar, or a sachet. In some embodiments, the dosage form contains an amount of a composition described herein to provide from about 1 g to about 30 g or about 100 g of the cation-binding polymer. In some embodiments, the dosage form contains an amount of a composition described herein to provide about 15 g to about 25 g, about 15 g to about 30 g, or about 25 g to about 30 g of the cation-binding polymer. For example and without limitation, the dosage form may include an amount of the composition to provide about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, or about 30 g, about 35 g, about 40 g, about 45 g, about 50 g, about 55 g, about 60 g, about 65 g, about 70 g, about 75 g, about 80 g, about 85 g, about 90 g, about 95 g, or about 100 g, or more of the cation-binding polymer. Regardless of the amount of polymer present in the dosage form, the dosage forms of the present disclosure may include up to about 0.90 equivalents of base, for example, a pharmaceutically and/or physiologically acceptable base, per equivalent of carboxylate groups in the polymer, for example, about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.50 equivalents, about 0.55 equivalents, about 0.60 equivalents, about 0.65 equivalents, about 0.70 equivalents, about 0.75 equivalents, about 0.80 equivalents, about 0.85 equivalents, or about 0.90 equivalents of base per equivalent of carboxylic acid groups in the polymer. In some embodiments, the base is present in an amount sufficient to provide from about 0.20 to about 0.25, about 0.20 to about 0.30, about 0.20 to about 0.35, about 0.20 to about 0.40, about 0.20 to about 0.45, about 0.20 to about 0.50, about 0.20 to about 0.55, about 0.20 to about 0.60, about 0.20 to about 0.65, about 0.20 to about 0.70, about 0.20 to about 0.75, about 0.20 to about 0.80, about 0.20 to about 0.85, about 0.20 to about 0.90, about 0.25 to about 0.30, about 0.25 to about 0.35, about 0.25 to about 0.40, about 0.25 to about 0.45, about 0.25 to about 0.50, about 0.25 to about 0.55, about 0.25 to about 0.60, about 0.25 to about 0.65, about 0.25 to about 0.70, about 0.25 to about 0.75, about 0.25 to about 0.80, about 0.25 to about 0.85, about 0.25 to about 0.90, about 0.30 to about 0.35, about 0.30 to about 0.40, about 0.30 to about 0.45, about 0.30 to about 0.50, about 0.30 to about 0.55, about 0.30 to about 0.60, about 0.30 to about 0.65, about 0.30 to about 0.70, about 0.30 to about 0.75, about 0.30 to about 0.80, about 0.30 to about 0.85, about 0.30 to about 0.90, about 0.35 to about 0.40, about 0.35 to about 0.45, about 0.35 to about 0.50, about 0.35 to about 0.55, about 0.35 to about 0.60, about 0.35 to about 0.65, about 0.35 to about 0.70, about 0.35 to about 0.75, about 0.35 to about 0.80, about 0.35 to about 0.85, about 0.35 to about 0.90, about 0.40 to about 0.45, about 0.40 to about 0.50, about 0.40 to about 0.55, about 0.40 to about 0.60, about 0.40 to about 0.65, about 0.40 to about 0.70, about 0.40 to about 0.75, about 0.40 to about 0.80, about 0.40 to about 0.85, about 0.40 to about 0.90, about 0.45 to about 0.50, about 0.45 to about 0.55, about 0.45 to about 0.60, about 0.45 to about 0.65, about 0.45 to about 0.70, about 0.45 to about 0.75, about 0.45 to about 0.80, about 0.45 to about 0.85, about 0.45 to about 0.90, about 0.50 to about 0.55, about 0.50 to about 0.60, about 0.50 to about 0.65, about 0.50 to about 0.70, about 0.50 to about 0.75, about 0.50 to about 0.80, about 0.50 to about 0.85, about 0.50 to about 0.90, about 0.55 to about 0.60, about 0.55 to about 0.65, about 0.55 to about 0.70, about 0.55 to about 0.75, about 0.55 to about 0.80, about 0.55 to about 0.85, about 0.55 to about 0.90, about 0.60 to about 0.65, about 0.60 to about 0.70, about 0.60 to about 0.75, about 0.60 to about 0.80, about 0.60 to about 0.85, about 0.60 to about 0.90, about 0.65 to about 0.70, about 0.65 to about 0.75, about 0.65 to about 0.80, about 0.65 to about 0.85, about 0.65 to about 0.90, about 0.70 to about 0.75, about 0.70 to about 0.80, about 0.70 to about 0.85, about 0.70 to about 0.90, about 0.75 to about 0.80, about 0.75 to about 0.85, about 0.75 to about 0.90, about 0.80 to about 0.85, about 0.80 to about 0.90, about or 0.85 to about 0.90 equivalents of base per equivalent of carboxylate groups in the polymer.

In some embodiments, the base component of the dosage form is one or more of: an alkali metal hydroxide, an alkali metal acetate, an alkali metal carbonate, an alkali metal bicarbonate, an alkali metal oxide, an alkali earth metal hydroxide, an alkali earth metal acetate, an alkali earth metal carbonate, an alkali earth metal bicarbonate, an alkali earth metal oxide, an organic base, choline, lysine, arginine, histidine, an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, calcium bicarbonate, calcium carbonate, calcium oxide, calcium hydroxide, magnesium oxide, magnesium carbonate, magnesium hydrochloride, sodium bicarbonate, and potassium citrate, or a combination thereof.

For oral administration, the disclosed polymers and/or compositions may be formulated readily by combining them with pharmaceutically acceptable carriers well known in the art. Such carriers enable the compositions of the disclosure to be formulated, preferably in capsules but alternatively in other dosage forms such as tablets, chewable tablets, pills, dragees, capsules, liquids, gel packs, gel blocks, syrups, slurries, suspensions, wafers, sachets, powders, dissolving tablets and the like, for oral ingestion by a subject, including a subject to be treated. In some embodiments, the compositions or capsules containing the compositions have an enteric coating. In other embodiments, the compositions or capsules containing the compositions do not have an enteric coating.

In some embodiments, a composition, formulation, and/or dosage form as described herein comprises a base and an unneutralized crosslinked polycarboxylate polymer as described herein (e.g., a cross-linked polyacrylic acid polymer), wherein the polymer further comprises calcium cations, wherein the calcium cations are counterions to about 5% to about 75% of the carboxylic acid groups in the polymer (alternately, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and is administered in an amount sufficient to provide from about 0.01 moles of carboxylate groups to about 0.5 moles or about 0.56 moles of carboxylate groups to the subject per day, for example, about 0.01 moles, about 0.02 moles, about 0.03 moles, about 0.04 moles, about 0.05 moles, about 0.06 moles, about 0.07 moles, about 0.08 moles, about 0.09 moles, about 0.1 moles, about 0.11 moles, about 0.12 moles, about 0.13 moles, about 0.14 moles, about 0.15 moles, about 0.16 moles, about 0.17 moles, about 0.18 moles, about 0.19 moles, about 0.2 moles, about 0.21 moles, about 0.22 moles, about 0.23 moles, about 0.24 moles, about 0.25 moles, about 0.26 moles, about 0.27 moles, about 0.28 moles, about 0.29 moles, about 0.3 moles, about 0.31 moles, about 0.32 moles, about 0.33 moles, about 0.34 moles, about 0.35 moles, about 0.36 moles, about 0.37 moles, about 0.38 moles, about 0.39 moles, about 0.4 moles, about 0.41 moles, about 0.42 moles, about 0.43 moles, about 0.44 moles, about 0.45 moles, about 0.46 moles, about 0.47 moles, about 0.48 moles, about 0.49 moles, or about 0.5 moles of carboxylate groups to the subject per day. In a preferred embodiment, the dosage forms are administered in an amount sufficient to provide from about 0.01 to about 0.25 moles of carboxylate groups per day. In a more preferred embodiment, the dosage forms are administered in an amount sufficient to provide from about 0.1 to about 0.25 moles of carboxylate groups per day.

In some embodiments, a composition, formulation, and/or dosage form as described herein comprises a base and a crosslinked polycarboxylate polymer as described herein (e.g., a cross-linked polyacrylic acid polymer), wherein the polymer further comprises calcium and magnesium cations, wherein the calcium and magnesium cations are counterions to about 5% to about 75% of the carboxylic acid groups in the polymer (alternately, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and is administered in an amount sufficient to provide from about 0.01 moles of carboxylate groups to about 0.5 moles or about 0.56 moles of carboxylate groups to the subject per day, for example, about 0.01 moles, about 0.02 moles, about 0.03 moles, about 0.04 moles, about 0.05 moles, about 0.06 moles, about 0.07 moles, about 0.08 moles, about 0.09 moles, about 0.1 moles, about 0.11 moles, about 0.12 moles, about 0.13 moles, about 0.14 moles, about 0.15 moles, about 0.16 moles, about 0.17 moles, about 0.18 moles, about 0.19 moles, about 0.2 moles, about 0.21 moles, about 0.22 moles, about 0.23 moles, about 0.24 moles, about 0.25 moles, about 0.26 moles, about 0.27 moles, about 0.28 moles, about 0.29 moles, about 0.3 moles, about 0.31 moles, about 0.32 moles, about 0.33 moles, about 0.34 moles, about 0.35 moles, about 0.36 moles, about 0.37 moles, about 0.38 moles, about 0.39 moles, about 0.4 moles, about 0.41 moles, about 0.42 moles, about 0.43 moles, about 0.44 moles, about 0.45 moles, about 0.46 moles, about 0.47 moles, about 0.48 moles, about 0.49 moles, or about 0.5 moles of carboxylate groups to the subject per day. In a preferred embodiment, the dosage forms are administered in an amount sufficient to provide from about 0.01 to about 0.25 moles of carboxylate groups per day. In a more preferred embodiment, the dosage forms are administered in an amount sufficient to provide from about 0.1 to about 0.25 moles of carboxylate groups per day.

In some embodiments, the dosage form comprises a base and an unneutralized crosslinked acrylic acid polymer as described herein, (e.g., a cross-linked polyacrylic acid polymer), wherein the polymer further comprises calcium cations, wherein the calcium cations are counterions to about 5% to about 75% of the carboxylic acid groups in the polymer (alternately, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and is administered in an amount sufficient to provide from about 1 g to about 30 g or about 1 g or up to about 100 g or more of polymer per day, for example, about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, about 15 g per day, about 16 g per day, about 17 g per day, about 18 g per day, about 19 g per day, about 20 g per day, about 21 g per day, about 22 g per day, about 23 g per day, about 24 g per day, about 25 g per day, about 26 g per day, about 27 g per day, about 28 g per day, about 29 g per day, about 30 g per day, about 35 g per day, about 40 g per day, about 45 g per day, about 50 g per day, about 55 g per day, about 60 g per day, about 65 g per day, about 70 g per day, about 75 g per day, about 80 g per day, about 85 g per day, about 90 g per day, about 95 g per day, or about 100 g of polymer per day or more.

In some embodiments, the dosage form comprises a base and a crosslinked polycarboxylate polymer as described herein, (e.g., a cross-linked polyacrylic acid polymer), wherein the polymer further comprises calcium and magnesium cations, wherein the calcium and magnesium cations are counterions to about 5% to about 75% of the carboxylic acid groups in the polymer (alternately, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and is administered in an amount sufficient to provide from about 1 g to about 30 g or about 100 g or more of polymer per day, for example, about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, about 15 g per day, about 16 g per day, about 17 g per day, about 18 g per day, about 19 g per day, about 20 g per day, about 21 g per day, about 22 g per day, about 23 g per day, about 24 g per day, about 25 g per day, about 26 g per day, about 27 g per day, about 28 g per day, about 29 g per day, about 30 g per day, about 35 g per day, about 40 g per day, about 45 g per day, about 50 g per day, about 55 g per day, about 60 g per day, about 65 g per day, about 70 g per day, about 75 g per day, about 80 g per day, about 85 g per day, about 90 g per day, about 95 g per day, or about 100 g of polymer per day or more.

In some embodiments, the dosage form is a sachet and contains a composition according to the present disclosure in sufficient amount to provide from about 1 g to about 30 g of the polymer. For example, a sachet may contain a composition according to the present disclosure in sufficient amount to provide about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, about 15 g, about 15.5 g, about 16 g, about 16.5 g, about 17 g, about 17.5 g, about 18 g, about 18.5 g, about 19 g, about 19.5 g, about 20 g, about 20.5 g, about 21 g, about 21.5 g, about 22 g, about 22.5 g, about 23 g, about 23.5 g, about 24 g, about 24.5 g, about 25 g, about 25.5 g, about 26 g, about 26.5 g, about 27 g, about 27.5 g, about 28 g, about 28.5 g, about 29 g, about 29.5 g, or about 30 g of polymer.

In some embodiments, the dosage form is a capsule containing an amount of a composition according to the present disclosure sufficient to provide from about 0.1 g to about 1 g of the polymer. For example, a capsule may contain an amount of a composition according to the present disclosure that is sufficient to provide about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, or about 1 g of polymer.

In some embodiments, the dosage form is a tablet that contains an amount of a composition according to the present disclosure to provide from about 0.3 g to about 1 g or 2 g of the polymer. For example, the tablet may contain about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, or about 1 g or 2 g of polymer. In some embodiments, a disclosed composition is formulated as a tablet that is spherical or substantially spherical.

In some embodiments, the dosage form is a sachet, flavored bar, gel block, gel pack, pudding, or powder that contains an amount of a composition according to the present disclosure to provide from about 1 g or about 2 g to about 30 g of the polymer. For example, the sachet, flavored bar, gel block, gel pack, pudding, or powder may contain an amount of a composition according to the present disclosure to provide about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, or about 30 g of the polymer.

In some embodiments, the dosage form is a suspension or an oral suspension that contains an amount of a composition according to the present disclosure to provide from about 1 g or about 2 g to about 30 g of the polymer. For example, the suspension or oral suspension may contain an amount of a composition according to the present disclosure to provide about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, or about 30 g of the polymer.

In some embodiments, dosage forms according to the present disclosure further include an additional agent. In related embodiments, the additional agent is one that causes, routinely causes, typically causes, is known to cause, or is suspected of causing an increase in an ion level in at least some subjects upon administration. For example and without limitation, the additional agent may be an agent known to cause an increase in serum potassium levels in at least some subjects upon administration. For example and without limitation, the additional agent may be an agent known to cause an increase in serum sodium levels in at least some subjects upon administration. In related embodiments, the additional agent may be one or more of: a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, VAP antagonists, amiloride, triamterine, a potassium supplement, heparin, a low molecular weight heparin, a non-steroidal anti-inflammatory drug, ketoconazole, trimethoprim, pentamide, a potassium sparing diuretic, amiloride, and/or triamterene. For example, in some embodiments, the additional agent may cause fluid retention and/or maldistribution in at least some subjects upon administration.

The present disclosure is also directed to formulations comprising a cross-linked cation-binding polymer (e.g., a cross-linked polyacrylic acid polymer). Also provided are powder formulations comprising a cation-binding polymer, water, a suspending agent and optionally an antimicrobial agent, wherein the amount of water does not prevent the powder from freely flowing. The present disclosure also is directed toward a powder formulation comprising a cation-binding polymer, a suspending agent, and a glidant, wherein at least about 40 wt. % cation-binding polymer is present in the composition based on the total weight of the formulation. The powder formulations may additionally comprise colorants, flavors, stabilizers, or other excipients. Such powder formulations may be useful to bind potassium in the gastrointestinal tract to treat hyperkalemia or the risk of hyperkalemia. Powder formulations of polymers may be advantageous in terms of their suitability for a wide range of delivery methods. For example, the powder formulations of polymers may be placed in food, liquid, or another appropriate delivery agent without affecting taste or texture. Suitable suspending agents include, for example, xanthan gum, polycarbophil, hydroxypropyl methyl cellulose (HPMC), povidone, methylcellulose, dextrin, sodium alginate, (poly)vinyl alcohol, microcrystalline cellulose, a colloidal silica, bentonite clay, or a combination thereof. The suspending agent can be present in a concentration ranging from about 0.25 wt. % to about 7.0 wt. %, including, for example, from about 0.3 wt. % to about 3.0 wt. % based on the total weight of formulation. In some embodiments, the suspending agent is xanthan gum, including wherein it is present at a concentration of 0.7 wt. % based on the total weight of formulation. In some embodiments, the powder formulation is free of an antimicrobial agent. In other embodiments, the powder formulation includes an antimicrobial agent (or preservative). Suitable antimicrobial agents include, for example, alpha-tocopherol, ascorbate, alkylparabens (e.g., methylparaben, ethylparaben, propylbaraben, butylparaben, pentylparaben, hexylparaben, benzylparaben), chlorobutanol, phenol, sodium benzoate, benzalkonium chloride, benzethonium chloride, chlorobutanol, phenyl ethyl alcohol, or a combination thereof. The antimicrobial agent may be present in a concentration ranging from about 0 wt. % to about 1.5 wt. %, from about 0.05 wt. % to about 1.5 wt. % and more specifically from about 0.5 wt. % to about 1.5 wt. % based on the total weight of formulation. In some embodiments, the combination of antimicrobial agents is methylparaben and propylparaben, including wherein the concentration of the methylparaben is about 0.05 wt. % to about 1.0 wt. % and the concentration of the propylparaben is about 0.01 wt. % to about 0.2 wt. % based on the total weight of formulation. The powder formulations may optionally include a glidant (or flow enhancing agent). Suitable glidants include colloidal silicon dioxide, (e.g., Cab-O-Sil™, M5), aluminum silicate, talc, powdered cellulose, magnesium trisilicate, silicon dioxide, kaolin, glycerol monostearate, metal stearates such as magnesium stearate, titanium dioxide, starch, or a combination thereof. The glidant may be present in a concentration ranging from about 0 wt. % to about 4.0 wt. %, including from about 0.1 wt. % to about 4 wt. % or from about 0.5 wt. % to about 2 wt. % based on the total weight of formulation. In some embodiments, the glidant is colloidal silicon dioxide, including at a concentration of 0.94 wt. % based on the total weight of formulation. Optionally, an opacity agent can be added to the formulation. Suitable opacity agents include titanium dioxide, zinc oxide, aluminum oxide, or a combination thereof. The opacity agent may be present in a concentration ranging from about 0 wt. % to about 0.5 wt. %, including from about 0 wt. % to about 0.4 wt. % based on the total weight of formulation. In some embodiments, the opacity agent is titanium dioxide, including at a concentration of 0.34 wt. % based on the total weight of the formulation. Another optional component of the formulations is a coloring agent. Suitable coloring agents include alumina, aluminum powder, annatto extract, natural and synthetic beta-carotene, bismuth oxychloride, bronze powder, calcium carbonate, canthaxanthin, caramel, carmine, chlorophyllin, copper complex, chromium hydroxide green, chromium oxides greens, cochineal extract, copper powder, potassium sodium copper chlorophyllin (chlorophyllin copper complex), dihydroxyacetone, ferric ammonium ferro cyanide (iron blue), ferric ferrocyanide (iron blue), guanine (pearl essence), mica, mica-based pearlescent pigment, pyrophyllite, synthetic iron oxide, talc, titanium dioxide, zinc oxide, FD&C Blue #1, FD&C Blue #2, FD&C Green #3, D&C Green #5, D&C Orange #5, FD&C Red #3, D&C Red #6, D&C Red #7, D&C Red #21, D&C Red #22, D&C Red #27, D&C Red #28, D&C Red #30, D&C Red #33, D&C Red #36, FD&C Red #40, FD&C Yellow #5, FD&C Yellow #6, D&C Yellow #10, or a combination thereof. The coloring agent may be present in a concentration ranging from about ° wt. % to about 0.1 wt. %, including from about ° wt. % to about 0.05 wt. % based on the total weight of formulation. In some embodiments, the coloring agent is a blend of coloring agents to provide a yellow, orange, or red color, including, for example, wherein the concentration of the blend is about 0.02 wt. % based on the total weight of the formulation. Another optional component of the formulations is a flavoring agent and/or sweetener. Suitable flavoring agents include, lime, lemon, orange, vanilla, citric acid, and combinations thereof.

The compositions of the present disclosure may be administered in combination with other therapeutic agents. The choice of therapeutic agents that may be co-administered with the compositions of the disclosure will depend, in part, on the condition being treated.

Compositions of the present disclosure may be administered in combination with a therapeutic agent that causes an increase, or is known to commonly cause an increase, in one or more ions in the subject. By way of example only, the crosslinked cation-binding polymer of the present disclosure may be administered with a therapeutic agent that causes an increase, or is known to commonly cause an increase, in the potassium and/or sodium level of a subject.

### Therapeutic Uses

The disclosed polymers, compositions comprising the disclosed polymers, and/or dosage forms comprising the disclosed polymers may be used to treat a subject with a disease and/or disorder. Additionally or alternatively, the disclosed polymers, compositions comprising the disclosed polymers and/or oral dosage forms comprising the disclosed polymers may be used to prevent a subject from becoming afflicted with a disease and/or disorder. In any of the methods of treatment or prevention described herein, a base may be co-administered along with the polymer, composition comprising a polymer, and/or dosage form comprising a polymer, either simultaneously (e.g., at the same time) or sequentially (e.g., before and/or after administration of the polymer). When administering the polymer in a dosage form, the base may be included in the same dosage form or separate from the dosage form containing the polymer.

The disclosed polymers, compositions comprising the disclosed polymers, and/or dosage forms comprising the disclosed polymers may be used in methods for the binding and/or removal of ions (e.g., potassium ions and/or sodium ions) and/or of fluid from a subject. As such, the disclosed polymers, compositions comprising the disclosed polymers, and/or dosage forms comprising the disclosed polymers may be useful in the treatment or prevention of diseases or disorders in which the removal of ions (e.g., potassium ions and/or sodium ions) and/or fluid from a subject is desired.

In some embodiments, the disclosed polymers, compositions comprising the disclosed polymers, and/or dosage forms comprising the disclosed polymers may be used to preferentially remove certain ions (e.g., potassium, sodium, or potassium and sodium) and/or fluid depending on the environment to which the disclosed polymers, compositions comprising the disclosed polymers, and/or dosage forms comprising the disclosed polymers are exposed.

Ions bound to the disclosed polymers and fluid binding capacity of the disclosed polymers may vary based on the type of subject to which it is administered (e.g., a healthy subject or a subject having a disease or disorder or at risk of having a disease or disorder). For healthy subjects, the concentration of potassium and sodium in the colon are typically in the range of from about 55 mM to about 75 mM and from about 20 mM to about 30 mM, respectively, for a ratio of K/Na of approximately 2. However, this ratio may be significantly changed in various disease states and/or in response to therapeutic agents. For example, in hyperaldosterone states such as primary aldosteronism (e.g., Conn's syndrome), or during high dose aldosterone administration, a further increase in the colonic K/Na ratio may be observed with fecal output of potassium increasing by approximately a factor of 3 or more. In end stage renal disease (ESRD), fecal potassium excretion is also known to increase. In contrast, in hypoaldosterone states, such as Addison's disease, congenital hypoaldosteronism, patients develop hyperkalemia and hyponatremia due to a decrease in colonic and renal potassium excretion and an increase in sodium excretion. Administration of spironolactone may increase urinary and fecal sodium excretion. Additionally, for example, in patients with Crohn's disease, celiac disease and ulcerative colitis the fecal sodium may rise to 50-100 mM and the fecal potassium may decrease to 15-20 mM. In these disease states the ratio of K/Na may be less than 0.3 mM.

While presently disclosed polymer compositions may primarily bind potassium in healthy subjects or in subjects with certain disease states or disorders, they may bind primarily sodium in subjects with other diseases or disorders, for example, subjects with aldosterone plasma level or with ulcerative colitis.

Further, ions bound to disclosed polymers and fluid binding capacity of disclosed polymers may vary as the polymers travel through the digestive tract. For example, when the disclosed polymers, compositions comprising the disclosed polymers, and/or dosage forms comprising the disclosed polymers reside in the colon for a significant fraction of the total gastrointestinal transit time, the local concentration of cations in the colon will have a significant effect on the concentrations of sodium, potassium and other cations bound to the polymer and excreted in the feces.

In some embodiments, polymers as disclosed herein, and/or compositions, formulations, and/or dosage forms containing the polymers, may be used in methods to treat congestive heart failure (CHF), chronic kidney disease (CKD), end stage renal disease (ESRD), hyperkalemia, hypernatremia, or hypertension.

In some embodiments, polymers as disclosed herein, and/or compositions, formulations, and/or dosage forms containing the polymers, may be used in methods for the removal of fluid from a subject.

In some embodiments, the polymers as disclosed herein, and/or compositions, formulations, and/or dosage forms containing the polymers, may be used in methods to treat or prevent fluid accumulation and/or maldistribution, and/or ion (e.g., sodium and/or potassium) accumulation and/or imbalances.

In some embodiments, polymers as disclosed herein, and/or compositions, formulations, and/or dosage forms containing the polymers, may be used in methods for treating diseases or disorders associated with increased retention of fluid and/or ion imbalances.

The polymers as disclosed herein, and/or compositions, formulations, and/or dosage forms containing the polymers, may be used to remove one or more ions selected from the group consisting of: sodium, potassium, calcium, magnesium, and/or ammonium.

Surprisingly, the polymers of the present disclosure, and compositions, formulations, and dosage forms of the present disclosure that comprise crosslinked cation-binding polymers comprising monomers containing carboxylic acid groups (e.g., polyacrylate polymers) and pKa decreasing groups and wherein the polymer further comprises: i) calcium cations, wherein the calcium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, or ii) calcium and magensium cations, wherein the calcium and magnesium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations (if present) are counterions to no more than about 5% of the carboxylate groups in the polymer, are optimized for maintaining the cation binding and/or removal properties of the polymer (e.g., for potassium and sodium) and/or the fluid binding and/or removal properties of the polymer in humans, while, in some embodiments, minimizing or eliminating changes in acid/base status (e.g., acid/base balance) in an individual to whom it is administered, for example, as measured by serum total bicarbonate, serum total CO₂, arterial blood pH, urine pH, urine phosphorus, urine ammonium, and/or anion gap. An acid/base status that does not change includes one that does not change outside the normal range or outside the normal range for the subject. As such, the polymers and compositions, formulations, and/or dosage forms containing the polymers as described herein are useful for the treatment of a variety of diseases or disorders, including those involving ion (e.g., potassium and/or sodium) and/or fluid imbalances (e.g., overloads).

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups and wherein the polymer further comprises calcium cations, wherein the calcium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may be used in methods for the removal of fluid and/or ions (e.g., potassium and/or sodium) from a subject, e.g., The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium cations, wherein the calcium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may be used in methods for the removal of fluid from a subject.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups and wherein the polymer further comprises calcium and magnesium cations, wherein the calcium and magnesium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations (if present) are counterions to no more than about 5% of the carboxylate groups in the polymer, may be used in methods for the removal of fluid and/or ions (e.g., potassium and/or sodium) from a subject, e.g., The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium and magnesium cations, wherein the calcium anr magnesium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may be used in methods for the removal of fluid from a subject.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium cations, wherein the calcium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may also be used in methods for treating diseases or disorders associated with increased retention of fluid and/or ion imbalances.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium and magnesium cations, wherein the calcium and magnesium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations (if present) are counterions to no more than about 5% of the carboxylate groups in the polymer, may also be used in methods for treating diseases or disorders associated with increased retention of fluid and/or ion imbalances.

The disclosed polymers, compositions comprising the disclosed polymers, formulations comprising the disclosed polymers, and/or dosage forms comprising the disclosed polymers which comprise repeat units containing carboxylic acid groups, (e.g., polyacrylate polymers) and pKa decreasing groups, and wherein the polymer further comprises calcium cations, wherein the calcium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may also be used in methods to treat end stage renal disease (ESRD), chronic kidney disease (CKD), congestive heart failure (CHF), hyperkalemia, hypernatremia, or hypertension.

The disclosed polymers, compositions comprising the disclosed polymers, formulations comprising the disclosed polymers, and/or dosage forms comprising the disclosed polymers which comprise repeat units containing carboxylic acid groups, (e.g., polyacrylate polymers) and pKa decreasing groups, and wherein the polymer further comprises calcium and magnesium cations, wherein the calcium and magnesium cations are counterions to about 5% to about 75% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations (if present) are counterions to no more than about 5% of the carboxylate groups in the polymer, may also be used in methods to treat end stage renal disease (ESRD), chronic kidney disease (CKD), congestive heart failure (CHF), hyperkalemia, hypernatremia, or hypertension.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium cations, wherein the calcium cations are counterions to about 50 to about 70% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), and wherein sodium cations (if present) are counterions to no more than about 5% of the carboxylate groups in the polymer, may be used to remove one or more ions selected from the group consisting of: sodium, potassium, calcium, magnesium, iron, and/or ammonium.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and pKa decreasing groups, and wherein the polymer further comprises calcium and magnesium cations, wherein the calcium and magnesium cations are counterions to about 50 to about 70% of the carboxylate groups in the polymer (alternatively, counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer), wherein the magnesium cations are counterions to no more than about 35% of the carboxylate groups in the polymer, and wherein sodium cations (if present) are counterions to no more than about 5% of the carboxylate groups in the polymer, may be used to remove one or more ions selected from the group consisting of: sodium, potassium, calcium, magnesium, iron, and/or ammonium.

In some embodiments, the polymers, compositions comprising the disclosed polymers, and/or dosage forms comprising the disclosed polymers as disclosed herein may be substantially coated with a coating (e.g., an enteric coating) that allows it to pass through the gut and open in the intestine where the polymer may absorb fluid and/or specific ions that are concentrated in that particular portion of the intestine. In other embodiments, the polymers, compositions comprising the disclosed polymers, and/or dosage forms comprising the disclosed polymers disclosed herein do not comprise such a coating. In some embodiments, the absorbent material (e.g., polymer as disclosed herein) may be encapsulated in a capsule. In one embodiment, the capsule may be substantially coated with a coating (e.g., an enteric coating) that allows it to pass through the gut and open in the intestine where the capsule may release the polymer to absorb fluid or specific ions that are concentrated in that particular position of the intestine. In another embodiment, the capsule does not contain such a coating. Individual particles of polymer or groups of particles may be encapsulated or alternatively, larger quantities of beads or particles may be encapsulated together.

In some embodiments, polymers as disclosed herein may be milled to give finer particles in order to increase drug loading of capsules, or to provide better palatability for formulations such as gels, bars, puddings, or sachets. In addition, milled particles or groups of particles, or unmilled polymeric material (e.g., beads) may be coated with various common pharmaceutical coatings. These coatings may or may not have enteric properties but will have the common characteristic that they will separate the polymer from the tissues of the mouth and prevent the polymer from adhering to tissue. For example, such coatings may include, but are not limited to: a single polymer or mixtures thereof, such as may be selected from polymers of ethyl cellulose, polyvinyl acetate, cellulose acetate, polymers such as cellulose phthalate, acrylic based polymers and copolymers or any combination of soluble, insoluble polymers or polymer systems, waxes and wax based coating systems.

In some embodiments, the polymers disclosed herein for administration to an individual or inclusion in a composition, formulation, or dosage form for administration to an individual, e.g., for use in a method of treatment as disclosed herein, are individual particles or particles agglomerated to form a larger particle (for example, flocculated particles), and have a diameter of about 1 to about 10,000 microns (alternatively, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns). In some embodiments, the particles or agglomerated particles have a diameter of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 7000, about 7500, about 8000, about 8500, about 9000, about 9500, or about 10,000 microns. In one embodiment, the particles with a diameter of about 1 micron to about 10 microns.

In certain exemplary embodiments, the crosslinked cation-binding polymer, as described, for example, for administration to an individual or inclusion in a composition, formulation, or dosage form for administration to an individual, e.g., for use in a method of treatment as disclosed herein, is a crosslinked acrylic acid polymer (e.g., derived from acrylic acid monomers or a salt thereof) that comprises calcium cations, wherein the calcium cations are counterions to about 5% to about 75% of the carboxylate groups in the polymer, and wherein sodium cations (if present) are counterions to no more than about 5% of the carboxylate groups in the polymer. For example, the polymer may be an acrylic acid polymer crosslinked with about 0.08 mol % to about 0.2 mol % and for example, may comprise an in vitro saline absorption capacity of at least about 20 times its weight (e.g., at least about 20 grams of saline per gram of polymer, or “g/g”), at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked acrylic acid polymer comprises individual particles or particles that are agglomerated (for example, flocculated) to form a larger particle, wherein the individual or agglomerated particle diameter is about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns. In some embodiments, administration of such a crosslinked polyacrylate polymer, comprises calcium cations, wherein the calcium cations are counterions to about 5% to about 75% of the carboxylate groups in the polymer, does not change or does not significantly change acid/base status (e.g., acid/base balance) in an individual to whom it is administered, for example, as measured by serum total bicarbonate, serum total CO₂, arterial blood pH, urine pH, urine phosphorus, urine ammonium, and/or anion gap. An acid/base status that does not change includes one that does not change outside the normal range or outside the normal range for the subject.

In certain exemplary embodiments, the crosslinked cation-binding polymer, as described, for example, for administration to an individual or inclusion in a composition, formulation, or dosage form for administration to an individual, e.g., for use in a method of treatment as disclosed herein, is a crosslinked polyacrylate polymer (e.g., derived from acrylic acid monomers or a salt thereof) that comprises calcium and magnesium cations, wherein the calcium and magnesium cations are counterions to about 5% to about 75% of the carboxylate groups in the polymer, wherein the magnesium cations are counterions to no more than about 35% of the carboxylate groups in the polymer, and wherein sodium cations (if present) are counterions to no more than about 5% of the carboxylate groups in the polymer. For example, the polymer may be a polyacrylate polymer crosslinked with about 0.08 mol % to about 0.2 mol % crosslinker or alternatively from about 0.025 mol % to about 3.0 mol %, including from about 0.025 mol % to about 0.3 mol %, from about 0.025 mol % to about 0.17 mol %, from about 0.025 mol % to about 0.34 mol %, or from about 0.08 mol % to about 0.2 mol % crosslinker or alternatively from about 0.025 mol.% to about 3.0 mol.% including, for example, from about 0.025 mol.% to about 0.3 mol.%, and for example, may comprise an in vitro saline absorption capacity (e.g., saline holding capacity) of at least about 20 times its weight (e.g., at least about 20 grams of saline per gram of polymer, or “g/g”), at least about at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked polyacrylate polymer comprises individual particles or particles that are agglomerated (for example, flocculated) to form a larger particle, wherein the individual or agglomerated particle diameter is about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 20 microns to about 125 microns, about 20 microns to about 150 microns, about 20 microns to about 200 microns, about 35 microns to about 150 microns, about 35 microns to about 125 microns, about 50 microns to about 125 microns, about 50 microns to about 200 microns, about 50 microns to about 1000 microns, about 500 microns to about 1000 microns, about 1000 to about 5000 microns, or about 5000 microns to about 10,000 microns). In some embodiments, the crosslinked polyacrylate polymer comprises individual particles or particles that are agglomerated (for example, flocculated) to form a larger particle, wherein the individual or agglomerated particle diameter is about 100 microns. In some embodiments, administration of such a crosslinked polyacrylate polymer, comprising calcium and magnesium cations, wherein the calcium and magnesium cations are counterions to about 5% to about 75% of the carboxylate groups in the polymer, wherein the magnesium cations are counterions to no more than about 35% of the carboxylate groups in the polymer, does not change or does not significantly change acid/base status (e.g., acid/base balance) in an individual to whom it is administered, for example, as measured by serum total bicarbonate, serum total CO₂, arterial blood pH, urine pH, urine phosphorus, urine ammonium, and/or anion gap. An acid/base status that does not change includes one that does not change outside the normal range or outside the normal range for the subject. In some embodiments, a crosslinked polyacrylate polymer, comprising calcium and magnesium cations, wherein the calcium and magnesium cations are counterions to about 5% to about 75% of the carboxylate groups in the polymer, wherein the magnesium cations are counterions to no more than about 35% of the carboxylate groups in the polymer, is administered with an added base (e.g., up to about 0.90 equivalents of added base per equivalents of carboxylate groups in the polymer), and such administration of the polymer and base does not change or does not significantly change acid/base status (e.g., acid/base balance) in an individual to whom it is administered, for example, as measured by serum total bicarbonate, serum total CO₂, arterial blood pH, urine pH, urine phosphorus, urine ammonium, and/or anion gap. An acid/base status that does not change includes one that does not change outside the normal range or outside the normal range for the subject.

In some embodiments, the polymer may be mixed with base in the same dosage form and may be in contact with fluid within the dosage from, such as suspensions or gels. To prevent interaction of the crosslinked cation-binding polymer and the base component before administration to a subject, pharmaceutical coatings known in the art can be used to coat the polymer, the base, or both to prevent or impede interaction of the polymer and the base. In some embodiments, the pharmaceutical coating may have enteric properties. As example, pharmaceutical coatings may include but are not limited to: a single polymeric coating or mixtures of more than one pharmaceutical coating, such as may be selected from polymers of ethyl cellulose, polyvinyl acetate, cellulose acetate; polymers such as cellulose phthalate, acrylic based polymers and copolymers, or any combination of soluble polymers, insoluble polymers and/or polymer systems, waxes and wax based coating systems. In alternate embodiments, the polymer and base are administered in separate dosage forms.

A subject (e.g., an individual or patient), as disclosed herein, includes a vertebrate, preferably a mammal, more preferably a human. Mammals include, but are not limited to, farm animals (such as cows), sport animals, pets (such as cats, dogs and horses), primates, and rodents (such as mice and rats). For purposes of treatment, prognosis and/or diagnosis, a subject includes any animal such as those classified as a mammal, including humans, domestic and farm animals, and zoo, wild, sports, or pet animals, such as dogs, horses, cats, cows, etc. Preferably, the subject for treatment, prognosis and/or diagnosis is human.

A disease or disorder includes any condition that would benefit from treatment with a composition as disclosed herein. This includes both chronic and acute diseases or disorders, including those pathological conditions which predispose the subject to the disease or disorder in question.

As used herein, treatment refers to clinical intervention in an attempt to alter the natural course of the subject being treated, and can be performed either for prophylaxis (e.g., prevention) or during the course of clinical pathology (e.g., after the subject is identified as having a disease or disorder or the symptoms of a disease or disorder). Desirable effects of treatment include preventing occurrence or recurrence of disease, alleviation of symptoms, diminishment of any direct or indirect pathological consequences of the disease or disorder, decreasing the rate of disease progression, amelioration or palliation of the disorder, and remission or improved prognosis. Terms such as treating/treatment/to treat or alleviating/to alleviate refer to both 1) therapeutic measures that cure, slow down, lessen symptoms of, and/or halt progression of a diagnosed disease or disorder (e.g., a pathologic condition or disorder) and 2) prophylactic or preventative measures that prevent and/or slow the development of a disease or disorder (e.g., a targeted pathologic condition or disorder). Thus, those in need of treatment may include those already with the disease or disorder; those prone to have the disease or disorder; and those in whom the disease or disorder is to be prevented.

An effective amount refers to an amount effective, at dosages and for periods of time necessary, to achieve the desired therapeutic or prophylactic result. A therapeutically effective amount of a composition disclosed herein, may vary according to factors such as the disorder, age, sex, and weight of the subject, and the ability of the composition to elicit a desired response in the individual. A therapeutically effective amount is also one in which any toxic or detrimental effects of the composition are outweighed by the therapeutically beneficial effects. A prophylactically effective amount refers to an amount effective, at dosages and for periods of time necessary, to achieve the desired prophylactic result. Typically but not necessarily, since a prophylactic dose is used in subjects prior to or at an earlier stage of disease, the prophylactically effective amount may be less than the therapeutically effective amount. [symbol] For example, a therapeutically or prophylactically effective amount includes administration of about 1 g to about 30 g, about 15 g to about 25 g, or about 15 g to about 30 g, for example, about 15 g per day of a disclosed cross-linked polymer to an individual. In various embodiments, base is co-administered at about 0.2 equivalents to about 0.95 equivalents, for example, about 0.5 equivalents to about 0.85 equivalents, about 0.7 equivalents to about 0.8 equivalents, or about 0.75 equivalents, with respect to carboxylic acid groups on the polymer. A therapeutically or prophylactically effective amount of polymer and base may be administered in a single dosage or multiple doses, for example, administered once per day or administered 2-4 or more times daily, e.g., divided into and administered as 1, 2, 3, 4, or more doses per day, or administered at intervals of 2, 3, 4, 5, or 6 days, weekly, bi-weekly, etc.

Pharmaceutically acceptable includes approved or approvable by a regulatory agency of the Federal or a state government or listed in the U.S. Pharmacopeia or other generally recognized pharmacopeia for use in animals, including humans. A pharmaceutically acceptable salt includes a salt of a compound that is pharmaceutically acceptable and that possesses the desired pharmacological activity of the parent compound. A pharmaceutically acceptable excipient, carrier or adjuvant includes an excipient, carrier or adjuvant that can be administered to a subject, together with at least one composition of the present disclosure, and which does not destroy the pharmacological activity thereof and is nontoxic when administered in doses sufficient to deliver a therapeutic or prophylactic amount of the composition. A pharmaceutically acceptable vehicle includes a diluent, adjuvant, excipient, or carrier with which at least one composition of the present disclosure is administered.

Compositions, comprising cross-linked cation binding polymers as disclosed herein can be used either alone or in combination with one or more other agents for administration to a subject (e.g., in a therapy or prophylaxis). As described herein, such combined therapies or prophylaxis include combined administration (where the composition and one or more agents are included in the same or separate formulations) and separate administration, in which case, administration of the composition disclosed herein can occur prior to, contemporaneous with and/or following, administration of the one or more other agents (e.g., for adjunct therapy or intervention). Thus, co-administered or co-administration includes administration of the compositions of the present disclosure before, during and/or after the administration of one or more additional agents or therapies.

In some embodiments, the polymers, compositions comprising the disclosed polymers, and/or dosage forms comprising the disclosed polymers are useful for treating a disease or disorder. For example, the disclosed polymers, compositions comprising the disclosed polymers, and/or dosage forms comprising the disclosed polymers are co-administered with a base, as described herein. In some embodiments in which a composition and/or dosage form comprising the polymer is administered, the base may be included in the same composition and/or dosage form as the polymer. In other embodiments, the base may be administered separately from the composition and/or dosage form. In some embodiments, the disease or disorder is one or more of: heart failure (for example, heart failure with or without chronic kidney disease, diastolic heart failure (heart failure with preserved ejection fraction), heart failure with reduced ejection fraction, cardiomyopathy, or congestive heart failure), a renal insufficiency disease, end stage renal disease, liver cirrhosis, chronic renal insufficiency, chronic kidney disease, fluid overload, fluid maldistribution, edema, pulmonary edema, peripheral edema, angioneurotic edema, lymphedema, nephrotic edema, idiopathic edema, ascites, (for example, general ascites or cirrhotic ascites), chronic diarrhea, excessive interdialytic weight gain, high blood pressure, hyperkalemia, hypernatremia, abnormally high total body sodium, hypercalcemia, tumor lysis syndrome, head trauma, an adrenal disease, Addison's disease, salt-wasting congenital adrenal hyperplasia, hyporeninemic hypoaldosteronism, hypertension, salt-sensitive hypertension, refractory hypertension, hyperparathyroidism, renal tubular disease, rhabdomyolysis, electrical burns, thermal burns, crush injuries, renal failure (for example, acute renal failure), acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, aspiration inhalation, neurogenic pulmonary edema, allergic pulmonary edema, high altitude sickness, Adult Respiratory Distress Syndrome, traumatic edema, cardiogenic edema, allergic edema, urticarial edema, acute hemorrhagic edema, papilledema, heatstroke edema, facial edema, eyelid edema, angioedema, cerebral edema, scleral edema, nephritis, nephrosis, nephrotic syndrome, glomerulonephritis, renal vein thrombosis, and/or premenstrual syndrome.

The disclosed polymers, compositions comprising the disclosed polymers, formulations comprising the disclosed polymers, and/or dosage forms comprising the disclosed polymers are useful for treating: hyperkalemia including, hyperkalemia caused by disease and/or use of certain drugs; patients at risk of developing high serum potassium concentrations through use of agents that cause potassium retention; chronic kidney disease and heart failure patients including, drug induced potassium retention; and/or drugs that interfere with potassium excretion including, for example, K-sparing diuretics, ACEs, ARBs, beta blockers, aldosterone antagonists (AAs), renin inhibitors, aldosterone synthase inhibitors, non-steroidal anti-inflammatory drugs, heparin, or trimethoprim.

The disclosed polymers, compositions comprising the disclosed polymers, formulations comprising the disclosed polymers, and/or dosage forms comprising the disclosed polymers are also useful for removal of potassium from a patient, wherein a patient is in need of such potassium removal. For example, patients experiencing hyperkalemia caused by disease and/or use of certain drugs benefit from such potassium removal. Further, patients at risk for developing high serum potassium concentrations through use of agents that cause potassium retention could be in need of potassium removal. The methods described herein are applicable to these patients regardless of the underlying condition that is causing the high serum potassium levels.

Dosing regimens for chronic treatment of hyperkalemia can increase compliance by patients, particularly for disclosed polymers, compositions comprising the disclosed polymers, formulations comprising the disclosed polymers, and/or dosage forms comprising the disclosed polymers that are taken in gram quantities. The present disclosure is also directed to methods of chronically removing potassium from an animal subject in need thereof, and in particular chronically treating hyperkalemia with a potassium binder such as a crosslinked cation binding polymer as described herein.

In some embodiments, the disclosed polymers, compositions comprising the disclosed polymers, formulations comprising the disclosed polymers, and/or dosage forms comprising the disclosed polymers can be administered on a periodic basis to treat a chronic condition. For example, such treatments may enable patients to continue using drugs that may cause hyperkalemia, such as potassium-sparing diuretics, ACEs, ARBs, aldosterone antagonists, β-blockers, renin inhibitors, non-steroidal anti-inflammatory drugs, heparin, trimethoprim, or combinations thereof. Also, use of the disclosed polymers, compositions comprising the disclosed polymers, formulations comprising the disclosed polymers, and/or dosage forms comprising the disclosed polymers may enable certain patient populations, who were unable to use certain above-described drugs, to use such drugs.

In some embodiments, the disclosed polymers, compositions comprising the disclosed polymers, formulations comprising the disclosed polymers, and/or dosage forms comprising the disclosed polymers are capable of removing less than about 5 mEq of potassium per day, or in the range of about 5 mEq to about 60 mEq of potassium per day.

In some embodiments, the disclosed polymers, compositions comprising the disclosed polymers, formulations comprising the disclosed polymers, and/or dosage forms comprising the disclosed polymers and methods described herein are used in the treatment of hyperkalemia in patients in need thereof, for example, when caused by excessive intake of potassium. Excessive potassium intake alone is an uncommon cause of hyperkalemia. More often, hyperkalemia is caused by indiscriminate potassium consumption in a patient with impaired mechanisms for the intracellular shift of potassium or renal potassium excretion.

The disclosed polymers, compositions comprising the disclosed polymers, formulations comprising the disclosed polymers, and/or dosage forms comprising the disclosed polymers can be co-administered with other active pharmaceutical agents. This co-administration can include simultaneous administration of the two agents in the same dosage form, simultaneous administration in separate dosage forms, and separate administration. For example, for the treatment of hyperkalemia, the crosslinked the disclosed polymers, compositions comprising the disclosed polymers, formulations comprising the disclosed polymers, and/or dosage forms comprising the disclosed polymers can be co-administered with drugs that cause the hyperkalemia, such as potassium sparing diuretics, angiotensin-converting enzyme inhibitors (ACEs), angiotensin receptor blockers (ARBs), beta blockers, aldosterone antagonists (AAs), renin inhibitors, non-steroidal anti-inflammatory drugs, heparin, or trimethoprim. In particular, the disclosed polymers, compositions comprising the disclosed polymers, formulations comprising the disclosed polymers, and/or dosage forms comprising the disclosed polymers can be co-administered with ACEs (e.g., captopril, zofenopril, enalapril, ramipril, quinapril, perindopril, lisinopril, benazipril, and fosinopril), ARBs (e.g., candesartan, eprosartan, irbesartan, losartan, olmesartan, telmisartan, and valsartan), AAs (e.g., spironolactone, eplerenone, canrenone), and renin inhibitors (e.g. aliskiren). In particular embodiments, the agents are simultaneously administered, wherein both the agents are present in separate compositions. In other embodiments, the agents are administered separately in time (e.g., sequentially).

Treating or treatment of hyperkalemia includes achieving a therapeutic benefit including, for example, an eradication, amelioration, or prevention of the underlying disorder being treated. For example, in a hyperkalemia patient, therapeutic benefit includes eradication or amelioration of the underlying hyperkalemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of the disclosed polymers, compositions comprising the disclosed polymers, formulations comprising the disclosed polymers, and/or dosage forms comprising the disclosed polymers to a patient experiencing hyperkalemia provides therapeutic benefit not only when the patient's serum potassium level is decreased, but also when an improvement is observed in the patient with respect to other disorders that accompany hyperkalemia, like renal failure. In some treatment regimens, the disclosed polymers, compositions comprising the disclosed polymers, formulations comprising the disclosed polymers, and/or dosage forms comprising the disclosed polymers may be administered to a patient at risk of developing hyperkalemia or to a patient reporting one or more of the physiological symptoms of hyperkalemia, even though a diagnosis of hyperkalemia may not have been made.

Further, patients suffering from chronic kidney disease and/or congestive heart failure can be in need of potassium removal because agents used to treat these conditions may cause potassium retention in a significant population of these patients. For these patients, decreased renal potassium excretion results from renal failure (especially with decreased glomerular filtration rate), often coupled with the ingestion of drugs that interfere with potassium excretion, for example, potassium-sparing diuretics, angiotensin-converting enzyme inhibitors (ACEs), angiotensin receptor blockers (ARBs), beta blockers, aldosterone antagonists (AAs), rennin inhibitors, aldosterone synthase inhibitors, non-steroidal anti-inflammatory drugs, heparin, or trimethoprim. For example, patients suffering from chronic kidney disease can be prescribed various agents that may slow the progression of the disease; for this purpose, angiotensin-converting enzyme inhibitors (ACEs), angiotensin receptor blockers (ARBs), and aldosterone antagonists are commonly prescribed. In these treatment regimens the angiotensin-converting enzyme inhibitor is captopril, zofenopril, enalapril, ramipril, quinapril, perindopril, lisinopril, benazipril, fosinopril, or combinations thereof and the angiotensin receptor blocker is candesartan, eprosartan, irbesartan, losartan, olmesartan, telmisartan, valsartan, or combinations thereof and the renin inhibitor is aliskiren. The aldosterone antagonists spironolactone, eplerenone, and canrenone can also cause potassium retention. Thus, it can be advantageous for patients in need of these treatments to also be treated with an agent that removes potassium from the body. The aldosterone antagonists typically prescribed are spironolactone, eplerenone, and the like.

In some embodiments, the polymers, compositions comprising the disclosed polymers, and/or dosage forms comprising the disclosed polymers, as disclosed herein, are useful for treating a disease or disorder involving an ion imbalance in a subject by administering to the subject an effective amount of a polymer, a composition comprising a disclosed polymer, and/or a dosage form comprising a disclosed polymer (e.g., an effective amount), as disclosed herein. For example, the disclosed polymers, compositions comprising the disclosed polymers, and/or dosage forms comprising the disclosed polymers are co-administered with a base, as described herein. In some embodiments, the disease or disorder is hyperkalemia. In some embodiments, the disease or disorder is hypernatremia. In some embodiments, the disease or disorder is an abnormally high sodium level. In some embodiments, the disease or disorder is an abnormally high potassium level. In some embodiments, the disease or disorder is hyponatremia, hypernatremia and hyperkalemia.

In some embodiments, the polymers, compositions comprising the disclosed polymers, and/or dosage forms comprising the disclosed polymers as disclosed herein are useful for treating a subject with heart failure by administering to the subject an effective amount of a polymer, composition comprising a disclosed polymer, and/or a dosage form comprising a disclosed polymer as disclosed herein. For example, the disclosed polymers, compositions comprising the disclosed polymers, and/or dosage forms comprising the disclosed polymers are co-administered with a base, as described herein. In some embodiments, the subject has both heart failure and chronic kidney disease. In some related embodiments, the methods further comprise reducing one or more symptoms of a fluid overload state in the subject. Symptoms of a fluid overload state in a subject are known to those skilled in the art, and may include, for example and without limitation, difficulty breathing when lying down, ascites, fatigue, shortness of breath, increased body weight, peripheral edema, and/or pulmonary edema. In some related embodiments, the subject may be on concomitant dialysis therapy. In some further related embodiments, the dialysis therapy may be reduced or discontinued after administration of the polymer, the composition comprising the disclosed polymer, and/or the dosage form comprising the disclosed polymer, as disclosed herein. In some related embodiments, the method further comprises identifying the subject as having heart failure before administering the polymer, composition comprising a disclosed polymer, and/or dosage form comprising a disclosed polymer. In some embodiments, administration of the disclosed polymers, compositions comprising the disclosed polymers, and/or dosage forms comprising the disclosed polymers, as described herein, improves or ameliorates at least one symptom of heart failure, for example, at least one symptom that impacts the subject's quality of life and/or physical function. For example, administration may result in body weight reduction, dyspnea improvement (for example, overall and dyspnea at exertion), six minute walk test improvement, and/or improvement or absence of peripheral edema. In some embodiments, administration of the disclosed polymers, compositions comprising the disclosed polymers, and/or dosage forms comprising the disclosed polymers, as described herein, results in reduction of patient classification by at least one heart failure class, according to the New York Heart Association Class I, II, III, IV functional classification system.

In some embodiments, the disclosed polymers, compositions comprising the disclosed polymers, formulations comprising the disclosed polymers, and/or dosage forms comprising the disclosed polymers, as disclosed herein, are useful for treating a subject with end stage renal disease (ESRD) by administering to the subject an effective amount of the polymer, composition, formulation, and/or dosage form, as disclosed herein. In some embodiments, the polymers, compositions, formulations, and/or dosage forms may be co-administered with a base, as described herein. In some related embodiments, the subject is on concomitant dialysis therapy. In some embodiments, the method reduces blood pressure in an ESRD subject on concomitant dialysis therapy, for example, pre-dialysis, post-dialysis, and/or interdialytic systolic and diastolic blood pressure may be reduced. In some embodiments, the method reduces interdialytic weight gain in an ESRD subject on concomitant dialysis therapy. In some embodiments, the subject also has heart failure. In some embodiments, one or more symptoms of intradialytic hypotension are improved after administration of a disclosed polymer, a composition comprising a disclosed polymer, a formulation, comprising a disclosed polymer, and/or a dosage form comprising a disclosed polymer, as disclosed herein. For example and without limitation, incidences of vomiting, fainting and/or drops in blood pressure levels are reduced or eliminated. In some embodiments, the subject experiences one or more of: a reduced frequency of emergency dialysis sessions, a reduced frequency of inadequate dialysis sessions, a reduced frequency of dialysis sessions on low-potassium dialysis bath, and/or reduced frequency or reduced severity of EKG signs during dialysis sessions. In some embodiments, one or more symptom of intradialytic hypotension are reduced or eliminated after administration of a polymer, a composition comprising a disclosed polymer, a composition comprising a disclosed polymer, a formulation comprising a disclosed polymer, and/or a dosage form comprising a disclosed polymer. Symptoms of intradialytic hypotension are known to those skilled in the art and may include, for example, vomiting, fainting, an abrupt decrease in blood pressure, seizures, dizziness, severe abdominal cramping, severe leg or arm muscular cramping, intermittent blindness, infusion, medication, and dialysis session interruption or discontinuation. In some embodiments, ESRD subjects may experience an improvement in physical function as expressed by increase in performance in the 6 Minute Walk Test.

In some embodiments, the disclosed polymers, compositions comprising the disclosed polymers, formulations comprising the disclosed polymers, and/or dosage forms comprising the disclosed polymers, as disclosed herein, are useful for treating a subject having chronic kidney disease. In some embodiments, the methods comprise administering to the subject an effective amount of the polymer, composition, formulation, and/or dosage form. In some embodiments, the polymers, compositions, formulations, and/or dosage forms may be co-administered with a base, as described herein. In some embodiments, the methods further comprise identifying the subject as having a chronic kidney disease before administration of a disclosed polymer, composition comprising the disclosed polymer, formulation comprising the disclosed polymer, and/or dosage form comprising a disclosed polymer, as disclosed herein. In some related embodiments, the methods further comprise reducing one or more symptoms of a fluid overload state in the subject. In some embodiments, a comorbidity of chronic kidney disease is reduced, alleviated, and/or eliminated after administration of a polymer, a composition comprising a disclosed polymer, a formulation comprising a disclosed polymer, and/or a dosage form comprising a disclosed polymer. Comorbidities of chronic kidney disease are known to those skilled in the art and include, for example, fluid overload, edema, pulmonary edema, hypertension, hyperkalemia, excess total body sodium, heart failure, ascites, and/or uremia. In some embodiments, CKD patients may experience prevention of doubling of serum creatinine over the duration of a study (e.g., 1 to 2 years), prevention of disease progression to dialysis, and/or prevention of death and CKD related hospitalizations and/or complications.

In some embodiments, the disclosed polymers, compositions comprising the disclosed polymers, formulations comprising the disclosed polymers, and/or dosage forms comprising the polymers as, disclosed herein, are useful for treating a subject having hypertension. In some embodiments, the methods comprise administering to the subject an effective amount of the polymer, composition, formulation, and/or dosage form. In some embodiments, the polymers, compositions, formulations, and/or dosage forms may be co-administered with a base, as described herein. In some embodiments, the methods further comprise identifying that the subject has hypertension before administering a disclosed polymer, composition comprising a disclosed polymer, formulation comprising a disclosed polymer, and/or dosage form comprising a disclosed polymer, as disclosed herein. As used herein, the term hypertension includes the various subtypes of hypertension known to those skilled in the art, for example and without limitation: primary hypertension, secondary hypertension, salt sensitive hypertension, refractory hypertension, and combinations thereof. In some embodiments, the method is effective in reducing the subject's blood pressure. In related embodiments, the method may further comprise determining a blood pressure level before, after, or both before and after administration of the polymer, composition comprising a disclosed polymer, and/or dosage form comprising a disclosed polymer as disclosed herein. For example, the method may further comprise determining the subject's diastolic blood pressure, systolic blood pressure, and/or mean arterial pressure (“MAP”) before, after, or both before and after administration of the disclosed polymer, composition comprising the disclosed polymer, formulation comprising the disclosed polymer, and/or dosage form comprising the disclosed polymer, as disclosed herein. In some embodiments, one or more symptom of a fluid overload state is reduced, improved, or alleviated by administering a disclosed polymer, composition comprising a disclosed polymer, formulation comprising a disclosed polymer, and/or dosage form comprising a disclosed polymer, as disclosed herein. In some related embodiments, the method may further comprise determining a fluid overload state symptom before, after, or both before and after administration of a disclosed polymer, composition comprising a disclosed polymer formulation comprising a disclosed polymer, and/or dosage form comprising a disclosed polymer as disclosed herein. For example, the method may further comprise observing an improvement in the subject's breathing while lying down, ascites, fatigue, shortness of breath, body weight, peripheral edema, and/or pulmonary edema. In some embodiments, the subject is on concomitant diuretic therapy. As used herein, the term diuretic therapy refers to administration of pharmaceutical compositions (e.g., diuretic agents), and non-chemical intervention, such as dialysis or restriction of fluid intake. Diuretic agents are known to those skilled in the art and include, for example, furosemide, bumetanide, torsemide, hydrochlorthiazide, amiloride and/or spironolactone. In some related embodiments, the diuretic therapy may be reduced or discontinued following administration of the disclosed polymer, composition comprising the disclosed polymer, formulation comprising the disclosed polymer, and/or dosage form comprising the disclosed polymer, as disclosed herein.

In some embodiments, the disclosed polymers, compositions comprising the disclosed polymers, formulations comprising the disclosed polymers, and/or dosage forms comprising the disclosed polymers, as disclosed herein, are useful for treating hyperkalemia in a subject. In some embodiments, the method comprises administering to the subject an effective amount of the polymer, composition, formulation, and/or dosage form as disclosed herein. In some embodiments, the polymers, compositions, formulations, and/or dosage forms may be co-administered with a base, as described herein. In some embodiments, the method further comprises identifying the subject as having hyperkalemia, or as having a risk of developing hyperkalemia, before administering the disclosed polymer, composition comprising a disclosed polymer, formulation, and/or dosage form comprising a disclosed polymer, as disclosed herein. In some embodiments, the method may further comprise determining a potassium ion level in the subject before administering the disclosed polymer, composition comprising the disclosed polymer, formulation comprising the disclosed polymer, and/or dosage form comprising a disclosed polymer, as disclosed herein. In some related embodiments, the potassium ion level may be within a normal range, slightly elevated, or elevated before administering the disclosed polymer, composition comprising the disclosed polymer, formulation comprising the disclosed polymer, and/or dosage form comprising the disclosed polymer, as disclosed herein. In some embodiments, the subject has been prescribed or will be administered a drug known to increase potassium levels. In some embodiments, the subject has already ingested a drug known to increase potassium levels. In some embodiments, the method may further comprise determining a second, reduced potassium ion level in the subject after administration of the disclosed polymer, composition comprising the disclosed polymer, formulation comprising the disclosed polymer, and/or dosage form comprising the disclosed polymer, as disclosed herein. In some embodiments, an acid/base status (e.g., acid/base balance) associated with the subject does not change, for example, as measured by, serum total bicarbonate, serum total CO₂, arterial blood pH, urine pH, urine phosphorus, urine ammonium, and/or anion gap, after administration of the disclosed polymer, composition comprising the disclosed polymer, formulation comprising the disclosed polymer, and/or dosage form comprising the disclosed polymer, as disclosed herein. An acid/base status that does not change includes one that does not change outside the normal range or outside the normal range for the subject.

In some embodiments, the disclosed polymers, compositions comprising the disclosed polymers, formulations comprising the disclosed polymers, and/or dosage forms comprising the disclosed polymers, as disclosed herein, are useful for treating a high sodium level, e.g., an abnormally high sodium level such as hypernatremia, in a subject. In some embodiments, the method comprises administering to the subject an effective amount of the polymer, composition, formulation, and/or dosage form as disclosed herein. In some embodiments, the disclosed polymers, compositions, formulations, and/or dosage forms may be co-administered with a base, as described herein. In some embodiments, the method further comprises identifying the subject as having a high sodium level, or as having a risk of developing a high sodium level, before administering the disclosed polymer, composition comprising the disclosed polymer, formulation comprising the disclosed polymer, and/or dosage form comprising the disclosed polymer, as disclosed herein. In some embodiments, the method may further comprise determining a sodium ion level, e.g., a total body sodium ion level, in the subject before administering the disclosed polymer, composition comprising the disclosed polymer, formulation comprising the disclosed polymer, and/or dosage form comprising the disclosed polymer, as disclosed herein. In some related embodiments, the sodium ion level, e.g., serum sodium ion level, may be within a normal range, slightly elevated, or elevated before administering the disclosed polymer, composition comprising the disclosed polymer, formulation comprising the disclosed polymer, and/or dosage form comprising the disclosed polymer, as disclosed herein. In some embodiments, the method may further comprise determining a second, reduced sodium ion level, e.g., a total body sodium ion level, in the subject after administration of the disclosed polymer, composition comprising the disclosed polymer, formulation comprising the disclosed polymer, and/or dosage form comprising the disclosed polymer, as disclosed herein. In some embodiments, an acid/base status (e.g., acid/base balance) associated with the subject does not change, for example, as measured by, serum total bicarbonate, serum total CO₂, arterial blood pH, urine pH, urine phosphorus, urine ammonium, and/or anion gap, after administration of the disclosed polymer, composition comprising the disclosed polymer, formulation comprising the disclosed polymer, and/or dosage form comprising the disclosed polymer, as disclosed herein. An acid/base status that does not change includes one that does not change outside the normal range or outside the normal range for the subject. In some embodiments, the subject has taken or will take a drug known to increase sodium levels, for example and without limitation: estrogen containing compositions, mineralocorticoids, osmotic diuretics (e.g., glucose or urea), vaptans (e.g., tolvaptan, lixivaptan), lactulose, cathartics (e.g., phenolphthalein), phenytoin, lithium, Amphotericin B, demeclocycline, dopamine, ofloxacin, orlistat, ifosfamide, cyclophosphamide, hyperosmolar radiographic contrast agents (e.g., gastrographin, renographin), cidofovir, ethanol, foscarnet, indinavir, libenzapril, mesalazine, methoxyflurane, pimozide, rifampin, streptozotocin, tenofir, triamterene, and/or cholchicine. In some embodiments, administration of the disclosed polymer, compositions comprising the disclosed polymer, formulations comprising the disclosed polymer, and/or dosage forms comprising the disclosed polymer may further comprise increasing a dose of one or more additional agents, for example, an agent known to cause an increase in sodium levels. In some embodiments, the method further comprises increasing a dose of one or more of: an aldosterone antagonist, an angiotensin II receptor blocker, and/or an angiotensin-converting enzyme inhibitor before, concomitantly, and/or after administering a disclosed polymer, a composition comprising a disclosed polymer, a formulation comprising a disclosed polymer, and/or a dosage form comprising a disclosed polymer. In some embodiments, administration of the disclosed polymer, composition comprising a disclosed polymer, formulation comprising a disclosed polymer, and/or dosage form comprising a disclosed polymer may further comprise decreasing a dose or discontinuing administration or co-administration of a diuretic.

In some embodiments, the disclosed polymers, compositions comprising the disclosed polymers, formulations comprising the disclosed polymers, and/or dosage forms comprising the disclosed polymers, as disclosed herein, are useful for treating a subject with a disease or disorder involving fluid overload (e.g., a fluid overload state such as heart failure, end stage renal disease, ascites, renal failure, nephritis, and nephrosis). In some embodiments, the method comprises administering to the subject an effective amount of the polymer, composition, formulation, and/or dosage form as disclosed herein. In some embodiments, the polymers, compositions, formulations, and/or dosage forms may be co-administered with a base, as described herein. In some embodiments, the subject may be on concomitant diuretic therapy. In some embodiments, the method may further comprise identifying a fluid overload state in the subject, or identifying a risk that the subject will develop a fluid overload state before administration of a disclosed polymer, composition comprising a disclosed polymer, formulation comprising a disclosed polymer, and/or dosage form comprising a disclosed polymer. Methods of identifying a fluid overload state or a risk of developing a fluid overload state are known to those skill in the art and may include, for example and without limitation: assessing difficulty breathing when lying down, ascites, fatigue, shortness of breath, increased body weight, peripheral edema, and/or pulmonary edema associated with the subject. In some embodiments, an acid/base status (e.g., acid/base balance) associated with the subject, for example, as measured by, serum total bicarbonate, serum total CO₂, arterial blood pH, urine pH, urine phosphorus, urine ammonium, and/or anion gap, does not change within about one day of administration of the disclosed polymer, composition comprising the disclosed polymer, formulation comprising the disclosed polymer, and/or dosage form comprising the disclosed polymer, as disclosed herein. An acid/base status that does not change includes one that does not change outside the normal range or outside the normal range for the subject.

In some embodiments, the disclosed polymers, compositions comprising the disclosed polymers, formulations comprising the disclosed polymers, and/or dosage forms comprising the polymers, as disclosed herein, are useful for treating a subject with a disease or disorder involving fluid maldistribution (e.g., a fluid maldistribution state such as pulmonary edema, angioneurotic edema, ascites, high altitude sickness, adult respiratory distress syndrome, uticarial edema, papille edema, facial edema, eyelid edema, cerebral edema, and scleral edema). In some embodiments, the method comprises administering to the subject an effective amount of the polymer, composition, formulation, and/or dosage form, as disclosed herein. In some embodiments, the polymers, compositions, formulations, and/or dosage forms may be co-administered with a base, as described herein. In some embodiments, the method may further comprise identifying a fluid maldistribution state or a risk of developing a fluid maldistribution state in the subject before administering to the subject a disclosed polymer, composition comprising a disclosed polymer, formulation comprising a disclosed polymer, and/or dosage form comprising a disclosed polymer.

In some embodiments, the disclosed polymers, compositions comprising the disclosed polymers, formulations comprising the disclosed polymers, and/or dosage forms comprising the disclosed polymers, as disclosed herein, are useful for treating edema in a subject. In some embodiments, the method comprises administering to the subject an effective amount of the polymer, composition, formulation, and/or dosage form, as disclosed herein. In some embodiments, the polymers, compositions, formulations, and/or dosage forms may be co-administered with a base, as described herein. In some embodiments, the method may further comprise identifying an edematous state or a risk of developing an edematous state in the subject before administering a disclosed polymer, composition comprising a disclosed polymer, formulation comprising a disclosed polymer, and/or dosage form comprising a disclosed, as disclosed herein. In some embodiments, the edematous state is nephritic edema, pulmonary edema, peripheral edema, lymphedema, and/or angioneurotic edema. In some embodiments, the subject is on concomitant diuretic therapy. In some related embodiments, the diuretic therapy may be reduced or discontinued after administration of the disclosed polymer, composition comprising the disclosed polymer, formulation comprising the disclosed polymer, and/or dosage form comprising the disclosed polymer, as disclosed herein. In some embodiments, the method may further comprise, before administering a disclosed polymer, composition comprising a disclosed polymer, formulation comprising a disclosed polymer, and/or dosage form comprising a disclosed polymer, as disclosed herein, determining one or more of: a baseline level of one or more ions (e.g., sodium, potassium, lithium and/or magnesium) in the subject, a baseline total body weight associated with the subject, a baseline total body water level associated with the subject, a baseline total extracellular water level associated with the subject (e.g., a measure of the degree of edema in a particular site as evidenced by depth of pitting or extent of x-ray changes, and/or a baseline total intracellular water level associated with the subject. In some embodiments, the method may further comprise, after administering a disclosed polymer, composition comprising a disclosed polymer, formulation comprising a disclosed polymer, and/or dosage form comprising a disclosed polymer, as disclosed herein, determining one or more of: a second level of one or more ions in the subject, a second total body weight associated with the subject, a second total body water level associated with the subject, a second total extracellular water level associated with the subject, and/or a second total intracellular water level associated with said subject. In some embodiments, the second level is lower than the corresponding baseline level. In some embodiments, an acid/base status (e.g, acid/base balance) associated with said subject, for example, as measured by, serum total bicarbonate, serum total CO₂, arterial blood pH, urine pH, urine phosphorus, urine ammonium, and/or anion gap, does not significantly change within about one day of administration of the disclosed polymer, composition comprising a disclosed polymer, formulation comprising a disclosed polymer, and/or dosage form comprising a disclosed polymer. An acid/base status that does not change includes one that does not change outside the normal range or outside the normal range for the subject. In some embodiments, a blood pressure level associated with the subject after administration of the disclosed polymer, composition comprising a disclosed polymer, formulation comprising a disclosed polymer, and/or dosage form comprising a disclosed polymer is substantially lower than a baseline blood pressure level associated with the subject determined before administration of the disclosed polymer, composition comprising a disclosed polymer, formulation comprising a disclosed polymer, and/or dosage form comprising a disclosed polymer. In some embodiments, one or more symptoms of edema are reduced and/or eliminated following administration of a disclosed polymer, composition comprising a disclosed polymer, formulation comprising a disclosed polymer, and/or dosage form comprising a disclosed polymer, as disclosed herein. Symptoms of edema are known to those skilled in the art; some non-limiting examples include: difficulty breathing when lying down, shortness of breath, peripheral edema, and leg edema.

In some embodiments, the disclosed polymers, compositions comprising the disclosed polymers, formulations comprising the disclosed polymers, and/or dosage forms comprising the disclosed polymers according to the present disclosure are useful for treating ascites in a subject. In some embodiments, the method comprises administering to the subject an effective amount of the polymer, composition, formulation, and/or a dosage form, as disclosed herein. In some embodiments, the disclosed polymers, compositions, formulations, and/or dosage forms may be co-administered with a base, as described herein. In some embodiments, the method may further comprise identifying an ascitic state or a risk of developing an ascitic state in the subject. In some embodiments, the subject is on concomitant diuretic therapy. In some related embodiments, the diuretic therapy may be reduced or discontinued after administration of the disclosed composition. In some embodiments, the subject may have taken, or will take, a drug known to increase potassium levels.

In some embodiments, the disclosed polymers, compositions comprising the disclosed polymers, formulations comprising the disclosed polymers, and/or dosage forms comprising the disclosed polymers, as disclosed herein, are useful for treating nephrotic syndrome in a subject. In some embodiments, the method comprises administering to said subject an effective amount of the polymer, composition, formulation, and/or dosage form, as disclosed herein. In some embodiments, the disclosed polymers, compositions, formulations, and/or dosage forms may be co-administered with a base, as described herein. In some embodiments, the method further comprises identifying the subject as having nephrotic syndrome, or as having a risk of developing nephrotic syndrome, before administering the disclosed polymer, composition comprising the disclosed polymer, formulation comprising the disclosed polymer, and/or dosage form comprising the disclosed polymer. In some embodiments, the method may further comprise determining one or more of: a level of one or more ions (e.g., sodium, potassium calcium, lithium, and/or magnesium) in the subject, a total body weight associated with the subject, a total body water level associated with the subject, a total extracellular water level associated with the subject, and/or a total intracellular water level associated with the subject before administering the disclosed polymer, composition comprising the disclosed polymer, formulation comprising the disclosed polymer, and/or dosage form comprising the disclosed polymer. In some embodiments, the method may further comprise determining a second, lower level of one or more of: a level of one or more ions in the subject, a total body weight associated with the subject, a total body water level associated with the subject, a total extracellular water level associated with the subject, and/or a total intracellular water level associated with the subject after administering the disclosed polymer, composition comprising the disclosed polymer, formulation comprising the disclosed polymer, and/or dosage form comprising the disclosed polymer. In some embodiments, an acid/base status (e.g, acid/base balance) associated with the subject, for example, as measured by, serum total bicarbonate, serum total CO₂, arterial blood pH, urine pH, urine phosphorus, urine ammonium, and/or anion gap, does not significantly change within about one day of administration of the disclosed polymer, composition comprising the disclosed polymer, formulation comprising the disclosed polymer, and/or dosage form comprising a disclosed polymer. An acid/base status that does not change includes one that does not change outside the normal range or outside the normal range for the subject. In some embodiments, a blood pressure level associated with the subject after administration of the disclosed polymer, composition comprising the disclosed polymer, formulation comprising the disclosed polymer, and/or dosage form comprising the disclosed polymer is substantially lower than a baseline blood pressure level associated with the subject before the administration(s). In some embodiments, one or more symptoms of fluid overload is alleviated, reduced, or eliminated after administration of the disclosed polymer, composition comprising the disclosed polymer, formulation comprising the disclosed polymer, and/or dosage form comprising the disclosed polymer. In some related embodiments, the symptom may be one or more of: difficulty breathing when lying down, shortness of breath, peripheral edema, and/or leg edema. In some embodiments, the subject may be on concomitant diuretic therapy. In some related embodiments, the diuretic therapy may be reduced or eliminated after administration of the disclosed polymer, composition comprising the disclosed polymer, formulation comprising the disclosed polymer, and/or dosage form comprising the disclosed polymer.

In some embodiments, methods according to the present disclosure may further comprise administering to the subject an additional agent such as mannitol, sorbitol, calcium acetate, sevelamer carbonate (Renvela®), lanthanum carbonate, and/or sevelamer hydrochloride.

In some embodiments, methods according to the present disclosure may further comprise administering to the subject an agent known to increase potassium levels. As used herein, the term “an agent known to increase potassium levels” refers to agents that are known to cause an increase, are suspected of causing an increase, or are correlated with an increase in potassium levels upon administration. For example and without limitation, agents known to cause an increase in potassium levels may include: a tertiary amine, spironolactone, eplerenone, canrenone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, and/or VAP antagonists. In some embodiments, administration of the polymers, compositions comprising the disclosed polymers, and/or dosage forms comprising the disclosed polymers may further comprise increasing a dose of one or more additional agents, for example, an agent known to cause an increase in potassium levels. In some embodiments, administration of the polymers, compositions comprising the disclosed polymers, and/or dosage forms comprising the disclosed polymers may further comprise decreasing a dose or discontinuing administration or co-administration of a diuretic.

In some embodiments, methods according to the present disclosure may further comprise administering to the subject an agent known to increase sodium levels. As used herein, the term “an agent known to increase sodium levels” refers to agents that are known to cause an increase, are suspected of causing an increase, or are correlated with an increase in sodium levels upon administration, including agents that increase the sodium content in the gastrointestinal tract, including, for example, sodium reuptake inhibitors, sodium transport inhibitors, or inhibitors of NHE3. For example and without limitation, agents known to cause an increase in sodium levels may include: estrogen containing compositions, mineralocorticoids, osmotic diuretics (e.g., glucose or urea), vaptans (e.g., tolvaptan, lixivaptan), lactulose, cathartics (e.g., phenolphthalein), phenytoin, lithium, Amphotericin B, demeclocycline, dopamine, ofloxacin, orlistat, ifosfamide, cyclophosphamide, hyperosmolar radiographic contrast agents (e.g., gastrographin, renographin), cidofovir, ethanol, foscarnet, indinavir, libenzapril, mesalazine, methoxyflurane, pimozide, rifampin, streptozotocin, tenofir, triamterene, and/or cholchicine. In some embodiments, administration of the polymers, compositions comprising the disclosed polymers, and/or dosage forms comprising the disclosed polymers may further comprise increasing a dose of one or more additional agents, for example, an agent known to cause an increase in sodium levels, including agents that increase the sodium content in the gastrointestinal tract, including, for example, sodium reuptake inhibitors, sodium transport inhibitors, or inhibitors of NHE3. In some embodiments, administration of the polymers, compositions comprising the disclosed polymers, and/or dosage forms comprising the disclosed polymers may further comprise decreasing a dose or discontinuing administration or co-administration of a diuretic.

In some embodiments, methods according to the present disclosure may further comprise determining a baseline level of one or more ions in a subject before administering a disclosed polymer, composition comprising a disclosed polymer, formulation comprising a disclosed polymer, and/or the dosage form comprising a disclosed polymer, as disclosed herein, and determining a second level of the one or more ions in the subject after administering the disclosed polymer, composition comprising the disclosed polymer, formulation comprising the disclosed polymer, and/or dosage form comprising the disclosed polymer, as disclosed herein. In related embodiments, a baseline level of potassium is determined in a subject. In another embodiment, a baseline level of sodium is determined in a subject. Thereafter, a disclosed polymer, composition comprising a disclosed polymer, formulation comprising a disclosed polymer, and/or dosage form comprising a disclosed polymer, as disclosed herein, is administered to the subject, followed by a determination of a second potassium and/or sodium level. In some embodiments, the second potassium and/or sodium level is lower than the baseline potassium level.

In some embodiments, methods according to the present disclosure may further comprise determining a baseline total body weight associated with a subject before administering a disclosed polymer, composition comprising a disclosed polymer, formulation comprising a disclosed polymer, and/or dosage form comprising a disclosed polymer, as disclosed herein, and determining a second total body weight associated with the subject after administering the disclosed polymer, composition comprising the disclosed polymer, formulation comprising the disclosed polymer, and/or dosage form comprising the disclosed polymer, as disclosed herein. In some embodiments, the second total body weight is lower than the baseline total body weight. Any suitable method for determining the total body weight associated with a subject may be used.

In some embodiments, methods according to the present disclosure may further comprise determining a baseline total water level, e.g., total body water level, associated with a subject before administering a disclosed polymer, composition comprising a disclosed polymer, formulation comprising a disclosed polymer, and/or dosage form comprising a disclosed polymer, as disclosed herein, and determining a second total water level, e.g., total body water level, associated with the subject after administering the disclosed polymer, composition comprising the disclosed polymer, and/or dosage form comprising the disclosed polymer, as disclosed herein. In some embodiments, the second total water level, e.g., total body water level, is lower than the baseline total water level, e.g., total body water level. Any suitable method for determining a total water level associated with a subject may be used, for example, by bioimpedance measurement, or through invasive procedures, such as central vein catheters for measurement of pulmonary wedge pressure.

In some embodiments, methods according to the present disclosure may further comprise determining a baseline total extracellular water level associated with a subject before administering a disclosed polymer, composition comprising a disclosed polymer, formulation comprising a disclosed polymer, and/or dosage form comprising a disclosed polymer, as disclosed herein, and determining a second total extracellular water level associated with the subject after administering the polymer, composition comprising the disclosed polymer, formulation comprising the disclosed polymer, and/or dosage form comprising the disclosed polymer, as disclosed herein. In some embodiments, the second total extracellular water level is lower than the baseline total extracellular water level. Any suitable method for determining a total extracellular water level associated with a subject may be used, for example, by bioimpedance measurement, or through invasive procedures, such as central vein catheters for measurement of pulmonary wedge pressure.

In some embodiments, methods according to the present disclosure may further comprise determining an acid/base status (e.g., acid/base balance) associated with a subject. Any method known in the art for determining an acid/base status (e.g., acid/base balance) may be employed. In some embodiments, methods according to the present disclosure comprise determining an acid/base status (e.g., acid/base balance) associated with a subject after administering a composition according to the present disclosure.

For example, an acid/base status (e.g., acid/base balance) may be measured by serum total bicarbonate, arterial blood pH, urine pH, urine phosphorus, urine ammonium, and/or anion gap. In an exemplary method, anion gap may be calculated by subtracting the serum concentrations of chloride and bicarbonate (anions) from the concentrations of sodium and potassium (cations) as follows:

Anion gap=([Na⁺]+[K⁺])−([Cl⁻]+[HCO₃⁻])

Alternatively, in another exemplary method, anion gap may be calculated by ignoring potassium concentration as follows:

Anion gap=[Na⁺]−([Cl⁻]+[HCO₃⁻])

In related embodiments, the acid/base status (e.g., acid/base balance) is within a normal range as set by a clinical laboratory.

In related embodiments, the acid/base status (e.g., acid/base balance) is within a normal range for the subject, and/or within a clinically acceptable range for the subject.

In some embodiments, an acid/base status (e.g., acid/base balance) associated with a subject after administering a disclosed polymer, composition comprising a disclosed polymer, formulation comprising a disclosed polymer, and/or dosage form comprising a disclosed polymer, according to the present disclosure, is closer to a normal level for the subject, closer to a clinically acceptable level, etc., than compared to a baseline acid/base status (e.g., acid/base balance) associated with the subject before administration of the polymer, composition, formulation, and/or dosage form.

In some embodiments, an acid/base status (e.g., acid/base balance) associated with the subject does not change or does not significantly change, for example, at the end of a time interval, or about 1 day, within about 18 hours, within about 12 hours, 10 hours, within about 9 hours, within about 8 hours, within about 7 hours, within about 6 hours, within about 5 hours, within about 4 hours, within about 3 hours, within about 2 hours, or within about 1 hour of administration of the composition. An acid/base status that does not change includes one that does not change outside the normal range or outside the normal range for the subject.

Methods for determining an ion level in a subject are known to those skilled in the art. Any suitable method for determining an ion level may be used. However, determination of serum sodium levels should be avoided as such levels tend not to fluctuate, even in hypernatremic subjects. If sodium ion levels are desired, another suitable method for determining such levels should preferably be used, such as determining a subject's total body sodium level.

In some embodiments, methods according to the present disclosure may further comprise determining a blood pressure level before, after, or both before and after administration of a disclosed polymer, composition comprising a disclosed polymer, formulation comprising a disclosed polymer, and/or dosage form comprising a disclosed polymer, according to the present disclosure. A subject's blood pressure level may be determined using any suitable method known in the art. For example and without limitation, a subject's blood pressure level may be determined by measuring the subject's systolic blood pressure, the subject's diastolic blood pressure, and/or the subject's mean arterial pressure (“MAP”). In some embodiments, the subject's blood pressure is lower after treatment than before treatment.

In some embodiments, the disclosed polymers, compositions comprising the disclosed polymers, formulations comprising the disclosed polymers, and/or dosage forms comprising the disclosed polymers, according to the present disclosure, are administered as needed to reduce an ion level in a subject, and/or to maintain an acceptable level of one or more ions in a subject, and/or to reduce a fluid overload state or fluid maldistribution state in a subject. In some embodiments, compositions according to the present disclosure are administered at a frequency from 1 time per every 3 days to about 4 times per day. Preferably, the compositions according to the present disclosure are administered from about 1 time per day to about 4 times per day; for example, once or twice per day.

### EXAMPLES

The following examples are for illustrative purposes only and are not to be construed as limiting in any manner.

### Example 1

This example demonstrates the preparation of an exemplary crosslinked cation-binding polymer comprising monomers that comprise carboxylic acid groups and pKa-decreasing groups including, for example, an electron-withdrawing substituent such as a halide atom (e.g., fluorine (F)), partially neutralized with sodium. Such an exemplary polymer may be prepared by an inverse suspension process or an oil-in-water process.

### A. Inverse Suspension Process

In an exemplary method for the preparation of an exemplary crosslined cation-binding polymer comprising monomers that comprise carboxylic acid groups and pKa-decreasing groups including, for example, an electron-withdrawing substituent such as a halide atom (e.g., fluorine (F)), an inverse suspension process may be used with the following components: a monomer (e.g., acrylic acid and/or 2-fluoroacrylic acid), solvent for the monomer (e.g., hydrophilic, for example, water), base for neutralization of monomer (e.g., NaOH), lipophilic (e.g., hydrophobic) solvent (e.g., Isopar™ L), suspending agent (e.g., fumed silica such as Aerosil R972), chelating agent (e.g., Versenex™-80), polymerization initiator (e.g., sodium persulfate), and cross-linking agent (e.g., TMPTA).

A monomer solution is prepared in a vessel as the aqueous phase by dissolving an unsaturated carboxylic acid monomer (e.g., acrylic acid and/or fluoroacrylic acid) in water and neutralizing with an aqueous alkali (e.g., NaOH) to a desired percentage neutralization (e.g., 70% to 95% neutralized). Just before addition of this aqueous, partially neutralized, monomer solution to the reactor, one or more polymerization initiators (e.g., sodium persulfate alone or a redox-couple, such as t-butylhydroperoxide paired with thiosulfate) are added under conditions that do not favor polymerization. Optionally, a chelating agent (e.g., Versenex™-80) can be added to the aqueous mixture ensure control of transition metal ions. An organic phase (e.g., Isopar™ L or toluene or n-heptane or cyclohexane) is placed into the main reactor (not the vessel with the aqueous monomer solution). A hydrophobic suspending agent (e.g., Aerosil R972) is dissolved or dispersed in the organic phase. A crosslinking agent is added. If the crosslinking agent is more soluble in the organic phase (e.g., divinylbenzene or 1,1,1-trimethylolpropane triacrylate—also called TMPTA), it is added to the reactor with the organic phase. If the crosslinking agent is more water soluble (e.g., highly-ethoxylated trimethylolpropane triacrylate—also called HE-TMPTA—or diacryl glycerol), the crosslinking agent is added to the aqueous phase. The aqueous phase is then added to the organic phase in the reactor, e.g., with mixing, and the reaction mixture is agitated to produce aqueous droplets of the appropriate size in the organic solvent. Simultaneously, oxygen is removed from the reaction mixture by bubbling an inert gas (e.g., nitrogen) through the reaction mixture. After adequate deoxygenation, the reaction will either begin (e.g., in the case of redox couples) or be started by increasing the temperature (e.g., in the case of sodium persulfate). A second addition of hydrophobic suspending agent may be added as the polymerization proceeds, e.g., to further stabilize the particles. Reaction is completed by maintaining an elevated temperature (e.g., 65° C.) for a time adequate to allow removal, e.g., reaction of substantially all of the monomer (e.g., 2 to 4 hours). Water may then be removed by azeotropic distillation and the crosslinked cation-binding polymeric material may be isolated by filtration or centrifugation to remove the remaining organic solvent. The polymeric material may be rinsed with fresh organic solvent and dried to the desired moisture and/or organic solvent content as measured by loss on further drying. In some embodiments, less than 500 ppm of the monomer remains after polymerization. The polymer may be rinsed to remove this residual monomer.

In an exemplary method, acrylic acid (140 g) was added dropwise to a solution of 124.35 g of 50% NaOH and 140 g of deionized water while keeping the temperature below 40° C. to prevent initiation of polymerization. 3.5 g of Versenex™ 80 and 0.70 g of a 10% solution of sodium persulfate were added. Meanwhile, 1200 g of Isopar™ L were charged into the main reactor. 0.80 g Aerosil R972 dissolved in 40 g of Isopar™ L and 0.50 g of TMPTA were added to the main reactor. The aqueous monomer solution was added to the reactor, which was then closed. Agitation was started at 330 RPM and argon was bubbled through the reaction mixture. After 70 minutes of bubbling argon, the reaction was heated rapidly at 4° C. increase per minute. When the temperature reached 50° C., another 0.80 g of Aerosil R972 in 40 g of Isopar™ L (that had been separately bubbled with argon) was added to the reaction mixture. The reaction exotherm heated the mixture to 80° C. over the next 15 minutes while the constant temperature bath was removing heat to keep the reaction mixture at 65° C. The reaction mixture cooled to 70° C. at approximately 60 minutes from the start of heating. The reaction mixture was kept at 65° C. to 70° C. for 4 hours. The reaction mixture was allowed to cool. The resulting crosslinked cation-binding polymer (partial sodium salt of polyacrylic acid) was isolated by filtration and dried in vacuum at 105° C. Similarly, a partial sodium salt of poly-2-fluoroacrylic acid can be prepared by adjusting the amount of monomer for the difference in molecular weight (e.g. 175 g of 2-fluoroacrylic acid rather than 140 g of acrylic acid). Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

### B. Oil-in Water Process

In another exemplary method for the preparation of an exemplary crosslined cation-binding polymer comprising monomers that comprise carboxylic acid groups and pKa-decreasing groups including, for example, an electron-withdrawing substituent such as a halide atom (e.g., fluorine (F)), an oil-in-water process may be used with the following components: a monomer (e.g., methyl-2-fluoroacrylate), base for hydrolysis and conversion to the sodium salt (e.g., NaOH), surfactant/suspension stabilizer (e.g., polyvinyl alcohol or polyvinyl alcohol-co-polyvinylacetate; PVA), polymerization initiator (e.g., lauroyl persulfate), and cross-linking agent (e.g., 1,7-octadiene and divinyl benzene), and water.

The polymerization can be carried out in a 1 L three-neck Morton-type round bottom flask equipped with an overhead mechanical stirrer with a Teflon paddle and a water condenser. An organic phase is prepared by mixing methyl-2-fluouracrylate (54 g), divinyl benzene (0.02 g), 1,7-octadiene (0.02 g) and lauroyl peroxide (0.6 g). An aqueous phase is prepared by dissolving PVA (3 g) and NaCl (11.25 g) in water (285.75 g). The organic and aqueous phases are then mixed in the flask and stirred at 300 rpm under nitrogen. The flask is then immersed in a 70° C. oil bath for 5 hours and then cooled to room temperature. The internal temperature during reaction is about 65° C. The solid product is then washed with water and collected by filtration. The white solid is then freeze-dried, affording dry solid beads. The polymethyl-2-fluoroacrylate beads are hydrolysed and converted to the sodium salt by suspending the beads in a NaOH solution (400 g, 10 wt. %) and stirring at 200 rpm. The mixture is heated in a 95° C. oil bath for 20 hours and then cooled to room temperature. The solid product is then washed with water and collected by filtration. After freeze-drying, beads of the sodium salt of poly2-fluoroacrylate sodium are obtained. Similarly, the potassium salt of poly2-fluoroacrylate can be prepared using the same method except for using a KOH solution rather than a NaOH solution for hydrolysis (for example, 500 g of a 10 wt % solution of KOH for 48.93 g of polymethylfluoroacrylate). Likewise, beads of the sodium salt of polyacrylic acid can be prepared from methacrylate monomer by adjusting the amount of monomer for the difference in molecular weight (e.g. 45 g of methacrylate rather than 54 g of methyl-2-fluoroacrylate). Similarly, copolymers of methylacrylate and 2-fluoroacrylate monomers can be prepared by adjusting the amount of monomer for the difference in molecular weight of methylacrylate and poly-2-fluoroacrylate.

### Example 2

This example demonstrates the preparation of an exemplary crosslinked cation-binding polymer comprising monomers that comprise carboxylic acid groups and pKa-decreasing groups including, for example, an electron-withdrawing substituent such as a halide atom (e.g., fluorine (F)), partially neutralized with sodium. Such an exemplary polymer may be prepared by an aqueous phase reaction of a partially neutralized carboxylic acid monomer.

In an exemplary method, crosslinked polyacrylic acid or crosslinked poly-2-fluoroacrylic acid or copolymers thereof may be prepared. A monomer solution is prepared in a reactor by dissolving an unsaturated carboxylic acid monomer (e.g., acrylic acid and/or 2-fluoroacrylic acid) in water and neutralizing with an aqueous alkali (e.g., NaOH) to a desired percentage neutralization (e.g., 70 to 95 percent neutralized). Optionally, a chelating agent (e.g., Versenex™ 80) may be added to control metal ions. A suitable crosslinking agent (e.g., 1,1,1-trimethylolpropane triacrylate or diacryl glycerol) is added to the reactor. A polymerization initiator is added to the reactor. The reactor is then closed and the reaction mixture is bubbled with an inert gas (e.g., nitrogen) and agitated until adequate removal of oxygen is achieved. The reaction is then initiated either by reaching an oxygen concentration where a redox couple produces radicals or by adding heat to cause a temperature dependent initiator (e.g., persulfate salts) to produce radicals. The reaction is allowed to proceed through the exothermic heating that occurs during reaction. After 2 to 6 hours, the reaction is completed and the gel-like mass of reaction product can be removed from the reactor and cut into appropriately sized pieces and dried. After drying, the particles can be separated by size or milled to produce the desired size or size distribution.

In another exemplary method, 140 g of acrylic acid was added dropwise to a solution of 124.35 g of 50% NaOH and 140 g of deionized water while keeping the temperature below 40° C. to prevent initiation of polymerization. Then, 3.5 g of Versenex™ 80 and 0.70 g of a 10% solution of sodium persulfate were added. The final addition was 0.50 g of TMPTA. The reactor was closed and the reaction mixture agitated at 200 RPM while argon was bubbled through the mixture. After 70 minutes of bubbling argon, the reaction was initiated by heating at a rate of a 4° C. temperature rise per minute. After 7 minutes, the reaction reached 55° C. and the entire reaction mixture became a gel. The agitation was stopped, allowing the gel to slowly settle to the bottom of the reactor. The temperature of the heating bath was maintained at 65° C. for another 4 hours. The gel was then cooled, cut into pieces, and dried in a vacuum at 105° C. Similarly, a partial sodium salt of poly-2-fluoroacrylic acid can be prepared adjusting the amount of monomer for the difference in molecular weight (e.g. 175 g of 2-fluoroacrylic acid rather than 140 g of acrylic acid). Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

In an alternative exemplary large scale continuous production method, a monomer feed mix of approximately 6.0 g TMPTA, 2.2 kg water, 0.4 kg sodium hydroxide, and 3.0 g sodium persulfate per kg of acrylic acid was deoxygenated and polymerization initiated with 0.6 g sodium ascorbate per kg of acrylic acid. The solution was then charged to a curing conveyor belt, where the sodium acrylate solution polymerized to a gel as it traveled on the conveyor belt. The polymer gel was then mechanically cut and granulated to reduce the polymer gel particle size and then the polymer was dried. The dried polymer was then milled and sieved to a desired particle size. Similarly, a partial sodium salt of crosslinked poly-2-fluoroacrylic acid can be prepared using the same amounts of reagents for each 1.25 kg of poly-2-fluoroacrylic acid rather than for each kg of acrylic acid. Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

### Example 3

This example demonstrates the conversion of an exemplary crosslinked cation-binding polymer comprising monomers that comprise carboxylic acid groups and pKa-decreasing groups including, for example, an electron-withdrawing substituent such as a halide atom (e.g., fluorine (F)), prepared as described in Example 1 or 2 to a crosslinked cation-binding polymer with a reduced degree of sodium substitution (e.g., an acidified polymer).

In an exemplary method, a polymer is weighed and the moles of neutralized carboxylate determined. For example, the content of different cations can be calculated based on knowledge of the polymer preparation procedure, or from elemental analysis of a sample, and this information is used to determine the number of moles of carboxylate present. The polymer is then washed with an excess (e.g., twice the number of moles of carboxylates, or more) of acid (preferably HCl or phosphoric acid, e.g. 1 N HCl or 4 M phosphoric acid), in batches, by column elution or in a continuous process. The resulting acidified polymer is rinsed with water to remove any excess of the 1 N acid and bring the polymer to a more neutral pH (e.g. pH 4 to 7) and dried in a vacuum at 60° C. to 100° C.

For example, 89.65 g of a polyacrylate produced of the method provided in Example 1 was placed into a beaker and stirred with 667 mL of 1 N HCl for 2 hours. The liquid was drained and the polymeric particles were returned to the vessel. A second aliquot of 667 mL of 1 N HCl was added and the mixture was stirred for 1 hour. The liquid was drained and a third rinse with 667 mL of 1 N HCl was performed for 1 hour. The liquid was drained and the polymeric material was placed into 667 mL of deionized water and stirred for 1 hour. The liquid was drained and another 667 mL of deionized water was added. The polymeric material was then stirred for 1 hour before draining the liquid. This water washing was continued until the pH of the rinse water was above 3. The crosslinked cation-binding polymer was then dried in a vacuum at 60° C. Similarly, a crosslinked poly-2-fluoroacrylic acid can be prepared by adjusting the amount of polymer and/or acid used for the difference in molecular weight per COOH group (e.g. for a polyfluoroacrylate prepared according to Example 1 or 2, using 112 g of polyfluoroacrylate rather than 89.65 g of polyacrylate). Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

Alternatively, one-hundred grams of a crosslinked cation-binding polymer comprising monomers that comprise carboxylic acid groups and pKa-decreasing groups, such as a partially neutralized crosslinked polyacrylate polymer (e.g., prepared as described in Example 1) was placed into a vessel. Next, about 2,250 milliliters of pure (e.g., trace metal or otherwise certified low metal) 1 M HCl was added to the vessel and then the polymer and the acid were stirred gently for two hours. The liquid was removed by decanting or filtration. If desired due to vessel size or for improved mass balance, the 2,250 milliliters of 1M HCl is divided into multiple batches and used sequentially. For instance, 750 milliliters were added, stirred with the polymer, and removed followed by two or more separate additions of 750 milliliters. The polymer was then rinsed with 2,250 milliliters of low metal content water to remove excess acid surrounding the polymer such as a polyacrylate. The crosslinked cation-binding polymer was then dried. Similarly, a crosslinked poly-2-fluoroacrylic acid can be prepared by adjusting the amount of polymer and/or acid for the difference in molecular weight per COOH monomer (e.g. for a poly2-fluoroacrylate prepared according to Example 1 or 2, using 125 g of poly2-fluoroacrylate rather than 100 g of polyacrylate). Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

Further alternatively, one-hundred grams of a crosslinked cation-binding polymer comprising monomers that comprise carboxylic acid groups and pKa-decreasing groups, such as a cross-linked polyacrylate polymer were placed into a filtration funnel or a column equipped with a bottom filter. The polymer was then rinsed with about 2,250 milliliters of pure (e.g., trace metal or otherwise certified low metal) 1 M HCl for about an hour or more. Next, the polymer was rinsed with 2,250 milliliters of low metal content water. The crosslinked cation-binding polymer was then dried. Similarly, a crosslinked poly-2-fluoroacrylic acid can be prepared by adjusting the amount of polymer and/or acid for the difference in molecular weight per COOH monomer (e.g. for a polyfluoroacrylate prepared according to Example 1 or 2, using 125 g of poly2-fluoroacrylate rather than 100 g of polyacrylate). Likewise, a partial sodium salt of a copolymer of acrylic acid and poly-2-fluoroacrylic acid may be prepared by adjusting the amount of monomer for the difference in molecular weight of acrylic acid and poly-2-fluoroacrylic acid.

Exemplary acidified polymers useful as crosslinked cation-binding polymers prepared according to this Example generally have a saline holding capacity of greater than about 40 g/g (see, e.g., Examples 8 to 10); and contain less than about 5,000 ppm of sodium, less than about 20 ppm of heavy metals, less than about 500 ppm of residual monomer, less than about 2,000 ppm of residual chloride, and less than about 20 wt. % of soluble polymer. Preferably, acidified polymers useful as crosslinked cation-binding polymers prepared according to this Example have a saline holding capacity of greater than about 40 g/g (see, e.g., Examples 8 to 10); and contain less than about 500 ppm of sodium, less than about 20 ppm of heavy metals, less than about 50 ppm of residual monomer, less than about 1,500 ppm of residual chloride, and less than about 10 wt. % of soluble polymer.

### Example 4

This example demonstrates the preparation of an exemplary substantially metal free (e.g., acid form) crosslinked cation-binding polymer comprising monomers that comprise carboxylic acid groups and pKa-decreasing groups including, for example, an electron-withdrawing substituent such as a halide atom (e.g., fluorine (F)), prepared as described in Example 1 or 2 to a crosslinked cation-binding polymer with a reduced degree of sodium substitution (e.g., an acidified polymer). Such an exemplary substantially metal free (e.g., acid form) crosslinked cation-binding polymer may be prepared by an aqueous process or an oil-in-water process and may include crosslinked polyacrylic acid, crosslinked poly-2-fluoroacrylic acid, or copolymers thereof.

### A. Aqueous Polymerization Process

In an exemplary method, substantially metal free (e.g., acid form) cross-linked polyacrylic acid polymer was prepared by placing 140 g of glacial acrylic acid (e.g., not neutralized as in Example 1) into a three to five liter reactor with 2,200 to 2,500 milliliters of dilute acid, such as 1 M HCl. A water soluble cross linking agent, such as 1,3-diglycerate diacrylate, in a ratio chosen to produce the desired saline holding capacity (e.g., 20-fold, 30-fold, 40-fold or more) and an initiator were added to the monomer solution. After sparging the reactor with an inert gas, (e.g., nitrogen) and agitating the reaction mixture, the reaction was started and allowed to proceed for two to four hours until substantially all of the monomer had reacted. The resultant mass of wet polymer was then cut into smaller pieces (e.g., 1-2 cm per side), dried in a vacuum or in an inert atmosphere, and then disrupted (e.g., by milling) to produce particles or powder.

140 g of acrylic acid was placed into a reactor and diluted with 326 g of deionized water followed by addition of 0.50 g of TMPTA and 0.70 g of a 10% solution of sodium persulfate. The reactor was closed and the reaction mixture was agitated at 250 RPM while argon was bubbled through the reaction mixture. After 70 minutes of bubbling argon, the reaction mixture was heated to produce approximately 4° C. increase in temperature per minute. After 7 minutes, the temperature reached approximately 50° C. and the entire reaction mixture became a gel that quickly settled to the bottom of the reactor when the agitation was stopped. Heating at 65° C. was continued for 2 hours and the gel was allowed to cool overnight. The gel was then cut into pieces and dried in a vacuum oven at 60° C. The resultant mass of polymer was then cut into smaller pieces (e.g., 1-2 cm per side), dried in a vacuum or in an inert atmosphere, and then disrupted (e.g., by milling) to produce particles or powder.

Alternatively, polycarbophil, a polyacrylic acid crosslinked with divinyl glycol may be used (see, e.g., USP monograph).

### B. Oil-In-Water Process

In an exemplary method for the preparation of substantially metal free (e.g., acid form) crosslinked cation-binding polymer comprising monomers that comprise carboxylic acid groups and pKa-decreasing groups including, for example, an electron-withdrawing substituent such as a halide atom (e.g., fluorine (F)), an oil-in-water process is used to produce a poly-2-fluoroacrylic acid. A stock aqueous solution of sodium chloride (4.95 g), water (157.08 g), polyvinylalcohol (1.65 g), Na2HPO4.7H20 (1.40 g), NaH2PO4-H20 (0.09 g), and NaNO2 (0.02 g) is prepared in a 500 mL 3-necked reaction flask with baffles. An organic phase of t-butyl-fluoroacrylate (30.00 g), divinylbenzene (0.01 g), octadiene (0.01 g), and lauroyl peroxide (0.24 g) is prepared. The organic phase is then added to the aqueous phase in the flask. The flask is then fitted with an overhead stirrer, and a condenser. Nitrogen is blown over the reaction for 10 minutes and a blanket of nitrogen maintained throughout the reaction. The stir rate is then set to 180 rpm and the bath temperature set to 70° C. After 12 hours the heat is increased to 85° C. for 2 hours and the reaction mixture then allowed to cool to room temperature. The beads are then isolated from the reaction flask and washed with isopropyl alcohol, ethanol and water. The poly(a-fluoroacrylate, t-butyl ester) beads are then dried at room temperature under reduced pressure. Into a 500 mL 3-necked reaction flask with baffles, is then weighed 28 g of the poly(2-fluoroacrylate, t-butyl ester) beads, 84 g of concentrated hydrochloric acid (3 times the weight of the beads) and 84 g water (3 times the weight of the beads). The flask is then fitted with an overhead stirrer, and a condenser. Nitrogen is blown over the reaction for 10 minutes and a blanket of nitrogen maintained throughout the reaction. The stir rate is set to 180 rpm and the bath temperature to 75° C. After 12 hours the heat is turned off and the reaction mixture allowed to cool to room temperature. The beads are then isolated from the reaction flask and washed with isopropyl alcohol, ethanol and water. The acid form polypoly-2-fluoroacrylic acid beads are then dried at room temperature under reduced pressure.

Similarly, beads of the polyacrylic acid can be prepared from the t-butylacrylate monomer by adjusting the amount of monomer for the difference in molecular weight (e.g. 26 g of t-butylacrylate rather than 30 g of t-butyl-2-fluoroacrylate). Likewise, copolymers of acrylic acid and fluoroacrylic acid can be prepared by adjusting the amount of monomer for the difference in molecular weight of t-butylacrylate and t-butyl-2-fluoroacrylate.

### Example 5

This example describes the preparation of crosslinked polycarboxylic acid polymers with calcium and/or magnesium counterions, for example calcium fluoropolyacrylate or magnesium polyacrylate, from (A. below) the acid form of the polymer and (B. below) from crosslinked carboxylic acid polymers with counterions other than calcium or magnesium or at concentration of calcium and magnesium higher than in the final polymer (e.g. sodium or potassium counterions, 100% calcium counterions) and (C. below) by polymerizing the monomer partially neutralized with a calcium or magnesium base base.

C. Preparation of Crosslinked Polycarboxylic Acid Polymer with Calcium or Magnesium Counterions from the Acid Form Polymer.

In an exemplary method for the preparation of calcium polyacrylate from polyacrylic acid prepared, for example, as described in Example 3 or 4, polyacrylate with calcium counterions was prepared by adding 60 L of deionized water to a 100 L jacketed vessel and then heating to 65° C. An appropriate amount of CaO (for example, 390 g of CaO for preparation of polymer containing 25% calcium counterions) was added slowly with stirring and the mixture stirred to create a solution or suspension. 4 kg of polyacrylic acid polymer (for example polyacrylic acid polymer prepared by a process substantially similar to Example 3 from NaCLP prepared by process substantially similar to Example 1) was then added to the solution and the mixture stirred at 65° C. for four hours. The resulting calcium containing polymer was then transferred to drying trays and dried in a tray drier at 100° C. until the water content was less than 5%.

Similarly, poly2-fluoroacrylic acid with calcium counterions can be prepared. In an exemplary method for the preparation of calcium 2-fluoropolyacrylate from 2-fluoropolyacrylic acid, including, for example, as described in any of Examples 21-26, fluoropolyacrylate with calcium counterions is prepared by adding 60 L of deionized water to a 100 L jacketed vessel and then heating to 65° C. An appropriate amount of CaO (for example, 390 g of CaO for preparation of polymer containing 25% calcium counterions) is added slowly with stirring and the mixture stirred to create a solution or suspension. Five kg of fluoroacrylic acid (for example fluoroacrylic acid prepared by a process substantially similar to any of Examples 21 to 26) is then added to the solution and the mixture stirred at 65° C. for four hours. The resulting calcium containing polymer is then transferred to drying trays and dried in a tray drier at 80° C. until the water content is less than 20%.

Cross-linked polyacrylate polymers with target levels of 25% magnesium counterions, 10% calcium counterions, and 25% calcium counterions were prepared from polyacrylic acid with 80% of the carboxyl groups with sodium counterions manufactured by a process substantially similar to Example 1, followed by enteric coating first with 30 wt % 93F19255 and then 1 wt % YS-1-19025. 352 g of the enteric coated polyacrylate particles were washed with 1M sodium bicarbonate and five 10 L rinses with water to remove the enteric coating. The water swollen beads were then washed three times with 1 N HCl, with a total of approximately 6 equivalents of acid, twice the number of equivalents of carboxyl groups on the polyacrylate polymer, to convert the polymer to the acid form. The acid was then washed from polymer with four water washes raising the pH from pH 1 to above 3. The total weight of the wet beads was 1782 g. 137 g of the wet beads were placed into each of 3 beakers. To produce polyacrylate with approximately 25% magnesium counterions, 1.05 g of MgO was placed on the polymer in one beaker, followed by 250 mL deionized water. Similarly, to produce polyacrylate with approximately 10 and 25% calcium counterions 0.58 g of CaO and 1.46 g CaO, respectively, were used. The beads and base were stirred and heated at 60° C. overnight. The polyacrylate beads were then dried in a vacuum oven. The cation content of each of the dried polyacrylates was measured by ICP using the method in Example 6. The counterion content of the polyacrylates was 25.2% magnesium (target 25% magnesium), 12.0% calcium (10% calcium target) and 24.0% calcium (25% calcium target).

D. Preparation of Crosslinked Carboxylic Acid Polymers with Counterions Other than Calcium or Magnesium or at Concentration of Calcium and Magnesium Higher than in the Final Polymer.

Alternatively, other calcium or magnesium bases (for example, CaCO₃) may be used for preparation of polyacrylate with calcium and/or magnesium counterions.

In an exemplary method, polyfluoroacrylic acid with calcium counterions may be produced from a polyfluoroacrylic acid with sodium counterions. For example poly2-fluoroacrylic acid with 25% calcium counterions may be manufactured according to methods of Example 1 or 2, adjusting the procedure to neutralize 25% of the 2-fluoroacrylic acid carboxyl groups with NaOH or with Example 1 or 2 to prepare 80% sodium poly2-fluoroacrylic acid and Example 3 using only enough acid in Example 3 to bring the sodium counterions down to a level where they are counterions to 25% of the carboxyl groups. The hydrated 25% sodium polyfluoroacrylate polymer is then equilibrated with a calcium salt (e.g. CaCl₂) solution to exchange the sodium with calcium. This equilibration may be repeated with fresh solutions of calcium salt to affect more complete exchange with sodium and to remove sodium prior to drying. Similarly a fluoropolyacrylic acid with calcium or magnesium counterions can be prepared from a sodium and/or potassium fluoropolyacrylate.

In another exemplary method, polyacrylic acid with calcium counterions including, for example, with calcium as counterion to about 5% to about 75% of the carboxyl groups, may be produced from calcium polycarbophil (see, e.g., USP monograph) by adding an appropriate amount of acid (e.g., hydrochloric acid, and/or phosphoric acid) to bring the percent of carboxyl groups with calcium counterion to the desired level (e.g., with calcium counterion to about 5% to about 75% of the carboxyl groups). A water and/or ethanol wash may then be used to remove free calcium and the material dried.

E. Preparation of Crosslinked Polycarboxylic Acid Polymer with Calcium or Magnesium Counterions Ca-CLP from Acrylic Acid Partially Neutralized with a Calcium or Magnesium Base.

In an exemplary method for the preparation of a calcium acrylate from acrylic acid partially neutralized with a calcium base, a 25% calcium neutralized crosslinked polyacrylate was prepared as follows: 150 g of acrylic acid was placed into a reactor and diluted with 540 g of deionized water. 25 mol % CaO (mol % is moles per mole of acrylic acid) was added with stirring and cooling. After dissolution of the CaO, 0.26 mol % TMPTA and 0.091 mol % sodium persulfate were added. The solution was stirred and bubbled with nitrogen and the temperature reduced to 18° C. Sodium ascorbate at 0.022 mol % was then added and nitrogen purge continued. As the exothermic reaction proceeded, the reactor was heated to 80° C. and the reaction was allowed to proceed for two hours. The gel was then cut into pieces and dried in an oven at 80-100° C.

In an exemplary method for the preparation of calcium fluoroacrylate from fluoroacrylic acid partially neutralized with a calcium base, a 25% calcium neutralized crosslinked poly2-fluoroacrylate is prepared as follows: 187 g of 2-fluroracrylic acid is placed into a reactor and diluted with 540 g of deionized water. 25 mol % CaO (mol % is moles per mole of fluoroacrylic acid) is added with stirring and cooling. After dissolution of the CaO, 0.26 mol % TMPTA and 0.091 mol % sodium persulfate are added. The solution is stirred and bubbled with nitrogen and the temperature reduced to 18° C. Sodium ascorbate at 0.022 mol % is then added and nitrogen purge continued. As the exothermic reaction proceeds, the reactor is heated to 80° C. and the reaction is allowed to proceed for two hours. The gel is then cut into pieces and dried in an oven at 80-100° C.

For example, a 25% calcium neutralized crosslinked polyacrylate was prepared as follows: 13.6 g of CaO and 442 g of water were placed into a reactor and stirred until all CaO dissolved. The reactor was then cooled to 21° C. and 140 g of acrylic acid added with stirring. After cooling to 26° C., 0.14 g of TMPTA and 0.18 g Versenex 80 were added and the solution was deoxygenated with argon sparging. 4.2 g of a 10 wt % solution of sodium persulfate and 2.1 g of a 1 wt % solution of tert-butylhydroperoxide were then added. After stirring for 2 minutes 1.05 g of a 10 wt % solution of sodium thiosulfate pentahydrate and 0.84 g of a 10 cwt % solution of sodium erythorbate were added to initiate the polymerization. After the temperature rose to 51° C. the reactor was heated at 65° C. for 3 hours. The gel was then removed from the reactor, torn and cut into pieces and dried in an oven.

For example, a 25% calcium neutralized crosslinked poly2-fluoroacrylate is prepared as follows: 13.6 g of CaO and 442 g of water are placed into a reactor and stirred until all CaO dissolved. The reactor is then cooled to 21° C. and 175 g of 2-fluoroacrylic acid added with stirring. After cooling to 26° C., 0.14 g of TMPTA and 0.18 g Versenex 80 are added and the solution deoxygenated with argon sparging. 4.2 g of a 10 wt % solution of sodium persulfate and 2.1 g of a 1 wt % solution of tert-butylhydroperoxide are then added. After stirring for 2 minutes 1.05 g of a 10 wt % solution of sodium thiosulfate pentahydrate and 0.84 g of a 10 wt % solution of sodium erythorbate are added to initiate the polymerization. After the temperature rises above 50° C. the reactor is heated at 65° C. for 3 hours. The gel is then removed from the reactor, torn and cut into pieces and dried in an oven at 60-80° C.

For example, a 10% calcium neutralized crosslinked polyacrylate was prepared as follows: 5.4 g of CaO, 330 g of water and 140 g acrylic acid were placed into a reactor and stirred for 30 minutes. Then 0.5 g of TMPTA and 0.7 g of a 10% solution of sodium persulfate were added and the mixture sparged with argon. After stirring for 20 minutes the bath was heated to 85° C. Polymer gel was observed at 55° C. (after 7 minutes). The bath temperature was adjusted at 65° C. and the reactor heated for 2 hours. The gel was left to cool overnight in the reactor and was then torn into pieces and dried in a vacuum oven.

For example, a 10% calcium polyfluoroacrylate is prepared as follows: 5.4 g of CaO, 330 g of water and 175 g 2-fluoroacrylic acid are placed into a reactor and stirred for 30 minutes. Then 0.5 g of TMPTA and 0.7 g of a 10% solution of sodium persulfate are added and the mixture sparged with argon. After stirring for 20 minutes the bath is heated to 85° C. Once a gel is observed, the bath temperature is adjusted at 65° C. and the reactor heated for 2 hours. The gel is left to cool overnight in the reactor and then torn into pieces and dried in a vacuum oven.

### Example 6

The content (e.g., percentage; %) of certain cations including, for example, calcium, sodium, magnesium, and/or potassium, on a polymer may be determined by ICP-OES, ICP-AES and/or ICP-MS, for example, with a ThermoElectron Finnegan Element 2 or a Perkin Elmer Elan 6000 instrument. The percentage of cations that are counterions to the carboxylate groups in the polymer determined in different ICP measurements may vary by ±5% or less. For example, the determination of 5% to 75% calcium or calcium and magnesium cations as counterions to carboxylate groups in the polymer may vary in different measurements by ICP [e.g., 5%±5% to 75%±5% (4.75 to 5.25 or 71.25 to 78.75)].

For example, the calcium and/or sodium content of a polymer prepared according to Example 5 can be determined by diluting a 250 mg sample of the polymer with 5% nitric acid solution to a total volume of 100 mL. After shaking overnight to extract the calcium and sodium cations from the polymer, an aliquot of the mixture can be diluted with a 1% nitric acid solution as necessary to bring the concentration of the cation within the range of a suitable calibration curve (e.g., a standard curve with a linear range). An appropriate internal standard (e.g., scandium, yttrium, germanium) is used to correct for matrix effects. Samples are diluted to within the range of the linear standard curve for analysis. Preferably the polymer is completely digested. To ensure complete digestion of the sample, an exemplary method is to fully digest the sample in nitric acid (e.g., until the solution becomes clear and colorless), for example by application of heat; using microwave digestion; using other acids or mixture of acids, hydrogen peroxide, or other reagents; or by other methods known in the art. For example, the polymer may be placed in a nitric acid, hydrochloric acid, and hydrogen peroxide medium and microwave digesting the sample using any method known to one of skill in the art.

In an exemplary method, a 250.08 mg sample of a calcium polyacrylate polymer prepared as described by any one or more of Examples 1, 3 and 5 was placed in a 100-mL polypropylene tube and a 5% nitric acid solution was added until the total volume of the sample was 100 mL. The tube was then shaken overnight to produce “Mixed Sample A.” A 250.11 mg sample of the same polymer used to prepare Mixed Sample A was placed in a 100-mL polypropylene tube and a 5% nitric acid solution was added until the total volume of the sample was 100 mL. The tube is then shaken overnight to produce “Mixed Sample B.” Next, three 0.100-mL aliquots Mixed Sample A were diluted with a 1% nitric acid solution to final volumes of 10.0 mL. This dilution was then repeated a second time for a total dilution of 10,000×. As an internal standard, 102 μL, 101 μL, and 100 μL of a 10,000 μg/mL standard solution of 99.999% scandium oxide in 5% nitric acid was added to the three aliquots, respectively. Separately, three 0.100-mL aliquots of Mixed Sample B were similarly diluted with 1% nitric acid to final volumes of 10.0, the dilution repeated a second time for a total dilution of 10,000×, and doped with 100 μL, 99.0 μL, and 100 μL of the standard scandium solution, respectively. Analysis of calcium content proceeded using a ThermoElectron Finnigan Element 2 ICP-AES instrument (equipped with software version 2.42) according to the manufacturer's specifications. Standards at 0, 100, 250, 500, 2500, and 5000 ug/L were analyzed to generate a standard curve. The six calcium concentration measurements (e.g., 55,449, 55,318, 54,761, 56,079, 56,375, and 55,949 μg/g, respectively) were determined (e.g., from the standard curve) by normalizing the intensity of the raw calcium measurement to the measurement of the internal scandium standard and correction for sample dilution. These six calcium concentration measurements were then converted into weight percent values (e.g., 5.54, 5.53, 5.48, 5.61, 5.64, and 5.59 wt. % Ca, respectively) and averaged to provide an overall calcium content of 5.6 wt. %. The percentage of carboxylate groups to which calcium serves as a counterion on a polymer (e.g., the “[x]% Ca-PAA” nomenclature) can be determined from the weight percent calcium measurement (wt. % Ca) by the following equation:

[x]% Ca-PAA=(72.06)(wt. % Ca)/(20.05−(0.19)(wt. % Ca))

For this example analysis, therefore, the polymer would be termed “21% Ca-PAA. PAA.”

For example, polymers of the present disclosure such as crosslinked polyacrylate polymers may have calcium concentration measurements (e.g., average calcium concentration measurements as determined by ICP-AES analysis) of about 13,723 μg of calcium to about 174,078 μg of calcium per gram of the polymer. This range approximately corresponds to a polymer in which calcium serves as a counterion to about 5% to about 75% of the carboxylate groups, as shown in Table 3. In some embodiments, polymers of the present disclosure such as crosslinked polyacrylate polymers may have calcium concentration measurements (e.g., average calcium concentration measurements as determined by ICP-AES analysis) of about 40,100 μg of calcium to about 89,100 μg of calcium per gram of the polymer. This range approximately corresponds to a polymer in which calcium serves as a counterion to about 15% to about 35% of the carboxylate groups, as shown in Table 3.

TABLE 3
Calcium Content for Various Exemplary Polyacrylate Polymers.

| % of Carboxylates | | | |
|---|---|---|---|
| with Ca | MW | Wt % Ca | μg/g Ca |
| 0 | 72.06 | 0.00 | 0 |
| 5 | 73.01 | 1.37 | 13723 |
| 10 | 73.97 | 2.71 | 27092 |
| 15 | 74.92 | 4.01 | 40122 |
| 20 | 75.87 | 5.28 | 52825 |
| 25 | 76.82 | 6.52 | 65214 |
| 30 | 77.77 | 7.73 | 77299 |
| 35 | 78.72 | 8.91 | 89092 |
| 40 | 79.68 | 10.06 | 100603 |
| 45 | 80.63 | 11.18 | 111843 |
| 50 | 81.58 | 12.28 | 122821 |
| 55 | 82.53 | 13.35 | 133545 |
| 60 | 83.48 | 14.40 | 144025 |
| 62 | 83.86 | 14.82 | 148150 |
| 65 | 84.43 | 15.43 | 154269 |
| 70 | 85.38 | 16.43 | 164284 |
| 72 | 85.77 | 16.82 | 168228 |
| 75 | 86.34 | 17.41 | 174078 |
| 80 | 87.29 | 18.37 | 183660 |
| 85 | 88.24 | 19.30 | 193034 |
| 90 | 89.19 | 20.22 | 202208 |
| 95 | 90.14 | 21.12 | 211189 |
| 100 | 91.09 | 22.00 | 219982 |

Additionally, for example, polymers of the present disclosure such as crosslinked polyacrylate polymers may have magnesium concentration measurements (e.g., average magnesium concentration measurements as determined by ICP-AES analysis) of about 8,365 μg of magnesium to about 55,980 μg of magnesium per gram of the polymer. This range approximately corresponds to a polymer in which magnesium serves as a counterion to about 5% to about 35% of the carboxylate groups, as shown in Table 4. In some embodiments, polymers of the present disclosure such as crosslinked polyacrylate polymers may have magnesium concentration measurements (e.g., average magnesium concentration measurements as determined by ICP-AES analysis) of about 24,717 μg of magnesium to about 55,980 μg of magnesium per gram of the polymer. This range approximately corresponds to a polymer in which magnesium serves as a counterion to about 15% to about 35% of the carboxylate groups, as shown in Table 4.

TABLE 4
Magnesium Content for Various Exemplary Polyacrylate Polymers.

| % of Carboxylates | | | |
|---|---|---|---|
| with Mg | MW | Wt % Mg | μg/g Mg |
| 0 | 72.06 | 0.00 | 0 |
| 5 | 72.62 | 0.84 | 8365 |
| 10 | 73.18 | 1.66 | 16603 |
| 15 | 73.73 | 2.47 | 24717 |
| 20 | 74.29 | 3.27 | 32709 |
| 25 | 74.85 | 4.06 | 40581 |
| 30 | 75.41 | 4.83 | 48338 |
| 35 | 75.96 | 5.60 | 55980 |
| 40 | 76.52 | 6.35 | 63511 |
| 45 | 77.08 | 7.09 | 70934 |
| 50 | 77.64 | 7.82 | 78249 |
| 55 | 78.19 | 8.55 | 85461 |
| 60 | 78.75 | 9.26 | 92570 |
| 62 | 78.97 | 9.54 | 95386 |
| 65 | 79.31 | 9.96 | 99580 |
| 70 | 79.87 | 10.65 | 106491 |
| 72 | 80.09 | 10.92 | 109229 |
| 75 | 80.42 | 11.33 | 113307 |
| 80 | 80.98 | 12.00 | 120029 |
| 85 | 81.54 | 12.67 | 126659 |
| 90 | 82.10 | 13.32 | 133199 |
| 95 | 82.65 | 13.97 | 139651 |
| 100 | 83.21 | 14.60 | 146016 |

The percentage of carboxylate groups to which magnesium serves as a counterion on a polymer (e.g., the “[x]% Mg-PAA” nomenclature) can be determined from the weight percent measurement (wt. % Mg) by the following equation:

[x]% Mg-PAA=(72.06)(wt. % Mg)/(12.15−(0.11(wt. % Mg))

Using Mixed Sample A and Mixed Sample B described in above, sodium content was determined by ICP-AES as follows. Three 1.0-mL aliquots of Mixed Sample A were each diluted to a final volume of 10.0 mL using a 1% nitric acid solution. To each was added 113 μL of a 10,000 μg/mL standard solution of 99.999% scandium oxide in 5% nitric acid. Similarly, three 1.00-mL aliquots of Mixed Sample B were diluted to final volumes of 10.0 mL and were doped with 115 μL, 115 μL, and 116 μL of the standard scandium solution. Analysis of sodium content proceeded using a ThermoElectron Finnigan Element 2 ICP-AES instrument (equipped with software version 2.42) according to the manufacturer's specifications. The six sodium concentration measurements (e.g., 327, 328, 328, 381, 381, and 381 μg/g, respectively) were determined by normalizing the intensity of the raw sodium measurement to the measurement of the internal scandium standard and correcting for dilution. These six raw sodium concentration measurements were then averaged (354 μg/g) wherein:

354 μg/g is equivalent to 0.035 wt % sodium.

The percentage of carboxylate groups to which sodium serves as a counterion (e.g., the “[x]% Na-PAA” nomenclature) on a polyacrylate polymer can be determined from the weight percent sodium measurement (wt. % Na) by the following equation:

[x]% Na-PAA=(72.06)(wt. % Na)/(23.0−(0.23)(wt. % Na))

For this example analysis, with an average sodium concentration of 354 μg of sodium per gram of polyacrylate polymer, or 0.035 wt. % sodium, sodium cations are counterions to about 0.11% of the carboxylate groups in the polymer.

Polymers of the present disclosure may have sodium concentration measurements (e.g., average sodium concentration measurements as determined by ICP-AES analysis) of about 0 μg of sodium to about 16,100 μg of sodium per gram of polyacrylate polymer. This range approximately corresponds to a polymer in which sodium serves as a counterion to about 0% to about 5% of the carboxylate groups.

In another exemplary method, the content of certain cations (e.g., calcium, sodium, magnesium, potassium or other cations) on a polyacrylate polymer may be determined by ICP-OES. For example, the calcium content of a polymer prepared according to Example 5 can be determined by diluting a measured mass of polyacrylate polymer with a known volume of a 5% aqueous solution of trace metal grade nitric acid. The sample is then digested by first heating the polyacrylate polymer mixture until gaseous NO₂ is apparent. While continuing to heat, a small measured aliquot of 30-40% hydrogen peroxide is added to the solution. The solution foams and may turn brown. Once the foaming subsides an additional aliquot of hydrogen peroxide is added and repeated until the foaming after hydrogen peroxide addition is minimal, no particulate is visible, and a clear and colorless solution including, for example, a fully digested sample, has been prepared. The total volume of hydrogen peroxide is recorded. Additional measured volumes of 5% nitric acid may be added during the digestion process to maintain an adequate volume of liquid. An appropriate volume of the digested polyacrylate polymer sample is diluted to a final volume of 10 mL with the 5% nitric acid solution to bring the concentration of the cation within the range of a suitable calibration curve; serial dilutions in 5% nitric acid can be made with the total dilution recorded. An internal scandium/cesium standard/ionization buffer was prepared from CsNO₃ and a scandium standard and was used in all analyses to normalize results and correct for matrix effects. The internal standard was prepared by adding 50 mg scandium standard (1000 μg/mL) and 1.48 g anhydrous CsNO3 to 1 L of 5% trace metal grade nitric acid. The internal was mixed with the sample online prior to injection into the ICP instrument. Standard solutions for construction of the standard curve were prepared at 0.2, 1, 5 and 25 μg/g Ca in 5% nitric acid. Samples were analyzed by ICP-OES on a Perkin Elmer Optima 5300 DV. Ca concentrations in μg/g were determined from the standard curve with correction for dilution, and converted to weight percent as described above.

In another exemplary method, the content of certain cations (e.g., calcium, sodium, magnesium, potassium or other cations) on a polymer may be determined by ICP-OES using microwave digestion of the sample in a nitric acid, hydrochloric acid, and hydrogen peroxide digestion medium. For example, the calcium content of a polymer prepared as described by any one or more of Examples 1, 3 and 5 can be determined by placing 50 mg of polymer with 0.800 mL trace metal grade nitric acid, 0.450 mL concentrated trace metal grade hydrochloric acid and 0.200 mL of 30% (w/w) hydrogen peroxide in a digestion vessel. The vessel is then placed in a MARS 5 (CEM Corp) microwave at 100% power for 2 minutes (to a temperature of 165° C.) followed by 3 minutes at 100% power (to a temperature of 175° C.) and then holding the sample at 175° C. for 10 minutes to digest the sample including, for example, to completely digest the sample. The digested polymer sample is then diluted to a final volume of 50 mL with purified water to bring the concentration of the cation within the range of the standard curve. Standard solutions for construction of the standard curve were prepared at 0 (blank), 100, 500 and 1000 ug/mL Ca in 4% (v/v) nitric acid. An internal standard solution was prepared containing 20 μg/mL yttrium and 100 μg/mL germanium in 4% trace metal grade nitric acid. The internal standard was used in all analyses to normalize results and correct for matrix effects. Samples were analyzed on a Thermo Electron iCAP 6000 ICP-OES. Ca concentrations in μg/g were determined from the standard curve with correction for dilution, and converted to weight percent as described above.

Likewise, sodium content in the same sample was analyzed by placing 50 mg of polymer with 0.800 mL trace metal grade nitric acid, 0.450 mL concentrated trace metal grade hydrochloric acid and 0.200 mL of 30% (w/w) hydrogen peroxide in a digestion vessel. The vessel is then placed in a MARS 5 (CEM Corp) microwave at 100% power for 10 minutes (to a temperature of 185° C.) followed by 5 minutes at 100% power (to a temperature of 195° C.) and then holding the sample at 195° C. for 15 minutes to digest the sample. The digested polymer sample is then diluted to a final volume of 50 mL with purified water to bring the concentration of the cation within the range of the standard curve. Standard solutions for construction of the standard curve were prepared at 0 (blank), 0.1, 0.5 and 1.0 μg/mL Na in 4% (v/v) nitric acid.

The methods and calculations described above can similarly be applied to other polymers adjusting the calculations as known to one who is skilled in the art. For example, for poly2-fluoroacrylate (PFAA) the calcium, magnesium and sodium calculations are shown below. For ICP analyses which give cation values corrected for dilution in ug/g, the ug/g values are converted to a wt % by dividing by 10,000. The percent of carboxylate groups to which calcium serves as a counterion on a polymer (e.g., the “[x]% Ca-PFAA” nomenclature) can be determined from the weight percent calcium measurement (wt. % Ca) by the following equation:

[x]% Ca-PFAA=(90.1)(wt. % Ca)/(20.05−(0.19)(wt. % Ca))

The percentage of carboxylate groups to which magnesium serves as a counterion on a polymer (e.g., the “[x]% Mg-PFAA” nomenclature) can be determined from the weight percent calcium measurement (wt. % Ca) by the following equation:

[x]% Mg-PFAA=(90.1)(wt. % Mg)/(12.15−(0.11(wt. % Mg))

The percentage of carboxylate groups to which sodium serves as a counterion on a polymer (e.g., the “[x]% Na-PFAA” nomenclature) can be determined from the weight percent calcium measurement (wt. % Ca) by the following equation:

[x]% Na-PFAA=(90.1)(wt. % Na)/(23−(0.23)(wt. % Na))

For example, polyfluoroacrylic acid polymers of the present disclosure may have calcium concentration measurements (e.g., average calcium concentration measurements as determined by ICP-AES analysis) of about 11,004 μg of calcium to about 143,924 μg of calcium per gram of the polymer. This range approximately corresponds to a polymer in which calcium serves as a counterion to about 5% to about 75% of the carboxylate groups, as shown in Table 5. In some embodiments, polymers of the present disclosure such as crosslinked polyacrylate polymers may have calcium concentration measurements (e.g., average calcium concentration measurements as determined by ICP-AES analysis) of about 32,333 μg of calcium to about 72,466 μg of calcium per gram of the polymer. This range approximately corresponds to a polymer in which calcium serves as a counterion to about 15% to about 35% of the carboxylate groups, as shown in Table 5.

TABLE 5
Calcium Content for Various Exemplary Polyfluoroacrylate Polymers.

| % of Carboxylates | | | |
|---|---|---|---|
| with Ca | MW | Wt % Ca | μg Ca/g polymer |
| 0 | 90.10 | 0.00 | 0 |
| 5 | 91.1 | 1.10 | 11004 |
| 10 | 92.0 | 2.18 | 21779 |
| 15 | 93.0 | 3.23 | 32333 |
| 20 | 93.9 | 4.27 | 42672 |
| 25 | 94.9 | 5.28 | 52804 |
| 30 | 95.8 | 6.27 | 62733 |
| 35 | 96.8 | 7.25 | 72466 |
| 40 | 97.7 | 8.20 | 82009 |
| 45 | 98.7 | 9.14 | 91368 |
| 50 | 99.7 | 10.05 | 100547 |
| 55 | 100.6 | 10.96 | 109552 |
| 60 | 101.6 | 11.84 | 118387 |
| 65 | 102.5 | 12.71 | 127058 |
| 70 | 103.5 | 13.56 | 135569 |
| 75 | 104.4 | 14.39 | 143924 |
| 80 | 105.4 | 15.21 | 152128 |
| 85 | 106.3 | 16.02 | 160184 |
| 90 | 107.3 | 16.81 | 168097 |
| 95 | 108.2 | 17.59 | 175870 |
| 100 | 109.2 | 18.35 | 183507 |

Additionally, for example, polymers of the present disclosure such as crosslinked polyacrylate polymers may have magnesium concentration measurements (e.g., average magnesium concentration measurements as determined by ICP-AES analysis) of about 6,701 μg of magnesium to about 45,238 μg of magnesium per gram of the polymer. This range approximately corresponds to a polymer in which magnesium serves as a counterion to about 5% to about 35% of the carboxylate groups, as shown in Table 6. In some embodiments, polymers of the present disclosure such as crosslinked polyacrylate polymers may have magnesium concentration measurements (e.g., average magnesium concentration measurements as determined by ICP-AES analysis) of about 19,859 μg of magnesium to about 45,238 μg of magnesium per gram of the polymer. This range approximately corresponds to a polymer in which magnesium serves as a counterion to about 15% to about 35% of the carboxylate groups, as shown in Table 6.

TABLE 6
Magnesium Content for Various Exemplary Polyluoroacrylate Polymers.

| % of Carboxylates | | | |
|---|---|---|---|
| with Mg | MW | Wt % Mg | μg/g Mg |
| 0 | 90.1 | 0.0 | 0 |
| 5 | 90.7 | 0.7 | 6701 |
| 10 | 91.2 | 1.3 | 13320 |
| 15 | 91.8 | 2.0 | 19859 |
| 20 | 92.3 | 2.6 | 26319 |
| 25 | 92.9 | 3.3 | 32701 |
| 30 | 93.4 | 3.9 | 39007 |
| 35 | 94.0 | 4.5 | 45238 |
| 40 | 94.6 | 5.1 | 51396 |
| 45 | 95.1 | 5.7 | 57482 |
| 50 | 95.7 | 6.3 | 63496 |
| 55 | 96.2 | 6.9 | 69441 |
| 60 | 96.8 | 7.5 | 75318 |
| 65 | 97.3 | 8.1 | 81127 |
| 70 | 97.9 | 8.7 | 86870 |
| 75 | 98.5 | 9.3 | 92548 |
| 80 | 99.0 | 9.8 | 98162 |
| 85 | 99.6 | 10.4 | 103713 |
| 90 | 100.1 | 10.9 | 109203 |
| 95 | 100.7 | 11.5 | 114631 |
| 100 | 101.3 | 12.0 | 120000 |

Polymers of the present disclosure may have sodium concentration measurements (e.g., average sodium concentration measurements as determined by ICP-AES analysis) of about 0 μg of sodium to about 12,610 μg of sodium per gram of polyacrylate polymer. This range approximately corresponds to a polymer in which sodium serves as a counterion to about 0% to about 5% of the carboxylate groups.

### Example 7

The effect of the percent of calcium counterions of the hydrated polycarboxylic acid polymer on adhesion including, for example, bioadhesion to skin, adhesion to stainless steel and glass, and on the morphology of the dried calcium acrylate particles was assessed.

For these studies, polyacrylic acid is prepared, for example, as described in Examples 1 and 3. Calcium oxide was then added to each sample in the amount shown in Table 7 to achieve different amounts of % calcium acrylate, e.g., 5 to 40%. After equilibration with stirring excess solution was drained from the calcium acrylate. The hydrated polymer was used for adhesiveness determinations.

To determine the actual % calcium substitution versus target % calcium substitution, samples of each calcium loaded bead preparation were placed in glass beakers and dried in a vacuum oven. These samples were then analyzed for calcium content using an Inductively Coupled Plasma Optical Emission Spectroscopy (ICP-OES) method, for example, as described in Example 6. Actual % calcium substitution versus target % calcium substitution is shown in Table 7.

TABLE 7
Ca-CLP Sample Preparation Summary

| | Weight of Added | |
|---|---|---|
| Target % Ca Substitution | CaO, g | Actual % Ca Substitution |
| 0 | 0.0000 | 1 |
| 5 | 0.0782 | 5 |
| 10 | 0.1570 | 11 |
| 15 | 0.2408 | 18 |
| 20 | 0.3173 | 24 |
| 25 | 0.3963 | 28 |
| 30 | 0.4698 | 32 |
| 35 | 0.5492 | 37 |
| 40 | 0.6317 | 41 |

The adhesiveness of the Ca acrylate samples was assessed using several substrates including, skin, glass, and steel. The adhesion results are summarized in Table 8. In the hydrated state, polyacrylic acid (0% calcium) was adhesive to skin, glass, and steel. In contrast, 10% to 40% Ca acrylate was not adhesive to skin, glass, and steel.

TABLE 8
Adhesiveness of Ca-CLP in Hydrated Stage vs. Degree of Ca Substitution

Adhesiveness in Hydrated State

| Target % Ca-CLP | Skin (finger) | Glass | Steel |
|---|---|---|---|
| 0 | Yes | Yes | Yes |
| 5 | Yes | Yes | No |
| 10 | No | No | No |
| 15 | No | No | No |
| 20 | No | No | No |
| 25 | No | No | No |
| 30 | No | No | No |
| 35 | No | No | No |
| 40 | No | No | No |

The morphology of dried Ca-CLP particles, with varying degree of calcium substitution was determined. Wet CLP particles were placed in glass beakers to dry in a vacuum oven. The morphology and adhesion to glass were assessed versus the degree of calcium substitution. Below 11% calcium substitution some CLP particles adhered so tightly to the glass that they could not be removed with a spatula. Below 24% calcium substitution firm pressure was required to remove some of the CLP particles from the surface. Between 24 and 32% calcium substitution some CLP particles adhered to the glass but could be dislodged with slight pressure. Above 32% calcium substitution none of the CLP particles adhered to the glass. In the dried state, the polyacrylate particles with up to 18% calcium substitution formed hard clumps while all samples with 24% calcium substitution and above formed individual particles.

Similarly, fluoroacrylic acid is prepared, for example by the method of Examples 21 to 26. Calcium oxide is then added to each sample in the amount shown in Table 9 to achieve different target amounts of % calcium acrylate, e.g., from 5 to 40%.

TABLE 9
Weight of calcium oxide added to achieve different target amounts of %
calcium acrylate

| Target % Ca Substitution | Weight of CaO, g |
|---|---|
| 0 | 0.0000 |
| 5 | 0.0782 |
| 10 | 0.1570 |
| 15 | 0.2408 |
| 20 | 0.3173 |
| 25 | 0.3963 |
| 30 | 0.4698 |
| 35 | 0.5492 |
| 40 | 0.6317 |

After equilibration with stirring excess solution was drained from the calcium fluoroacrylate. The hydrated polymer is then used for adhesiveness determination using skin, stainless steel and glass as described above. The morphology of the fluoroacrylate particles after drying is also assessed to determine whether the particles dry in clumps or as individual particles.

### Example 8

The saline holding capacity of a cross-linked polyelectrolyte polymer, such as a cross-linked polyacrylate polymer, may be determined by known methods in the art.

In an exemplary method, saline holding capacity for polyacrylic acid was determined with a 0.15 M sodium phosphate buffered solution as follows. A pH seven buffer of sodium phosphate tribasic (Na₃PO₄.12H₂O; MW 380.124) was prepared by dissolving 19.0062 grams in about 950 milliliters pure water and adjusting the pH to a final pH of 7±0.1 with 1N HCl before final dilution to one liter resulting in a solution with a sodium concentration of 0.15 M. Next, an amount of cross-linked cation-binding polyelectrolyte, for example, cross-linked polyacrylic acid particles (e.g., polyacrylic acid prepared according to Examples 1-4) (e.g., 0.1±0.025 grams), were transferred to a tared filter tube and the mass of the polymer was recorded as in W1. Next, the tube was returned to the balance to record the weight of the tube plus the sample as W2. An excess (e.g., more than seventy times the mass of polymer) amount of the pH 7.0 buffer (e.g., ten milliliters) was then transferred to the tube containing the polyacrylic acid sample. The tube was then placed on a flat bed shaker with shaking. At two, four and six hours free liquid was aspirated from the tube and a fresh aliquot (e.g. 10 mL) added. After six hours of shaking, all excess fluid was removed from the tube (e.g., no visible fluid in the tube) the tube and sample were weighed and recorded as W3. The saline holding capacity (SHC) was calculated by dividing the mass of the fluid absorbed by the mass of the dry crosslinked polyacrylate polymer, for example, SHC (g/g)=(W3−W2)/(W1). According to the present disclosure, cross-linked cation-binding polymers, including polyacrylic acid particles prepared according to the methods disclosed herein, had a saline holding capacity of 20 g/g, 30 g/g, 40 g/g, or more. Alternatively stated, such cross-linked cation-binding polymers, including where the polyelectrolyte is polyacrylic acid, can absorb 20-fold, 30-fold, 40-fold, or more of their mass in a saline solution.

The saline holding capacity of a cross-linked polyelectrolyte polymer, such as a cross-linked polyfluoroacrylic acid polymer, may be determined by known methods in the art.

In an exemplary method, saline holding capacity for poly2-fluoroacrylic acid is determined with a 0.15 M sodium phosphate buffered solution as follows. A pH seven buffer of sodium phosphate tribasic (Na₃PO₄.12H₂O; MW 380.124) is prepared by dissolving 19.0062 grams in about 950 milliliters pure water and adjusting the pH to a final pH of 7±0.1 with 1N HCl before final dilution to one liter resulting in a solution with a sodium concentration of 0.15 M. Next, an amount of cross-linked cation-binding polyelectrolyte, for example, cross-linked poly2-fluoroacrylic acid particles (e.g., poly2-fluoroacrylic acid prepared according to Examples 1-4) (e.g., 0.1±0.025 grams), is transferred to a tared filter tube and the mass of the polymer was recorded as in W1. Next, the tube is returned to the balance to record the weight of the tube plus the sample as W2. An excess (e.g., more than seventy times the mass of polymer) amount of the pH 7.0 buffer (e.g., ten milliliters) is then transferred to the tube containing the poly2-fluoroacrylic acid sample. The tube is then placed on a flat bed shaker with shaking. At two, four and six hours free liquid is decanted or aspirated from the tube and a fresh aliquot (e.g. 10 mL) added. After six hours of shaking, all excess fluid is removed from the tube (e.g., no visible fluid in the tube) the tube and sample weighed and recorded as W3. The saline holding capacity (SHC) is calculated by dividing the mass of the fluid absorbed by the mass of the dry poly2-fluoroacrylic acid polymer, for example, SHC (g/g)=(W3−W2)/(W1). According to the present disclosure, cross-linked cation-binding polymers, including poly2-fluoroacrylic acid particles prepared according to the methods disclosed herein, had a saline holding capacity of 20 g/g, 30 g/g, 40 g/g, or more. Alternatively stated, such cross-linked cation-binding polymers, including where the polyelectrolyte is poly2-fluoroacrylic acid, can absorb 20-fold, 30-fold, 40-fold, or more of their mass in a saline solution.

### Example 9

The saline holding capacity of a cross-linked polyelectrolyte polymer, such as a cross-linked polyacrylate polymer, may be determined by known methods in the art.

In an exemplary method, a saline absorption capacity (e.g., saline holding capacity) cross-linked cation-binding polyelectrolytes with non-hydrogen counterions, for example cross-linked calcium polyacrylate is determined by first rinsing the polymer with hydrochloric acid to convert it to the acid form. The saline absorption capacity (e.g., saline holding capacity) of the acid form is then determined.

For example the saline absorption capacity (e.g., saline holding capacity) of calcium polyacrylate particles can be determined as follows. Phosphate equilibration buffer of 50 mM phosphate, 154 mM NaCl was prepared by dissolving 19.5 g trisodium phosphate dodecahydrate (Na₃PO₄. 12H₂O, molecular weight 380.12) in approximately 950 mL of deionized water with pH adjustment to 7.0 with 1N HCl and then diluting to a final volume of 1000 mL with deionized water. A disposable polypropylene chromatography tube was weighed and then 0.1 g of calcium polyacrylate particles were transferred to the tube and reweighed. 10 mL 1N HCl was added to the tube containing the sample and the tube was placed on a flat bed shaker and shaken for 30 minutes. The fluid was drained from the tube by gravity. Another 10 mL of 1N HCl was transferred into the tube and shaken for another 30 minutes. The fluid was drained from the tube by gravity. Another 10 mL of 1N HCl was transferred to the tube and shaken for 60 minutes. The fluid was drained from the tube by gravity. 10 mL of deionized water was transferred to the tube and the water immediately suctioned off using a vacuum flask and vacuum pump or a faucet-mounted water aspirator, until there was no visible fluid in the tube. This water rinse step was repeated two more times. 10 milliliters of the pH 7.0 phosphate buffer was transferred to the tube and shaken for 15 minutes. Using a vacuum flask and vacuum pump, or a faucet-mounted water aspirator, the fluid that was not absorbed into the polyacrylate particles was suctioned off so that there was no visible fluid in the tube. 10 mL of the pH 7.0 phosphate buffer was transferred to the tube and the polymer was permitted to swell for 30 minutes then fluid suctioned off until there was no visible fluid in the tube. This was repeated twice with 15 minute swell times and then with a 3 hour swell time so that the total swelling time was four hours. After suctioning off the fluid the tube with swollen polymer was weighed.

The amount of phosphate buffered saline absorbed by the polymer was determined by subtracting the original weight of the tube with dry polymer from the weight of the tube with swollen polymer.

The saline absorption capacity (e.g., saline holding capacity) was determined by dividing the amount of saline absorbed by the polymer by the weight of the polymer used in the test (g/g polymer).

Similarly, in an exemplary method, a saline absorption capacity (e.g., saline holding capacity) for cross-linked cation-binding polyelectrolytes with non-hydrogen counterions, for example cross-linked calcium poly2-fluoroacrylate is determined by first rinsing the polymer with hydrochloric acid to convert the polymer to the acid form. The saline absorption capacity (e.g., saline holding capacity) of the acid form is then determined.

For example the saline absorption capacity (e.g., saline holding capacity) of calcium polyfluoroacrylate particles can be determined as follows. Phosphate equilibration buffer of 50 mM phosphate, 154 mM NaCl is prepared by dissolving 19.5 g trisodium phosphate dodecahydrate (Na₃PO₄.12H₂O, molecular weight 380.12) in approximately 950 mL of deionized water with pH adjustment to 7.0 with 1N HCl and then diluting to a final volume of 1000 mL with deionized water. A disposable polypropylene chromatography tube is weighed and then 0.1 g of calcium polyfluoroacrylate particles are transferred to the tube and reweighed. 10 mL 1N HCl is added to the tube containing the sample and the tube is placed on a flat bed shaker and shaken for 30 minutes. The fluid is drained from the tube by gravity. Another 10 mL of 1N HCl is transferred into the tube and shaken for another 30 minutes. The fluid is drained from the tube by gravity. Another 10 mL of 1N HCl is transferred to the tube and shaken for 60 minutes. The fluid is drained from the tube by gravity. 10 mL of deionized water is transferred to the tube and the water immediately suctioned off using a vacuum flask and vacuum pump or a faucet-mounted water aspirator, until there is no visible fluid in the tube. This water rinse step is repeated two more times. 10 milliliters of the pH 7.0 phosphate buffer is transferred to the tube and shaken for 15 minutes. Using a vacuum flask and vacuum pump, or a faucet-mounted water aspirator, the fluid that is not absorbed into the polyfluoroacrylic acid particles is suctioned off so that there is no visible fluid in the tube. 10 mL of the pH 7.0 phosphate buffer is transferred to the tube and the polymer is permitted to swell for 30 minutes then fluid suctioned off until there is no visible fluid in the tube. This is repeated twice with 15 minute swell times and then with a 3 hour swell time, so that the total swelling time is four hours. After suctioning off the fluid the tube with swollen polymer is weighed.

The amount of phosphate buffered saline absorbed by the polymer is determined by subtracting the original weight of the tube with dry polymer from the weight of the tube with swollen polymer.

The saline absorption capacity (e.g., saline holding capacity) is determined by dividing the amount of saline absorbed by the polymer by the weight of the polymer used in the test (g/g polymer).

### Example 10

The saline holding capacity of a cross-linked polyelectrolyte polymer, such as a cross-linked polyacrylate polymer, may be determined by known methods in the art.

In an exemplary method, a saline absorption capacity (e.g., saline holding capacity) of crosslinked cation-binding polymers comprising monomers containing carboxyate groups, wherein the polymers further comprise calcium and/or magnesium cations (e.g., calcium cations or magnesium cations or a mixture thereof), wherein the calcium and/or magnesium cations are counterions to the carboxylate groups in the polymer is measured using a centrifugal method. According to this method, the centrifuge retention capacity (CRC) of the polymer (e.g., calcium poly2-fluoroacrylate or magnesium polyfluoroacrylate) is determined without first treating the polymer with acid.

For example the saline holding capacity of calcium poly2-fluoroacrylate particles may be determined. A pH 7 175 mM sodium phosphate dibasic buffer is prepared with 24.84 g of sodium phosphate dibasic (anhydrous) in 1 L purified water, with pH adjustment to pH 7.0 with concentrated HCl. The weight of a centrifuge tube is determined (Wtube). 100±10 mg of the calcium poly2-fluoroacrylate particles are weighed and added to centrifuge tube and the tube reweighed (Wtube+sample). 25 mL of uptake buffer is then added to centrifuge tube and the tube capped and shaken vigorously. The tube is then shaken on a wrist-action shaker for at least 8 hours. The tube is then centrifuged for 10 minutes at 3500 relative centrifugal force and the supernatant removed with a disposable transfer pipette taking care not to remove the gel. Excess water can be removed by wicking with an absorbent paper wick. The tube with the swollen gel particles is reweighed (Wtube+swollen gel) and the saline holding capacity determined as:

Saline holding capacity (w/w)=(Wt(tube+swollen gel)−W(tube))/(W(tube+sample)−W(tube)).

### Example 11

This example describes the effects of polyacrylic acid polymer, e.g., prepared as described in Examples 1 and 3, and Ca-CLP (prepared by addition of calcium counterions to a polyacrylic acid polymer during manufacture, e.g., as described in Example 5) on fecal and urinary ion excretion and fecal mass in rats.

In an exemplary study with a polyacrylic acid polymer with calcium cations as couterions to the carboxylate groups on the polymer described in Table 10 were prepared as described by any one or more of Examples 1, 3 and 4. Each of these Ca-CLP polymers were tested in groups of six rats to determine the effect of the percent calcium on the Ca-CLP on the fecal excretion of Na, K Ca, Mg, urinary excretion of P, and on the mass of feces excreted.

TABLE 10
CLP Forms Manufactured

| Group | CLP Form |
|---|---|
| 1 | None (control) |
| 2 | polyacrylic acid |
| | polymer |
| 3 | 6.9% Ca-CLP |
| 4 | 10.5% Ca-CLP |
| 5 | 34.1% Ca-CLP |
| 6 | 31.1% Ca-CLP |
| 7 | 41.6% Ca-CLP |
| 8 | 62% Ca-CLP |
| 9 | 71% Ca-CLP |
| 10 | 76% Ca-CLP |

Ca-CLP was prepared as described by any one or more of Examples 1, 3 and 5, and the mixture was stirred and left to react overnight at room temperature. The mixture was then placed into a vacuum oven and heated at approximately 60° C.

Ca-CLP or polyacrylic acid polymer was mixed at a level of 5% into pulverized LabDiet 5012 and the mixture was processed through a food blender several times until the food/CLP powder was uniform in color and size. Daily measurements of rat weight and 24-hour food intake, water intake, urine output, and fecal output were recorded. Dosing started on Day 1. On Days 4, 5 and 6 24-hour feces and urine were collected for ICP-AES analysis of fecal Na, fecal K and urine P. Samples were digested for ICP-AES analysis by placing the sample in flask, adding an aqueous solution of 5% trace metal grade concentrated nitric acid, and heating to boiling. 30% hydrogen peroxide was then added in small aliquots until the solution was clear and vigorous foaming from addition of hydrogen peroxide had ceased. The digested samples were analyzed by ICP/AES (inductively coupled plasma atomic emission spectroscopy) for fecal sodium, fecal potassium, and urinary phosphate. Changes in fecal sodium and potassium excretion levels from control (rats on rat chow and no polymer) were calculated (e.g., control fecal sodium was subtracted from fecal sodium in the treatment groups).

As shown in Table 11, fecal sodium excretion was relatively independent of the percentage of calcium counterions on the CLP until a decrease between 34-42%. With and an approximately linear decrease from 42 to 71% while Fecal K decreased approximately linearly between 0-40% Ca-CLP and then remained relatively constant.

The effect of different amounts of calcium counterion on Ca-CLP on daily urinary phosphorus (P) excretion and fecal measures is also shown in Table 11 as change from control ((e.g., control urinary phosphorus excretion levels were subtracted from urinary phosphorus levels from treatment groups).

Urinary phosphorus declined in an approximately linearly with was percent calcium counterion on Ca-CLP. Urinary excretion of phosphorus is a measure of the acid/base status of the rat with increasing urine phosphorus correlating with a shift to a more acidic state.

The effect of the percent calcium counterion on Ca-CLP on fecal weight was determined and the change from control daily fecal weights are shown in Table 11 (e.g., control fecal weight was subtracted from fecal weights in treatment groups). Fecal weight was approximately constant between 0 and 31% Ca-CLP. Between 31 and 42% Ca-CLP the fecal weight dropped significantly and then became approximately constant to 71% Ca-CLP.

TABLE 11
Change From Control in Daily Fecal Sodium, Fecal Potassium, Urinary
Phosphorous, and Fecal Weight in Rats Administered polyacrylic acid
polymer or Ca-CLP

| | Δ Fecal Na | Δ Fecal K | Δ Urinary P | Δ Fecal Fluid |
|---|---|---|---|---|
| | Excretion | Excretion | Excretion | Excretion |
| CLP Form | (mg/d) | (mg/d) | (mg/d) | (g/day) |
| polyacrylic | 30.8 | 84.2 | 29.5 | 6.0 |
| acid polymer | | | | |
| 6.9% Ca-CLP | 28.9 | 78.7 | 22.6 | 5.9 |
| 10.5% Ca-CLP | 41.0 | 62.3 | 28.8 | 6.3 |
| 31.1% Ca-CLP | 40.9 | 29.3 | 12.1 | 6.0 |
| 34.1% Ca-CLP | 32.7 | 16.6 | 8.9 | 3.5 |
| 41.6% Ca-CLP | 19.6 | 6.8 | 4.0 | 1.4 |
| 62% Ca-CLP | 13.6 | 5.8 | 5.6 | 1.6 |
| 71% Ca-CLP | 13.2 | 8.9 | 1.2 | 1.8 |

### Example 12

This example describes the effects of poly2-fluoroacrylic acid polymer (e.g., prepared as described by any one or more of Examples 1, 3, 21, 22 and 26) and calcium poly2-fluoroacrylate polymer (e.g., prepared by addition of calcium counterions to poly2-fluoroacrylic acid as described in any one or more of Examples 5 and 27) on fecal and urinary ion excretion and fecal mass in rats.

In an exemplary study, calcium poly2-fluoroacrylate with calcium counterions at levels described in Table 12 are prepared using the methods described in Examples 4 and 5 including drying in a vacuum oven. Each of these calcium poly2-fluoroacrylate polymers are tested in groups of six rats to determine the effect of the percent calcium on the calcium poly2-fluoroacrylate on the fecal excretion of Na, K, Ca, Mg, urinary excretion of P, and on the mass of feces excreted.

TABLE 12
Treatment Groups

| | Target % Calcium Counterion on |
|---|---|
| Group | Poly2-fluoroacrylate |
| 1 | Control |
| 2 | 0 |
| 3 | 10 |
| 4 | 20 |
| 5 | 40 |
| 6 | 60 |
| 7 | 80 |

Calcium poly2-fluoroacrylate or poly2-fluoroacrylic acid is mixed at a level of 5% into pulverized LabDiet 5012 and the mixture is processed through a food blender several times until the food/CLP powder is uniform in color and size. Daily measurements of rat weight and 24-hour food intake, water intake, urine output, and fecal output are recorded. Dosing starts on Day 1. On Days 4, 5 and 6 24-hour feces and urine are collected for ICP-AES analysis of fecal Na, fecal K, fecal Ca, and fecal Mg and urine P. Samples are digested for ICP-AES analysis by placing the sample in flask, adding an aqueous solution of 5% trace metal grade concentrated nitric acid, and heating to boiling. 30% hydrogen peroxide is then added in small aliquots until the solution is clear and vigorous foaming from addition of hydrogen peroxide has ceased. The digested samples are analyzed by ICP/AES (inductively coupled plasma atomic emission spectroscopy) for fecal sodium, fecal potassium, fecal calcium, fecal magnesium and urinary phosphate.

The effect of different amounts of calcium counterion on calcium polyfluoroacrylate on fecal sodium, fecal potassium, fecal magnesium, fecal calcium, daily urinary phosphorus excretion and fecal mass are determined, and the change from control calculated.

### Example 13

This example describes the effects of polyacrylic acid polymer (e.g., prepared as described in Examples 1 and 3) and calcium polyacrylate polymer (e.g., prepared by addition of calcium counterions to polyacrylic acid during manufacturing as described in Example 5) with and without added CaCO₃ on fecal and urinary ion excretion and fecal mass in rats.

In this exemplary study, the effect of calcium polyacrylate or polyacrylic acid with or without calcium carbonate base (administered as TUMS®) on the fecal excretion of Na and K, fecal mass, and urinary excretion of P was studied in rats comparing CLP neutralization by calcium added during manufacturing (6.9% calcium polyacrylate or 25% calcium polyacrylate) and by CaCO₃ mixed into the feed as Tums (0 to 0.75 equivalents). 6.9% calcium polyacrylate and 25% calcium polyacrylate were prepared as described by any one or more of Examples 1, 3 and 5 with vacuum drying of the polyacrylate.

Multiple groups of 6 rats were fed diets containing polymer (polyacrylic acid, 6.9% calcium polyacrylate, or 25% calcium polyacrylate) mixed with TUMS® (0 to 0.75 equivalents of calcium carbonate) as 5 w/w % of their daily diet. Each group received a different treatment as described in Table 13. The diet was prepared by mixing calcium polyacrylate or polyacrylic acid, TUMS® where required, and pulverized LabDiet 5012 and then processing the mixture with a food blender several times until the powder was uniform in color and size.

Daily measurements of rat weight, food intake, water intake, urine output, and fecal output were recorded. Dosing started on Day 1. On days 4, 5, and 6 24-hour feces and urine were collected for ICP-AES analysis of fecal Na, fecal K and urine P. Samples were digested for ICP by placing each sample into a flask, adding an aqueous solution of 5% trace metal grade concentrated nitric acid, and heating to boiling. 30% hydrogen peroxide was then added in small aliquots until the solutions were clear and the vigorous foaming after additions of hydrogen peroxide had ceased. The digested samples were analyzed by ICP for fecal sodium, fecal potassium, and urinary phosphate. Changes in excretion over control (rats on rat chow and no polymer) fecal sodium, and potassium excretion levels were calculated and are shown in Table 13 (e.g., control fecal sodium and potassium excretion levels were subtracted from fecal sodium and potassium levels from treatment groups).

As shown in Table 13, Administration of polyacrylic acid/polyacrylate with and without base increased fecal excretion of both sodium and potassium compared to control for all formulations tested. Increasing the degree of polyacrylic acid neutralization had no significant effect on fecal sodium excretion whereas fecal potassium excretion decreased approximately linearly with increases in neutralization. Neutralization of polyacrylic acid by addition of calcium counterions at 6.9% and 25% during manufacture and/or by addition of CaCO₃ had similar effect on fecal sodium and potassium excretion and on urinary P excretion.

As shown in Table 13, co-administration of polyacrylic acid or calcium polyacrylate with base decreased urinary phosphorous levels. Urinary excretion of phosphorus is a measure of the acid/base status of the rat with increasing urine phosphorus correlating with a shift to a more acidic state. When polyacrylic acid was administered without base high urinary phosphorous values were observed. When polyacrylic acid or calcium polyacrylate was administered with increasing amounts of CaCO₃ base the urinary excretion of phosphorus was decreased and in the range of 65 to 72% base the urinary phosphorus was not different from controls. Neutralization of polyacrylic acid by addition of calcium counterions at 6.9% and 25% during manufacture and/or by addition of CaCO₃ had similar effect on urinary P excretion.

TABLE 13
Effect of Added CaCO₃ on Daily Fecal Sodium and Potassium Excretion
and Urinary Phosphorous Excretion in Rats Administered Polyacrylic Acid or
Calcium Polyacrylate

| | Equivalents | Total | | | |
|---|---|---|---|---|---|
| Percent of | of Base/ | Polyacrylate | | | |
| Carboxylates | Equivalent of | Carboxyl | Fecal Na | Fecal K | Urinary P |
| with Ca | COOH added | Neutralization¹ | Excretion | Excretion | Excretion |
| Counterions | as CaCO₃ | (%) | (mg/d) | (mg/d) | (mg/d) |
| 0 | 0 | 0 | 27.5 | 83.0 | 27.7 |
| 0 | 0.5 | 50 | 26.9 | 35.3 | 8.5 |
| 0 | .75 | 75 | 27.6 | 23.9 | −1.9 |
| 0 | .62 | 62 | 27.5 | 35.8 | 4.5 |
| 6.9 | 0 | 6.9 | 28.9 | 78.7 | 22.6 |
| 6.9 | .65 | 71.9 | 31.1 | 18.8 | −2.8 |
| 25 | 0 | 25 | 22.7 | 53.3 | 13.9 |
| 25 | .4 | 65 | 23.2 | 33.9 | 0.9 |
| 25 | .50 | 75 | 20.8 | 22.3 | −5.3 |

¹Total carboxyl neutralization is the sum of the percent of Ca counterions added during manufacturing and the equivalents of base mixed into the feed.

The addition of CaCO₃ to the rat feed decreases the fecal excretion of sodium and potassium. At an equivalent total polyacrylate neutralization ratio fecal sodium and potassium excretion and urinary P excretion were similar regardless of whether the neutralization was from calcium base added during manufacture or calcium base added as CaCO₃(Tums®).

A comparison of the data in Examples 11 and 12 show that for low levels of total neutralization (0% to about 30-35%), base added as CaCO₃ or base added as counterions during manufacture had similar effects on fecal sodium and potassium excretion. At higher levels of calcium counterions added during manufacture (from approximately 30-35% calcium polyacrylate to 71% calcium polyacrylate), fecal excretion of sodium and potassium decreased to a greater extent than for the same total neutralization of polyacrylic acid obtained with polyacrylic acid, 6.9% calcium polyacrylate or 25% calcium polyacrylate with added CaCO₃. The effect of neutralization of urinary phosphorus excretion did not depend whether the calcium counterions were added during manufacture or base was mixed into the formulation as CaCO_{3.}

Changes in fecal weight compared to control are shown in Table 14 (control fecal weight was subtracted from fecal weight in the treatment groups).

TABLE 14
Effect of Added CaCO₃ on Daily Fecal Excretion in Rats
Administered 0-25% Ca Polyacrylate

| Percent of | Equivalents of | | |
|---|---|---|---|
| Carboxylates | Base/Equivalent | Total Polyacrylate | Δ Fecal Fluid |
| with Ca | of COOH added as | Carboxyl | Excretion |
| Counterions | CaCO₃ | Neutralization (%)¹ | (g/day) |
| 0 | 0 | 0 | 6.8 |
| 0 | 0.5 | 50 | 4.5 |
| 0 | .75 | 75 | 3.7 |
| 0 | .62 | 62 | 3.8 |
| 6.9 | 0 | 6.9 | 6.4 |
| 6.9 | .65 | 7 | 3.3 |
| 25 | 0 | 25 | 5.8 |
| 25 | .4 | 65 | 3.0 |
| 25 | .50 | 75 | 1.7 |

¹Total carboxyl neutralization is the sum of the percent of Ca counterions added during manufacturing and the equivalents of base mixed into the feed.

A comparison of the data in Examples 11 and 12 is provided in Table 15 for ease of comparison. At low levels of reacted calcium (0% to about 30-35%) on the polymer, base added as CaCO₃ or calcium counterions added during manufacturing had similar effects on fecal sodium and potassium excretion. At higher levels of calcium counterions added during manufacturing (about 30-35% to 75%) fecal excretion of sodium and potassium were decreased compared to mixtures of CaCO₃ with low levels of reacted Ca+CaCO₃ or polyacrylic acid. Base added as CaCO₃ or calcium counterions added during manufacturing had similar effects on urinary P excretion.

TABLE 15
Comparison of Fecal Na and K Excretion from Tables 8 and 9 for for
Total CLP Carboxyl Neutralization Between 31 and 75% Neutralization

| | | Total | | |
|---|---|---|---|---|
| Percent of | Equivalents of | Polyacrylate | | |
| Carboxylates | Base/Equivalent | Carboxyl | Fecal Na | Fecal K |
| with Ca | of COOH added | Neutralization¹ | Excretion | Excretion |
| Counterions | as CaCO₃ | (%) | (mg/d) | (mg/d) |
| 31.1 | 0 | 31.1 | 32.8 | 16.6 |
| 34.1 | 0 | 34.1 | 40.9 | 29.3 |
| 41.6 | 0 | 41.6 | 19.6 | 6.8 |
| 0 | 0.5 | 50 | 26.9 | 35.3 |
| 62% | 0 | 62 | 13.6 | 5.8 |
| 0 | 0.62 | 62.5 | 27.5 | 35.8 |
| 25% Ca-CLP | 0.4 | 65 | 23.2 | 33.9 |
| 71% | 0 | 71 | 13.2 | 8.9 |
| 6.9% | 0.65 | 71.9 | 31.1 | 18.8 |
| 0 | 0.75 | 75 | 27.6 | 23.9 |
| 25% | 0.5 | 75 | 20.8 | 22.3 |

¹Total carboxyl neutralization is the sum of the percent of Ca counterions added during manufacturing and the equivalents of base mixed into the feed.

### Example 14

This example describes the effects of poly2-fluoroacrylic acid polymer (e.g., prepared as described by any one or more of Examples 1, 3, 21, 22 and 26) and calcium poly2-fluoroacrylate polymer (e.g., prepared by addition of calcium counterions to poly2-fluoroacrylic acid as described in any one or more of Examples 5 and 27) with and without added CaCO₃ on fecal and urinary ion excretion and fecal mass in rats.

In this exemplary study, the effect of calcium poly2-fluoroacrylate or poly2-fluoroacrylic acid with or without calcium carbonate base on the fecal excretion of Na and K, fecal mass, and urinary excretion of P is studied in rats comparing polyacrylate neutralization by calcium added during manufacturing (10% calcium poly2-fluoroacrylate or 25% calcium poly2-fluoroacrylate) and by CaCO₃ mixed into the feed (0 to 1.0 equivalents). 10% calcium poly2-fluoroacrylate and 25% calcium poly2-fluoroacrylate are prepared as described by any one or more of Examples 1, 3 and 5 with vacuum drying of the poly2-fluoroacrylate.

Multiple groups of 6 rats are fed diets containing polymer (poly2-fluoroacrylic acid, 10% calcium poly2-fluoroacrylate, or 25% calcium poly2-fluoroacrylate) mixed with 0 to 1.0 equivalents of calcium carbonate as 5 w/w % of their daily diet. Each group receives a different treatment as described in Table 16. The diet is prepared by mixing calcium poly2-fluoroacrylate or poly2-fluoroacrylic acid, calcium carbonate where required, and pulverized LabDiet 5012 and then processing the mixture with a food blender several times until the powder is uniform in color and size.

TABLE 16
Treatment Groups

| | Equivalents of | |
|---|---|---|
| Percent of | Base/Equivalent | |
| Carboxylates | of COOH | Total Polyacrylate |
| with Ca | added as | Carboxyl |
| Counterions | CaCO₃ | Neutralization¹ (%) |
| Control | | |
| 0 | 0 | 0 |
| 0 | 0.25 | 25 |
| 0 | 0.5 | 50 |
| 0 | 0.75 | 75 |
| 0 | 1.0 | 100 |
| 10 | 0 | 10 |
| 10 | 15 | 25 |
| 10 | .40 | 50 |
| 10 | .50 | 75 |
| 25 | 0 | 25 |
| 25 | .25 | 50 |
| 25 | .50 | 75 |

Daily measurements of rat weight, food intake, water intake, urine output, and fecal output are recorded. Dosing started on Day 1. On days 4, 5, and 6 24-hour feces and urine are collected for ICP-AES analysis of fecal Na, K, Mg and Ca and urine P. Samples are digested for ICP by placing each sample into a flask, adding an aqueous solution of 5% trace metal grade concentrated nitric acid, and heating to boiling. 30% hydrogen peroxide is then added in small aliquots until the solutions are clear and the vigorous foaming after additions of hydrogen peroxide had ceased. The digested samples are analyzed by ICP for fecal Na, K, Mg, and Ca and urinary P. The cation excretion values, and fecal mass, and change in these values from from control (rats on rat chow and no polymer) are determined.

### Example 15

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

An open-label clinical trial was performed in twelve healthy human subjects with a polyacrylic acid polymer with calcium cations as counterions to carboxylate groups in the polymer. Each patient received an equivalent of 15 g polyacrylic acid polymer/day as either 25% Ca-CLP (n=6) or 60% Ca-CLP (n=6), divided into three doses, administered one hour prior to meals. Subjects remained in the clinical research unit for the duration of the study.

25% Ca-CLP and 60% Ca-CLP were prepared as described by any one or more of Examples 1, 3 and 5. After cation exchange to load the polyacrylate with calcium, the polymer was milled to break up the bead structure and reduce the particle size. The Ca-CLP powder was mixed into pudding immediately prior to dosing. The subjects were required to eat the entire pudding aliquot.

The clinical trial evaluated whether administration of Ca-CLP when compared to a baseline period (1) altered fecal excretion of sodium, potassium, or phosphorous (2) altered measures of acidosis including serum total bicarbonate, urine pH and urine phosphorus, (3) altered serum potassium levels and (4) altered fecal weight.

After a 5 day baseline period, Ca-CLP was administered in pudding, 3 times a day for a total of 7 days (a total of 14 doses). For 25% Ca-CLP the dose was 16 g (5.33 g tid). For 60% Ca-CLP the dose was 18 g (6 g tid). A dose of 16 g of 25% Ca-CLP and 18 g 60% Ca-CLP each delivered an equivalent number of moles of cation exchange carboxyl groups as 15 g of polyacrylic acid polymer (208 mEq).

Diet was controlled with all participants having identical meals. Subjects were requested to consume all of their meals.

Subjects fasted for at least eight hours at screening and four hours at admission prior to the collection of blood and urine samples for clinical laboratory tests. Fasting was not required prior to collection of urine and blood samples taken during the study. Water ad libitum was allowed during the periods of fasting.

Twenty four hour daily stool and urine samples were collected daily and evaluated for stool weight, fecal electrolytes, urine pH, and urine phosphorus. Daily serum samples were evaluated for serum potassium and total bicarbonate. Fecal samples were evaluated by ICP for the concentration of sodium, potassium, calcium and magnesium. All urine specimens were collected and volume recorded. Urine samples were pooled for each 24-hour period and an aliquot sampled for sodium, potassium, calcium, phosphorous and magnesium analysis.

Daily parameters for the treatment period were compared to baseline, with daily parameters for days 3-6 averaged and compared to the average for treatment days 10-13. The average change from baseline in fecal excretion of sodium and potassium are shown in Table 17. An increase in percent calcium counterion from 25 to 60% resulted in a decrease in fecal sodium and potassium excretion and a larger drop in serum potassium.

TABLE 17
Average change from baseline in fecal sodium, fecal potassium and
serum potassium

| Ca | Fecal | | |
|---|---|---|---|
| Counterion | Na | Fecal K | Serum K |
| (%) | (mg/d) | (mg/d) | (mmol/L) |
| 25 | 1005 | 2129 | −0.15 |
| 25% Ca-CLP | | | |
| 60 | 169 | 1103 | −0.43 |
| 60% Ca-CLP | | | |

Measures of acidosis included urine pH, total serum CO₂, and urine phosphate. The average change from baseline in these parameters for Days 7-13 are shown in Table 18. The change from baseline in serum bicarbonate and urine pH were similar for 25% Ca-CLP and 60% Ca-CLP. The urinary phosphorus excretion decreased by a factor of 10 with an increase from 25% to 60% calcium counterion. This was paralleled by a decrease in fecal phosphorus excretion by a factor of 10 between 25% and 60% calcium counterion.

TABLE 18
Average Change from Baseline in Acidosis Parameters for Days 7-13

| | | | Urinary | |
|---|---|---|---|---|
| Ca | | | Phosphorus | Fecal Phosphorus |
| Counterion | Total serum | | Excretion | Excretion |
| (%) | CO₂ (mmol/) | Urine pH | (mg/day) | (mg/day) |
| 25 | −0.9 | −0.48 | 277 | −223 |
| 25% Ca-CLP | | | | |
| 60 | −0.7 | −0.63 | 27 | −19 |
| 60% Ca-CLP | | | | |

Administration of Ca-CLP led to an increase in fecal weight as shown in Table 19. This increase in fecal weight was not associated with diarrhea but is expected to be due to water entrapped in the superabsorbent polymer. An increase in the percent of calcium counterion from 25 to 60% decreased the fecal weight.

TABLE 19
Average Change from Baseline in Fecal Weight for Days 7-13

| Ca Counterion | |
|---|---|
| (%) | Fecal Wt (g) |
| 25 | 197 |
| 25% Ca-CLP | |
| 60 | 87 |
| 60% Ca-CLP | |

Similar studies may be conducted with a polyfluoroacrylic acid polymer with calcium cations as counterions to carboxylate groups in the polymer, alone or in combination with a base (e.g., calcium carbonate).

### Example 16

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

An open-label clinical trial was performed in twenty four healthy human subjects with a polyacrylic acid polymer with calcium cations as counterions to carboxylate groups in the polymer. Each patient received an equivalent of 15 g polyacrylic acid polymer/day as either 25% Ca-CLP or 50% Ca-CLP, divided into three doses, administered one hour prior to meals. Subjects remained in the clinical research unit for the duration of the study.

TABLE 20
Dose Groups

| | Number | | | Dose of | |
|---|---|---|---|---|---|
| | of | CLP | Dose | CLP | Dose of |
| Group | Subjects | formulation¹ | Administration | (g)² | CaCO₃ (g) |
| 1 | 6 | polyacrylic | Capsules with | 15 | 0 |
| | | acid polymer + | water | | |
| | | 75% CaCO₃ | | | |
| 2 | 6 | 25% Ca-CLP + | Powder mixed | 16 | 5.2 |
| | | 50% CaCO₃ | in pudding | | |
| 3 | 6 | 25% Ca-CLP + | Capsules with | 16 | 3.6 |
| | | 35% CaCO₃ | water | | |
| 4 | 6 | 50% Ca-CLP | Powder mixed | 17 | 0 |
| | | | in pudding | | |

¹25% Ca-CLP is polyacrylic acid polymer with 25% of the carboxyl groups reacted with calcium base; 50% CaCO₃ is CaCO₃ added to the formulation at a mass that will neutralize 50% of the carboxyl groups in an equivalent dose of polyacrylic acid polymer.
²All CLP doses gave an equivalent dose of carboxyl groups as 15 g of polyacrylic acid polymer

25% Ca-CLP and 50% Ca-CLP were prepared as described by any one or more of Examples 1, 3, and 5. After cation exchange to load the polyacrylate with calcium, the polymer was milled to break up the bead structure and reduce the particle size. The Ca-CLP powder was mixed into pudding immediately prior to dosing. The subjects were required to eat the entire pudding aliquot.

The clinical trial evaluated whether administration of CLP when compared to a baseline period (1) altered fecal excretion of sodium, potassium or phosphorus (2) altered measures of acidosis including serum total bicarbonate, urine pH and urine phosphorus, (3) altered serum potassium levels, and (4) altered fecal weight.

After a 5 day baseline period, CLP was administered in capsules with water or in pudding, twice a day (before breakfast and before dinner) for a total of 7 days (a total of 14 doses) as shown in Table 20. Groups 1 and 3 had the CLP formulation administered in capsules with water and Groups 2 and 4 had the CLP formulation mixed into pudding immediately prior to administration. All groups were administered an equivalent number of moles of cation exchange carboxyl groups as 15 g of polyacrylic acid polymer (208 mEq).

Diet was controlled with all participants having identical meals. Subjects were requested to consume all of their meals.

Subjects fasted for at least eight hours at screening and four hours at admission prior to the collection of blood and urine samples for clinical laboratory tests. Fasting was not required prior to urine and blood samples taken during the study. Water ad libitum was allowed during the periods of fasting.

Twenty four hour daily stool and urine samples were collected daily and evaluated for stool weight, fecal electrolytes, urine pH, and urine phosphorus. Daily serum samples were evaluated for serum potassium and total bicarbonate. Fecal samples were evaluated by ICP for the concentration of sodium, potassium, calcium, and magnesium. All urine specimens were collected and volume recorded. Urine samples were pooled for each 24-hour period and an aliquot sampled for sodium, potassium, calcium, phosphorous, and magnesium analysis.

Daily parameters for the treatment period were compared to baseline, with daily parameters for days 3-6 averaged and compared to the average for treatment days 10-13. The average change from baseline in fecal excretion of sodium and potassium are shown in Table 21. All groups had an increase in fecal sodium and potassium excretion compared to baseline.

TABLE 21
Average change from baseline in fecal sodium, fecal potassium and
serum potassium

| | | Fecal Na | Fecal K | Serum K |
|---|---|---|---|---|
| Group | CLP formulation^{1,2} | (mg/d) | (mg/d) | (mmol/L) |
| 1 | polyacrylic acid | 534 | 1258 | −0.2 |
| | polymer + 75% CaCO₃ | | | |
| 2 | 25% Ca-CLP + | 342 | 1415 | −0.2 |
| | 50% CaCO₃ | | | |
| 3 | 25% Ca-CLP + | 671 | 1907 | 0.0 |
| | 35% CaCO₃ | | | |
| 4 | 50% Ca-CLP | 419 | 1518 | −0.1 |

¹25% Ca-CLP is polyacrylic acid polymer with 25% of the carboxyl groups reacted with calcium base; 50% CaCO₃ is CaCO₃ added to the formulation at a mass that will neutralize 50% of the carboxyl groups in an equivalent dose of polyacrylic acid polymer.
²All CLP doses gave an equivalent dose of carboxyl groups as 15 g of polyacrylic acid polymer

Measures of acidosis included urine pH, total serum CO₂ and urine phosphate. The average change from baseline in these parameters for Days 7-13 are shown in Table 22.

TABLE 22
Average Change from Baseline in Acidosis Parameters for Days 7-13

| | | Total | | Urinary | Fecal |
|---|---|---|---|---|---|
| | | serum | | Phosphorus | Phosphorus |
| | CLP | CO₂ | Urine | Excretion | Excretion |
| Group | formulation^{1,2} | (mmol/) | pH | (mg/day) | (mg/day) |
| 1 | polyacrylic | −0.3 | −0.3 | 110 | −101 |
| | acid polymer + | | | | |
| | 75% CaCO₃ | | | | |
| 2 | 25% Ca-CLP + | 0.7 | −0.2 | 148 | −160 |
| | 50% CaCO₃ | | | | |
| 3 | 25% Ca-CLP + | −0.9 | −0.9 | 115 | −127 |
| | 35% CaCO₃ | | | | |
| 4 | 50% Ca-CLP | −1.0 | −0.4 | 150 | −81 |

¹25% Ca-CLP is polyacrylic acid polymer with 25% of the carboxyl groups reacted with calcium base; 50% CaCO₃ is CaCO₃ added to the formulation at a mass that will neutralize 50% of the carboxyl groups in an equivalent dose of polyacrylic acid polymer.
²All CLP doses gave an equivalent dose of carboxyl groups as 15 g of polyacrylic acid polymer

Administration of CLP led to an increase in fecal weight as shown in Table 23. This increase in fecal weight was not associated with diarrhea but is expected to be due to water entrapped in the superabsorbent polymer.

TABLE 23
Average Change from Baseline in Fecal Weight for Days 7-13

| Group | CLP formulation^{1,2} | Fecal Wt (g) |
|---|---|---|
| 1 | polyacrylic acid polymer + | 159 |
| | 75% CaCO₃ | |
| 2 | 25% Ca-CLP + | |
| | 50% CaCO₃ | 95 |
| 3 | 25% Ca-CLP + | |
| | 35% CaCO₃ | 197 |
| 4 | 50% Ca-CLP | 102 |

¹25% Ca-CLP is polyacrylic acid polymer with 25% of the carboxyl groups reacted with calcium base; 50% CaCO₃ is CaCO₃ added to the formulation at a mass that will neutralize 50% of the carboxyl groups in an equivalent dose of polyacrylic acid polymer.
²All CLP doses gave an equivalent dose of carboxyl groups as 15 g of polyacrylic acid polymer.

Similar studies may be conducted with a polyfluoroacrylic acid polymer with calcium cations as counterions to carboxylate groups in the polymer, alone or in combination with a base (e.g., calcium carbonate).

### Example 17

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

An open-label clinical trial is performed in twenty four healthy human subjects with a polyacrylic acid polymer with calcium cations as counterions to carboxylate groups in the polymer. Each patient receives an equivalent of 15 g or 30 g polyfluoroacrylic acid/day as either 25% Ca polyfluoroacrylate (n=6) or 60% calcium polyfluoroacrylate (n=6), divided into two doses, administered one hour prior to breakfast and at bedtime. Subjects remained in the clinical research unit for the duration of the study.

25% calcium polyflouroacrylate and 60% calcium polyflouroacrylate are prepared as described by any one or more of Examples 1, 3, 5 and 21-30. After cation exchange to load the polyacrylate with calcium, the polymer is milled to break up the bead structure and reduce the particle size. The calcium polyflouroacrylate powder is mixed into pudding immediately prior to dosing. The subjects are required to eat the entire pudding aliquot.

The clinical trial evaluated whether administration of calcium polyflouroacrylate when compared to a baseline period (1) altered fecal excretion of sodium, potassium, or phosphorous (2) altered measures of acidosis including serum total bicarbonate, urine pH and urine phosphorus, (3) altered serum potassium levels and (4) altered fecal weight.

After a 5 day baseline period, calcium polyflouroacrylate is administered in pudding, 3 times a day for a total of 7 days (a total of 14 doses). For the 25% calcium polyflouroacrylate cohorts the doses are 15.8 g (7.9 g bid) and 31.6 g (15.9 g bid) (see, Table 24). For the 60% calcium polyflouroacrylate cohorts the doses are 18.2 g (9.1 g bid) and 36.3 g (18.2 g bid) (see, Table 24).

TABLE 24
Dose regiment for each cohort

| | | | Daily Dose¹ |
|---|---|---|---|
| Cohort | n | Polyfluoroacrylate Form | (g) |
| 1 | 6 | 25% Ca polyfluoroacrylate | 15.8 |
| 2 | 6 | 25% Ca polyfluoroacrylate | 31.5 |
| 3 | 6 | 60% Ca polyfluoroacrylate | 18.2 |
| 4 | 6 | 60% Ca polyfluoroacrylate | 36.3 |

Diet is controlled with all participants having identical meals. Subjects are requested to consume all of their meals.

Subjects fast for at least eight hours at screening and four hours at admission prior to the collection of blood and urine samples for clinical laboratory tests. Fasting is not required prior to collection of urine and blood samples taken during the study. Water ad libitum is allowed during the periods of fasting.

Twenty four hour daily stool and urine samples are collected daily and evaluated for stool weight, fecal electrolytes, urine pH, and urine phosphorus. Daily serum samples are evaluated for serum potassium and total bicarbonate. Fecal samples are evaluated by ICP for the concentration of sodium, potassium, calcium and magnesium. All urine specimens are collected and volume recorded. Urine samples are pooled for each 24-hour period and an aliquot sampled for sodium, potassium, calcium, phosphorous and magnesium analysis.

Daily parameters for the treatment period are compared to baseline, with daily parameters for days 3-6 averaged and compared to the average for treatment days 10-13. The average change from baseline in stool weight, fecal Na, K, Mg, Ca and P, urine pH, urine phosphorus, serum potassium and total bicarbonate are determined.

Similar studies may be conducted with a polyfluoroacrylic acid polymer with calcium cations as counterions to carboxylate groups in the polymer, alone or in combination with a base (e.g., calcium carbonate).

### Example 18

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

An open-label clinical trial is performed in 36 healthy human subjects. Each patient receives 20 g polyfluoroacrylic acid/day or an equivalent of 20 g polyfluroacrylic acid/day as either 10% calcium polyflouroacrylate 25% calcium polyflouroacrylate or 50% calcium polyflouroacrylate (see, Table 25). The daily dose is divided into two doses, administered one hour prior to breakfast and before bedtime. Subjects remain in the clinical research unit for the duration of the study.

TABLE 25
Treatment Groups

| | Number of | | Dose of | Dose of |
|---|---|---|---|---|
| Group | Subjects | CLP formulation¹ | polyacrylate (g)² | CaCO₃ (g) |
| 1 | 6 | Polyfluoroacrylic | 20 | |
| | | acid + 75% CaCO₃ | | |
| 2 | 6 | Polyfluoroacrylic | 20 | |
| | | acid + 100% CaCO₃ | | |
| 3 | 6 | 10% calcium | 20.4 | |
| | | polyfluoroacrylate + | | |
| | | 65% CaCO₃ | | |
| 4 | 6 | 10% calcium | 21.1 | |
| | | polyfluoroacrylate + | | |
| | | 90% CaCO₃ | | |
| 5 | 6 | 25% calcium | 21.1 | |
| | | polyfluoroacrylate + | | |
| | | 25% CaCO₃ | | |
| 6 | 6 | 25% calcium | 22.1 | |
| | | polyfluoroacrylate + | | |
| | | 50% CaCO₃ | | |
| 7 | 6 | 50% calcium | 22.1 | 0 |
| | | polyfluoroacrylate+ | | |

10% calcium polyfluoroacrylate, 25% calcium polyfluoroacrylate and 50% calcium polyfluoroacrylate are prepared as described by any one or more of Examples 1, 3, 5 and 21-30. After cation exchange to load the polyacrylate with calcium, the polymer is milled to break up the bead structure and reduce the particle size. The calcium fluoroacrylate powder is mixed into pudding immediately prior to dosing. The subjects are required to eat the entire pudding aliquot.

The clinical trial evaluates whether administration of polyfluoroacrylate when compared to a baseline period (1) alters fecal excretion of sodium, potassium or phosphorus (2) alters measures of acid-base balance including serum total bicarbonate, urine pH and urine phosphorus, (3) alters serum potassium levels, and (4) alters fecal weight.

After a 5 day baseline period, polyfluoroacrylate is administered in capsules with water or in pudding, twice a day (before breakfast and before bedtime) for a total of 7 days (a total of 14 doses) as shown in Table 25. For all groups the polyfluoroacrylate formulation is mixed into pudding immediately prior to administration. All groups are administered an equivalent number of moles of cation exchange carboxyl groups as 20 g of polyfluroracrylic acid.

Diet is controlled with all participants having identical meals. Subjects are requested to consume all of their meals.

Subjects fast for at least eight hours at screening and four hours at admission prior to the collection of blood and urine samples for clinical laboratory tests. Fasting is not required prior to urine and blood samples taken during the study. Water ad libitum is allowed during the periods of fasting.

Twenty four hour daily stool and urine samples are collected daily and evaluated for stool weight, fecal electrolytes, urine pH, and urine phosphorus. Daily serum samples are evaluated for serum potassium and total bicarbonate. Fecal samples are evaluated by ICP for the concentration of sodium, potassium, calcium, and magnesium. All urine specimens are collected and volume recorded. Urine samples are pooled for each 24-hour period and an aliquot sampled for sodium, potassium, calcium, phosphorous, and magnesium analysis.

Daily parameters for the treatment period are compared to baseline, with daily parameters for days 3-6 averaged and compared to the average for treatment days 10-13. The average change from baseline in serum potassium, fecal weight, fecal excretion of Na, K, Ca, and Mg, urine phosphorus, serum CO₂, and urine pH are determined.

### Example 19

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

This example demonstrates the treatment of heart failure patients with a cross-linked polyelectrolyte polymer such as a crosslinked cation-binding polymer comprising monomers that comprise carboxylic acid groups (e.g., crosslinked polyacrylic acid polymer such as Ca-CLP).

In an exemplary method, patients with heart failure including, for example heart failure associated with chronic kidney disease (e.g., patients classified as class III or IV according to the New York Heart Association Classification scheme shown in Table 26) are treated with Ca-CLP (e.g., a cross-linked polyacrylic acid polymer that comprises calcium cations, and wherein the calcium cations are counterions to about 5% to about 30% or about 15% to about 35% of the carboxylate groups in the polymer, including, for example, 25% Ca-CLP, prepared as described by any one or more of Examples 1, 3, 5 and 21-30. Alternatively, patients may be treated with a combination of Ca-polyfluoroacrylate polymer plus a base (e.g., calcium carbonate), wherein the polymer is about 5% to about 75% neutralized with calcium (e.g., wherein the calcium cations are counterions to about 5% to about 75% of the carboxylate groups in the polymer), for example, wherein the polymer is about 60% neutralized with calcium, and the base is present at about 10% to about 50%, for example, about 25%, relative to the number of carboxylate groups in the polymer, administered before, with or after treatment with the polymer.

Serum chemistry, clinical signs and symptoms of heart failure, urinary electrolytes, thirst evaluation and other assessments may be evaluated throughout the treatment. Assessments which evaluate signs and symptoms of heart failure include the New York Heart Association Class (Table 26), changes in dyspnea as assessed by the patient's response to a single question using responses on a Likert scale ranging from “much worse” to “much better,” the six minute walk test and a patient reported outcome instrument (Kansas City Cardiomyopathy Questionnaire). Dyspnea may be evaluated using a quantitative patient self-assessment of breathing status compared to baseline with answers on a 7-point Likert scale ranging from “much worse” to “much better.” Additionally, the six-minute walk test is a well-accepted measure of heart failure status, with patients able to walk shorter and shorter distances as heart failure progresses. Further, the Kansas City Cardiomyopathy Questionnaire (KCCQ) is a disease-specific instrument for measuring health related quality of life in patients with congestive heart failure. The scale for each of the quality of life parameters is 0 to 100, with 100 being the best quality of life. Fluid status may also be evaluated by total body weight and extremity edema. Additionally, mean total serum CO₂ and serum bicarbonate may be measured as a measure of acid/base status.

TABLE 26
New York Heart Association Classification of Heart Failure Patients

| | |
|---|---|
| Class I | No limitation of physical activity. Ordinary physical activity |
| (mild) | does not cause undue fatigue, palpitation, dyspnea |
| | (shortness of breath), or angina pain. |
| Class II | Slight limitation of physical activity. Comfortable at rest, but |
| (mild) | ordinary physical |
| | activity results in fatigue, palpitation, dyspnea, or angina pain. |
| Class III | Marked limitation of physical activity. Comfortable at rest, |
| (moderate) | but less than ordinary activity causes fatigue, palpitation, |
| | dyspnea, or angina pain. |
| Class IV | Unable to carry out any physical activity without discomfort. |
| (severe) | Symptoms of cardiac insufficiency at rest. If any physical |
| | activity is undertaken, discomfort is increased. |

Treatment with Ca-CLP results in significant and clinically meaningful improvement of signs and symptoms in NYHA class III/IV heart failure patients including, for example, a reduction in NYHA class (e.g., a reduction in class from IV or III to II or I) a reduction of body weight, improvement in subjective symptoms (dyspnea) and quality of life (Kansas City Cardiomyopathy Questionnaire scores), and improvements in objective measures of physical function (6 Minute Walk Test) and clinical signs and symptoms (NYHA Classification; extremity edema) without resulting in a change in the subject's acid/base status.

Similar studies may be conducted with a polyfluoroacrylic acid polymer with calcium cations as counterions to carboxylate groups in the polymer, alone or in combination with a base (e.g., calcium carbonate).

### Example 20

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

Clinical studies may be conducted to evaluate a crosslinked cation-binding polymer comprising monomers that comprise carboxylic acid groups and pKa-decreasing groups including, for example, an electron-withdrawing substituent such as a halide atom (e.g., fluorine (F)) for the treatment of patients with chronic kidney disease (CKD).

In an exemplary method, patients with chronic kidney disease (e.g., patients classified as CKD stage II, Ill or IV according to the National Kidney Foundation Kidney Disease Outcomes Quality Initiative (NKF KDOQI) Guidelines shown in Table 27), who develop hyperkalemia on maximized kidney sparing treatment with Angiotensin-converting Enzyme Inhibitor (ACEI) and/or Angiotensin II Receptor Blocker (ARB) drugs, with or without spironolactone are treated with Ca-CLP CLP (e.g., a cross-linked polyacrylic acid polymer that comprises calcium cations, and wherein the calcium cations are counterions to about 50% to about 70% or about 50% to about 80% of the carboxylate groups in the polymer, including, for example, 50% Ca-CLP, prepared as described by any one or more of Examples 1, 3, 5 and 21-30. Alternatively, patients may be treated with a combination of Ca-polyfluoroacrylate polymer plus a base (e.g., calcium carbonate), wherein the calcium cations are counterions to about 5% to about 75% of the carboxylate groups in the polymer, for example, about 60%, and the base is present at about 10% to about 50%, for example, about 25%, relative to the number of carboxylate groups in the polymer, administered before, with or after treatment with the polymer. Such treated patients may include hypertensive patients with nephropathy due to type 2 diabetes mellitus (T2DM) who develop hyperkalemia on maximized kidney sparing treatment with Angiotensin-converting Enzyme Inhibitor (ACEI) and/or Angiotensin II Receptor Blocker (ARB) drugs, with or without spironolactone.

TABLE 27
National Kidney Foundation Kidney Disease Outcomes Quality Initiative
(NKF KDOQI) Guidelines

| | | GFR |
|---|---|---|
| Stage | Description | (mL/min/1.73 m² |
| 1 | Kidney damage with normal or ↑ GFR | ≧90 |
| 2 | Kidney damage with mild ↓ GFR | 60-89 |
| 3 | Moderate ↓ GFR | 30-59 |
| 4 | Severe ↓ GFR | 15-29 |
| 5 | Kidney failure | <15 |
| | | (or dialysis) |

Chronic kidney disease is defined as either kidney damage or GFR <60 mL/min/1.73 m² for ≧3 months. Kidney damage is defined as pathologic abnormalities or markers of damage, including abnormalities in blood or urine tests or imaging studies.

Blood pressure, serum chemistry, kidney function parameters (e.g. glomerular filtration rate, serum concentrations of creatinine and BUN), urinary electrolytes, urinary albumin/creatinine ratio, urinary protein excretion, clinical signs and symptoms of chronic kidney disease, and other assessments may be evaluated throughout the treatment. Assessments which evaluate signs and symptoms of chronic kidney disease include the CKD stages according to the National Kidney Foundation Kidney Disease Outcomes Quality Initiative (NKF KDOQI) Guidelines (Table 27), and physical signs and symptoms of fluid overload, e.g. edema of the extremities or abdomen, blood and urinary laboratory parameters.

In an exemplary clinical trial, inclusion criteria includes: patients that are 21 to 80 years old at screening, have Type 2 diabetes mellitus (T2DM) which has been treated with oral medications or insulin for at least one year prior to screening, have chronic kidney disease with an eGFR 15−<60 mL/min/1.73 m2 at screening, have urine albumin/creatinine ratio (ACR) of ≧30 mg/g at screening, have serum potassium values of >5.1 mEq/L at randomization to Ca-CLP, receive an ACEI and/or ARB for at least 28 days prior to screening, have an average systolic blood pressure ≧140−<180 mmHg OR average diastolic blood pressure ≧90 −<110 mmHg (sitting) at both screening and randomization. Exclusion criteria includes: patients that do not have type 1 diabetes mellitus, serum hemoglobin A1c>12% at S1, diabetic gastroparesis, non-diabetic chronic kidney disease, history of bowel obstruction, swallowing disorders, severe gastrointestinal disorders or major gastrointestinal surgery (e.g., colectomy), any of the following events having occurred within 2 months prior to screening: unstable angina as judged by the Investigator, unresolved acute coronary syndrome, cardiac arrest or clinically significant ventricular arrhythmias, transient ischemic attack or stroke, use of any intravenous cardiac medication; prior kidney transplant, or anticipated need for transplant during study participation, use loop and thiazide diuretics or other antihypertensive medications (calcium channel blocker, beta-blocker, alpha-blocker, or centrally acting agent) that have not been stable for at least 28 days prior to screening or not anticipated to remain stable during study participation; use of polymer-based drugs (e.g., sevelamer, sodium polystyrene sulfonate, colesevelam, colestipol, cholestyramine), phosphate binders (e.g., lanthanum carbonate), or other potassium binders, or their anticipated need during study participation; use of potassium sparing medications, including aldosterone antagonists (e.g., spironolactone), drospirenone, potassium supplements, bicarbonate or baking soda in the last 7 days prior to screening, inability to consume the investigational product, or, in the opinion of the Investigator, inability to comply with the protocol or in the opinion of the Investigator, any medical condition, uncontrolled systemic disease, or serious intercurrent illness that would significantly decrease study compliance or jeopardize the safety of the patient or affect the validity of the trial results. Chronic kidney disease patients selected for inclusion in the clinical trial, more specifically hypertensive patients with nephropathy due to type 2 diabetes mellitus (T2DM) are treated with maximal doses of Angiotensin-converting Enzyme Inhibitor (ACEI) and/or Angiotensin II Receptor Blocker (ARB) drugs, with or without spironolactone during a four week run in period. Those patients who develop hyperkalemia are then randomized to receive different doses of Ca-CLP for eight weeks. Patients with serum potassium levels >5.1 mEq/L but less than 5.5 mEq/L are administered the lowest Ca-CLP dose, patients with serum potassium levels >5.5 mEq/L but less than 6.0 mEq/L are administered a medium Ca-CLP dose, and patients with serum potassium levels >6.0 mEq/L are administered a high dose of Ca-CLP dose. Ca-CLP doses can be adjusted up or down based on follow up serum potassium levels. Outcome measures include the mean change in serum potassium from baseline to treatment week 4 and 8, proportion of patients maintaining the starting Ca-CLP dose at week 4 and 8, proportion of patients requiring Ca-CLP titration, proportion of patients who maintain serum potassium (K⁺) in the range of 3.5-5.5 mEq/L by visit and during the entire study treatment period, proportion of patients who maintain serum K⁺ in the range of 4.0-5.0 mEq/L by visit and during the entire study treatment period, proportion of patients who discontinue from the study due to high serum potassium withdrawal criteria, mean change in blood pressure from screening to week 4 and 8, mean change in urine albumin to creatinine ratio (ACR) from screening to week 4 and 8, proportion of patients with 35% reduction in urine ACR from screening to week 4 and 8, proportion of patients with urine ACR 500 mg/g at screening who achieve ACR <500 mg/g at week 4 and 8, physical signs and symptoms of fluid overload, e.g. edema of the extremities or abdomen, blood and urinary laboratory parameters.

Treatment with Ca-CLP results in significant and clinically meaningful improvement of signs and symptoms in CKD stage II, Ill or IV patients including, for example, a improvement in CKD stage (e.g., a improvement in class from IV to III, or III to II, or I) a reduction of body weight, improvement in subjective symptoms (edema) and serum and urinary laboratory parameters without resulting in a change in the subject's acid/base status.

### Example 21

This example demonstrates the preparation of an exemplary crosslinked cation-binding polymer comprising 2-fluoroacrylic acid monomers that comprise carboxylic acid groups and pKa-decreasing groups including, for example, an electron-withdrawing substituent such as a halide atom (e.g., fluorine (F)).

In an exemplary method, to a reaction vessel are charged 2-fluoroacrylic acid ethylenebisacrylamide and water, followed by a magnetic stir bar. The mixture is stirred at 45° C. for 20 minutes and 2,2′-azobis[2-(2-imidazolin-2-yl)propane]dihydrochloride is added. Different levels of crosslinker are used in these studies, ranging from 2.5 wt % to 20 wt % (1.6 mol % to 13.4 mol %). Intermediate crosslinker ranges include: 5 wt % (3.2 mol %) and 10 wt % (6.4 mol %). The solutions gel and are kept at 45° C. for 4 hours, then cooled to room temperature. Each gel is transferred to a polypropylene tube and water is added. The gel is crushed with a spatula, and further milled with an Ultra-Turrax. The tube is then capped and centrifuged at 3000 rpm for 30 minutes and the supernatant solution is decanted off. To the gel is added 1.0M HCl and the tube is capped and tumbled for 30 minutes. The tube is centrifuged at 3000 rpm for 30 minutes and supernatant solution is decanted off. The same tumbling-centrifuging procedure is repeated once with 1.0M HCl and three times with nanopure water. The 2-fluoroacrylate-ethylenebisacrylamide copolymer gel is freeze-dried for three days.

### Example 22

This example demonstrates the preparation of an exemplary crosslinked cation-binding polymer comprising 2-fluoroacrylic acid monomers and acrylic acid monomers that comprise carboxylic acid groups and pKa-decreasing groups including, for example, an electron-withdrawing substituent such as a halide atom (e.g., fluorine (F)).

In an exemplary method, to a reaction vessel containing a magnetic stir bar are charged 2-fluoroacrylic acid, ethylenebisacrylamide (final 10 wt %, ˜5 mol %) and water, and the mixture is stirred until all solids dissolved. Acrylic acid is added to final 2-fluoroacrylic acid:acrylic acid ratios of 90:10, 80:20, 70:30, 60:40 and 50:50, followed by 2,2′-azobis[2-(2-imidazolin-2-yl)propane]dihydrochloride. The mixture is stirred at 45° C. for 3 hours, then cooled to room temperature. The gel is purified according to the same procedure as used for 2-fluoroacrylic acid polymer.

### Example 23

This example demonstrates the preparation of an exemplary crosslinked cation-binding polymer comprising methyl 2-fluoroacrylate monomers that comprise carboxylic acid groups and pKa-decreasing groups including, for example, an electron-withdrawing substituent such as a halide atom (e.g., fluorine (F)) and divinylbenzene crosslinker.

In an exemplary method, the polymerization is carried out in a three-neck Morton-type round bottom flask equipped with an overhead mechanical stirrer with a Teflon paddle and a water condenser. An organic phase is prepared by mixing methyl 2-fluoroacrylate and divinylbenzene at a weight ratio of 90:10 (10 wt % crosslinker, 8.2 mol %), followed by lauroyl peroxide, and an aqueous phase is prepared by dissolving polyvinyl alcohol and sodium chloride (NaCl) in water. The organic and aqueous phases are then mixed in the flask and stirred at 300 rpm under nitrogen. The flask is immersed in a 70° C. oil bath for 3 hours, and cooled to room temperature. The internal temperature during the reaction is about 65° C. The solid product is washed with water and collected by decanting off supernatant solution. The white solid is freeze-dried, affording dry solid polymethyl 2-fluoroacrylate particles (or beads). Hydrolysis is carried out in the same setup as for the polymerization. Polymethyl 2-fluoroacrylate particles from above are suspended in KOH solution and stirred at 300 rpm. The mixture is heated in a 95° C. oil bath for 20 hours and cooled to room temperature. The solid product is washed with water and collected by decanting off the supernatant solution. After freeze-drying, K(poly-2-fluoroacrylic acid) particles are obtained. These particles are in the form of beads.

### Example 24

This example demonstrates the preparation of an exemplary crosslinked cation-binding polymer comprising methyl 2-fluoroacrylate monomers that comprise carboxylic acid groups and pKa-decreasing groups including, for example, an electron-withdrawing substituent such as a halide atom (e.g., fluorine (F)) and divinylbenzene and 1,7-octadiene crosslinkers.

In an exemplary method, multiple suspension polymerizations are carried out in a manner substantially similar to Example 23 using various combinations of methyl 2-fluoroacrylate and the crosslinkers divinylbenzene and 1,7-octadiene. The amounts of the organic phase reagents range from: methyl 2-fluoroacrylate, 80 wt % to 95 wt %; divinylbenzene, 0 wt % to 20 wt % (16.7 mol %); and 1,7-octadiene, 0 wt % to 15 wt % (14.3 mol %). The ratios of methyl 2-fluoroacrylate, divinylbenzene and 1,7-octadiene (and crosslinker wt % and mol %) include: 95:5:0 (5 wt %, 4.0 mol %), 90:10:0 (10 wt %, 8.2 mol %), 90:8:2 (10 wt %, 8.4 mol %), 90:5:5 (10 wt %, 8.8 mol %), 90:2:8 (10 wt %, 9.2 mol %), 90:0:10 (10 wt %, 8.8 mol %), 85:0:15 (15 wt %, 14.3 mol %) and 80:20:0 (20 wt %, 16.7 mol %).

### Example 25

This example demonstrates the preparation of an exemplary crosslinked cation-binding polymer comprising methyl 2-fluoroacrylate monomers that comprise carboxylic acid groups and pKa-decreasing groups including, for example, an electron-withdrawing substituent such as a halide atom (e.g., fluorine (F)) and divinylbenzene and 1,7-octadiene crosslinkers.

In an exemplary method, the polymers are prepared as follows. A polymerization is carried out in a three-neck Morton-type round bottom flask equipped with an overhead mechanical stirrer with a Teflon paddle and a water condenser. An organic phase is prepared by mixing methyl 2-fluoroacrylate, divinylbenzene and 1,7-octadiene (wt ratio of 90:5:5) and lauroyl peroxide, and an aqueous phase is prepared by dissolving polyvinyl alcohol and NaCl in water. The organic and aqueous phases are then mixed in the flask, and stirred at 300 rpm under nitrogen. The flask is immersed in a 70° C. oil bath for 5 hours, and cooled to room temperature. The internal temperature during reaction is about 65° C. The solid product is washed with water and collected by filtration. The white solid is freeze-dried, affording dry solid polymethyl 2-fluoroacrylate beads. Hydrolysis is carried out in the same setup as for the polymerization. Polymethyl 2-fluoroacrylate beads from the polymerization reaction are suspended in a NaOH solution and stirred at 200 rpm. The mixture is heated in a 95° C. oil bath for 20 hours and cooled to room temperature. The solid product is washed with water and collected by filtration. After freeze-drying, (sodium 2-fluoroacrylate)-divinylbenzene-1,7-octadiene copolymer beads (Na(poly-2-fluoroacrylic acid)) are obtained.

### Example 26

This example demonstrates the preparation of an exemplary crosslinked cation-binding polymer comprising t-butyl 2-fluoroacrylate monomers that comprise carboxylic acid groups and pKa-decreasing groups including, for example, an electron-withdrawing substituent such as a halide atom (e.g., fluorine (F)) and divinylbenzene and 1,7-octadiene crosslinkers.

In an exemplary method, a stock aqueous solution of NaCl, water, polyvinyl alcohol, (Na₂HPO₄.7H₂O) and NaNO₂ is prepared. A stock solution of the organic components that consists of t-butyl 2-fluoroacrylate, divinylbenzene, 1,7-octadiene (final crosslinker 7.4 wt %, 8.9 mol %) and LOA is prepared. Components are weighed manually into a 3-necked reaction flask with baffles. The flask is fitted with an overhead stirrer, and a condenser. Nitrogen is blown over the reaction for 10 minutes and a blanket of nitrogen is maintained throughout the reaction. The stir rate is set to 180 rpm. The bath temperature is set to 70° C. After 12 hours the heat is increased to 85° C. for 2 hours and the reaction is allowed to cool to room temperature. The beads are isolated from the reaction flask and are washed with isopropyl alcohol, ethanol and water. The poly-t-butyl 2-fluoroacrylate butyl ester beads are dried at room temperature under reduced pressure. Next, into a 3-necked reaction flask with baffles, is weighed poly-t-butyl 2-fluoroacrylate beads and concentrated hydrochloric acid (3 times the weight of bead, 3 moles of hydrochloric acid to 1 t-butyl-ester), and water (3 times bead). The flask is fitted with an overhead stirrer, and a condenser. Nitrogen is blown over the reaction for 10 minutes and a blanket of nitrogen is maintained throughout the reaction. The stir rate is set to 180 rpm. The bath temperature is set to 75° C. After 12 hours the heat turned off and the reaction is allowed to cool to room temperature. The beads are isolated from the reaction flask and are washed with isopropyl alcohol, ethanol and water. The H(poly-2-fluoroacrylic acid) beads are dried at room temperature under reduced pressure.

### Example 27

This example demonstrates the conversion of an exemplary crosslinked cation-binding polymer comprising 2-fluoroacrylic acid monomers that comprise carboxylic acid groups and pKa-decreasing groups including, for example, an electron-withdrawing substituent such as a halide atom (e.g., fluorine (F)), to a calcium form of the polymer.

In an exemplary method, the polymers from Examples 21-26 are converted to the calcium form by exposing the polymer salts to excess calcium chloride (CaCl₂) to yield insoluble cross-linked calcium 2-fluoroacrylate-divinylbenzene-1,7-octadiene copolymer. Alternatively, the polyfluoroacrylate polymers from Examples 21-26 and 29 can be converted to forms containing counterions other than calcium (e.g., magnesium) or to forms containing lower amounts of calcium counterions (e.g., counterions to about 5% to 75% of the carboxylate groups) using methods outlined in Example 5. The final Ca(poly-2-fluoroacrylic acid) product is washed with ethanol and water.

### Example 28

This example demonstrates the preparation of an exemplary composition comprising a crosslinked cation-binding polymer comprising 2-fluoroacrylic acid monomers that comprise carboxylic acid groups and pKa-decreasing groups including, for example, an electron-withdrawing substituent such as a halide atom (e.g., fluorine (F)) and polyol.

In an exemplary method, a composition comprising a crosslinked cation-binding polymer comprising 2-fluoroacrylic acid monomers and polyol is prepared by charging D-sorbitol followed by water to a 3-necked round bottom flask equipped with a magnetic stirrer and nitrogen inlet adapter. The mixture is stirred until a clear solution is obtained. Ca(poly-2-fluoroacrylic acid) prepared as described by any one or more of Examples 5 and 27 is added in one portion to the sorbitol solution and the resultant slurry is stirred at ambient temperature (20-25° C.) for three hours. Various amounts of sorbitol solutions ranging from 2 w/w % to 45 w/w % can be added to the polymer, the times of mixing range from 1.5 to 3 h, and samples are dried by lyophilization or air drying under vacuum. The solids are filtered off and dried under reduced pressure to the desired water content. The solids are analyzed for sugar alcohol content and loss on drying.

The samples prepared above are placed in storage at temperatures ranging from 5° C. to 40° C., with typical conditions being 5-8° C., 20-25° C. and 40° C., for times from 0 to 12 weeks. For samples stored at 5° C. and ambient temperature, the samples are transferred to a vial, which is placed in a Sure-Seal bag and sealed, and then placed in a second Sure-Seal bag with a desiccant (calcium sulfate) in the second bag, which is also sealed. For the samples at higher temperatures, the samples are placed in vials and stored at the stated temperatures. At various times (1 week, 3 weeks, 5 weeks, 7 weeks, etc.), aliquots of the samples are removed from storage and tested for their weight, moisture content, loss on drying and free inorganic fluoride.

The potassium binding capacity of the poly-2-fluoroacrylic acid sorbitol compositions may then be analyzed. In an exemplary method, the materials used are potassium chloride (Reagent Plus grade, >99%, Sigma #P4504 or equivalent); de-ionized water greater than 18 megaöhm resistivity; IC potassium standard (1,000 ppm, Alltech Cat#37025 or equivalent); ion chromatography (IC) potassium standard, 1000 ppm from a secondary source (e.g. Fisher Scientific #CS-K2-2Y); and methane sulfonic acid (MSA, 99.5%; Aldrich #471356). The MSA is used to make the IC mobile phase if the apparatus used is unable to generate the mobile phase electrolytically.

A quality control check and a linear curve may be prepared for analysis of the poly-2-fluoroacrylic acid sorbitol compositions by ion chromatography Briefly, potassium standard solutions (100, 250, 500 ppm) for IC are prepared by diluting a stock 1000 ppm potassium chloride solution with distilled (DI) water. A stock potassium chloride solution may be prepared by dissolving 14.91 g potassium chloride in 800 mL of water. A graduated cylinder is used and water is added to make a 1 L solution. This solution is the 200 mM potassium chloride solution for the binding assay.

The QC check standard is obtained by diluting a second source certified 1000 ppm potassium standard with DI water to achieve 250 ppm concentration.

A sample solution of the poly-2-fluoroacrylic acid sorbitol compositions may then be prepared. Briefly, two samples of Ca(poly-2-fluoroacrylic acid) prepared by the method of Example 25 are placed into separate screw top vials. Using the equation below, the amount of 200 mM KCl solution to add to the vial is calculated:

$$\frac{\frac{M}{100} \times [ 100 - S \times ( 1 - \frac{W}{100} ) - W ]}{20}$$

where M is H(poly-poly-2-fluoroacrylic acid) sample weight (mg), S is sorbitol content based on dry weight of H(poly-2-fluoroacrylic acid), and W is loss on drying (%). The calculated volume of 200 mM KCl solution is added to each vial using a 10 mL pipettor. The vials are capped tightly. Two blank vials containing 15 mL of 200 mM KCl solution are prepared. The vials are tumbled on a rotary tumbler for two hours at about 35 rpm. After two hours, the vials are removed from the tumbler. The contents are allowed to settle for 5 minutes. Each sample (2-10 mL) and a blank are filtered over a 0.45 micron filter. Each filtered sample is diluted 1:20 by adding 500 μL of each sample or blank to 9500 μL of water. The diluted filtrate is analyzed for potassium content using IC.

Next, the sample may be analyzed by ion chromatography. Briefly, if a 20 mM MSA mobile phase could not be generated electrolytically, the 20 mM stock MSA mobile phase is made by diluting MSA in water. The ion chromatographyhas the following settings: injection volume: 5 μL; flow rate: 1 mL/min; column temperature: 35° C.; sample compartment temperature: ambient; run time: 20 min; and CD25 settings: current 88 mA, cell temperature 35° C., autorange. Each blank and sample is injected twice.

Any suitable IC system may be used, such as, for example: A Dionex IC System 2000 equipped with AS50 autosampler, conductivity Detector CD25 and DS3 flow cell. The column is a CS12A 250×4 mm ID analytical column, Dionex #016181 coupled with a CG12A 50×4 mm ID guard column (optional), Dionex#046074. The suppressor used is a Dionex CSRS-Ultra II (4 mm) Suppressor, Dionex #061563. The software used for data acquisition is Dionex Chromeleon Chromatography Software. The eluent cartridge is a Dionex #058902 to generate the methane sulfonic acid (MSA) mobile phase electrolytically.

The concentration of potassium is reported in mM. The equation below is used to calculate the binding capacity of each sample:

Binding capacity (mmol/g)=(c_{Blank}−cₛₐₘₚₗₑ)

where c_{Blank} is average concentration of potassium in the 20-fold diluted blank by IC analysis (mM), and cₛₐₘₚₗₑ is average concentration of potassium in the 20-fold diluted sample solution by IC analysis (mM). The average of the duplicates is reported. The deviation of each individual value is a maximum of 10% from the mean. When a larger deviation is obtained, the assay is repeated.

### Example 29

This example demonstrates the preparation of an exemplary composition comprising a sorbitol-loaded poly-2-fluoroacrylic acid.

In an exemplary method, in an appropriately sized reactor with appropriate stirring and other equipment, a 90:5:5 weight ratio mixture of organic phase of monomers is prepared by mixing methyl 2-fluoroacrylate, divinylbenzene and 1,7-octadiene. One part of LOA is added as an initiator of the polymerization reaction. A stabilizing aqueous phase is prepared from water, polyvinyl alcohol, sodium phosphate dibasic heptahydrate and sodium phosphate monobasic monohydrate (phosphates), NaCl, and sodium nitrite. The aqueous and monomer phases are mixed together under nitrogen at atmospheric pressure, while maintaining the temperature below 30° C. The reaction mixture is gradually heated while stirring continuously. Once the polymerization reaction has started, the temperature of the reaction mixture is allowed to rise to a maximum of 95° C. After completion of the polymerization reaction, the reaction mixture is cooled and the aqueous phase is removed. Water is added, the mixture is stirred, and the solid material is isolated by filtration. The solid is then washed with water to yield a crosslinked (methyl 2-fluoroacrylate)-divinylbenzene-1,7-octadiene polymer. The (methyl 2-fluoroacrylate)-divinylbenzene-1,7-octadiene copolymer is hydrolyzed with an excess of aqueous sodium hydroxide solution at 90° C. for 24 hours to yield (sodium 2-fluoroacrylate)-divinylbenzene-1,7-octadiene polymer. After hydrolysis, the calcium form of the beads is prepared by exposing the sodium 2-fluoroacrylate-divinylbenzene-1,7-octadiene polymer to an excess of aqueous calcium chloride to yield calcium 2-fluoroacrylate-divinylbenzene-1,7-octadiene polymer. The calcium form of the polymer is filtered and washed with water. The wet polymer is slurried with 25-30% w/w aqueous solution of sorbitol at ambient temperature to yield sorbitol-loaded polymer. Excess sorbitol is removed by filtration. The resulting polymer is dried at 20-30° C. until the desired moisture content (10-25 w/w/%) is reached. This provides a sorbitol loaded, cross-linked Ca(poly-2-fluoroacrylic acid) polymer.

### Example 30

This example demonstrates the preparation of an exemplary powder composition comprising a calcium form of a poly-2-fluoroacrylic acid polymer (e.g., Ca(poly-2-fluoroacrylic acid) as disclosed herein.

In an exemplary method, the active pharmaceutical ingredient (API), cross-linked Ca(poly-2-fluoroacrylic acid), and the powder formulation are prepared essentially as described in Examples 5 and 27. The excipients used in the powder composition are available from commercial sources and meet the specifications defined in the current compendial monograph. Thereafter the stabilized polymer is split in about half and mixed with other ingredients as described below.

For example, one powder formulation is prepared by mixing reagents such that the final wt % (and function) are: polymer (API) 56.97%, sorbitol (API stabilizer) 23.55%, water (API stabilizer) 17.47%, xanthan gum (suspending agent) 0.70%, colloidal silicon dioxide (glidant) 0.94%, yellow dye (coloring agent) 0.02%, and titanium dioxide (opacity) 0.34%, totaling 100.00%. The mixture is screened and then the second about half of the stabilized polymer is added to the mixture. The entire mixture is thoroughly mixed and then screened again. The powder formulation may be reconstituted with water at, for example, a ratio of 1:5 (powder/water), such that a 15 g dose of API will be 75 mL of water. On the other hand, the formulated powder can be mixed with soft foods such as applesauce, yogurt or pudding for administration. The powder is packaged in 60 cc wide mouth, white high density polyethylene (HDPE) bottles with 15 g of the polymer per bottle.

For example, a second powder formulation is prepared having an antimicrobial agent added. The ingredients for the second powder formulation are: polymer (API) 56.89%, sorbitol (API stabilizer) 23.52%, water (API stabilizer) 17.45%, xanthan gum (suspending agent) 0.70%, colloidal silicon dioxide (glidant) 0.94%, dye or dye blend (coloring agent) 0.02%, methylparaben (antimicrobial) 0.11%, propylparaben (antimicrobial) 0.03%, and titanium dioxide (opacity) 0.34%, totaling 100.00%.

### Example 31

Studies may be conducted to evaluate a calcium form of a poly-2-fluoroacrylic acid polymer (e.g., Ca(poly-2-fluoroacrylic acid) as disclosed herein, including, for example, to evaluate its potassium binding capacity.

In an exemplary method, potassium binding by Ca(poly-2-fluoroacrylic acid) is evaluated in ex vivo human fecal and colonic extracts. Fecal samples, and colonic samples obtained through use of a colostomy bag, are provided by human volunteers. The samples are centrifuged, and the resulting supernatant is isolated for use as a test medium in the binding study. Ca(poly-2-fluoroacrylic acid) is added to the extract samples at 20 mg/mL, and incubated for 24 hours at 37° C. Binding of potassium, as well as other cations present in the extracts is determined per gram of Ca(poly-2-fluoroacrylic acid).

Fecal samples are collected in one-gallon Ziploc bags and immediately mixed and transferred into centrifuge tubes. The colostomy bag contents are shipped on dry ice, thawed, mixed and transferred into centrifuge tubes. The fecal and colonic samples are centrifuged at 21,000 rpm for 20 hours at 4°. The resulting supernatant is pooled per subject, and filtered using a Nalgene 0.2 μm disposable filter unit. The fecal and colonic extracts are then either used fresh, or are frozen at −20° C. until needed.

Cation binding of Ca(poly-2-fluoroacrylic acid) in fecal and colonic extracts is then determined. Briefly, fecal and colonic extracts are thawed in a room temperature water bath and stirred on a magnetic stir plate. Penicillin G/Streptomycin (Gibco, 15140-122) (1/100 volume of 100× stock solution) and sodium azide (1/1000 volume of 10% stock solution) are added to each extract sample to discourage bacterial or fungal growth during the assay. Ca(poly-2-fluoroacrylic acid) is added to 16×100 mm glass tubes in duplicate, with each tube receiving 140 to 170 mg of dried, accurately weighed sample. While stirring, fecal or colonic extract is dispensed into the tubes to create a final concentration of 20 mg of test sample per mL of extract. Each extract is additionally dispensed into duplicate tubes containing no test sample. All tubes are sealed and incubated for 24 hours at 37° C., rotating on a rotisserie mixer. Following incubation, 25 4 of each sample is diluted into 475 4 of Milli-Q purified water (1:20 dilution). The diluted samples are then filtered by centrifugation at 13,200 rpm through Microcon YM-3 filter units (3000 MWCO) for 1 hour. Filtrates are transferred to a 1 mL 96-well plate and submitted for analysis of cation concentrations by ion chromatography.

Cation concentrations in fecal and colonic extract are determined by an ion chromatography method. Briefly, cation concentrations in the fecal and colonic extract samples are analyzed using a strong cation-binding column set (viz., Dionex CG16 50×5 mm ID and CS16 250×5 mm ID), on a Dionex ICS2000 system equipped with a Dionex WPS3000 auto sampler, DS3 conductivity flow cell and CSRS-Ultra II 4 mm Suppressor. The ion chromatography detection method included an isocratic elution using 30 mM of methanesulfonic acid at a flow rate of 1 mL/minute, and the total run time is 30 minutes per sample.

Cation binding is calculated as (Cstart−Ceq)/20*valency of the ion, where Cstart is the starting concentration of cation in the fecal or colonic extract (in mM), Ceq is the concentration of cation remaining in the sample at equilibrium after exposure to the test agent (in mM), and 20 corresponds to the concentration of the test agent (in mg/mL). Multiplying by the valency of the ion (1 for potassium, ammonium and sodium; 2 for calcium and magnesium) gives a binding value expressed in milliequivalents (mEq) of ion bound per gram of test agent. A₁₁ samples are tested in duplicate with values reported as an average (Avg), +/−standard deviation (SD).

### Example 32

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

In an exemplary method, pigs with normal renal function are used as a model to assess the pharmacological effects of Ca(poly-2-fluoroacrylic acid) in binding and removing potassium from the gastrointestinal tract. A pig model is used based on the well known similarities between the pig and human gastrointestinal tracts. The pigs are fed a diet supplemented with Ca(poly-2-fluoroacrylic acid) at a concentration of 1 gram per kilogram of body weight per day. As a control, pigs are fed the diet without H(poly-2-fluoroacrylic acid).

Ca(poly-2-fluoroacrylic acid) is prepared as described by any one or more of Examples 5, 27 and 29. Optionally, animals may be treated with a combination of Ca-polyfluoroacrylate polymer plus a base (e.g., calcium carbonate), wherein the polymer is about 5% to about 75% neutralized with calcium, for example, about 60%, and the base is present at about 10% to about 50%, for example, about 25%, relative to the number of carboxyl groups in the polymer, administered before, with or after treatment with the polymer. Ferric oxide is added as an indigestible marker. The ferric oxide is used as a daily visible marker to determine the passage rate of the digesta through the gastrointestinal tract of each animal.

Fourteen approximately nine-week old grower barrows weighing approximately 25 kg are used in this study. At the start of the experiment, fourteen pigs are weighed and randomized by weight into control and treatment groups. The experiment is divided into two feeding periods. The first period is the acclimation period, days (D(−7) to D(−1)), and the second is the test period, (D(1) to D(9)). Before the acclimation period, the pigs are fed a standard production diet. During the acclimation period, pigs are progressively offered increasing amounts of the control diet as a ratio to a standard production grower diet. On the same day the pigs are fed the ferric oxide, the seven test pigs are switched to the test diet. The control pigs remained on the control (acclimation) diet. The test diet is fed for ten days (D(1) to D(10)). Throughout the entire study, daily feed allowance for individual pigs is divided in two equal sizes and offered at approximately 08:30 and 15:30. The pigs are trained to clean up their daily feed allowance once it is provided; any feed that is not eaten is weighed and removed before the next feeding.

Urine collection begins with the offering of the ferric oxide bolus on D(1). Each day's sample is kept separate for each pig. Following the completion of urine collection, the daily samples for each pig are thawed, mixed well and sub-sampled. The sub-sample of at least 10 mL of each pig's 24-hour sample is analyzed for electrolyte concentrations as described below.

Fecal collection begins with the offering of the ferric oxide bolus on D(1). Each day's sample is kept separate for each pig.

The levels of urine electrolytes are determined. Briefly, urine samples are thawed, diluted 30 fold in 50 mM hydrochloric acid and then filtered (Whatman 0.45 micron PP filter plate, 1000×g for 10 minutes). The cation concentrations in these urine samples are analyzed using a strong cation-binding column set (Dionex CG16 50×5 mm ID and CS16 250×5 mm ID), on a Dionex ICS2000 system equipped with a Dionex AS50 auto sampler, DS3 conductivity flow cell and CSRS-Ultra II 4 mm Suppressor. The ion chromatography detection method included an isocratic elution using 31 mM methanesulfonic acid at a flow rate of 1 mL/minute, and the total run time is 33 minutes per sample.

The levels of fecal electrolytes are determined. Briefly, to a 15 mL conical tube, 200 mg of feces and 10 mL of 1M hydrochloric acid is added. The fecal mixture is incubated for approximately 40 hours on a rotisserie mixer at room temperature. A sample of fecal supernatant is isolated after centrifugation (2000×g, 15 minutes) and then filtered (Whatman 0.45 micron PP filter plate, 1000×g for 10 minutes). The filtrate is diluted 2 fold with Milli-Q water.

Diluted filtrate cation content is measured by inductively coupled plasma optical emission spectrometry (ICP-OES) using a Thermo Intrepid II XSP Radial View. Samples are infused into the spray chamber using a peristaltic pump and CETAC ASX-510 autosampler. An internal standard, yttrium (10 ppm in 1M hydrochloric acid), is employed for correcting variation in sample flow as well as plasma conditions. The emission line that is used for quantifying potassium is 7664 nm (internal standard 437.4 nm).

Fecal electrolytes are calculated in milliequivalents per day (mEq/day) using the following equation:

$$mEq \text{/} day = [ \frac{( mEq \text{/} L electrolyte \times assay volume ( L ) )}{( grams feces in assay )} ] \times [ \frac{Total feces ( grams )}{Day} ]$$

In the above equation, mEq/L electrolyte is the concentration of an electrolyte reported by ICP spectrometry after adjusting for dilution factor and valence, and total feces per day is the amount, in grams, of feces collected in a 24 hour period after lyophilization.

Urinary electrolytes are calculated in mEq electrolyte excreted per day (mEq/day) using the following equation: (mEq electrolyte per L)*(24 hour urine volume). Data is presented using means±standard deviation, and/or by scatter plot. Statistical analysis is performed with the aid of computer programs such as GraphPad Prism, version 4.03. For urine and fecal analyses, probability (p) values are calculated using a two-tailed t-test to compare the H(poly-2-fluoroacrylic acid) treated group to the non-treatment control group. Statistical significance is indicated if the calculated p value is less than 0.05.

For fecal analysis, the mean result from each group is determined by averaging the combined mEq/day electrolyte values from treatment days three through day eight for each animal and then averaging this result for each treatment group. This methodology is also employed for urinary electrolytes, but the average for each animal is from treatment (1) through day (8).

### Example 33

Studies may be conducted to evaluate a crosslinked cation binding polymer comprising monomers that comprise carboxylic acid groups including, where the carboxylic acid groups may further comprise pKa decreasing groups, and calcium cations, wherein the calcium cations are counterions to about 5% to about 75% of the carboxylate groups in the polymer, and wherein the polymer optionally comprises no more than about 5% sodium cations as counterions to the carboxylate groups in the polymer. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base.

The objective of the study is to evaluate the equivalence of once a day, two times a day and three times a day dosing of the Ca(poly-2-fluoroacrylic acid) polymer from Examples 5, 27 and 29. After a four day period to control diet, 12 healthy volunteers are randomized in an open-label, multiple-dose crossover study. The polymer is administered orally as an aqueous suspension of 30 grams (g) once a day for six days, 15 g twice a day for six days, and 10 g three times a day for 6 days in a randomly assigned order based upon 1 of 6 dosing sequences. Laboratory and adverse event assessments are performed throughout the study to monitor safety and tolerability. Subjects are required to consume a controlled diet for the duration of the study. Feces and urine are collected over 24 hour intervals on certain study days to assess potassium excretion.

Subjects are healthy adult males or females without a history of significant medical disease, 18 to 55 years of age, with a body mass index between 19 and 29 kg/m2 at the screening visit, serum potassium level >4.0 and <5.0 mEq/L, and serum magnesium, calcium, and sodium levels within normal range. Females of childbearing potential must be non-pregnant and non-lactating and must have used a highly effective form of contraception before, during, and after the study.

Another study is performed to assess the safety and efficacy of a binding polymer that is the same as described above in this example, but without the sorbitol loading. Thirty-three healthy subjects (26 male and 7 female) between the ages of 18 and 55 years receive single and multiple doses of polymer or placebo in a double-blind, randomized, parallel-group study. Eight subjects each are randomly assigned to one of four treatment groups receiving polymer or matching placebo. The subjects receive 1, 5, 10, or 20 g of polymer or placebo as a single dose on study day 1, followed by three times daily dosing for eight days following seven days of diet control. Subjects are required to consume a controlled diet for the duration of the study.

### Example 34

Additional clinical studies are conducted to evaluate a crosslinked cation-binding polymer comprising monomers that comprise carboxylic acid groups and pKa-decreasing groups including, for example, an electron-withdrawing substituent such as a halide (e.g., fluorine (F)), and calcium cations, wherein the calcium cations are counterions to about 5% to about 75% of the carboxylate groups in the polymer, and wherein the polymer optionally comprises no more than 5% sodium cations as counterions to carboxylate groups in the polymer, for the treatment of hyperkalemia in patients with chronic heart failure (HF). Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base (e.g., calcium carbonate) that is administered before, with or after administration of the polymer.

In an exemplary method, eligible patients are 18 years of age, have a history of chronic HF, an indication to initiate spironolactone therapy, per the investigator's clinical judgment, a serum K⁺ concentration of 4.3-5.1 mEq/L at screening. In addition, they must have either (i) CKD [with estimated glomerular filtration rate (eGFR) determined by a local laboratory of <60 mL/min] and are receiving one or more HF therapies (ACE-Is, ARBs, beta-blockers); or (ii) a documented history of hyperkalaemia that leads to discontinuation of therapy with an AA, ACE-I, ARB, or beta-blocker within 6 months prior to the baseline visit.

Patients are excluded if they have severe GI disorders, major GI surgery, bowel obstruction, swallowing disorders, significant primary valvular disease, known obstructive or restrictive cadiomyopathy, uncontrolled or unstable arrhythmia, episode of unstable angina within 3 months prior to baseline, acute coronary syndrome, transient ischaemic attack, a QTc value of >500 ms (using Bazett's correction formula), recent or anticipated cardiac surgery or intervention, kidney transplantation or need for transplantation, receiving dialysis or anticipated need for dialysis during the study, sustained systolic blood pressure >170 or <90 mmHg, elevated liver enzymes (more than three times the upper limit of normal), or any condition that has the potential to interfere with study compliance or jeopardize the safety of the patient.

Patients who complete screening and satisfy the eligibility criteria proceed to baseline assessments, which include review of medical and medication histories, a physical examination, including weight, resting vital signs, and 12-lead electrocardiogram (ECG), determination of serum K⁺, and clinical laboratory tests (including serum chemistry, haematology, and urinalysis); in addition, women of child-bearing potential will have a serum pregnancy test.

Following baseline assessments, patients who continue to meet eligibility criteria are randomized 1:1 to treatment with Ca(poly-2-fluoroacrylic acid) polymer prepared as described by any one or more of Examples 1, 3, 5, and 21-30, or placebo in a blinded fashion. Patients are instructed to take 15 g of study drug orally in the morning and evening (for a total daily dose of 30 g) and to mix study drug (supplied as a powder) with water or a low-potassium food prior to administration. Patients are also instructed to start spironolactone at a dose of 25 mg/day. After 2 weeks (e.g., on Day 15), spironolactone is increased to 50 mg/day if the patient's serum K⁺ is >3.5 to 5.1 mEq/L; the dose remains at 25 mg/day if the serum K⁺ level is >5.1 to 5.5 mEq/L; and patients are discontinued from the study if their serum K⁺ is 3.5 or >5.5 mEq/L. Optionally, subjects may be treated with a combination of Ca-polyfluoroacrylate polymer plus a base (e.g., calcium carbonate), wherein the polymer is about 5% to about 75% neutralized with calcium, for example, about 60%, and the base is present at about 10% to about 50%, for example, about 25%, relative to the number of carboxyl groups in the polymer, administered before, with or after treatment with the polymer.

Prohibited medications during the study include polymer-based drugs, other phosphate or K⁺ binders, K⁺ sparing medications, antacids, calcium or K⁺ supplements, and intravenous cardioactive medications.

Throughout the 4-week treatment period, assessments of efficacy and safety are performed routinely. Serum K⁺ is monitored at each clinic visit on Days 3, 7, 14, 17, 21, and 28. Serum chemistry, body weight, and vital signs are assessed on Days 7, 14, 21, and 28; haematology on Days 14 and 28; and 12-lead ECGs and assessments of concomitant medications and adverse events (AEs) are performed at each clinic visit.

The primary endpoint of the study is the change from baseline in serum potassium.

Treatment with crosslinked polyfluoroacrylate polymer and a base may result in significant and clinically meaningful improvement in signs and symptoms of hyperkalemia in patients with chronic heart failure.

### Example 35

Additional clinical studies are conducted to evaluate a crosslinked cation-binding polymer comprising monomers that comprise carboxylic acid groups and pKa-decreasing groups including, for example, an electron-withdrawing substituent such as a halide (e.g., fluorine (F)), and calcium cations, wherein the calcium cations are counterions to about 5% to about 75% of the carboxylate groups in the polymer, and wherein the polymer optionally comprises no more than 5% sodium cations as counterions to carboxylate groups in the polymer, for the treatment of hyperkalemia. Exemplary polymers with calcium cations as counterions to carboxylate groups in the polymer include a polyfluoroacrylate (PFAA) polymer such as poly-2-fluoroacrylate polymer with calcium cations as counterions to carboxylate groups in the polymer and such calcium polymers may be tested or used in studies including, optionally with a base (e.g., calcium carbonate) that is administered before, with or after administration of the polymer.

In an exemplary method, hyperkalemia in patients with hypertension and diabetic nephropathy is treated. At the time of screening, eligible patients are >30 years of age, have Type 2 diabetes mellitus (T2DM) diagnosed after age 30 which has been treated with oral medications or insulin for at least one year, have chronic kidney disease (estimated GFR 15−<60 mL/min/1.73m2 based on serum creatinine measurement), urine ACR 30 mg/g, laboratory serum K⁺ value of 4.5-5.0 mEq/L AND serum K⁺ value>5.0-<6.0 mEq/L at randomization to treatment, have an average systolic blood pressure ≧140-<180 mmHg OR average diastolic blood pressure ≧90-<110 mmHg (sitting) and be receiving an angiotensin-converting-enzyme inhibitor (ACEI) and/or angiotensin receptor blocker (ARB) for at least 28 days. Females of child-bearing potential must be non-lactating, must have a negative serum pregnancy test at screening, and must use a highly effective form of contraception for at least 3 months before study drug administration, during the study, and for one month after study completion.

Patients are excluded if they have Type 1 diabetes mellitus, hemoglobin A1c >12% at screening or emergency treatment for T2DM within the last 3 months, diabetic gastroparesis, non-diabetic chronic kidney disease, history of bowel obstruction, swallowing disorders, severe gastrointestinal disorders or major gastrointestinal surgery (e.g., cholectomy), current diagnosis of NYHA Class III or IV heart failure, body mass index (BMI) 40 kg/m2, any of the following events occurring within 2 months prior to screening: unstable angina as judged by the Investigator, unresolved acute coronary syndrome, cardiac arrest or clinically significant ventricular arrhythmias, transient ischemic attack or stroke, use of any intravenous cardiac medication, prior kidney transplant, or anticipated need for transplant during study participation, active cancer, currently on cancer treatment or history of cancer in the past two years except for nonmelanocytic skin cancer which is considered cured, history of alcoholism or drug/chemical abuse within 1 year, liver enzymes [alanine aminotransferase (ALT), aspartate aminotransferase (AST)]>3 times upper limit of normal, loop and thiazide diuretics or other antihypertensive medications (calcium channel blocker, beta-blocker, alpha-blocker, or centrally acting agent) that have not been stable for at least 28 days prior to screening or not anticipated to remain stable during study participation, current use of lithium, or any medical condition, uncontrolled systemic disease, or serious intercurrent illness that would significantly decrease study compliance or jeopardize the safety of the patient. Other exclusions include current use of lithium or the use of potassium sparing medications, including aldosterone antagonists (e.g., spironolactone), potassium supplements, bicarbonate or baking soda in the last 7 days prior to screening.

Following baseline assessments, patients who continue to meet eligibility criteria are divided into 3 cohorts: Cohort 1 discontinues ACEI/ARB, starts Losartan (100 mg/d) for 3 weeks, and adds spironoloctone after 2 weeks. Cohort 2 continues current ACEI/ARB for 3 weeks and adds spironolactone after 2 weeks. Cohort 3 (subjects with IC′ at baseline >5 mg/L at screening) continues ACEI/ARB and are immediately randomized. All cohorts are randomized 1:1 by K⁺ levels to 2 groups for initial polymer treatment with Ca(poly-2-fluoroacrylic acid) polymer prepared as described by any one or more of Examples 1, 3, 5, and 21-30, or placebo. Subjects with K⁺ levels >5.0-5.5 receive 3 starting polymer doses of 10, 20 and 30 g/d. Subjects with K⁺ levels >5.5<6.0 receive 3 starting polymer doses of 20, 30 and 40 g/d. Subsequently, all patients receive at least 8 weeks of polymer treatment. Optionally, subjects may be treated with a combination of Ca-polyfluoroacrylate polymer plus a base (e.g., calcium carbonate), wherein the polymer is about 5% to about 75% neutralized with calcium, for example, about 60%, and the base is present at about 10% to about 50%, for example, about 25%, relative to the number of carboxyl groups in the polymer, administered before, with or after treatment with the polymer.

Prohibited medications during the study include other polymer-based drugs (e.g., sevelamer, sodium polystyrene sulfonate, colesevelam, colestipol, cholestyramine), phosphate binders (e.g., lanthanum carbonate), or other potassium binders, or their anticipated need during study participation.

The primary endpoint of the study is the mean change in serum potassium from baseline to week 4 or prior to initiation of study drug. The secondary endpoint of the study is the mean change in serum potassium from baseline to week 8 or prior to the initiation of study drug.

Treatment with crosslinked polyfluoroacrylate polymer and a base may result in significant and clinically meaningful improvement in signs and symptoms of hyperkalemia in patients with hypertension and diabetic nephropathy.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entireties.

What is claimed:

1. A crosslinked cation-binding polymer comprising:
a. monomers that comprise carboxylate groups and pKa decreasing groups; and
b. calcium cations;
wherein the calcium cations are counterions to about 5% to about 75% of the carboxylate groups in said polymer, and
wherein the polymer optionally comprises no more than about 5% sodium cations as counterions to the carboxylate groups in said polymer.

2. The composition of claim 1, wherein the polymer is crosslinked with about 4.0 mol % to about 20.0 mol % of one or more crosslinkers.

3. The composition of claim 2, wherein the polymer is crosslinked with about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers.

4. The composition of claim 1, wherein the polymer is crosslinked with about 0.025 mol % to about 3.0 mol % of one or more crosslinkers.

5. The composition of claim 4, wherein the polymer is crosslinked with about 0.025 mol % to about 0.3 mol %, about 0.025 mol % to about 0.17 mol %, about 0.025 mol % to about 0.34 mol %, or about 0.08 mol % to about 0.2 mol % of one or more crosslinkers.

6. The crosslinked cation-binding polymer of claim 1, wherein the pKa-decreasing group is an electron-withdrawing substituent.

7. The crosslinked cation-binding polymer of claim 1, wherein the electron-withdrawing substituent is located adjacent to the carboxylic acid group of the monomer.

8. The crosslinked cation-binding polymer of claim 1, wherein the electron-withdrawing substituent is located in the alpha or beta position of the carboxylic acid group of the monomer.

9. The crosslinked cation-binding polymer of claim 1, wherein the electron-withdrawing substituent is a hydroxyl group, an ethereal group, an ester group or a halide atom.

10. The crosslinked cation-binding polymer of claim 9, wherein the halide atom is fluorine (F).

11. The polymer of claim 1, wherein the calcium cations are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of of the carboxylate groups in said polymer.

12. The polymer of claim 1, wherein counterions that are not calcium or sodium are hydrogen.

13. The polymer of any of claims 1-12, wherein the counterions to said carboxylate groups consist of said calcium counterions and hydrogen counterions.

14. A crosslinked cation-binding polymer comprising:
a. monomers that comprise carboxylate groups and pkA decreasing groups; and
b. calcium cations and magnesium cations,
wherein the calcium cations and magnesium cations are counterions to about 5% to about 75% of the carboxylate groups in said polymer,
wherein the magnesium cations are counterions to no more than about 35% of the carboxylate groups in the polymer, and
wherein the polymer optionally comprises no more than about 5% sodium cations as counterions to the carboxylate groups in said polymer.

15. The composition of claim 14, wherein the polymer is crosslinked with about 5.0 mol % to about 20.0 mol % of one or more crosslinkers.

16. The composition of claim 15, wherein the polymer is crosslinked with about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers.

17. The composition of claim 14, wherein the polymer is crosslinked with about 0.025 mol % to about 3.0 mol % of one or more crosslinkers.

18. The composition of claim 17, wherein the polymer is crosslinked with about 0.025 mol % to about 0.3 mol %, about 0.025 mol % to about 0.17 mol %, about 0.025 mol % to about 0.34 mol %, or about 0.08 mol % to about 0.2 mol % of one or more crosslinkers.

19. The crosslinked cation-binding polymer of claim 14, wherein the pKa-decreasing group is an electron-withdrawing substituent.

20. The crosslinked cation-binding polymer of claim 14, wherein the electron-withdrawing substituent is located adjacent to the carboxylic acid group of the monomer.

21. The crosslinked cation-binding polymer of claim 14, wherein the electron-withdrawing substituent is located in the alpha or beta position of the carboxylic acid group of the monomer.

22. The crosslinked cation-binding polymer of claim 14, wherein the electron-withdrawing substituent is a hydroxyl group, an ethereal group, an ester group or a halide atom.

23. The crosslinked cation-binding polymer of claim 22, wherein the halide atom is fluorine (F).

24. The polymer of claim 14, wherein the calcium cations and magnesium cations are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer.

25. The polymer of claim 14, wherein counterions that are not calcium, magnesium or sodium are hydrogen.

26. The polymer of any of claims 14-25, wherein the counterions to said carboxylate groups consist of said calcium counterions, magnesium counterions and hydrogen counterions.

27. The polymer of any one of claims 1-26, wherein the monomer is acrylic acid, an acrylic acid derivative, or a salt thereof.

28. The polymer of any of claims 1-27, wherein the polymer is crosslinked with a crosslinker selected from diethelyeneglycol diacrylate (diacryl glycerol), triallylamine, tetraallyloxyethane, allylmethacrylate, 1,1,1-trimethylolpropane triacrylate (TMPTA), derivatives of TMPTA, divinyl benzene, 1,7-ocatadiene and divinyl glycol.

29. The polymer of claim 28, wherein the crosslinked polymer is derived from acrylic acid monomers and TMPTA.

30. The polymer of any one of claims 1-29, wherein no less than about 70% of the polymer has a particle size of about 212 microns to about 500 microns.

31. The polymer of any one of claims 1-29, wherein no less than about 70% of the polymer has a particle size of about 75 microns to about 150 microns.

32. The polymer of any one of claims 1-29, wherein no less than about 70% of the polymer has a particle size of about 100 microns.

33. The polymer of any one of claims 1-29, wherein no less than about 70% of the polymer has a particle size of about 75 microns or less.

34. A composition comprising the polymer of any of claims 1-33.

35. The composition of claim 34 further comprising an additional base.

36. The composition of claim 35, wherein the additional base is a pharmaceutically acceptable base, a salt thereof, or a combination thereof.

37. The composition of claim 35, wherein the additional base is selected from an alkali metal hydroxide, an alkali metal acetate, an alkali metal carbonate, an alkali metal bicarbonate, an alkali metal oxide, an alkaline earth metal hydroxide, an alkaline earth metal acetate, an alkaline earth metal carbonate, an alkaline earth metal bicarbonate, an alkaline earth metal oxide, an organic base, choline, lysine, arginine, histidine, an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a benzoate, an oxide an oxalate, a hydroxide, an amine, a hydrogen citrate, calcium bicarbonate, calcium carbonate, calcium oxide, calcium hydroxide, magnesium oxide, magnesium hydroxide, magnesium carbonate, magnesium bicarbonate, aluminum carbonate, aluminum hydroxide, sodium bicarbonate, potassium citrate, and combinations thereof.

38. The composition of any of claims 34-37, wherein the polymer is a polyacrylate polymer.

39. The composition of any of claims 35-37, wherein the additional base is calcium carbonate.

40. A composition comprising a crosslinked polyacrylate polymer comprising acrylic acid repeat units that comprise carboxylic acid groups, pKa decreasing groups, and calcium cations, wherein said calcium cations are counterions to about 5% to about 75% of the carboxylate groups in said polymer, and wherein the polymer optionally comprises no more than about 5% sodium cations as counterions to the carboxylate groups in said polymer.

41. The composition of claim 40, wherein the polymer is crosslinked with about 5.0 mol % to about 20.0 mol % of one or more crosslinkers.

42. The composition of claim 41, wherein the polymer is crosslinked with about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers.

43. The composition of claim 40, wherein the polymer is crosslinked with about 0.025 mol % to about 3.0 mol % of one or more crosslinkers.

44. The composition of claim 43, wherein the polymer is crosslinked with about 0.025 mol % to about 0.3 mol %, about 0.025 mol % to about 0.17 mol %, about 0.025 mol % to about 0.34 mol %, or about 0.08 mol % to about 0.2 mol % of one or more crosslinkers.

45. The composition of claim 40, wherein the calcium cations are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer.

46. The composition of any of claims 40-44, wherein the counterions to said carboxylate groups consist of said calcium counterions and hydrogen counterions.

47. A composition comprising a crosslinked polyacrylate polymer comprising acrylic acid repeat units that comprise carboxylic acid groups, pKa decreasing groups, and calcium cations and magnesium cations, wherein said calcium and magnesium cations are counterions to about 5% to about 75% of the carboxylate groups in said polymer, wherein the magnesium cations are counterions to no more than about 35% of the carboxylate groups in the polymer, and wherein the polymer optionally comprises no more than about 5% sodium cations as counterions to the carboxylate groups in said polymer.

48. The composition of claim 47, wherein the polymer is crosslinked with about 5.0 mol % to about 20.0 mol % of one or more crosslinkers.

49. The composition of claim 48, wherein the polymer is crosslinked with about 4.0 mol % to about 10.0 mol %, 4.0 mol % to about 15.0 mol %, 8.0 mol % to about 10.0 mol %, 8.0 mol % to about 15.0 mol %, 8.0 mol % to about 20.0 mol %, or 12.0 mol % to about 20.0 mol % of one or more crosslinkers.

50. The composition of claim 47, wherein the polymer is crosslinked with about 0.025 mol % to about 3.0 mol % of one or more crosslinkers.

51. The composition of claim 50, wherein the polymer is crosslinked with about 0.025 mol % to about 0.3 mol %, about 0.025 mol % to about 0.17 mol %, about 0.025 mol % to about 0.34 mol %, or about 0.08 mol % to about 0.2 mol % of one or more crosslinkers.

52. The composition of claim 47, wherein the calcium cations and magnesium cations are counterions to about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 35% to about 40%, about 35% to about 45%, about 35% to about 50%, about 35% to about 55%, about 35% to about 60%, about 35% to about 65%, about 35% to about 70%, about 35% to about 75%, about 40% to about 45%, about 40% to about 50%, about 40% to about 55%, about 40% to about 60%, about 40% to about 65%, about 40% to about 70%, about 40% to about 75%, about 45% to about 50%, about 45% to about 55%, about 45% to about 60%, about 45% to about 65%, about 45% to about 70%, about 45% to about 75%, about 50% to about 55%, about 50% to about 60%, about 50% to about 65%, about 50% to about 70%, about 50% to about 75%, about 55% to about 60%, about 55% to about 65%, about 55% to about 70%, about 55% to about 75%, about 60% to about 65%, about 60% to about 70%, about 60% to about 75%, about 65% to about 70%, about 65% to about 75% or about 70% to about 75% of the carboxylate groups in the polymer.

53. The composition of any of claims 47-51, wherein the counterions to said carboxylate groups consist of said calcium counterions, magnesium counterions and hydrogen counterions.

54. The composition of any one of claims 40-53, wherein remaining counterions to said carboxylate groups consist of hydrogen cations.

55. The composition of any one of claims 40-54 further comprising calcium carbonate.

56. The composition of any one of claims 40-55, wherein no less than about 70% of the polymer has a particle size of about 212 microns to about 500 microns.

57. The composition of any one of claims 40-55, wherein no less than about 70% of the polymer has a particle size of about 75 microns to about 150 microns.

58. The composition of any one of claims 40-55, wherein no less than about 70% of the polymer has a particle size of about 100 microns.

59. The composition of any one of claims 40-55, wherein no less than about 70% of the polymer has a particle size of about 75 microns or less.

60. A dosage form comprising the polymer of any of claims 1-33 or the composition of any of claims 34-59.

61. The dosage form of claim 60 further comprising an additional base.

62. The dosage form of claim 61, wherein the additional base is selected from an alkali metal hydroxide, an alkali metal acetate, an alkali metal carbonate, an alkali metal bicarbonate, an alkali metal oxide, an alkaline earth metal hydroxide, an alkaline earth metal acetate, an alkaline earth metal carbonate, an alkaline earth metal bicarbonate, an alkaline earth metal oxide, an organic base, choline, lysine, arginine, histidine, an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a benzoate, an oxide, an oxalate, a hydroxide, an amine, a hydrogen citrate, calcium bicarbonate, calcium carbonate, calcium oxide, calcium hydroxide, magnesium oxide, magnesium hydroxide, magnesium carbonate, magnesium bicarbonate, aluminum carbonate, aluminum hydroxide, sodium bicarbonate, potassium citrate, and combinations thereof.

63. The dosage form of any of claims 60-62, further comprising one or more pharmaceutically acceptable excipients.

64. The dosage form of any of claims 60-63, wherein the dosage form is a tablet, a chewable tablet, a capsule, a suspension, an oral suspension, a powder, a gel block, a gel pack, a confection, a chocolate bar, a flavored bar, a pudding, or a sachet.

65. The dosage form of any of claims 60-64, wherein the dosage form is a sachet comprising about 1 g to about 30 g of the polymer.

66. The dosage form of any of claims 60-64, wherein the dosage form is a sachet comprising about 4 g to about 15 g of the polymer.

67. The dosage form of any of claims 60-64, wherein the dosage form is a sachet comprising about 8 g to about 15 g of the polymer.

68. The dosage form of any of claims 60-64, wherein the dosage form is a sachet comprising about 8 g of the polymer.

69. The dosage form of any of claims 60-64, wherein the dosage form is a capsule comprising about 0.1 g to about 1 g of the polymer.

70. The dosage form of any of claims 60-64, wherein the dosage form is a capsule containing about 0.25 g to about 0.75 g of the polymer.

71. The dosage form of any of claims 60-64, wherein the dosage form is a capsule comprising about 0.5 g of the polymer.

72. The dosage form of any of claims 60-64, wherein the dosage form is a tablet comprising about 0.1 g to about 1.0 g of the polymer.

73. The dosage form of any of claims 60-64, wherein the dosage form is a tablet comprising about 0.3 g to about 0.8 g of the polymer.

74. The dosage form of any of claims 60-64, wherein the dosage form is a sachet, flavored bar, gel block, gel pack, pudding, or powder comprising about 1 g to about 30 g of the polymer.

75. The dosage form of any of claims 60-64, wherein the dosage form is a sachet, flavored bar, gel block, gel pack, pudding, or powder comprising about 4 g to about 20 g of the polymer.

76. The dosage form of any of claims 60-64, wherein the dosage form is a sachet, flavored bar, gel block, gel pack, pudding, or powder comprising about 4 g to about 8 g of the polymer.

77. The dosage form of any of claims 60-64, wherein the dosage form is a suspension comprising about 0.04 g of the polymer per mL of suspension to about 1 g of the polymer per mL of suspension.

78. The dosage form of any of claims 60-64, wherein the dosage form is a suspension comprising about 0.1 g of the polymer per mL of suspension to about 0.8 g of the polymer per mL of suspension.

79. The dosage form of any of claims 60-64, wherein the dosage form is a suspension comprising about 0.3 g of the polymer per mL of suspension.

80. The dosage form of any of claims 60-64, wherein the dosage form is a suspension comprising about 1 g to about 30 g of the polymer.

81. The dosage form of any of claims 77-80, wherein the suspension is an oral suspension.

82. The dosage form of any of claims 60-81, further comprising one or more additional agent.

83. The dosage form of claim 82, wherein the one or more additional agent is known to increase serum potassium.

84. The dosage form of claim 82, wherein the one or more additional agent is selected from a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, VAP antagonists, amiloride, triamterine, a potassium supplement, heparin, a low molecular weight heparin, a non-steroidal anti-inflammatory drug, ketoconazole, trimethoprim, pentamide, a potassium sparing diuretic, amiloride, triamterene, and combinations thereof.

85. A method of treating hyperkalemia in a subject, the method comprising administering to said subject an effective amount of the polymer of any of claims 1-33, the composition of any of claims 34-59, or the dosage form of any of claims 60-84.

86. A method of treating hyperkalemia in a subject, the method comprising:
a. identifying the subject as having, or as having a risk of developing, hyperkalemia; and
b. administering to said subject an effective amount of the polymer of any of claims 1-33, the composition of any of claims 34-59, or the dosage form of any of claims 60-84.

87. The method of claim 85 or 86, wherein the hyperkalemia is acute hyperkalemia or chronic hyperkalemia.

88. The method of claim 85 or 86, wherein the hyperkalemia is induced by a drug.

89. The method of claim 88, wherein the drug is a RAAS blocker, a beta blocker, trimethoprim, a renin inhibitor, an aldosterone synthase inhibitor, a non-steroidal anti-inflammatory drug, or heparin.

90. The method of claim 85 or 86, further comprising, after administering the polymer or composition, determining a potassium level in said subject, wherein the potassium level is within a normal potassium level range for the subject.

91. The method of any of claims 85-90, further comprising co-administering to said subject one or more of: mannitol, sorbitol, calcium acetate, sevelamer carbonate, sevelamer hydrochloride, a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, VAP antagonists, amiloride, triamterine, a potassium supplement, heparin, a low molecular weight heparin, a non-steroidal anti-inflammatory drug, ketoconazole, trimethoprim, pentamide, a potassium sparing diuretic, amiloride, triamterene, and combinations thereof.

92. The method of any of claims 85-91, further comprising:
a. before administering the polymer or composition, determining a baseline level of potassium in the subject; and
b. after administering the polymer or composition, determining a second level of potassium in the subject,
wherein the second level of potassium is substantially less than said baseline level of potassium.

93. The method of any of claims 85-92, wherein an acid/base status associated with the subject does not significantly change within about 1 day of administration of the polymer or composition.

94. A method of treating heart failure in a subject, the method comprising administering to the subject an effective amount of the polymer of any of claims 1-33, the composition of any of claims 34-59, or the dosage form of any of claims 60-84.

95. A method of treating heart failure in a subject, the method comprising:
a. identifying a subject as having heart failure; and
b. administering to the subject an effective amount of the polymer of any of claims 1-33, the composition of any of claims 34-59, or the dosage form of any of claims 60-84.

96. The method of claim 94 or 95 further comprising:
a. before administering said polymer or composition, determining one or more of: a baseline level of one or more ions in the subject, a baseline total body weight associated with the subject, a baseline total body water level associated with the subject, a baseline total extracellular water level associated with the subject, and a baseline total intracellular water level associated with the subject; and
b. after administering said polymer or composition, determining one or more of: a second level of one or more ions in the subject, a second total body weight associated with the subject, a second total body water level associated with the subject, a second total extracellular water level associated with the subject, and a second total intracellular water level associated with the subject,
wherein the second level is substantially lower than the baseline level.

97. The method of claim 96, wherein the one or more ions are selected from sodium, potassium, calcium, lithium, and magnesium.

98. The method of any of claims 94-97, wherein an acid/base status associated with said subject does not significantly change within about 1 day of administration of the polymer or composition.

99. The method of any of claims 94-98, wherein a blood pressure level associated with the subject after administration of the polymer or composition is substantially lower than a baseline blood pressure level associated with the subject before administration of the polymer or composition.

100. The method of claim 99, wherein the blood pressure level is one or more of: a systolic blood pressure level, a diastolic blood pressure level, and a mean arterial pressure level.

101. The method of any of claims 94-100, wherein a symptom of fluid overload associated with said subject, determined after administration of the polymer or composition, is reduced compared to a baseline level determined before administration of the composition.

102. The method of claim 101, wherein the symptom is one or more of: difficulty breathing when lying down, difficulty breathing with normal physical activity, ascites, fatigue, shortness of breath, increased body weight, peripheral edema, and pulmonary edema.

103. The method of any of claims 94-102, wherein the subject is on concomitant diuretic therapy.

104. The method of claim 103, wherein the diuretic therapy is reduced or discontinued after administration of the composition.

105. The method of any of claims 94-104, further comprising co-administering to the subject an agent known to increase serum potassium levels.

106. The method of claim 105, wherein the agent is one or more of: a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, VAP antagonists, amiloride, triamterine, a potassium supplement, heparin, a low molecular weight heparin, a non-steroidal anti-inflammatory drug, ketoconazole, trimethoprim, pentamide, a potassium sparing diuretic, amiloride, triamterene, and combinations thereof.

107. The method of claim 106, wherein a dose of the agent is increased after administration of the polymer or composition.

108. The method of any of claims 94-107, wherein the subject is co-administered a blood pressure medication.

109. The method of claim 108, wherein a dose of the blood pressure medication is reduced after administration of the polymer or composition.

110. A method of treating chronic kidney disease in a subject, the method comprising administering to the subject an effective amount of the polymer of any of claims 1-33, the composition of any of claims 34-59, or the dosage form of any of claims 60-84.

111. A method of treating chronic kidney disease in a subject, the method comprising:
a. identifying the subject as having a chronic kidney disease; and
b. administering to the subject an effective amount of the polymer of any of claims 1-33, the composition of any of claims 34-59, or the dosage form of any of claims 60-84.

112. The method of claim 110 or 111, wherein a symptom of fluid overload is reduced after administration of the polymer or composition.

113. The method of claim 112, wherein the symptom is one or more of: difficulty breathing at rest, difficulty breathing during normal physical activity, edema, pulmonary edema, hypertension, peripheral edema, leg edema, ascites, and/or increased body weight.

114. The method of claim any of claims 110-113, wherein a blood pressure level associated with the subject after administration of the polymer or composition is substantially lower than a baseline blood pressure level associated with the subject before administration of the composition.

115. The method of claim 114, wherein the blood pressure level is one or more of:
a systolic blood pressure level, a diastolic blood pressure level, and a mean arterial pressure level.

116. The method of any of claims 110-115, wherein a co-morbidity of chronic kidney disease is reduced or alleviated after administration of the composition.

117. The method of claim 116, wherein the co-morbidity is one or more of: fluid overload, edema, pulmonary edema, hypertension, hyperkalemia, excess total body sodium, and uremia.

118. The method of any of claims 110-117, further comprising:
a. before administering the polymer or composition, determining one or more of: a baseline level of one or more ions in the subject, a baseline total body weight associated with the subject, a baseline total body water level associated with the subject, a baseline total extracellular water level associated with the subject, and a baseline total intracellular water level associated with the subject; and
b. after administering the polymer or composition, determining one or more of: a second level of said one or more ions in the subject, a second total body weight associated with the subject, a second total body water level associated with the subject, a second total extracellular water level associated with the subject, and a second total intracellular water level associated with the subject
wherein the second level is substantially less than the baseline level.

119. The method of claim 118, wherein the one or more ions are selected from sodium, potassium, calcium, lithium, magnesium, and ammonium.

120. The method of any of claims 110-119, wherein an acid/base status associated with said subject does not significantly change within about 1 day of administration of the polymer or composition.

121. A method of treating a disease or disorder in a subject, the method comprising administering to the subject the polymer of any of claims 1-33, the composition of any of claims 34-59, or the dosage form of any of claims 60-84.

122. A method of treating a disease or disorder in a subject, the method comprising:
a. identifying a disease or a disorder in the subject, or identifying a risk that the subject will develop a disease or disorder; and
b. administering to the subject an effective amount of the polymer of any of claims 1-33, the composition of any of claims 34-59, or the dosage form of any of claims 60-84.

123. The method of claim 121 or 122, wherein the disease or disorder is one or more of: heart failure, a renal insufficiency disease, end stage renal disease, liver cirrhosis, chronic renal insufficiency, chronic kidney disease, fluid overload, fluid maldistribution, edema, pulmonary edema, peripheral edema, lymphedema, nephrotic edema, idiopathic edema, ascites, cirrhotic ascites, interdialytic weight gain, high blood pressure, hyperkalemia, hypernatremia, abnormally high total body sodium, hypercalcemia, tumor lysis syndrome, head trauma, an adrenal disease, hyporeninemic hypoaldosteronism, hypertension, salt-sensitive hypertension, refractory hypertension, renal tubular disease, rhabdomyolysis, crush injuries, renal failure, acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, allergic pulmonary edema, high altitude sickness, Adult Respiratory Distress Syndrome, traumatic edema, cardiogenic edema, acute hemorrhagic edema, heatstroke edema, facial edema, eyelid edema, angioedema, cerebral edema, scleral edema, nephritis, nephrosis, nephrotic syndrome, glomerulonephritis, and/or renal vein thrombosis.

124. The method of any of claims 121-123, wherein said polymer or composition is administered from 1 time every 3 days to about 4 times per day.

125. The method of any of claims 121-123, wherein said polymer or composition is administered from 1 to 4 times per day.

126. The method of any of claims 121-123, wherein said polymer or composition is administered from 1 to 2 times per day.
